(19) United States
(12) Patent Application Publication
(10) Pub. No.: US 2025/0290074 A1
(43) Pub. Date: Sep. 18, 2025

(54) TARGETED TREATMENT OF SPLICEOPATHY-INDUCED NEUROLOGICAL DISORDERS

(71) Applicant: Auttx, LLC, Newton, MA (US)

(72) Inventors: Isabelle Draper, Thezan-les-Bezier (FR); Alan S. Kopin, Wellesley, MA (US); Duncan Brown, Berkeley, CA (US)

(21) Appl. No.: 19/227,239

(22) Filed: Jun. 3, 2025

Related U.S. Application Data
(63) Continuation of application No. PCT/US2023/083231, filed on Dec. 8, 2023.
(60) Provisional application No. 63/431,367, filed on Dec. 9, 2022.

(51) Int. Cl. C12N 15/113

(52) U.S. Cl. CPC C12N 15/113; C12N 2310/11; C12N 2310/315; C12N 2310/321; C12N 2310/3341; C12N 2320/33

(57) ABSTRACT

Disclosed are methods of treating a subject with a neurological disease associated with a splicing defect caused by TDP-43 proteinopathies, comprising administering to said subject an agent to increase expression levels and/or stability of hnRNP L, thereby attenuating and/or repairing the splicing defect. The disclosure also relates to nucleic acids targeting heterogeneous nuclear ribonucleoprotein L (hnRNP L), and their use.

### CROSS-REFERENCE

This application is a continuation of International Application No. PCT/US2023/083231, filed Dec. 8, 2023, which claims the benefit of U.S. Provisional Application No. 63/431,367, filed Dec. 9, 2022, the contents of which are expressly incorporated herein by reference in their entirety including any drawings.

### INCORPORATION BY REFERENCE OF SEQUENCE LISTING

The present application is being filed along with a Sequence Listing in electronic format. The Sequence Listing is provided as a file entitled 052991-502001WO.xml, created Dec. 5, 2023, which is 95,008 bytes in size. The information in the electronic format of the Sequence Listing is incorporated by reference in its entirety.

### FIELD

The present disclosure relates to treatment of neurological disorders involving cryptic exons, poison exon and/or intron retentions, including Amyotrophic Lateral Sclerosis (ALS), TDP-proteinopathies and other cryptic exon-induced neurological disease (CEIND) or poison exon-induced neurological diseases (PEIND). The disclosure also relates to nucleic acids targeting heterogeneous nuclear ribonucleoprotein L (hnRNP L), and their use.

### BACKGROUND

Splicing is an important and often essential post-transcriptional process. Alternative splicing may generate different transcripts, each leading to a unique protein isoform when translated. For example, normal brain function can greatly depend on correct alternative splicing. Amyotrophic Lateral Sclerosis (ALS) and a growing list to neurological diseases can be caused by inclusion of cryptic/poison exons. These are referred to cryptic exon induced neurological disease (CEIND) and poison exon induced neurological disease (PEIND). As such, there exists a need for treatments of spliceopathy-induced neurological disorders such as cryptic/poison exon-induced disease, including Amyotrophic Lateral Sclerosis (ALS), Frontotemporal Dementia (FTD) and other TDP-43-related diseases, e.g., Alzheimer's disease (AD).

### SUMMARY

The disclosure provides compounds, compositions, gene therapy options and methods for the treatment, e.g., reduction of symptoms, of ALS/CEIND/PEIND as one cause of these disorders may include mutation or altered expression of the RNA Binding Proteins (RBPs), including, but not limited to, heterogeneous nuclear ribonucleoprotein L (hnRNP L) and TDP-43, that regulate cryptic exon expression or recruitment. Notably, these factors may regulate cryptic exon recruitment (inhibit) during splicing; hnRNP L and TDP-43 both repress cryptic exon. The inclusion of cryptic exon(s) in the transcribed product can lead to the development of diseases. TDP-43 proteinopathies may include neurological diseases due to a change of TDP-43 function, especially mislocalization and aggregation of TDP-43 proteins. Agents can be delivered to treat TDP-43 proteinopathies using delivery vehicles including, e.g., liposomes, virus, nanoparticles, or other methods known in the art.

In one aspect, the present disclosure provides a method of treating a subject with a neurological disease associated with a splicing defect/cryptic exon recruitment/inclusion caused by TAR DNA-binding protein 43 (TDP-43) proteinopathies, comprising administering to the subject an agent to increase expression levels and/or stability of heterogeneous nuclear ribonucleoprotein L (hnRNP L), thereby repairing the splicing defect. In some aspects, the present disclosure provides a method of treating a subject with a neurological disease associated with a splicing defect/cryptic exon recruitment/inclusion. In some embodiments, the neurological disease is not a TDP-43 proteinopathy. In some embodiments, the neurological disease includes autism. In some embodiments, the neurological disease includes fragile X syndrome. In some embodiments, the neurological disease includes a disorder identified as associated with a cryptic or poison exon in Stephan J Sanders et al., “A framework for the investigation of rare genetic disorders in neuropsychiatry” Review Nat Med. 2019 October; 25(10):1477-1487. In some embodiments, the method comprises administering to the subject an agent to increase expression levels and/or stability of heterogeneous nuclear ribonucleoprotein L (hnRNP L). A neurological disease associated with a splicing defect/cryptic exon recruitment/inclusion may include a poison exon-induced neurological disease (PEIND). In some embodiments, the agent is capable of increasing expression levels of hnRNP L to at least a certain percentage (e.g., 10%, 20%, 25%, 30%, 40%, 50%, 60%, 70%, 80%, 90%, 95%, 99%, 100%, 2-fold, 5-fold, 10-fold, or more), compared to normal control levels in a healthy subject or a subject without the neurological disease.

In some embodiments, the neurological disease is associated with a splicing defect/cryptic or poison exon recruitment/inclusion caused by one or more TDP-43 proteinopathies. In some embodiments, the one or more TDP-43 proteinopathies are due to a loss of or altered TDP-43 function. In some embodiments, the TDP-43 comprises an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 1 or 2. In some embodiments, the TDP-43 consists of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 1 or 2. In some embodiments, the subject shows a loss of or altered TDP-43 function due to (i) a mutation in the TDP-43 gene or open reading frame, and/or (ii) an altered TDP-43 function following either (or a combination of) nuclear clearance, cytoplasmic inclusions, nuclear inclusions, aggregation, abnormal modification (e.g., ubiquitination), neuronal propagation in a “prion-like” manner, etc. The mutation in the TDP-43 gene/open reading frame, as described herein, includes at least one or a combination of: substitution, deletion, insertion, duplication, inversion, translocation, changes in different alleles, variations in one allele, nonsense, missense, splicing, truncation, etc. In some embodiments at least one TDP-43 mutation is selected from the group consisting of: G298S, M337V, Q343R, and A315T. In some embodiments, at least one TDP-43 mutation is selected from the group consisting of: D169G, K263E, N267S, G287S, G290A, S292N, G294A, G294V, G295R, G295S, G298S, M311V. A315T, A321V, A321G, Q331K, S332N, G335D, M337V, Q343R, N345K, G348C, G348V. N352S/T, R361S, P363A, Y374X, N378D, S379P, S379C, A382P, A382T, 1383V, G384R. N390D, N390S, and S393L. In some embodiments, the loss of/altered TDP-43 function promotes cryptic exon inclusion. In some embodiments, the loss of/altered TDP-43 function reduces expression levels and/or stability of the Stathmin-2 (STMN2) gene. In some embodiments, the loss of/altered TDP-43 function reduces expression levels and/or stability of the STMN2 gene. In some embodiments, the STMN2 gene described herein encodes a STMN2 protein comprising an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 22 or 23. In some embodiments, the loss of/altered TDP-43 function reduces expression levels and/or stability of the Unc-13 homolog A (UNC13A) gene. In some embodiments, the loss of/altered TDP-43 function reduces expression levels and/or stability of the UNC13A gene. In some embodiments, the UNC13A gene described herein encodes a UNC13A protein comprising an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 103. In some embodiments, the loss of/altered TDP-43 function reduces expression levels and/or stability of the Unc-13 homolog B (UNC13B) gene. In some embodiments, the loss of/altered TDP-43 function reduces expression levels and/or stability of the UNC13B, gene. In some embodiments, the UNC13B gene described herein encodes a UNC13B protein comprising an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 104. In some embodiments, the STMN2 gene described herein encodes a STMN2 protein consisting of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 22 or 23. In some embodiments, the loss of/altered TDP-43 function reduces expression levels and/or stability of the SORT1 gene. In some embodiments, the SORT1 gene described herein encodes a SORT1 protein comprising an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 14 or 15. In some embodiments, the SORT1 gene described herein encodes a SORT1 protein consisting of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 14 or 15. In some embodiments, the loss of/altered TDP-43 function reduces expression levels and/or stability of the GPSM2 gene. In some embodiments, the GPSM2 gene described herein encodes a GPSM2 protein comprising an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 16. In some embodiments, the GPSM2 gene described herein encodes a GPSM2 protein consisting of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 16. In some embodiments, the loss of/altered TDP-43 function reduces expression levels and/or stability of the ATG4B gene. In some embodiments, the ATG4B gene described herein encodes a ATG4B protein comprising an amino acid sequence having at least 50%, 60%, 65%, 70%, 75%, 80%, 85%, 90%, 91%, 92%, 93%, 94%, 95%, 96%, 97%, 98%, 99%, or 100% sequence identity to any one of SEQ ID NOs: 17-21. In some embodiments, the ATG4B gene described herein encodes a ATG4B protein consisting of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to any one of SEQ ID NOs: 17-21. For example, the expression levels and/or stability of the STMN2, SORT1, GPSM2, or ATG4B gene may be reduced to about 90%, 80%, 70%, 60%, 50%, 40%, 30%, 20%, 10%, 5%, or less, than the normal or average/mean expression levels and/or stability in a healthy subject or a subject without the neurological disease. In some embodiments, the expression levels of the UNC13A, UNC13B, STMN2, SORT1, GPSM2, or ATG4B gene, either on the RNA level or the protein level, are completely inhibited (i.e., 0% of normal control levels; may be caused by, e.g., knocking out or a deletion of the UNC13A, UNC13B, STMN2, SORT1, GPSM2, or ATG4B gene). In some embodiments, the loss of/altered TDP-43 function a splicing defect in a UNC13A, UNC13B, STMN2, SORT1, GPSM2, or ATG4B gene. In some embodiments, the loss of/altered TDP-43 function reduces or inhibits neurite and/or axon growth. For example, the neurite and/or axon growth may be reduced to about 90%, 80%, 70%, 60%, 50%, 40%, 30%, 20%, 10%, 5%, or less, than the normal or average/mean neurite and/or axon growth in a healthy subject or a subject without the neurological disease. In some embodiments, the neurite and/or axon growth is completely inhibited (i.e., 0% of normal control levels).

In some embodiments, the neurological disease described herein comprises at least one of cryptic exon-induced neurological diseases (CEIND). Such cryptic exon-induced neurological diseases (CEIND) comprise Amyotrophic Lateral Sclerosis (ALS), Frontotemporal lobar degeneration (FTLD), Frontotemporal Dementia (FTD), Autism Spectrum Disorder (ASD, e.g., as described in Jaganathan et al., Cell 176, 535-548, Jan. 24, 2019), myotonic dystrophy type 1 or 2 (DM1/DM2), Alzheimer's disease (AD), Lewy body dementia (LBD), Pick's disease, Hippocampal sclerosis, Corticobasal degeneration, Huntington disease, Parkinson's disease, Argyrophilic grain disease, Chronic traumatic encephalopathy (CTE), Perry syndrome, Alexander disease, Multisystem proteinopathy (MSP), intellectual disability, ADHD, dyslexia, epilepsy, bipolar disorder, depression and schizophrenia, parkinsonism-dementia complex of Guam, dementia pugilistica, diffuse neurofibrillary tangles with calcification, Down's syndrome, familial British dementia, familial Danish dementia, frontal lobe dementia, Gerstmann-Straussler-Scheinker disease, globular glial tauopathies, white matter tauopathy with globular glial inclusions, Guadeloupian parkinsonism with dementia, Guadeloupian progressive supranuclear palsy, multiple system atrophy, neurodegeneration with brain iron accumulation, Hallervorden-Spatz disease, pantothenate kinase-associated neurodegeneration, neurofibrillary tangle-predominant dementia. Niemann-Pick disease, prosencephalitic parkinsonism, prion protein cerebral amyloid angiopathy, progressive supranuclear palsy, SLCI A6-related mental retardation, subacute sclerosing panencephalitis, limbic predominant, age-related TDP-43 encephalopathy (LATE), and inclusion body myopathy. In some embodiments, the neurological disease described herein comprises Amyotrophic Lateral Sclerosis (ALS), Frontotemporal Dementia (FTD), myotonic dystrophy type 1 (DM1), Pick's disease, hippocampal sclerosis, corticobasal degeneration, argyrophilic grain disease, or Huntington disease. In some embodiments, the neurological disease comprises Amyotrophic Lateral Sclerosis (ALS) or Frontotemporal Dementia (FTD), Alzheimer's disease, chronic traumaticencephalopathy (CTE), limbic predominant, age-related TDP-43 encephalopathy (LATE), or inclusion body myopathy. Other CEIND or PEIND may be treated, such as Fragile X Syndrome, or those found in Stephan J Sanders et al., Review Nat Med. 2019 October; 25(10):1477-1487.

In some embodiments, the increased expression levels and/or stability of hnRNP L reduces the splicing defect caused by TDP-43 proteinopathies. For example, the splicing defect may be reduced to about 90%, 80%, 70%, 60%, 50%, 40%, 30%, 20%, 10%, 5%, or less, than the defect levels without the increased expression levels and/or stability of hnRNP L.

In some embodiments, the agent comprises at least one of a small molecule, a nucleic acid, an enzyme, a protein, a polypeptide, an antibody or a functional fragment thereof, an aptamer, a small interfering RNA, a microRNA, a long noncoding RNA (lncRNA), a small hairpin RNA, an antisense nucleic acid (e.g., ASO), tricyclo-DNA (tcDNA), locked nucleic acid (LNA), peptide nucleic acid (PNA) or phosphorodiamidate morpholino oligomer (PMO). In some embodiments, the agent comprises an hnRNP L polypeptide or a polynucleotide encoding the polypeptide. In some embodiments, the agent described herein comprises an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 3 or 4. In some embodiments, the agent described herein consists of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 3 or 4. In some embodiments, the agent comprises a small molecule compound selected from the group consisting of ascochlorin (ASC), vertihemipterin A, 4-O-methyl ascochlorin (MAC), vertuhemipterin A aglycone, AS-6,8′-hydroxyascochlorin, cylindrol A5,8,9′-dehydroascochlorin, ascofuranol, LL-Z1272ζ (8′-acetoxyascochlorin), ascofuranone (AF) and AF analogs or derivatives, as described in West et al. European Journal of Medicinal Chemistry 2017; 141:676-689, the content of which is incorporated by reference herein in its entirety. In some embodiments, the agent comprises a small molecule compound comprising at least one chemical structure of Formulas 1 to 77, and compounds described in WO2019053159 and WO2017119515, both of which incorporated by references herein in their entities. In some embodiments, the agent comprises ascochlorin (ASC), an ascochlorin derivative, or an ascochlorin analogue. Ascochlorin and derivatives thereof can be found in or produced by fungal species, for example Acremonium sp., Acremonium lacunae, Acremonium egyptiacum, Ascochyta viciae, Ascochyta viciae, Cephalosporium diospyri, Colletotrichum nicotianae, Cylindrocladium sp., Cylindrocladium ilicicola, Cylindrocarpon lucidum, Fusarium sp., Nectria galligena, Nectria coccinea, Nigrosabulum globosum, Verticillium hemipterigenum, or Verticillium sp.

In some embodiments, the agents comprise neostigmine bromide, irinotecan, captopril, proscillaridin, digoxin or 0179445-0000 (DSigDB).

In some embodiments, the agents comprise vandetanib, amantadine, Phenethyl Isothiocyanate, Astemizole, Lansoprazole, Docetaxel, or Paclitaxel.

In some embodiments, the agents comprise an FDA-approved compound that elevates the levels of hnRNP L.

In some embodiments, the agents comprise an European Medicines Agency (EMA)-approved compound that elevates the levels of hnRNP L.

In some embodiments, the agents described herein comprise a small molecule that elevates the levels of hnRNP L. In some embodiments, the agents comprise a combination of small molecules that elevates the levels of hnRNP L.

In another aspect, the present disclosure provides a method of increasing expression levels and/or stability of hnRNP L in a subject, comprising administering to the subject an agent comprising a small molecule compound selected from the group consisting of ascochlorin (ASC), vertihemipterin A, 4-O-methyl ascochlorin (MAC), vertuhemipterin A aglycone, AS-6,8′-hydroxyascochlorin, cylindrol A5,8′,9′-dehydroascochlorin, ascofuranol, LL-Z1272ζ (8′-acetoxyascochlorin), ascofuranone (AF) and AF derivatives described in West et al. European Journal of Medicinal Chemistry 2017; 141:676-689, the content of which is incorporated by reference herein in its entirety, and other compounds described herein, as well as bioactive ascochlorin analogs described in Subko et al. Marine Drugs 2021; doi.org/10.3390/md19020046, as well as described in references DOI: 10.1055/a-2099-4932; doi.org/10.3390/jof8070669; doi.org/10.25135/rnp.329.2204.2437; doi.org/10.1016/j.dsr.2023.104114; doi.org/10.3390/md19020046; the content of which is incorporated by reference herein in its entirety, and other compounds described herein. In some embodiments, the agent comprises a small molecule compound comprising at least one chemical structure of Formulas 1 to 77. In some embodiments, the agent comprises ascochlorin (ASC), an ascochlorin derivative, or an ascochlorin analogue. In some embodiments, the agent is capable of increasing expression levels of hnRNP L to at least a certain percentage (e.g., 10%, 20%, 25%, 30%, 40%, 50%, 60%, 70%, 80%, 90%, 95%, 99%, 100%, 2-fold, 5-fold, 10-fold, or more), compared to normal control levels in a healthy subject or a subject without the neurological disease.

In another aspect, the present disclosure provides a composition comprising an agent capable of increasing expression levels and/or stability of hnRNP L. In some embodiments, the agent is capable of increasing expression levels of hnRNP L to at least a certain percentage (e.g., 10%, 20%, 25%, 30%, 40%, 50%, 60%, 70%, 80%, 90%, 95%, 99%, 100%, 2-fold, 5-fold, 10-fold, or more), compared to control levels. The control may be a baseline or control level.

In another aspect, the present disclosure provides a composition comprising an agent capable of increasing expression levels and/or stability of hnRNP L, for treating a subject with a neurological disease associated with a splicing defect caused by TDP-43 proteinopathies. In some embodiments, the agent is capable of increasing expression levels of hnRNP L to at least a certain percentage (e.g., 10%, 20%, 25%, 30%, 40%, 50%, 60%, 70%, 80%, 90%, 95%, 99%, 100%, 2-fold, 5-fold, 10-fold, or more), compared to normal control levels in a healthy subject or a subject without the neurological disease.

In some embodiments, the subject has a loss of/altered TDP-43 function. In some embodiments, the subject shows a loss of/altered TDP-43 function due to (i) a mutation in the TDP-43 gene/open reading frame; and/or (ii) an altered wild type TDP-43 function following either (or a combination of) nuclear clearance, cytoplasmic inclusions, nuclear inclusions, aggregation, abnormal modification (e.g., ubiquitination), neuronal propagation in a “prion-like” manner, etc. In some embodiments, the loss of/altered TDP-43 function promotes cryptic exon inclusion. In some embodiments, the loss of/altered TDP-43 function reduces expression levels of the UNC13A or UNC13B gene. In some embodiments, the UNC13A gene encodes a UNC13A protein comprising an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 103. In some embodiments, the UNC13B gene encodes a UNC13B protein comprising an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 104. In some embodiments, the loss of/altered TDP-43 function reduces expression levels of the UNC13A, UNC13B, STMN2, SORT1, GPSM2, or ATG4B gene. In some embodiments, the SORT1 gene described herein encodes a SORT1 protein comprising an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 14 or 15. In some embodiments, the SORT1 gene described herein encodes a SORT1 protein consisting of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 14 or 15. In some embodiments, the GPSM2 gene described herein encodes a GPSM2 protein comprising an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 16. In some embodiments, the GPSM2 gene described herein encodes a GPSM2 protein consisting of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 16. In some embodiments, the ATG4B gene described herein encodes a ATG4B protein comprising an amino acid sequence having at least 50%, 60%, 65%, 70%, 75%, 80%, 85%, 90%, 91%, 92%, 93%, 94%, 95%, 96%, 97%, 98%, 99%, or 100% sequence identity to any one of SEQ ID NOs: 17-21. In some embodiments, the ATG4B gene described herein encodes a ATG4B protein consisting of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to any one of SEQ ID NOs: 17-21. In some embodiments, the STMN2 gene described herein encodes a STMN2 protein comprising an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 22 or 23. In some embodiments, the UNC13A gene encodes a UNC13A protein comprising an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 103. In some embodiments, the UNC13B gene encodes a UNC13B protein comprising an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 104. For example, the expression levels and/or stability of the UNC13A, UNC13B, STMN2, SORT1, GPSM2, or ATG4B gene may be reduced to about 90%, 80%, 70%, 60%, 50%, 40%, 30%, 20%, 10%, 5%, or less, than the normal or average/mean expression levels and/or stability in a healthy subject or a subject without the neurological disease. In some embodiments, the expression levels of the UNC13A, UNC13B STMN2, SORT1, GPSM2, or ATG4B gene, either on the RNA level or the protein level, are completely inhibited (i.e., 0% of normal control levels; may be caused by, e.g., knocking out or a deletion of the UNC13A, UNC13B, STMN2, SORT1, GPSM2, or ATG4B gene). In some embodiments, the loss of/altered TDP-43 function promotes a splicing defect in a UNC13A, UNC13B, STMN2, SORT1, GPSM2, or ATG4B gene. In some embodiments, the loss of/altered TDP-43 function inhibits neurite and/or axon growth. For example, the neurite and/or axon growth may be reduced to about 90%, 80%, 70%, 60%, 50%, 40%, 30%, 20%, 10%, 5%, or less, than the normal or average/mean neurite and/or axon growth in a healthy subject or a subject without the neurological disease. In some embodiments, the neurite and/or axon growth is completely inhibited (i.e., 0% of normal control levels).

In some embodiments, the neurological disease comprises at least one of cryptic exon-induced neurological diseases (CEIND), as described herein, such as Amyotrophic Lateral Sclerosis (ALS), Autism Spectrum Disorder (ASD), Frontotemporal lobar degeneration (FTLD), Frontotemporal Dementia (FTD), myotonic dystrophy type 1 (DM1) or type 2 (DM2), Alzheimer's disease, Lewy body dementia (LBD), Pick's disease, Hippocampal sclerosis, Corticobasal degeneration, Huntington disease, Parkinson's disease, Argyrophilic grain disease, Chronic traumatic encephalopathy (CTE), Perry syndrome, Alexander disease, Multisystem proteinopathy (MSP), intellectual disability. ADHD, dyslexia, epilepsy, bipolar disorder, depression and schizophrenia, parkinsonism-dementia complex of Guam, dementia pugilistica, diffuse neurofibrillary tangles with calcification, Down's syndrome, familial British dementia, familial Danish dementia, frontal lobe dementia, Gerstmann-Straussler-Scheinker disease, globular glial tauopathies, white matter tauopathy with globular glial inclusions, Guadeloupian parkinsonism with dementia, Guadeloupian progressive supranuclear palsy, multiple system atrophy, myotonic dystrophy, neurodegeneration with brain iron accumulation, Hallervorden-Spatz disease, pantothenate kinase-associated neurodegeneration, neurofibrillary tangle-predominant dementia. Niemann-Pick disease, prosencephalitic parkinsonism, prion protein cerebral amyloid angiopathy, progressive supranuclear palsy, SLCI A6-related mental retardation, subacute sclerosing panencephalitis, limbic predominant, age-related TDP-43 encephalopathy (LATE), and inclusion body myopathy. In some embodiments, the neurological disease comprises Amyotrophic Lateral Sclerosis (ALS), Frontotemporal Dementia (FTD), myotonic dystrophy type 1 (DM1), Pick's disease, hippocampal sclerosis, corticobasal degeneration, argyrophilic grain disease, and Huntington disease. In some embodiments, the neurological disease comprises Amyotrophic Lateral Sclerosis (ALS), Frontotemporal Dementia (FTD), Alzheimer's disease, chronic traumaticencephalopathy (CTE), limbic predominant, age-related TDP-43 encephalopathy (LATE), or inclusion body myopathy. Some neurological diseases may include intellectual disability, ADHD, dyslexia, epilepsy, bipolar disorder, Alzheimer's disease, Parkinson's disease, depression and schizophrenia. Other CEIND or PEIND may be treated such as Fragile X Syndrome (FXS) or those included in Sanders et al . . . “A framework for the investigation of rare genetic disorders in neuropsychiatry” Review Nat Med. 2019 October; 25 (10): 1477-1487.

In some embodiments, the increased expression levels and/or stability of hnRNP L reduces the splicing defect caused by TDP-43 proteinopathies. In some embodiments the increased levels and/or stability of hnRNP L partially of completely rescues the normal full-length transcript level, or protein level, of a TDP-43 target gene.

In some embodiments, the agent comprises at least one of a small molecule, a nucleic acid, an enzyme, a protein, a polypeptide, an antibody or a functional fragment thereof, an aptamer, a small interfering RNA, a microRNA, a long noncoding RNA (lncRNA), a small hairpin RNA, an antisense nucleic acid (e.g., ASO), a tricyclo-DNA (tcDNA), locked nucleic acid (LNA), a peptide nucleic acid (PNA), or a phosphorodiamidate morpholino oligomer (PMO). In some embodiments, the agent comprises an hnRNP L polypeptide or a polynucleotide encoding the polypeptide. In some embodiments, the agent described herein comprises an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 3 or 4. In some embodiments, the agent described herein consists of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 3 or 4. In some embodiments, the agent comprises a small molecule compound selected from the group consisting of ascochlorin (ASC), vertihemipterin A, 4-O-methyl ascochlorin (MAC), vertuhemipterin A aglycone, AS-6,8′-hydroxyascochlorin, cylindrol A5,8′,9′-dehydroascochlorin, ascofuranol, LL-Z1272ζ (8′-acetoxyascochlorin), ascofuranone (AF) and AF analogs or derivatives described in West et al. European Journal of Medicinal Chemistry 2017; 141:676-689, the content of which is incorporated by reference herein in its entirety, and other compounds described herein, as well as bioactive ascochlorin analogs described in Subko et al. Marine Drugs 2021; doi.org/10.3390/md19020046, as well as described in references DOI: 10.1055/a-2099-4932; doi.org/10.3390/jof8070669; doi.org/10.25135/rnp.329.2204.2437; doi.org/10.1016/j.dsr.2023.104114; doi.org/10.3390/md19020046; the content of which is incorporated by reference herein in its entirety, and other compounds described herein. In some embodiments, the agent comprises a small molecule compound comprising at least one chemical structure of Formulas 1 to 77. In some embodiments, the agent comprises ascochlorin (ASC), an ascochlorin derivative, or an ascochlorin analogue.

In another aspect, the present disclosure provides a pharmaceutical composition for treating a subject with a neurological disease associated with a splicing defect caused by TDP-43 proteinopathies, comprising the composition described herein and a pharmaceutically acceptable carrier.

In another aspect, the present disclosure provides a kit comprising a composition or a pharmaceutical composition described herein.

In another aspect, the present disclosure provides a method of treating a subject with a hnRNP L proteinopathy-dependent neurological disease, comprising administering to the subject an agent to increase expression levels and/or stability of hnRNP L, thereby repairing said splicing defect.

In another aspect, the present disclosure provides a method of treating a subject with a cryptic exon-poison exon-, or intron retention-dependent neurological disease, comprising administering to the subject an agent to increase expression levels and/or stability of hnRNP L, thereby repairing said cryptic/poison exon or intron retention defect.

In some embodiments, the agent, the composition, the pharmaceutical composition, or the kit comprises about 0.1 to about 500 mg/kg ASC, or other compounds or molecules (e.g., ASC derivatives or analogs) described herein. In some embodiments, the formulation comprises about 0.1 to about 500 mg/kg, about 0.1 to about 400 mg/kg, about 0.1 to about 300 mg/kg, about 0.1 to about 200 mg/kg, about 0.1 to about 100 mg/kg, about 0.1 to about 50 mg/kg, about 0.1 to about 20 mg/kg, about 0.1 to about 10 mg/kg, about 0.1 to about 5 mg/kg, about 0.5 to about 100 mg/kg, about 0.5 to about 50 mg/kg, about 0.5 to about 20 mg/kg, about 0.5 to about 10 mg/kg, about 0.5 to about 5 mg/kg, about 1 to about 100 mg/kg, about 1 to about 50 mg/kg, about 1 to about 20 mg/kg, about 1 to about 10 mg/kg, about 1 to about 5 mg/kg, about 5 to about 10 mg/kg, about 5 to about 20 mg/kg, about 5 to about 50 mg/kg, about 5 to about 100 mg/kg, or other amount of ASC, or other compounds or molecules described herein. In some embodiments, the formulation comprises about 0.1 to about 100 mg/kg ASC, or other compounds or molecules (e.g., ASC derivatives or analogs) described herein. In some embodiments, the formulation comprises about 0.5 to about 50 mg/kg ASC, or other compounds or molecules (e.g., ASC derivatives or analogs e.g., AF or AF derivatives) described herein. In some embodiments, the formulation comprises about 1 to about 500 mg/kg, about 10 to about 400 mg/kg, about 10 to about 300 mg/kg, about 10 to about 200 mg/kg, about 10 to about 100 mg/kg, about 50 to about 400 mg/kg, about 50 to about 300 mg/kg, about 50 to about 200 mg/kg, about 50 to about 100 mg/kg, about 100 to about 400 mg/kg, about 100 to about 300 mg/kg, about 100 to about 200 mg/kg, or other amount of ASC, or other compounds or molecules described herein. Other dosages may also be used, such as those illustrated in FIG. 16. For example, a 50 mg dose of 4-O-methylascochlorin may be administered orally 3 time per day (e.g., after every meal).

In some embodiments, the agent, the composition, or the pharmaceutical composition describe herein is administrated locally or systematically to the subject. For example, the agent may be administered by topical, oral, nasal, subcutaneous, intrathecal, intravenous (IV) routes, nasogastric tube, percutaneous endoscopic gastrostomy (PEG) tube, and/or injection into the central nervous system, e.g., using gene therapy.

HnRNP L is an important splicing factor expressed in the brain. It is a highly conserved protein that recognizes CA-repeat sequences and CA-rich motifs on targets (FIGS. 3A and 3B).

The splicing defect or spliceopathy may be detected, e.g., using whole genome sequencing and/or identification of aberrant splice variants in a sample of RNA or corresponding cDNA. Examples of such splicing defects or the spliceopathies include, but are not limited to, exon (all or part) skipping, in-frame deletion, exon (all or part) inclusion, intron (all or part) retention, or the usage of cryptic 5′ or 3′ splice sites, or the usage of cryptic splice-polyadenylation. Some such splicing defects may occur in STMN2. Additionally, the splicing defect or the spliceopathy may also include altered relative abundance of alternatively splice variants. For example, the ratio of a predominant brain splice variant vs. a minor brain splice variant may be in an abnormal value/amount. In another example, the ratio of an embryonic or fetal splice variant vs. an adult splice variant may be in an abnormal value/amount or in an abnormal ratio. In another example, tissue-specific normal variants may be expressed in inappropriate tissues, such as muscle-specific variants expressed in brain. Such non-neuronal splice variants expressed in neuronal tissue indicate an abnormality that is indicative of neurological disorders. Detection of the spliceopathies (aberrant splicing) in subject tissues or cells can be achieved using minimally invasive procedures. For example, defects may be detected in the RNA extracted from the patient's peripheral blood lymphocytes, using cDNA-SSCP-HD analysis. In some embodiments, other biological fluids (e.g., saliva, urine, perspiration) or biosamples (e.g. buccal swabs, nasal swabs) may be used for detection of such defects.

A spliceopathy rescue agent may be defined as an agent that restores or compensates functional defects caused by splicing defects or spliceopathies. For example, a spliceopathy rescue agent may restore the altered splicing and thus inhibit expression of abnormal mRNA variants or protein isoforms and/or improve expression of normal forms of mRNA or protein. A spliceopathy rescue agent may rescue levels of a full-length RNA corresponding to the misspliced transcript, without directly affecting the splicing event. A spliceopathy rescue agent may also restore the tissue specificity, e.g., tissue specific expression, of the target gene. Alternatively, a spliceopathy rescue agent may not directly influence the altered splicing, but compensate a defective function caused by the altered splicing.

Examples of a spliceopathy rescue agent that alters a gene splicing profile include, but are not limited to, those documented in the literature (e.g., Martinez-Montiel et al., Alternative Splicing as a Target for Cancer Treatment, Int. J. Mol. Sci. 2018, 19:545; Bates et al., Pharmacology of Modulators of Alternative Splicing, Pharmacol Rev 2017, 69:63-79; Hui et al., EMBO J. 2005; 24:1988-1998; Lin et al., Science. 2015; 349:650-655; Lec et al. Acta Neuropathologica. 2017; 134:65-78; Das et al. RNA Biology. 2019; 16:155-159; McCloy et al. RNA 2018; 24:761-768; Mohagheghi et al. Human Molecular Genetics, 2016; 25:534-545; Klim et al. Nature Neuroscience 2019; 22:167-179, which are incorporated herein by reference in their entirety). Non-limiting exemplary spliceopathy rescue agents include a small molecule, a nucleic acid, an enzyme, a protein, a polypeptide, an antibody or a functional fragment thereof, an aptamer, an RNA-based compound (e.g., a small interfering RNA, a microRNA and a small hairpin RNA), an antisense nucleic acid, a PNA, a CRISPR/Cas construct and the like, whether these are natural or synthetic. The rescue agent may include a small molecule, a nucleic acid, an enzyme, a protein, a polypeptide, an antibody or a functional fragment thereof, an aptamer, a small interfering RNA, a microRNA, a long noncoding RNA (lncRNA), a small hairpin RNA, an antisense nucleic acid (e.g., ASO), a tricyclo-DNA (tcDNA), locked nucleic acid (LNA), a peptide nucleic acid (PNA), or a phosphorodiamidate morpholino oligomer (PMO).

An exemplary small molecule includes ascochlorin, an ascochlorin derivative, or an ascochlorin analogue. An ascochlorin derivative may include a chemical compound derived from ascochlorin as a product of a chemical reaction (e.g., Cylindrol A5,4-O-methylascochhlorin (MAC)). By comparison, an ascochlorin analog may be structurally similar to ascochlorin. For instance, ascofuranone, an ascofuranone derivative or an ascofuranone analog are non-limiting examples of ascochlorin analogues. Exemplary ascochlorin derivative compounds include an ascochlorin glycoside Vertihemipterin A, a aglycone thereof, 4′,5′-dihydro-4′-hydroxyascochlorin, 8′-hydroxyascochlorin; LL-Z1272delta, 8′,9′-dehydroascochlorin, ascofuranone, ascofuranol, AS-6, Cylindrol A5,4-O-methylascochhlorin (MAC), colletochlorin, neostigmine bromide, irinotecan, captopril, proscillaridin, digoxin, 0179445-0000 (DSigDB), or other agents described herein, including various FDA- or EMA-approved compounds that increase hnRNPL.

Particularly preferred are compounds characterized as having minimal or absence of clinical toxicity. For example, MAC has been tested in clinical trials (see, for example, U.S. Pat. No. 3,995,061, 1976) and was well tolerated. The ascochlorin derivatives 4-O-methyl-ascochlorin (MAC) and 4-O-ethyl-ascochlorin display low toxicity as assessed by high LD50 after IP or oral administration (see, for example, Hosokawa T et al., U.S. Pat. No. 3,995,061, 1976). Suitable compound include 4-O-methylascochlorin (MAC), 4-O-ethylascochlorin, and other derivatives/analogs, including AS-6, ascofuranone (AF) and AF-like analogs/ubiquinol mimics isolated via novel routes of synthesis using structure activity relationships (SAR) (e.g., AF-like analogues, as described in West et al., Eur J Med Chem. 2017 Dec. 1; 141:676-689), or ASC, AF and derivatives that display anti-trypanosoma, anti-vibrio, or antiparasitic activities, ascochlorin glycoside Vertihemipterin A, a aglycone thereof, 4′,5′-dihydro-4′-hydroxyascochlorin, 8′-hydroxyascochlorin; LL-Z1272delta, 8′,9′-dehydroascochlorin, as well as bioactive ascochlorin analogs described in Subko et al. Marine Drugs 2021; doi.org/10.3390/md19020046, the content of which is incorporated by reference herein in its entirety, and other compounds described herein. Other suitable compounds include cefacetrile, cefotaxime, ciproflaxin, netilimicine or a fluoroquinolone/quinolone compound (see, for example, Kang et al., J Proteome Res. 2006; 5:2620-31).

The hnRNP L binding site may be located within an intron, or within the exon, adjacent to a site of alternative splicing of the target UNC13A, UNC13B, STMN2, SORT1, GPSM2, or ATG4B-associated gene in a subject having a splicing defect. More specifically, the gene may have an hnRNP L binding site within 5000, 4000, 3000, 2000, 1000, 500, 400, 300, 200, 100 or 50 base pairs of a site of alternative splicing. Further, the gene may have an hnRNP L binding site within 4000, 3000, 2000, 1000, 900, 800, 700, 600, 500, 400, 300, 200, 100 or 50 base pairs of an RBFox1/A2BP1 binding site.

Preferably, exemplary target genes in which the subject to be treated has a splicing defect include, but are not limited to, genes bearing hnRNP L binding sites, e.g., UNC13A, UNC13B, STMN2, SORT1, GPSM2, and ATG4B.

Alternatively, the subject to be treated has a splicing defect in any genes that is a target of hnRNP L as described herein. More particularly, the subject may have a splicing defect in genes, which have a high-scoring hnRNP L motif within 500 bp of one of the Castle splice sites (see, for example, Castle, et al., Nature Genetics 40 (12): 1416-25, 2008) (338 genes).

As described above, the target gene (e.g., UNC13A, UNC13B, STMN2, SORT1, GPSM2, or ATG4B) in which the subject has a splicing defect may be characterized as having an hnRNP L binding site within the intron, or within the exon, adjacent to a site of alternative splicing. More specifically, the gene may have an hnRNP L binding site within 5000, 4000, 3000, 2000, 1000, 500, 400, 300, 200, 100 or 50 base pairs of a site of alternative splicing. In another example, the gene may have an hnRNP L binding site within 4000, 3000, 2000, 1000, 900, 800, 700, 600, 500, 400, 300, 200, 100 or 50 base pairs of the binding site of a splicing factor which is partner of hnRNP L in a splicing complex.

As described above, exemplary genes in which the subject to be treated has a splicing defect include UNC13A, UNC13B, STMN2, SORT1, GPSM2, or ATG4B gene, e.g., a subject with ALS or FTD, or other neurological diseases described herein having a splicing defect resulting from decreased expression or function or abnormal localization of TDP-43 which in turn alters gene splicing of its target genes, including UNC13A, UNC13B, STMN2, SORT1, GPSM2, ATG4B, and others known in the art.

Alternatively, as described above, the subject has a splicing defect in any gene that is a target of hnRNP L as described herein. For example, the subject may have a splicing defect in any of UNC13A, UNC13B, STMN2, SORT1, GPSM2, or ATG4B gene that is a target of hnRNP L. As described above, the subject has a mutation in the target gene which results in spliceopathy.

Disclosed herein, in some embodiments, are agents. The agent may be included or used in a method or composition. In some embodiments, the agent comprises at least one of a small molecule, a nucleic acid, an enzyme, a protein, a polypeptide, an antibody or a functional fragment thereof, an aptamer, a small interfering RNA, a microRNA, a long noncoding RNA (lncRNA), a small hairpin RNA, an antisense nucleic acid (e.g., ASO), a tricyclo-DNA (tcDNA), locked nucleic acid (LNA), a peptide nucleic acid (PNA), or a phosphorodiamidate morpholino oligomer (PMO). In some embodiments, the agent comprises a compound. In some embodiments, the agent comprises an oligonucleotide. In some embodiments, the agent comprises an ASO. In some embodiments, the ASO increases expression of hnRNP L. In some embodiments, the ASO targets a poison exon of hnRNP L, or targets an intron immediately upstream of the poison exon. In some embodiments, the ASO targets the 5′ or 3′ untranslated region (UTR) of an hnRNP L mRNA. In some embodiments, the poison exon comprises the following sequence:

GGTCGCAGTGTATGTTTGATGGGACGCCATCTTTCAGAACTGTGCTAACTCACTGTTGAA GCGTCCAATG (SEQ ID NO: 102). In some embodiments, the ASO comprises the base sequence of any one of SEQ ID NOs: 47-101, or a sequence having 1 or 2 insertions, substitutions, or deletions relative to any one of SEQ ID NOs: 47-101. In some embodiments, the ASO comprises the base sequence of any one of SEQ ID NOs: 47-69, or a sequence having 1 or 2 insertions, substitutions, or deletions relative to any one of SEQ ID NOs: 47-69. In some embodiments, the ASO comprises the base sequence of an ASO that increased max hnRNP L expression by at least 25%, at least 30%, at least 35%, at least 40%, at least 45%, at least 50%, at least 55%, at least 60%, at least 65%, or at least 70% in Table 9B, or a sequence thereof having 1 or 2 insertions, substitutions, or deletions. In some embodiments, the ASO comprises the base sequence of an ASO that had an EC50 value below 15 nM, below 14 nM, below 13 nM, below 12 nM, below 11 nM, below 10 nM, below 9 nM, below 8 nM, below 7 nM, below 6 nM, below 5 nM, below 4 nM, or below 3 nM in Table 9B, or a sequence thereof having 1 or 2 insertions, substitutions, or deletions. In some embodiments, the ASO comprises the base sequence of an ASO that increased hnRNP L by at least 25%, at least 30%, at least 35%, at least 40%, at least 45%, at least 50%, at least 55%, at least 60%, at least 65%, at least 70%, at least 75%, or at least 80% in Table 10, or a sequence thereof having 1 or 2 insertions, substitutions, or deletions. In some embodiments, the ASO comprises a DNA oligonucleotide. In some embodiments, the ASO comprises a nucleoside modification. In some embodiments, the nucleoside modification comprises 2′-O-methoxyethyl (MOE). In some embodiments, the nucleoside modification comprises 5′-methyl C. In some embodiments, the ASO comprises an internucleoside linkage modification. In some embodiments, the internucleoside linkage modification comprises a phosphorothioate linkage.

Each embodiment disclosed herein is contemplated as being applicable to each of the other disclosed embodiments.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

All references, e.g., journal articles, protein or nucleic acid sequence accession numbers, cited U.S. patents. U.S. patent application publications and PCT patent applications designating the U.S., are hereby incorporated by reference in their entirety.

### BRIEF DESCRIPTION OF THE DRAWINGS

FIG. 1 is a set of diagrams illustrating normal RNA splicing. A diagram in the upper panel shows a normal process of RNA splicing, involving a spliceosome and RNA binding protein snRNPs to cleave off introns from pre-mRNAs to produce mature mRNAs. The bottom panel illustrates that normal brain function is often highly dependent on correct alternative splicing. Alternative splicing can generate different transcripts/proteins.

FIG. 2 includes a set of graphs illustrating that hnRNP L, as a splicing factor, is widely expressed and distributed in the human brain.

FIGS. 3A and 3B are diagrams, adapted from Hui et al. EMBO J 2015; 24:1988-1998, showing the structure and the function of hnRNP L, a highly conserved splicing factor that recognizes CA-repeat sequences and CA-rich motifs on targets.

FIG. 4 is a diagram showing TDP-43 acting as a repressor of cryptic or poison exons. Ling et al., Science 2015 Aug. 7; 349 (6248): 650-655.

FIGS. 5A and 5B show that aberrant splicing leads to severe pathologies such as Amyotrophic Lateral Sclerosis (ALS) and Frontotemporal Dementia (FTD). FIG. 5A shows a RT-PCR detection strategy, while primers were designed to amplify only the cryptic exon splice junction. TDP-43 disruption or mutations may lead to cryptic exon inclusion in GPSM2 and ATG4B RNAS.

FIG. 5B shows DNA fragments detected at 199 base pairs (bp) (GPSM2) and at 215 bp (ATG4B) for all cases that display TDP-43 proteinopathies: control cases (“C”) did not display these fragments. MTG, middle temporal gyrus; MC, motor cortex. Adapted from Ling et al., Science. 2015 Aug. 7; 349 (6248): 650-655.

FIG. 6 is a chart with descriptions illustrating TDP-43 aggregate morphology that exemplify FTLD-TDP Types A, B, C, D, and E. Lee et al., Acta Neuropathol 2017 July; 134(1):65-78.

FIG. 7 illustrates how transcriptome integrity may be protected by different hnRNPs. Adapted from Das et al., RNA Biology 2019, VOL. 16, NO. 2, 155-159.

FIG. 8 includes charts illustrating that hnRNP L, TDP-43 and PTBP are useful repressors of cryptic/poison (e.g., pathogenic) exons. Adapted from McClory et al., RNA 2018, 24:761-768.

FIGS. 9A and 9B show that TDP-43 and hnRNP L may repress a cryptic or poison exon associated with Frontotemporal dementia (FTD). In FIG. 9A, loss of TDP-43 may promote inclusion of toxic exon 17b, leading to a toxic SORT1 isoform elevated in FTLD-TDP. In FIG. 9B, knockdown of hnRNP L and TDP-43 led to significant inclusion of endogenous SORT1 Ex17b in Neuro 2a cells. Adapted from Mohagheghi et al., Human Molecular Genetics 2016, Vol. 25, No. 3 534-545.

FIG. 10A-10C include details of some published studies. Some embodiments include any aspect of FIG. 10A-10C that pertains to hnRNP L.

FIG. 11 shows TDP-43 mutations/loss of function-induced missplicing of gene target STMN2, as seen in Amyotrophic Lateral Sclerosis (ALS). Adapted from Klim et al., Nature Neuroscience 2019, Vol. 22, February 167-179. The illustrated STMN2 gene has hnRNP L binding sites in close proximity to the cryptic exon, as shown.

FIGS. 12A and 12B show multiple molecular changes in cells after TDP-43 knockdown. In FIG. 12B, TDP-43 knockdown in SH-SY5Y cells resulted in significant changes in proteins in nuclear and cytoplasmic fractions, including an increased expression of hnRNP L levels by about 2.8 fold (2.8×). In FIG. 12B, TDP-43 aggregation in HEK293 cells led to multiple changes, including increased expression of hnRNP L levels by about 2 fold (2×). Miheve et al., Scientific Reports 2016, volume 6, Article number: 33996. FIG. 12C is a chart showing some interaction that may occur between hnRNP L and TDP-43. Adapted from Bampton et al., Acta Neuropathologica 2020.140:599-623.

FIG. 13 includes images illustrating a cellular model for sporadic ALS using patient-derived induced pluripotent stem cells useful to test compounds or approved drugs to identify potential compounds to reduce TDP-43 aggregation. Adapted from Burkhardt et al., Mol Cell Neurosci. 2013 September; 56:355-364.

FIG. 14 is a gel image showing a comparison of hnRNP L protein levels in rat primary cortical neurons with or without ASC treatments at different doses. The rat neurons were treated with 10⁻⁷ M or 10⁻⁶ M ASC, or not treated as control (“Cont.”), and then lysed for western blot detection of protein expression levels of hnRNP L (the upper panel). The arrow points to hnRNP L protein bands (appears as a triplet). The lower panel shows staining for GAPDH as a loading control. FIG. 14B is a gel image showing elevation of hnRNP L levels after ASC treatment in SH-SY5Y human neuroblastoma cells (CLT results).

FIGS. 15A and 15B show selection of ascochlorin (ASC) derivatives or analogs for low toxicity compounds. FIG. 15A shows multiple structures for ASC derivatives or analogs. FIG. 15B shows toxicity study results with different drugs administered via different routes.

FIG. 16 is a chart showing reports of absence of significant toxicity with administration of ascochlorin/derivatives in rodent disease models.

FIG. 17 is a chart illustrating that a compound (e.g., small molecule, ASO)-mediated increase in hnRNP L may compensate for TDP-43 deficiency in FTD, ALS and other neurological disorders. Under a normal situation (left panel), a subject may have normal expression levels of hnRNP L and TDP-43. A diseased subject (middle panel) may have decreased expression levels of TDP-43 (or due to mutation and/or aggregation of TDP-43). To treat such diseased subject (right panel), ASC or its derivatives or analogs, or other small molecules (e.g., FDA- or EMA-approved compounds) or other agents (e.g., nucleic acids) described herein may be administered to increase expression levels of hnRNP L, thus compensating the loss-of-function of TDP-43.

FIG. 18 is a schematic representation exemplifying an AAV-9 gene therapy construct for delivering hnRNP L to the central nervous system and/or for rescuing cell-based models of disease. The illustrated AAV genome includes a chicken beta (Cβ) actin promoter, CMV enhancer, synthetic intron, hnRNP L CDS, and eRBG polyA and AAV2 ITRs at both ends.

FIG. 19 is a schematic representation exemplifying lentiviral hnRNP L particles for rescuing cell-based models and/or in vivo of Cryptic Exon Induced Neurological Disease (CEIND) and/or Poison Exon Induced Neurological Disease (PEIND).

FIG. 20 is a sequence logo representing a binding motif.

FIG. 21 shows mapping of hnRNP L ASOs and hnRNP L bDNA probes for quantifying hnRNP L in response to the ASO treatment, in the branched DNA (bDNA) assay that was performed.

FIG. 22 shows mapping of hnRNP L ASOs to hnRNP L MANE reference transcripts and genome.

FIG. 23 shows mapping of bDNA probes to an hnRNP L RNA.

FIG. 24 shows ASOs which tile hnRNP L poison exon 6A and 5′ and 3′ flanking sequences. Percentages reflect hnRNP L transcript elevation above baseline (which equals 100%).

FIG. 25A-25E illustrate a concentration-dependent increase in hnRNP L transcript after treatment with ASOs targeting hnRNP L poison exon 6A.

FIG. 26 illustrates experiments done on two different days showing the effect of 30 nM ASO on hnRNP L transcript levels. ASO that target the 5′UTR were inactive and only tested once. The plot includes 2 sets of bar graphs for each ASO: a graph of the DRC analysis on the left, and a graph of the time course analysis on the right, for each ASO.

FIGS. 27-31 include diagrams showing details that may relate to diseases or genes relevant to increasing levels of hnRNP L (e.g., Autism Spectrum Disorder, Fragile X Syndrome).

### DETAILED DESCRIPTION

Splicing is in many instances an essential post-transcriptional process. Alternative splicing may generate different transcripts and/or proteins. Normal brain can greatly depend on correct alternative splicing. HnRNP L is an important splicing factor expressed in the brain. Computational analysis identifies multiple targets of hnRNP L in brain. Aberrant splicing (spliceopathy) can also induce neurological diseases, such as Amyotrophic Lateral Sclerosis (ALS), Frontotemporal Dementia (FTD) and Alzheimer's Disease. Amyotrophic Lateral Sclerosis (ALS) is a neurodegenerative disease characterized by the loss of motor neurons. ALS patients usually have progressive paralysis. Current treatment for ALS is limited to supportive care. Although the causes of many ALS and FTD case remains unknown, pathological findings and family-based linkage studies have demonstrated overlap in molecular pathways. A major constituent of inclusions in many sporadic cases of ALS and FTD, TDP-43, is a predominantly nuclear DNA/RNA-binding protein with functional roles in transcriptional regulation, splicing, pre-microRNA processing, stress granule formation and messenger RNA transport and stability.

The present application provides, at least, compositions and methods of using compounds such as antisense oligonucleotides (ASOs), or a small molecule such as ascochlorin (and/or derivatives and analogs) as a pharmacological modifier of abnormal splicing. Ascochlorin and/or its derivatives can promote the maintenance of normal brain physiology by targeting hnRNP L and/or components of the coordinated hnRNP L-regulated pathway(s). The compounds and methods described herein provide pharmacological leads to help treat and additional neurological and psychiatric disorders.

Disclosed herein, in some embodiments, are agents. The agent may be included or used in a method or composition. In some embodiments, the agent comprises at least one of a small molecule, a nucleic acid, an enzyme, a protein, a polypeptide, an antibody or a functional fragment thereof, an aptamer, a small interfering RNA, a microRNA, a long noncoding RNA (lncRNA), a small hairpin RNA, an antisense nucleic acid (e.g., ASO), a tricyclo-DNA (tcDNA), locked nucleic acid (LNA), a peptide nucleic acid (PNA), or a phosphorodiamidate morpholino oligomer (PMO). In some embodiments, the agent comprises a compound. In some embodiments, the agent comprises an oligonucleotide. In some embodiments, the agent comprises an ASO.

Some compositions, compounds (e.g., oligonucleotides), and methods described herein, are useful for increasing hnRNP L as a means to normalize pathological transcriptome alteration. The effect of increasing hnRNP L may have beneficial effects in subjects that have a splicing defect, or also in subjects that do not have a splicing defect. Some embodiments relate to a composition comprising an antisense oligonucleotide (ASO) that targets hnRNP L and when administered to or expressed in a cell, increases expression of the hnRNP L. In some embodiments, the composition reduces an amount or ratio of hnRNP L mRNA that includes a poison exon.

The compounds, compositions, and methods may be useful for treating a TDP-43 related proteinopathy, or a disorder unrelated to TDP-43 where splicing is affected. The compounds, compositions, and methods may be useful for treating any disease or disorder where hnRNP L levels are low, or where hnRNP L levels are desired to be elevated. Some embodiments relate to a method of increasing an hnRNP L measurement (e.g., an hnRNP L protein, or hnRNP L RNA, measurement) by administering a compound or composition described herein.

In some embodiments, increased hnRNP L partially/completely restores full length levels of TDP-43 misspliced gene/RNA target without a direct effect on the splicing event. In some embodiments, increased hnRNP L partially/completely restores protein levels of TDP-43 gene target without an effect on its RNA levels or splicing.

### TDP-43 Proteinopathies

TAR DNA-binding protein-43 (TDP-43), initially identified in 1995 as a suppressor of HIV-1 (HIV-1) gene expression, is a highly conserved and ubiquitously expressed RNA/DNA-binding protein belonging to the heterogeneous nuclear ribonucleoprotein (hnRNP) family. TDP-43 is pivotal in multiple cellular functions including regulation of RNA metabolism, mRNA transport, microRNA maturation and stress granule formation.

In line with its nuclear and cytoplasmic functions, TDP-43 can shuttle between the nucleus and the cytoplasm, but under normal physiological conditions, localization is predominantly nuclear. Of relevance to brain function, TDP-43 appears to be critical for normal development of central neuronal cells in early stages of embryogenesis. Given the extensive role of TDP-43 in cellular processes, particularly in the development of the central nervous system, dysfunction of TDP-43-related pathways has been recognized as an important pathogenic mechanism in neurodegenerative disease.

In 2006, hyperphosphorylated and ubiquitinated TDP-43 cytoplasmic inclusions were identified as a pathological feature of amyotrophic lateral sclerosis (ALS) and frontotemporal lobar disease (FTLD). Pathogenic missense mutations in the TARDBP gene, which encodes the TDP-43 protein, were subsequently identified as causative genetic mutations in both ALS and FTLD, although in a small percentage of familial cases. Interestingly, the vast majority of patients with ALS and FTLD do not harbor mutations in the TARDBP gene yet demonstrate widespread abnormalities involving TDP-43. The pathophysiological heterogeneity of ALS and FTLD phenotypes may suggest that multiple pathogenic pathways contribute to mislocalization and aggregation of TDP-43. Over the past decade. TDP-43 deposition has been associated with an increasing number of neurodegenerative diseases, where it has been identified as the primary pathogenic factor, resulting in these disorders being designated as “TDP-43 proteinopathies.” For reviews on TDP-43 proteinopathies, see de Boer et al. J Neurol Neurosurg Psychiatry 2020; 0:1-10; Kwong et al. Neurosignals 2008; 16:41-51; Tan et al. Brain. 2015; 138(Pt 10):3110-3122, the content of which are incorporated by reference herein to their entities.

TDP-43 proteinopathies encompass a wide range of neurodegenerative diseases and phenotypes, which may be inherited in a Mendelian pattern or be apparently sporadic. A large number of genes have been associated with TDP-43 proteinopathies, including, at least, UNC13A, UNC13B, STMN2, SORT1, GPSM2, ATG4B, GRN, C9orf72, VCP, and other genes listed in the specification and in Table 1 of de Boer et al. J Neurol Neurosurg Psychiatry 2020; 0:1-10). Genetic abnormalities are associated with multiple phenotypes and diseases. Specifically, the C9orf72 hexanucleotide expansion may cause ALS, FTLD, ALS-FTLD, Alzheimer's disease (AD) phenotypes and atypical Parkinsonism. The mechanisms underlying these pleiotropic effects are unclear, although genetic and/or environmental factors impacting on gene expression have been proposed but remain to be identified. The notion of ALS being a multistep disease, 16 with fewer steps required in familial ALS (fALS), 17 underscores the importance of epigenetic and environmental factors in ALS pathogenesis.

Although several TDP-43 proteinopathy genes encode RNA-binding proteins, the functions of the other associated genes are diverse. TDP-43 pathology may arise through multiple different mechanisms. Identifying the relationship between these dysfunctional genetic and molecular processes may be critical for development of effective therapies.

### TAR DNA-Binding Protein 43 (TDP-43)

Provided herein, in some embodiments, is a TAR DNA-binding protein 43 (TDP-43, transactive response DNA binding protein 43 kDa, a.k.a. TARDBP or ALS10). TDP-43 is a protein that in humans is encoded by the TARDBP gene. TDP-43 is 414 amino acid residues long. It includes 4 domains: an N-terminal domain spanning residues 1-76 (NTD) with a fold that forms a dimer or oligomer; 2 highly conserved folded RNA recognition motifs spanning residues 106-176 (RRM1) and 191-259 (RRM2), respectively, required to bind target RNA and DNA, and an unstructured C-terminal domain encompassing residues 274-414 (CTD), which contains a glycine-rich region, which is involved in protein-protein interactions and harbors most of the mutations associated with familial amyotrophic lateral sclerosis. See Lukavsky et al. Nature Structural & Molecular Biology 2013; 20:1443-1449; and Conicella et al. Structure 2016; 24:1537-1549. The full-length protein is a dimer, formed due to a self-interaction between two NTD domains, where the dimerization can be propagated to form higher-order oligomers. The protein sequence also has a nuclear localization signal (NLS, residues 82-98), a nuclear export signal (NES residues 239-250) and 3 putative caspase-3 cleavage sites (residues 13, 89, 219). Some information for TDP-43 is at the World Wide Web website of uniprot.org/uniprot/Q13148, under the reference number UniProtKB-Q13148. Reference numbers such as UniProt reference numbers provided herein are generally current as of the earliest date of filing.

Human TDP-43 Amino Acid Sequence (Isoform 1): MSEYIRVTEDENDEPIEIPSEDDGTVLLSTVTAQFPGACGLRYRNPVSQCMRGVRLVEGILHA PDAGWGNLVYVVNYPKDNKRKMDETDASSAVKVKRAVQKTSDLIVLGLPWKTTEQDLKE YFSTFGEVLMVQVKKDLKTGHSKGFGFVRFTEYETQVKVMSQRHMIDGRWCDCKLPNSKQ SQDEPLRSRKVFVGRCTEDMTEDELREFFSQYGDVMDVFIPKPFRAFAFVTFADDQIAQSLCG EDLIIKGISVHISNAEPEHNSERQLERSGREGGNPGGFGNQQGFQNSRQGAQLGNNQGSNM GGGMNFGAFSINPAMMAAAQAALQSWQMMGMLASQQEQSQPSGENQNQGNMQBEBNQ AFGSGNNSXSGSNSGAIGWGSASEAGSGSGFNGGFGSSMDSKSSGWGM (SEQ ID NO: 1; NCBI Reference Sequence: NP_031401.1; UniProtKB-Q13148-1; some sites for mutations indicated in ALS are underlined). NCBI, Ensembl, UniProt, or other database references are as of the date of first inclusion in a filing (e.g., provisional or PCT filing) herein.

### Alternative Human TDP-43 Amino Acid Sequence Produced by Alternative Splicing (Isoform 2):

(SEQ ID NO: 2)
MPQMLAGEIWCMLSTIQKVKKDLKTGHSKGFGFVRFTEYETQVKVMSQRH
MIDGRWCDCKLPNSKQSQDEPLRSRKVFVGRCTEDMTEDELREFFSQYGD
VMDVFIPKPFRAFAFVTFADDQIAQSLCGEDLIIKGISVHISNAEPKHNS
NRQLERSGRFGGNPGGFGNQGGFGNSRGGGAGLGNNQGSNMGGGMNFGAF
SINPAMMAAAQAALQSSWGM
MGMLASQQNQSGPSGNNQNQGNMQREPNQAFGSGNNSYSGSNSGAAIGWG
SASNAGSGSGFNGGFGSSMDSKSSGWGM.

In some embodiments, the TDP-43 described herein comprises an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 1 or 2. In some embodiments, the TDP-43 described herein consists of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 1 or 2.

The term “sequence identity” used in the present specification refers to the amount (in percentage) of characters which match exactly between two different sequences (e.g., nucleic acid sequences, or amino acid sequences). Gaps are usually not counted and the measurement is relational to the shorter of the two sequences. The sequence identity of two sequences may be calculated by any of methods and/or software known in the art, such as the NCBI BLAST algorithm (e.g., default algorithm as of the date of this filing).

Other isoforms or natural variants of TDP-43 can be found in the UNIPROT database (entry number UniProtKB-Q13148) at World Wide Web website of uniprot.org/uniprot/Q13148. Exemplary isoforms include, e.g., UNIPROT database entry numbers Q13148, A0A087WX67, A0A087WXV3, A0A087WTG4, A0A0A0N0M3, A0A087WV68, A0A087WX29, A0A087WTZ4, A0A087WW61, G3V162, K7EN94, A0A087WZM1, A0A087WXQ5, A0A087WZC9, K7EL26, K7ENM9, A0A087WVX6, A0A1W2PNU8, A0A0A0MSV7, B1AKP7, K7EJ99, K7EJM5, A0A087WYY0, A0A087WYE7, and A0A087X260.

TDP-43 has been shown to bind both DNA and RNA and have multiple functions in transcriptional repression, pre-mRNA splicing, RNA stability, translational regulation and other cellular functions. Recent work has characterized the transcriptome-wide binding sites revealing that thousands of RNAs are bound by TDP-43 in neurons.

TDP-43 was originally identified as a transcriptional repressor that binds to chromosomally integrated trans-activation response element (TAR) DNA and represses HIV-1 transcription.

In spinal motor neurons TDP-43 has also been shown in humans to be a low molecular weight neurofilament (hNFL) mRNA-binding protein. It has also shown to be a neuronal activity response factor in the dendrites of hippocampal neurons suggesting possible roles in regulating mRNA stability, transport and local translation in neurons.

Recently, it has been demonstrated that zinc ions are able to induce aggregation of endogenous TDP-43 in cells. Moreover, zinc could bind to RNA binding domain of TDP-43 and induce the formation of amyloid-like aggregates in vitro.

TDP-43 protein is a key element of the non-homologous end joining (NHEJ) enzymatic pathway that repairs DNA double-strand breaks (DSBs) in pluripotent stem cell-derived motor neurons. TDP-43 is rapidly recruited to DSBs where it acts as a scaffold for the further recruitment of the XRCC4-DNA ligase protein complex that then acts to seal the DNA breaks. In TDP-43-depleted human neural stem cell-derived motor neurons, as well as in sporadic ALS patients' spinal cord specimens there is significant DSB accumulation and reduced levels of NHEJ. See Mitra et al. Proc Natl Acad Sci USA. 2019; 116:4696-4705.

A hyper-phosphorylated, ubiquitinated and cleaved form of TDP-43—known as pathologic TDP-43—is the major disease protein in ubiquitin-positive, tau-, and alpha-synuclein-negative frontotemporal dementia (FTLD-TDP, previously referred to as FTLD-U; see Mackenzie et al. Acta Neuropathologica. 2011; 122:111-113) and in amyotrophic lateral sclerosis (ALS). See Mackenzie and Rademakers Curr Opin Neurol 2008; 21:693-700. Abnormalities of TDP-43 also occur in an important subset of Alzheimer's disease patients, correlating with clinical and neuropathologic features indexes. Misfolded TDP-43 is found in the brains of older adults over age 85 with limbic-predominant age-related TDP-43 encephalopathy (LATE), a form of dementia.

Mutations in the TARDBP gene are associated with neurodegenerative disorders including frontotemporal lobar degeneration and amyotrophic lateral sclerosis (ALS). See Kwong et al. Acta Neuropathologica. 2007; 114:63-70. In particular, the TDP-43 mutants M337V and Q331K are being studied for their roles in ALS. See Sreedharan et al. Science. 2008; 319:1668-1672; Gendron et al. Journal of Alzheimer's Disease 2013; 33 (suppl 1): S35-45: Babić et al. Behavioural Neurology 2019:2909168. Cytoplasmic TDP-43 pathology is the dominant histopathological feature of multisystem proteinopathy. The N-terminal domain, which contributes importantly to the aggregation of the C-terminal region, has a novel structure with two negatively charged loops. A recent study has demonstrated that cellular stress can trigger the abnormal cytoplasmic mislocalization of TDP-43 in spinal motor neurons in vivo, providing insight into how TDP-43 pathology may develop in sporadic ALS patients (Svahn et al. Acta Neuropathologica 2018; 136:445-459).

Aggregation and loss of nuclear TDP-43 are pathological hallmarks of ALS. In ALS patients. TDP-43 mislocalization is associated with accumulation of insoluble TDP-43 proteins. Proteasome inhibition in human motor neurons (hMNs) induced TDP-43 mislocalization and insolubility.

Nearly all of the described ALS-associated TDP-43 mutations include dominant missense mutations within the glycine-rich domain, suggesting that altering the function of this domain is sufficient to induce neurodegeneration. TDP-43 proteinopathies were documented in a wide range of other neurodegenerative diseases, including Alzheimer's disease, other tauopathies and Lewy body disorders characterized by α-synuclein inclusions. The extent of TDP-43 pathology in these other diseases is limited in terms of both the amount and distribution of TDP-43 compared with cases of primary ALS and FTLD-TDP. Thus, the formation of TDP-43 pathology, although a primary event in FTLD-TDP and ALS, may be a secondary event in these other diseases, and it remains to be determined whether abnormal TDP-43 exacerbates the extent of neurodegeneration in these patients. The major disease-specific findings in FTLD-TDP and ALS include abnormal ubiquitination and phosphorylation of TDP-43, the presence of sarko-syl-insoluble TDP-43 inclusions, the presence of truncated 20-25-kDa TDP-43 C-terminal fragments (CTFs; particularly in the cerebral cortex), mislocalization of TDP-43 protein, and loss of normal nuclear TDP-43 expression.

For a review of normal and abnormal post-translational modifications, ubiquitination, and phosphorylation to TDP-43, see Lee et al. Nat Rev Neurosci. 2012; 13(1):38-50, which is incorporated herein by reference in its entirety. Specifically. TDP-43 contains two RNA-recognition motifs [RNA-recognition motif 1 (RRM1) and RRM2], a carboxyl-terminal glycine-rich domain, a bipartite nuclear localization signal (NLS) and a nuclear export signal (NES). Mutations have been linked to sporadic and familial forms of amyotrophic lateral sclerosis (ALS) and frontotemporal lobar degeneration (FTLD). These are almost exclusively found within or immediately adjacent to the glycine-rich domain with the exception of an Asp169Gly mutation within exon 4 (the site at which TDP-43 cleavage putatively occurs is shown by an arrow). TDP-43 phosphorylation sites (e.g., Ser 379, Ser403+Ser404, and Ser409+Ser 410), when heavily phosphorylated, may also contribute to the disease-specific TDP-43 biochemical signature (these sites are indicated by asterisks). A list of exemplary TDP-43 mutations is shown in Table 1 below. A subject such as a subject to be treated may have a mutation as shown in Table 1.

TABLE 1
Exemplary TDP-43 mutations

| TDP-43 Domains | TDP-43 Mutations |
|---|---|
| RRM1 | D169G |
| RRM2 | K263E, N267S |
| Glycine-rich domain | G287S, G290A, S292N, G294A/V, G295R/S, |
| | G298S, M311V, A315T, A321V/G, Q331K, |
| | S332N, G335D, M337V, Q343R, N345K, |
| | G348C/V, N352S/T, R361S, P363A, Y374X, |
| | N378D, S379P/C, A382P/T, I383V, G384R, |
| | N390D/S, S393L |

Further mutations at phosphorylation sites on TDP-43, e.g., S403, S404, S409, and S410, to affect TDP-43 functions. Such mutations may be introduced into TDP-43.

Recent studies show that almost all cases of ALS and many other tauopathies share a common neuropathology characterized by the deposition of TDP-43-positive protein inclusions. Multiple diseases or disorders are associate with cryptic exons and/or intron retentions caused by TDP-43 (e.g, cytoplasmic accumulation of TDP-43), including Amyotrophic Lateral Sclerosis (ALS) and other cryptic exon-induced neurological diseases (CEIND). These diseases or disorders are referred to as ALS/CEIND in this application, such as Frontotemporal lobar degeneration (FTLD), Frontotemporal Dementia (FTD), myotonic dystrophy type 1 (DM1), Alzheimer's disease, Autism Spectrum Disorder (ASD), Lewy body dementia (LBD), Pick's disease, Hippocampal sclerosis, Corticobasal degeneration, Huntington disease, Parkinson's disease, Argyrophilic grain disease, Chronic traumatic encephalopathy (CTE), Perry syndrome, Alexander disease, Multisystem proteinopathy (MSP), intellectual disability, ADHD, dyslexia, epilepsy, bipolar disorder, depression and schizophrenia, parkinsonism-dementia complex of Guam, dementia pugilistica, diffuse neurofibrillary tangles with calcification, Down's syndrome, familial British dementia, familial Danish dementia, frontal lobe dementia, Gerstmann-Straussler-Scheinker disease, globular glial tauopathies, white matter tauopathy with globular glial inclusions, Guadeloupian parkinsonism with dementia, Guadeloupian progressive supranuclear palsy, multiple system atrophy, myotonic dystrophy, neurodegeneration with brain iron accumulation, Hallervorden-Spatz disease, pantothenate kinase-associated neurodegeneration, neurofibrillary tangle-predominant dementia, Niemann-Pick disease, prosencephalitic parkinsonism, prion protein cerebral amyloid angiopathy, progressive supranuclear palsy, SLCI A6-related mental retardation, subacute sclerosing panencephalitis and Fragile X Syndrome (FXS). For other TDP-43-related diseases and disorders, see U.S. Patent Publication No. 20200330497A1, which is incorporated herein by reference in its entirety.

Diagnosis of these ALS/CEIND related to TDP-43 can be done by multiple methods known in the art. For example, the amount of TDP-43 in the cytoplasm may be determined by contacting cells with an antibody specific for TDP-43. In some embodiments, TDP-43 is accumulated in the cytoplasm if at least 1%, at least 5%, at least 10%, at least 20%, at least 30%, at least 40%, at least 50%, at least 60%, at least 70%, at least 80%, at least 90%, or more, of total TDP-43 in the cells of the sample is in the cytoplasm of the cells. In other embodiments, TDP-43 is accumulated in the cytoplasm if the amount of TDP-43 in the cytoplasm in the cells of the sample is greater than a sample from a healthy subject. Various biological fluids (e.g., saliva, urine, perspiration, etc.) or biosamples (e.g, buccal swabs, nasal swabs, etc.) known in the art may be used for the diagnosis.

Splicing Factor Heterogeneous Nuclear Ribonucleoprotein L (hnRNP L or HNRNPL)

Provided herein, in some embodiments, is a heterogeneous nuclear RNP (hnRNP or HNRNP) such as hnRNP L. HnRNAs may include mRNA precursors or mature mRNAs associated with specific proteins to form heterogeneous ribonucleoprotein (hnRNP) complexes. HnRNP L is present in the nucleoplasm as part of an HNRP complex. HNRP proteins have also been identified outside of the nucleoplasm. Exchange of hnRNP for mRNA-binding proteins accompanies transport of mRNA from the nucleus to the cytoplasm. Since HNRP proteins have been shown to shuttle between the nucleus and the cytoplasm, they also have cytoplasmic functions.

TDP-43 and hnRNP L are major repressors of toxic cryptic exons. Loss of TDP-43 level or loss of TDP-43 functions (e.g., by aggregation) may be rescued by increased hnRNP L level. By increasing hnRNP L level and/or function, the TDP-43 proteinopathies may be attenuated or alleviated.

Three transcript variants encoding different isoforms have been found for this gene: isoform 1 (NCBI Reference Sequence: NP_001524.2) and isoform 2, which is a fragment of the isoform 1 (with a deletion of position 1 to position 133 amino acid of the isoform 1). Human hnRNP L is encoded by the HNRNPL gene. The information for hnRNP L is at the World Wide Web website of uniprot.org/uniprot/P14866, under the reference number UniProtKB-P14866.

Human hnRNP L Isoform 1 Amino Acid Sequence:

MSRRLLPRAEKRRRRLEQRQQPDEQRRRSGAMVKMAAAGGGGGGGRYYG
GGSEGGRAPKRLKTDNAGDQHGGGGGGGGGAGAAGGGGGGENYDDPHKT
PASPVVHIRGLIDGVVEADLVEALQEFGPISYVVVMPKKRQALVEFEDV
LGACNAVNYAADNQIYIAGHPAFVNYSTSQKISRPGDSDDSRSVNSVLL
FTILNPIYSITTDVLYTICNPCGPVQRIVIFRKNGVQAMVEFDSVQSAQ
RAKASLNGADIYSGCCTLKIEYAKPTRLNVFKNDQDTWDYTNPNLSGQG
DPGSNPNKRQRQPPLLGDHPAEYGGPHGGYHSHYHDEGYGPPPPHYEGR
RMGPPVGGHRRGPSRYGPQYGHPPPPPPPPEYGPHADSPVLMVYGLDQS
KMNCDRVFNVFCLYGNVEKVKFMKSKPGAAMVEMADGYAVDRAITHLNN
NFMFGQKLNVCVSKQPAIMPGQSYGLEDGSCSYKDFSESRNNRFSTPEQ
AAKNRIQHPSNVLHFFNAPLEVTEENFFEICDELGVKRPSSVKVFSGKS
ERSSSGLLEWESKSDALETLGFLNHYQMKNPNGPYPYTLKLCFSTAQHA
S
(SEQ ID NO: 3; NCBI Reference Sequence:
NP_001524.2).

Human hnRNP L Isoform 2 (Deletion of 1-133 AA of Isoform 1) Amino Acid Sequence:

(SEQ ID NO: 4)
MPKKRQALVEFEDVLGACNAVNYAADNQIYIAGHPAFVNYSTSQKISRP
GDSDDSRSVNSVLLFTILNPIYSITTDVLYTICNPCGPVQRIVIFRKNG
VQAMVEFDSVQSAQRAKASLNGADIYSGCCTLKIEYAKPTRLNVFKNDQ
DTWDYTNPNLSGQGDPGSNPNKRQRQPPLLGDHPAEYGGPHGGYHSHYH
DEGYGPPPPHYEGRRMGPPVGGHRRGPSRYGPQYGHPPPPPPPPEYGPH
ADSPVLMVYGLDQSKMNCDRVFNVFCLYGNVEKVKFMKSKPGAAMVEMA
DGYAVDRAITHLNNNFMFGQKLNVCVSKQPAIMPGQSYGLEDGSCSYKD
FSESRNNRFSTPEQAAKNRIQHPSNVLHFFNAPLEVTEENFFEICDELG
VKRPSSVKVFSGKSERSSSGLLEWESKSDALETLGFLNHYQMKNPNGPY
PYTLKLCFSTAQHAS.

In some embodiments, the hnRNP L described herein comprises an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 3 or 4. In some embodiments, the hnRNP L described herein consists of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 3 or 4.

A sequence logo representing the binding motif appears in FIG. 20. This logo differs slightly from that in Hui, et al. EMBO J. 2005 Jun. 1; 24(11):1988-1998

A binding motif may be included in an hnRNP L protein.

In some embodiments, the hnRNP L binding motif or binding site, described herein, comprises a consensus nucleic acid sequence shown in the sequence plot in FIG. 20. For example, such hnRNP L binding motif or binding site may comprise an 8-mer nucleic acid sequence of ACACACAC, ACATACAC, ATACACAC, ATATACAC, ACGCACAC, ACGTACAC, ATGCACAC, ATGTACAC, or X₁X₂X₃X₄X₅CAX₆, wherein X₁ is A, C, or T, X₂ is C or T, X₃ is A or G, X₄ is C or T, X₅ is A, G, or T, and X₆ is C or T.

Any 8-character sequence (8-mer) can be given a log-likelihood score comparing the probability that the sequence is an example of an hnRNP L binding site to the probability that the sequence arose simply by chance. These log-scaled scores are summed across all positions of the motif, corresponding to the products of their probabilities. An 8-mer having a log-likelihood score of at least 10 means that, across the 8 positions of the motif, the probability of seeing the observed 8-mer is at least 1024 (or 210) times more likely if it is an example of the binding motif than if it were an example of random sequence where each nucleotide is equally likely to occur. Similarly, a score of at least 6 means that the sequence is at least 64 (or 26) times more likely to be an example of the motif than not.

### Sortilin 1 (SORT1)

Provided herein, in some embodiments, is sortilin 1 (SORT1). SORT1 encodes a member of the VPS10-related sortilin family of proteins. The encoded preproprotein is proteolytically processed by furin to generate the mature receptor. This receptor plays a role in the trafficking of different proteins to either the cell surface, or subcellular compartments such as lysosomes and endosomes. Expression levels of this gene may influence the risk of myocardial infarction in human patients. Alternative splicing results in multiple transcript variants.

Diseases associated with SORT1 include low density lipoprotein cholesterol level quantitative traitlocus 6 (LDLCQ6) and myocardial infarction. Among its related pathways are lysosome and clathrin derived vesicle budding. Gene Ontology (GO) annotations related to this gene include enzyme binding and nerve growth factor receptor activity. An important paralog of this gene is SORL1.

### Human SORT1 Isoform 1 Amino Acid Sequence:

MERPWGAADGLSRWPHGLGLLLLLQLLPPSTLSQDRLDAPPPPAAPLPR
WSGPIGVSWGLRAAAAGGAFPRGGRWRRSAPGEDEECGRVRDFVAKLAN
NTHQHVFDDLRGSVSLSWVGDSTGVILVLTTFHVPLVIMTFGQSKLYRS
EDYGKNFKDITDLINNTFIRTEFGMAIGPENSGKVVLTAEVSGGSRGGR
IFRSSDFAKNFVQTDLPFHPLTQMMYSPQNSDYLLALSTENGLWVSKNF
GGKWEEIHKAVCLAKWGSDNTIFFTTYANGSCKADLGALELWRTSDLGK
SFKTIGVKIYSFGLGGRFLFASVMADKDTTRRIHVSTDQGDTWSMAQLP
SVGQEQFYSILAANDDMVFMHVDEPGDTGFGTIFTSDDRGIVYSKSLDR
HLYTTTGGETDFTNVTSLRGVYITSVLSEDNSIQTMITFDQGGRWTHLR
KPENSECDATAKNKNECSLHIHASYSISQKLNVPMAPLSEPNAVGIVIA
HGSVGDAISVMVPDVYISDDGGYSWTKMLEGPHYYTILDSGGIIVAIEH
SSRPINVIKFSTDEGQCWQTYTFTRDPIYFTGLASEPGARSMNISIWGF
TESFLTSQWVSYTIDFKDILERNCEEKDYTIWLAHSTDPEDYEDGCILG
YKEQFLRLRKSSVCQNGRDYVVTKQPSICLCSLEDFLCDFGYYRPENDS
KCVEQPELKGHDLEFCLYGREEHLTTNGYRKIPGDKCQGGVNPVREVKD
LKKKCTSNFLSPEKQNSKSNSVPIILAIVGLMLVTVVAGVLIVKKYVCG
GRFLVHRYSVLQQHAEANGVDGVDALDTASHTNKSGYHDDSDEDLLE
(SEQ ID NO: 14: NCBI Reference Sequence:
NP_002950.3).

### Alternative Human SORT1 Isoform 2 Amino Acid Sequence:

MTFGQSKLYRSEDYGKNFKDITDLINNTFIRTEFGMAIGPENSGKVVLT
AEVSGGSRGGRIFRSSDFAKNFVQTDLPFHPLTQMMYSPQNSDYLLALS
TENGLWVSKNFGGKWEEIHKAVCLAKWGSDNTIFFTTYANGSCTDLGAL
ELWRTSDLGKSFKTIGVKIYSFGLGGRFLFASVMADKDTTRRIHVSTDQ
GDTWSMAQLPSVGQEQFYSILAANDDMVFMHVDEPGDTGFGTIFTSDDR
GIVYSKSLDRHLYTTTGGETDFTNVTSLRGVYITSVLSEDNSIQTMITF
DQGGRWTHLRKPENSECDATAKNKNECSLHIHASYSISQKLNVPMAPLS
EPNAVGIVIAHGSVGDAISVMVPDVYISDDGGYSWTKMLEGPHYYTILD
SGGIIVAIEHSSRPINVIKFSTDEGQCWQTYTFTRDPIYFTGLASEPGA
RSMNISIWGFTESFLTSQWVSYTIDFKDILERNCEEKDYTIWLAHSTDP
EDYEDGCILGYKEQFLRLRKSSVCQNGRDYVVTKQPSICLCSLEDFLCD
FGYYRPENDSKCVEQPELKGHDLEFCLYGREEHLTTNGYRKIPGDKCQG
GVNPVREVKDLKKKCTSNFLSPEKQNSKSNSVPIILAIVGLMLVTVVAG
VLIVKKYVCGGRFLVHRYSVLQQHAEANGVDGVDALDTASHTNKSGYHD
DSDEDLLE
(SEQ ID NO: 15; NCBI Reference Sequence:
NP_001192157.1).

In some embodiments, the SORT1 described herein comprises an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 14 or 15. In some embodiments, the SORT1 described herein consists of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 14 or 15.

Other isoforms or natural variants of SORT1 can be found in the UNIPROT database (entry number UniProtKB-Q99523) at World Wide Web website of uniprot.org/uniprot/Q99523. Exemplary isoforms include, e.g., UNIPROT database entry numbers A0A0J9YVU2, A0A0J9YY30, A0A0J9YX61, and A0A0J9YVX1.

### G-Protein-Signaling Modulator 2 (GPSM2)

Provided herein, in some embodiments, is G-protein-signaling modulator 2 (GPSM2), also called LGN for its 10 Leucine-Glycine-Asparagine repeats. GPSM2 is a protein that in humans is encoded by the GPSM2 gene. Heterotrimeric G proteins transduce extracellular signals received by cell surface receptors into integrated cellular responses. GPSM2 belongs to a group of proteins that modulate activation of G proteins. It plays an important role in mitotic spindle pole organization via its interaction with NUMA1. It is required for cortical dynein-dynactin complex recruitment during metaphase. It further plays a role in metaphase spindle orientation and asymmetric cell divisions. GPSM2 has a guanine nucleotide dissociation inhibitor (GDI) activity.

Mutations in GPSM2 have been identified in people with profound congenital non-syndromic deafness designated as DFNB82. Subsequent brain imaging of these individuals has revealed frontal polymicrogyria, abnormal corpus callosum, and gray matter heterotopia, consistent with a diagnosis of Chudley-Mccullough syndrome (CMCS). CMCS is an autosomal recessive neurologic disorder characterized by early-onset sensorineural deafness and specific brain anomalies on MRI, including hypoplasia of the corpus callosum, enlarged cysterna magna with mild focal cerebellar dysplasia, and nodular heterotopia. Some patients have hydrocephalus. Psychomotor development is normal.

### Human GPSM2 Isoform 1 Amino Acid Sequence:

(SEQ ID NO: 16; NCBI Reference Sequence:
NP_001307967.1)
MEENLISMREDHSFHVRYRMEASCLELALEGERLCKSGDCRAGVSFFEAA
VQVGTEDLKTLSAIYSQLGNAYFYLHDYAKALEYHHHDLTLARTIGDQLG
EAKASGNLGNTLKVLGNFDEAIVCCQRHLDISRELNDKVGEARALYNLGN
VYHAKGKSFGCPGPQDVGEFPEEVRDALQAAVDFYEENLSLVTALGDRAA
QGRAFGNLGNTHYLLGNFRDAVIAHEQRLLIAKEFGDKAAERRAYSNLGN
AYIFLGEFETASEYYKKTLLLARQLKDRAVEAQSCYSLGNTYTLLQDYEK
AIDYHLKHLAIAQELNDRIGEGRACWSLGNAYTALGNHDQAMHFAEKHLE
ISREVGDKSGELTARLNLSDLQMVLGLSYSTNNSIMSENTEIDSSLNGVR
PKLGRRHSMENMELMKLTPEKVQNWNSEILAKQKPLIAKPSAKLLFVNRL
KGKKYKTNSSTKVLQDASNSIDHRIPNSQRKISADTIGDEGFFDLLSRFQ
SNRMDDQRCCLQEKNCHTASTTTSSTPPKMMLKTSSVPVVSPNTDEFLDL
LASSQSRRLDDQRASFSNLPGLRLTQNSQSVLSHLMTNDNKEADEDFFDI
LVKCQGSRLDDQRCAPPPATTKGPTVPDEDFFSLILRSQGKRMDEQRVLL
QRDQNRDTDFGLKDFLQNNALLEFKNSGKKSADH.

In some embodiments, the GPSM2 described herein comprises an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 16. In some embodiments, the GPSM2 described herein consists of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 16.

Other isoforms or natural variants of GPSM2 can be found in the UNIPROT database (entry number UniProtKB-P81274) at World Wide Web website of uniprot.org/uniprot/P81274. Exemplary isoforms include, e.g., UNIPROT database entry numbers Q5T1N9, H0Y4A4, A0A2R8Y6E3, B0QZD0, B0QZC9, A0A2R8YCX1, A0A2R8Y896, and A0A2R8Y673.

### Cysteine Protease ATG4B

Provided herein, in some embodiments, is a cysteine protease such as ATG4B. ATG4B is a cysteine protease required for the cytoplasm to vacuole transport (Cvt) and autophagy. ATG4B cleaves the C-terminal amino acid of ATG8 family proteins MAP1LC3, GABARAPL1, GABARAPL2 and GABARAP, to reveal a C-terminal glycine. Autophagy is the process by which endogenous proteins and damaged organelles are destroyed intracellularly. Autophagy is postulated to be essential for cell homeostasis and cell remodeling during differentiation, metamorphosis, non-apoptotic cell death, and aging. Reduced levels of autophagy have been described in some malignant tumors, and a role for autophagy in controlling the unregulated cell growth linked to cancer has been proposed. This gene encodes a member of the autophagin protein family. The encoded protein is also designated as a member of the C-54 family of cysteine proteases. Alternate transcriptional splice variants, encoding different isoforms, have been characterized for ATG4B. One main function of ATG4 is to cleave the pre-protein of ATG8, leading to the non-lipidated soluble (−I) form which can be processed further by ATG3, ATG7, ATG5-12 into the lipidated form (−II) anchored to the autophagic membrane.

### Human ATG4B Isoform 1 Amino Acid Sequence:

(SEQ ID NO: 17; NCBI Reference Sequence:
NP_037457.3)
MDAATLTYDTLRFAEFEDFPETSEPVWILGRKYSIFTEKDEILSDVASRL
WFTYRKNFPAIGGTGPTSDTGWGCMLRCGQMIFAQALVCRHLGRDWRWTQ
RKRQPDSYFSVLNAFIDRKDSYYSIHQIAQMGVGEGKSIGQWYGPNTVAQ
VLKKLAVFDTWSSLAVHIAMDNTVVMEEIRRLCRTSVPCAGATAFPADSD
RHCNGFPAGAEVTNRPSPWRPLVLLIPLRLGLTDINEAYVETLKHCFMMP
QSLGVIGGKPNSAHYFIGYVGEELIYLDPHTTQPAVEPTDGCFIPDESFH
CQHPPCRMSIAELDPSIAVGFFCKTEDDFNDWCQQVKKLSLLGGALPMFE
LVELQPSHLACPDVLNLSLDSSDVERLERFFDSEDEDFEILSL.

### Human ATG4B Isoform 2 Amino Acid Sequence:

(SEQ ID NO: 18; NCBI Reference Sequence:
BAC86110.1)
MAHSVPSDSRTSRRPTTRPHAARGAPRGSRRPGRTPKWRLPRISARAPYR
LRRLRRHTYWPPRRPVAASRCWPVGATPLGSVGGRTGKMDAATLTYDTLR
FAEFEDFPETSEPVWILGRKYSIFTEKDEILSDVASRLWFTYRKNFPAIG
GTGPTSDTGWGCMLRCGQMIFAQALVCRHLGRDWRWTQRKRQPDSYFSVL
NAFIDRKDSYYSIHQIAQMGVGEGKSIGQWYGPNTVAQVLKKLAVFDTWS
SLAVHIAMDNTVVMEEIRRLCRTSVPCAGATAFPADSDRHCNGFPAGAEV
TNRPSPWRPLVLLIPLRLGLTDINEAYVETLKHCFMMPQSLGVIGGKPNS
AHYFIGYVGEELIYLDPHTTQPAVEPTDGCFIPDESFHCQHPPCRMSIAE
LDPSIAVGFFCKTEDDFNDWCQQVKKLSLLGGALPMFELVELQPSHLACP
DVLNLSLGESCQVQILLM.

### Human ATG4B Isoform 3 Amino Acid Sequence:

(SEQ ID NO: 19; NCBI Reference Sequence:
BAB55042.1)
MLRCGQMIFAQALVCRHLGRDWRWTQRKRQPDSYFSVLNAFIDRKDSYYS
IHQIAQMGVGEGKSIGQWYGPNTVAQVLKKLAVFDTWSSLAVHIAMDNTV
VMEEIRRLCRTSVPCAGATAFPADSDRHCNGFPAGAEVTNRPSPWRPLVL
LIPLRLGLTDINEAYVETLKHCFMMPQSLGVIGGKPNSAHYFIGYVGEEL
IYLDPHTTQPAVEPTDGCFIPDESFHCQHPPCRMSIAELDPSIAVGKQGR
LVRSLIPWAPRPSSWCAAVLGAAVVMCGTP.

### Human ATG4B Isoform 4 Amino Acid Sequence:

(SEQ ID NO: 20; NCBI Reference Sequence:
XP_005247050.2)
MLRCGQMIFAQALVCRHLGRDWRWTQRKRQPDSYFSVLNAFIDRKDSYYS
IHQIAQMGVGEGKSIGQWYGPNTVAQVLKKLAVFDTWSSLAVHIAMDNTV
VMEEIRRLCRTSVPCAGATAFPADSDRHCNGFPAGAEVTNRPSPWRPLVL
LIPLRLGLTDINEAYVETLKHCFMMPQSLGVIGGKPNSAHYFIGYVGEEL
IYLDPHTTQPAVEPTDGCFIPDESFHCQHPPCRMSIAELDPSIAVGFFCK
TEDDFNDWCQQVKKLSLLGGALPMFELVELQPSHLACPDVLNLSLGESCQ
VQVGSLGDSSDVERLERFFDSEDEDFEILSL.

### Human ATG4B Isoform 5 Amino Acid Sequence:

(SEQ ID NO: 21; NCBI Reference Sequence:
BAC86110.1)
MDAATLTYDTLRFAEFEDFPETSEPVWILGRKYSIFTEKDEILSDVASRL
WFTYRKNFPAIGGTGPTSDTGWGCMLRCGQMIFAQALVCRHLGRDWRWTQ
RKRQPDSYFSVLNAFIDRKDSYYSIHQIAQMGVGEGKSIGQWYGPNTVAQ
VLKKLAVFDTWSSLAVHIAMDNTVVMEEIRRLCRTSVPCAGATAFPADSD
RHCNGFPAGAEVTNRPSPWRPLVLLIPLRLGLTDINEAYVETLKHCFMMP
QSLGVIGGKPNSAHYFIGYVGEELIYLDPHTTQPAVEPTDGCFIPDESFH
CQHPPCRMSIAELDPSIAVGFFCKTEDDFNDWCQQVKKLS
LLGGALPMFELVELQPSHLACPDVLNLSLGESCQVQILLM.

In some embodiments, the ATG4B protein described herein comprises an amino acid sequence having at least 50%, 60%, 65%, 70%, 75%, 80%, 85%, 90%, 91%, 92%, 93%, 94%, 95%, 96%, 97%, 98%, 99%, or 100% sequence identity to any one of SEQ ID NOs: 17-21. In some embodiments, the ATG4B protein described herein consists of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to any one of SEQ ID NOs: 17-21.

Other isoforms or natural variants of ATG4B can be found in the UNIPROT database (entry number UniProtKB-Q9Y4P1) at World Wide Web website of uniprot.org/uniprot/Q9Y4P1. Exemplary isoforms include, e.g., UNIPROT database entry numbers C9JIK8, C9J1C1, H0Y2Y0, and F2Z2K8.

### Unc-13 Homolog a (UNC13a)

Provided herein, in some embodiments, is a Unc-13 homolog (UNC13) protein. UNC13 proteins may bind to phorbol esters and diacylglycerol and play important roles in neurotransmitter release at synapses. In some embodiments, the UNC13 is Unc-13 Homolog A (UNC13A). Single nucleotide polymorphisms in the UNC13A gene may be associated with sporadic amyotrophic lateral sclerosis. Diseases associated with UNC13A may include Amyotrophic Lateral Sclerosis 1 and Febrile Seizures. Among its related pathways are synaptic vesicle pathway and 16p11.2 proximal deletion syndrome. Gene Ontology (GO) annotations related to UNC13A include obsolete protein N-terminus binding and diacylglycerol binding. A paralog of UNC13A is UNC13B. UNC13A may play a role in vesicle maturation during exocytosis as a target of the diacylglycerol second messenger pathway. UNC13A may be involved in neurotransmitter release by acting in synaptic vesicle priming prior to vesicle fusion and participates in the activity-dependent refilling of readily releasable vesicle pool (RRP). UNC13A may be essential for synaptic vesicle maturation in excitatory/glutamatergic, and not in inhibitory/GABA-mediated synapses. UNC13A may facilitate neuronal dense core vesicles fusion as well as controls the location and efficiency of their synaptic release. UNC13A may be involved in secretory granule priming in insulin secretion. UNC13A may play a role in dendrite formation by melanocytes.

### Human UNC13A Sequence (UniProt Q9UPW8):

(SEQ ID NO: 103)
MSLLCVGVKKAKFDGAQEKFNTYVTLKVQNVKSTTIAVRGSQPSWEQDFM
FEINRLDLGLTVEVWNKGLIWDTMVGTVWIPLRTIRQSNEEGPGEWLTLD
SQVIMADSEICGTKDPTFHRILLDTRFELPLDIPEEEARYWAKKLEQLNA
MRDQDEYSFQDEQDKPLPVPSNQCCNWNYFGWGEQHNDDPDSAVDDRDSD
YRSETSNSIPPPYYTTSQPNASVHQYSVRPPPLGSRESYSDSMHSYEEFS
EPQALSPTGSSRYASSGELSQGSSQLSEDFDPDEHSLQGSDMEDERDRDS
YHSCHSSVSYHKDSPRWDQDEEELEEDLEDFLEEEELPEDEEELEEEEEE
VPDDLGSYAQREDVAVAEPKDFKRISLPPAAPGKEDKAPVAPTEAPDMAK
VAPKPATPDKVPAAEQIPEAEPPKDEESFRPREDEEGQEGQDSMSRAKAN
WLRAFNKVRMQLQEARGEGEMSKSLWFKGGPGGGLIIIDSMPDIRKRKPI
PLVSDLAMSLVQSRKAGITSALASSTLNNEELKNHVYKKTLQALIYPISC
TTPHNFEVWTATTPTYCYECEGLLWGIARQGMRCTECGVKCHEKCQDLLN
ADCLQRAAEKSSKHGAEDRTQNIIMVLKDRMKIRERNKPEIFELIQEIFA
VTKTAHTQQMKAVKQSVLDGTSKWSAKISITVVCAQGLQAKDKTGSSDPY
VTVQVGKTKKRTKTIYGNLNPVWEENFHFECHNSSDRIKVRVWDEDDDIK
SRVKQRFKRESDDFLGQTIIEVRTLSGEMDVWYNLDKRTDKSAVSGAIRL
HISVEIKGEEKVAPYHVQYTCLHENLFHFVTDVQNNGVVKIPDAKGDDAW
KVYYDETAQEIVDEFAMRYGVESIYQAMTHFACLSSKYMCPGVPAVMSTL
LANINAYYAHTTASTNVSASDRFAASNFGKERFVKLLDQLHNSLRIDLSM
YRNNFPASSPERLQDLKSTVDLLTSITFFRMKVQELQSPPRASQVVKDCV
KACLNSTYEYIFNNCHELYSREYQTDPAKKGEVLPEEQGPSIKNLDFWSK
LITLIVSIIEEDKNSYTPCLNQFPQELNVGKISAEVMWNLFAQDMKYAME
EHDKHRLCKSADYMNLHFKVKWLYNEYVTELPAFKDRVPEYPAWFEPFVI
QWLDENEEVSRDFLHGALERDKKDGFQQTSEHALFSCSVVDVFSQLNQSF
EIIKKLECPDPQIVGHYMRRFAKTISNVLLQYADIISKDFASYCSKEKEK
VPCILMNNTQQLRVQLEKMFEAMGGKELDAEASDILKELQVKLNNVLDEL
SRVFATSFQPHIEECVKQMGDILSQVKGTGNVPASACSSVAQDADNVLQP
IMDLLDSNLTLFAKICEKTVLKRVLKELWKLVMNTMEKTIVLPPLTDQTM
IGNLLRKHGKGLEKGRVKLPSHSDGTQMIFNAAKELGQLSKLKDHMVREE
AKSLTPKQCAVVELALDTIKQYFHAGGVGLKKTFLEKSPDLQSLRYALSL
YTQATDLLIKTFVQTQSAQGLGVEDPVGEVSVHVELFTHPGTGEHKVTVK
VVAANDLKWQTSGIFRPFIEVNIIGPQLSDKKRKFATKSKNNSWAPKYNE
SFQFTLSADAGPECYELQVCVKDYCFAREDRTVGLAVLQLRELAQRGSAA
CWLPLGRRIHMDDTGLTVLRILSQRSNDEVAKEFVKLKSDTRSAEEGGAA
PAP.

In some embodiments, the UNC13A protein described herein comprises an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 103. In some embodiments, the UNC13A protein described herein consists of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 103.

### Human UNC13B Sequence (UniProt O14795):

(SEQ ID NO: 104)
MSLLCVRVKRAKFQGSPDKFNTYVTLKVQNVKSTTVAVRGDQPSWEQDFM
FEISRLDLGLSVEVWNKGLIWDTMVGTVWIALKTIRQSDEEGPGEWSTLE
AETLMKDDEICGTRNPTPHKILLDTRFELPFDIPEEEARYWTYKWEQINA
LGADNEYSSQEESQRKPLPTAAAQCSFEDPDSAVDDRDSDYRSETSNSFP
PPYHTASQPNASVHQFPVPVRSPQQLLLQGSSRDSCNDSMQSYDLDYPER
RAISPTSSSRYGSSCNVSQGSSQLSELDQYHEQDDDHRETDSIHSCHSSH
SLSRDGQAGFGEQEKPLEVTGQAEKEAACEPKEMKEDATTHPPPDLVLQK
DHFLGPQESFPEENASSPFTQARAHWIRAVTKVRLQLQEIPDDGDPSLPQ
WLPEGPAGGLYGIDSMPDLRRKKPLPLVSDLSLVQSRKAGITSAMATRTS
LKDEELKSHVYKKTLQALIYPISCTTPHNFEVWTATTPTYCYECEGLLWG
IARQGMRCSECGVKCHEKCQDLLNADCLQRAAEKSCKHGAEDRTQNIIMA
MKDRMKIRERNKPEIFEVIRDVFTVNKAAHVQQMKTVKQSVLDGTSKWSA
KITITVVCAQGLQAKDKTGSSDPYVTVQVSKTKKRTKTIFGNLNPVWEEK
FHFECHNSSDRIKVRVWDEDDDIKSRVKQRLKRESDDFLGQTIIEVRTLS
GEMDVWYNLEKRTDKSAVSGAIRLQISVEIKGEEKVAPYHVQYTCLHENL
FHYLTDIQGSGGVRIPEARGDDAWKVYFDETAQEIVDEFAMRYGIESIYQ
AMTHFACLSSKYMCPGVPAVMSTLLANINAYYAHTTASTNVSASDRFAAS
NFGKERFVKLLDQLHNSLRIDLSTYRNNFPAGSPERLQDLKSTVDLLTSI
TFFRMKVQELQSPPRASQVVKDCVKACLNSTYEYIFNNCHDLYSRQYQLK
QELPPEEQGPSIRNLDFWPKLITLIVSIIEEDKNSYTPVLNQFPQELNVG
KVSAEVMWHLFAQDMKYALEEHEKDHLCKSADYMNLHFKVKWLHNEYVRD
LPVLQGQVPEYPAWFEQFVLQWLDENEDVSLEFLRGALERDKKDGFQQTS
EHALFSCSVVDVFTQLNQSFEIIRKLECPDPSILAHYMRRFAKTIGKVLM
QYADILSKDFPAYCTKEKLPCILMNNVQQLRVQLEKMFEAMGGKELDLEA
ADSLKELQVKLNTVLDELSMVFGNSFQVRIDECVRQMADILGQVRGTGNA
SPDARASAAQDADSVLRPLMDFLDGNLTLFATVCEKTVLKRVLKELWRVV
MNTMERMIVLPPLTDQTGTQLIFTAAKELSHLSKLKDHMVREETRNLTPK
QCAVLDLALDTIKQYFHAGGNGLKKTFLEKSPDLQSLRYALSLYTQTTDT
LIKTFVRSQTTQGSGVDDPVGEVSIQVDLFTHPGTGEHKVTVKVVAANDL
KWQTAGMFRPFVEVTMVGPHQSDKKRKFTTKSKSNNWAPKYNETFHFLLG
NEEGPESYELQICVKDYCFAREDRVLGLAVMPLRDVTAKGSCACWCPLGR
KIHMDETGLTILRILSQRSNDEVAREFVKLKSESRSTEEGS.

In some embodiments, the UNC13B protein described herein comprises an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 104. In some embodiments, the UNC13B protein described herein consists of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 104.

### Stathmin-2 (STMN2)

Provided herein, in some embodiments, is a Stathmin-2 (STMN2, a.k.a., SCG10), a protein that in humans is encoded by the STMN2 gene. The information for TDP-43 is at the World Wide Web website of uniprot.org/uniprot/Q93045, under the reference number UniProtKB-Q93045. Human STMN2 proteins include an isoform 1 having a longer transcript and an isoform 2, which lacks an alternate exon resulting in a frameshift in the 3′ coding region, due to alternative splicing. The encoded isoform 2 is shorter and has a distinct C-terminus, compared to isoform 1.

### Human STMN2, Isoform 1, Amino Acid Sequence:

MAKTAMAYKEKMKELSMLSLICSCFYPEPRNINIYTYDDMEVKQINKRAS
GQAFELILKPPSPISEAPRTLASPKKKDLSLEEIQKKLEAAEERRKSQEA
QVLKQLAEKREHEREVLQKALEENNNFSKMAEEKLILKMEQIKENREANL
AAIIERLQEKLVKFISSELKESIESQFLELQREGEKQ
(SEQ ID NO: 22; NCBI Reference Sequence:
NP_001186143.1).

### Human STMN2, Isoform 2, Amino Acid Sequence:

MAKTAMAYKEKMKELSMLSLICSCFYPEPRNINIYTYDDMEVKQINKRAS
GQAFELILKPPSPISEAPRTLASPKKKDLSLEEIQKKLEAAEERRKSQEA
QVLKQLAEKREHEREVLQKALEENNNFSKMAEEKLILKMEQIKENREANL
AAIIERLQEKERHAAEVRRNKELQVELSG
(SEQ ID NO: 23; NCBI Reference Sequence:
NP_008960.2).

STMN2 is a regulator of microtubule stability and is necessary for normal neurite growth. It has an enriched neuronal expression. STMN2 is altered in postmortem ALS spinal cord. The expression of STMN2 RNA was significantly reduced after TDP-43 knockdown, while the closely related STMN1 RNA remained unchanged (Klim et al. Nature Neuroscience 2019; 22:167-179). TDP-43 regulates STMN2 levels. Either TDP-43 depletion or mislocalization and insolubility (e.g., in ALS patients) may promote cryptic splicing of STMN2, resulting in a premature stop codon within the cryptic splice-form and the following reduced levels of STMN2 RNA.

In some embodiments, the STMN2 protein described herein comprises an amino acid sequence having at least 50%, 60%, 65%, 70%, 75%, 80%, 85%, 90%, 91%, 92%, 93%, 94%, 95%, 96%, 97%, 98%, 99%, or 100% sequence identity to any one of SEQ ID NO: 22 or 23. In some embodiments, the STMN2 protein described herein consists of an amino acid sequence having at least 50%, 60%, 65%, 70%, 75%, 80%, 85%, 90%, 91%, 92%, 93%, 94%, 95%, 96%, 97%, 98%, 99%, or 100% sequence identity to any one of SEQ ID NO: 22 or 23.

Other isoforms or natural variants of STMN2 can be found in the UNIPROT database (entry number UniProtKB-Q93045) at World Wide Web website of uniprot.org/uniprot/Q93045.

### Additional Genes

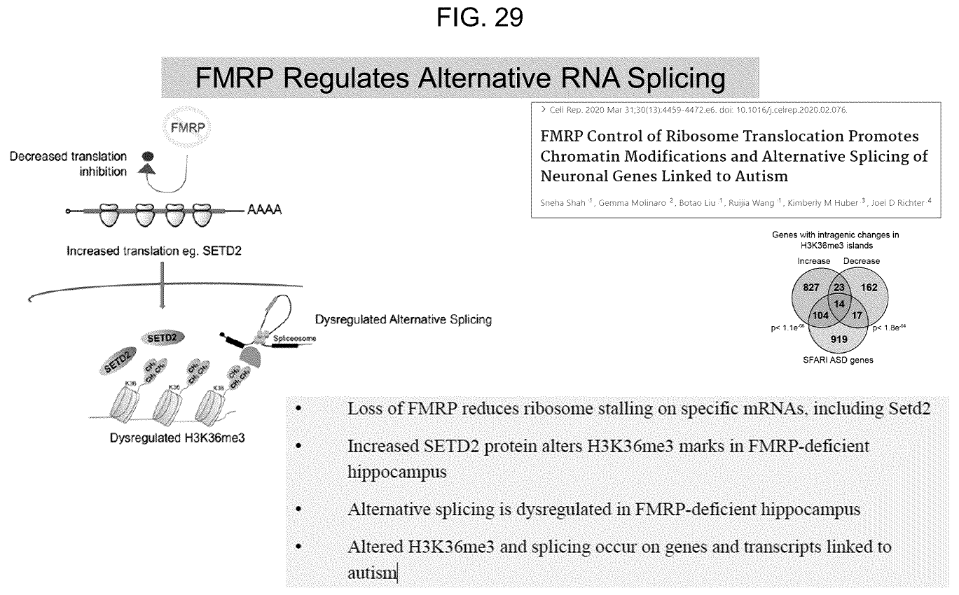

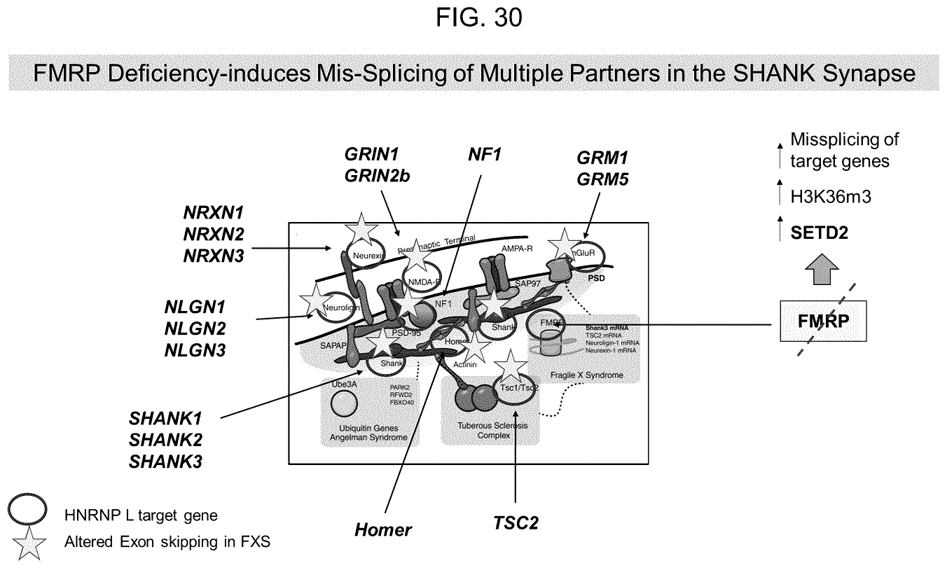

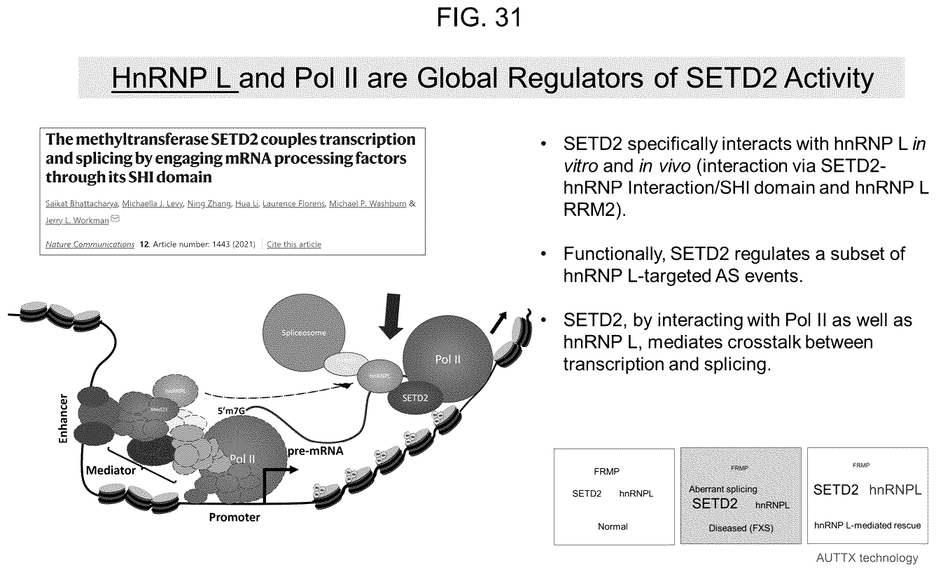

Some additional genes and aspects related to splicing defects and other disorders are shown in FIG. 29-33. In some embodiments, a subject (e.g., a subject to be treated herein) has a mutation or defect in a gene in FIG. 29-31.

Neurological diseases induced by RNA Biding Proteins (RBP) may be targeted by a compound or composition herein. In some aspects, a mutation in a gene encoding RBP RBFOX1 may results in any of several neurologic disorders, including mental retardation, epilepsy or autism. Any of these disorders may be treated herein. Neurexins, neuroligins and shank genes may contain Rbfox sites near alternatively spliced exons. Neurexins, Neuroligins and Shank genes are also candidate targets of hnRNP L, and may be affected by hnRNP L upregulation. HnRNP L and RBFOX1 interact, and so RBFOX1 may be affected by hnRNP L upregulation. RBFOX1-induced neurological disease/ASD may be targeted by a treatment, compound, or composition herein.

### Diseases and Disorders

Described herein, in some embodiments, are diseases or disorders. A subject may have a disease or disorder, and be administered a composition herein. The disease or disorder may be treated by a composition herein. Any aspect of a disease or disorder provided herein may be improved, reduced, prevented, or slowed by treatment.

Diseases and disorders described herein include neurological diseases related to reduced TDP-43 levels, or associated with a splicing defect caused by TDP-43 proteinopathies, comprises at least one selected from the group consisting of: Amyotrophic Lateral Sclerosis (ALS), Frontotemporal Dementia (FTD), Pick's disease, hippocampal sclerosis, corticobasal degeneration, argyrophilic grain disease, Alzheimer's disease and Huntington disease. In some embodiments, the diseases and disorders comprise a disease or disorder associated to decreased expression levels and/or stability of hnRNP L and/or aggregation and/or mislocalization of hnRNP L (e.g., due to a mutation or deletion in the HNRNPL gene). In some embodiments, the diseases and disorders comprise a disease or disorder associated to decreased expression levels of and/or aggregation and/or mislocalization of TDP-43 (e.g., due to a mutation or deletion in the TARDBP gene). Some exemplary ALS/CEIND in this application include Frontotemporal lobar degeneration (FTLD), Frontotemporal Dementia (FTD), Autism Spectrum Disorder (ASD), myotonic dystrophy type 1 (DM1) or type 2 (DM2), Alzheimer's disease, Lewy body dementia (LBD), Pick's disease, Hippocampal sclerosis, Corticobasal degeneration, Huntington disease, Parkinson's disease, Argyrophilic grain disease, Chronic traumatic encephalopathy (CTE), Perry syndrome, Alexander disease, Multisystem proteinopathy (MSP), intellectual disability, ADHD, dyslexia, epilepsy, bipolar disorder, depression and schizophrenia, parkinsonism-dementia complex of Guam, dementia pugilistica, diffuse neurofibrillary tangles with calcification, Down's syndrome, familial British dementia, familial Danish dementia, frontal lobe dementia, Gerstmann-Straussler-Scheinker disease, globular glial tauopathies, white matter tauopathy with globular glial inclusions, Guadeloupian parkinsonism with dementia, Guadeloupian progressive supranuclear palsy, multiple system atrophy, neurodegeneration with brain iron accumulation, Hallervorden-Spatz disease, pantothenate kinase-associated neurodegeneration, neurofibrillary tangle-predominant dementia, Niemann-Pick disease, prosencephalitic parkinsonism, prion protein cerebral amyloid angiopathy, progressive supranuclear palsy, SLCI A6-related mental retardation, subacute sclerosing panencephalitis, Alzheimer's disease, chronic traumaticencephalopathy (CTE), limbic predominant, age-related TDP-43 encephalopathy (LATE), and inclusion body myopathy.

Various biological fluids (e.g., saliva, urine, perspiration, etc.) or biosamples (e.g. buccal swabs, nasal swabs, etc.) known in the art may be used for diagnosis and/or monitoring of the diseases or disorders described herein and/or for assessing pharmaceutical effects of the compositions described herein to these diseases or disorders.

### Amyotrophic Lateral Sclerosis (ALS)

In some embodiments, a disease or disorder includes Amyotrophic Lateral Sclerosis (ALS), also called Lou Gehrig's disease, which is a progressive nervous system disease that affects nerve cells in the brain and spinal cord, causing loss of muscle control. ALS often begins with muscle twitching and weakness in a limb, or slurred speech. Eventually, ALS affects control of the muscles needed to move, speak, cat and breathe. There is no cure for this fatal disease. Signs and symptoms of ALS vary greatly from person to person, depending on which neurons are affected. Signs and symptoms may include:
Difficulty walking or doing normal daily activities
Tripping and falling
Weakness in leg, feet or ankles
Hand weakness or clumsiness
Slurred speech or trouble swallowing
Muscle cramps and twitching in arms, shoulders and tongue
Inappropriate crying, laughing or yawning
Cognitive and behavioral changes

ALS often starts in the hands, feet or limbs, and then spreads to other parts of body. As the disease advances and nerve cells are destroyed, the muscles get weaker. This eventually affects chewing, swallowing, speaking and breathing. There's generally no pain in the early stages of ALS, and pain is uncommon in the later stages. ALS doesn't usually affect bladder control or senses.

ALS affects the nerve cells that control voluntary muscle movements such as walking and talking (motor neurons). ALS causes the motor neurons to gradually deteriorate, and then die. Motor neurons extend from the brain to the spinal cord to muscles throughout the body. When motor neurons are damaged, they stop sending messages to the muscles, so the muscles can't function. As the disease progresses, ALS causes complications, such as: breathing problems, speaking problems, eating problems, and dementia.

ALS is inherited in 5% to 10% of people. For the rest, the cause isn't known.

### Established Risk Factors for ALS Include:

Heredity. Five to 10 percent of the people with ALS inherited it (familial ALS). In most people with familial ALS, their children have a 50-50 chance of developing the disease.
Age. ALS risk increases with age, and is most common between the ages of 40 and the mid-60s.
Sex. Before the age of 65, slightly more men than women develop ALS. This sex difference disappears after age 70.
Genetics. Some studies examining the entire human genome found many similarities in the genetic variations of people with familial ALS and some people with noninherited ALS. These genetic variations might make people more susceptible to ALS.
   Some Tests that are Used to Diagnose ALS Include:
Electromyogram (EMG) inserts a thin electrode into the muscle to detect electrical activity and see how well the muscles are working.
Nerve conduction velocity (NCV) passes a small shock through a nerve to measure the speed of nerve signals.
Spinal tap inserts a needle into the spinal canal and removes fluid (cerebrospinal fluid) for testing.
X-rays, CT scans and MRI create images of the brain and spinal cord to look for any abnormalities.
Blood and urine tests look for abnormal levels of certain substances in the blood and urine.
Muscle or nerve biopsy removes a small sample of tissue, which is sent to a lab for testing.

### Frontotemporal Dementia (FTD)

In some embodiments, a disease or disorder includes a Frontotemporal Dementia (FTD), an umbrella term for a group of uncommon brain disorders that primarily affect the frontal and temporal lobes of the brain. These areas of the brain are generally associated with personality, behavior and language. In frontotemporal dementia, portions of these lobes shrink (atrophy). Signs and symptoms vary, depending on which part of the brain is affected. Some people with frontotemporal dementia have dramatic changes in their personality and become socially inappropriate, impulsive or emotionally indifferent, while others lose the ability to use language properly.

In frontotemporal dementia, the frontal and temporal lobes of the brain shrink. In addition, certain substances accumulate in the brain. What causes these changes is usually unknown. There are genetic mutations that have been linked to frontotemporal dementia. But more than half of the people who develop frontotemporal dementia have no family history of dementia.

Recently, researchers have confirmed shared genetics and molecular pathways between frontotemporal dementia and amyotrophic lateral sclerosis (ALS). More research needs to be done to understand the connection between these conditions, however.

Frontotemporal dementia is often misdiagnosed as a psychiatric problem or as Alzheimer's disease. But frontotemporal dementia tends to occur at a younger age than does Alzheimer's disease. Frontotemporal dementia often begins between the ages of 40 and 65.

Signs and symptoms of frontotemporal dementia can be different from one individual to the next. Signs and symptoms get progressively worse over time, usually over years. Clusters of symptom types tend to occur together, and people may have more than one cluster of symptom types. The most common signs of frontotemporal dementia involve extreme changes in behavior and personality, Speech and language problems, and/or movement disorders.

### Behavioral Changes Include:

Increasingly inappropriate social behavior
Loss of empathy and other interpersonal skills, such as having sensitivity to another's feelings
Lack of judgment
Loss of inhibition
Lack of interest (apathy), which can be mistaken for depression
Repetitive compulsive behavior, such as tapping, clapping or smacking lips
A decline in personal hygiene
Changes in eating habits, usually overeating or developing a preference for sweets and carbohydrates
Eating inedible objects
Compulsively wanting to put things in the mouth.

Some subtypes of frontotemporal dementia lead to language problems or impairment or loss of speech. Primary progressive aphasia, semantic dementia and progressive agrammatic (nonfluent) aphasia are all considered to be frontotemporal dementia. Problems caused by these conditions include:
Increasing difficulty in using and understanding written and spoken language, such as having trouble finding the right word to use in speech or naming objects
Trouble naming things, possibly replacing a specific word with a more general word such as “it” for pen
No longer knowing word meanings.
Having hesitant speech that may sound telegraphic
Making mistakes in sentence construction.

Rarer subtypes of frontotemporal dementia are characterized by problems with movement, similar to those associated with Parkinson's disease or amyotrophic lateral sclerosis (ALS). Movement-related problems may include:
Tremor
Rigidity
Muscle spasms
Poor coordination
Difficulty swallowing
Muscle weakness
Inappropriate laughing or crying.

Frontotemporal dementia (FTD) describes a clinical syndrome associated with shrinking of the frontal and temporal anterior lobes of the brain. Originally known as Pick's disease, the name and classification of FTD has been a topic of discussion for over a century. The current designation of the syndrome groups together Pick's disease, primary progressive aphasia, and semantic dementia as FTD. Some doctors propose adding corticobasal degeneration and progressive supranuclear palsy to FTD and calling the group Pick Complex. These designations will continue to be debated. As it is defined today, the symptoms of FTD fall into two clinical patterns that involve either (1) changes in behavior, or (2) problems with language. The first type features behavior that can be either impulsive (disinhibited) or bored and listless (apathetic) and includes inappropriate social behavior; lack of social tact; lack of empathy; distractibility; loss of insight into the behaviors of oneself and others; an increased interest in sex; changes in food preferences; agitation or, conversely, blunted emotions; neglect of personal hygiene; repetitive or compulsive behavior, and decreased energy and motivation. The second type primarily features symptoms of language disturbance, including difficulty making or understanding speech, often in conjunction with the behavioral type's symptoms. Spatial skills and memory remain intact. There is a strong genetic component to the disease; FTD often runs in families.

### Frontotemporal Lobar Degeneration (FTLD)

In some embodiments, a disease or disorder includes frontotemporal lobar degeneration (FTLD), a pathological process that occurs in frontotemporal dementia (FTD). It is characterized by atrophy in the frontal lobe and temporal lobe of the brain, with sparing of the parietal and occipital lobes. Common proteinopathies that are found in FTLD include the accumulation of tau proteins and TDP-43 proteins. Mutations in the C9orf72 gene have been established as a major genetic contribution of FTLD, although defects in the GRN and MAPT genes are also associated with it.

There are 3 main histological subtypes found at post-mortem: FTLD-tau, FTLD-TDP (or FTLD-U), and FTLD-FUS. FTLD-tau is characterised by tau-positive inclusion bodies often referred to as Pick-bodies. Examples of FTLD-tau include: Pick's disease, corticobasal degeneration, progressive supranuclear palsy. FTLD-TDP (or FTLD-U) is characterised by ubiquitin and TDP-43 positive, tau negative, FUS negative inclusion bodies. FTLD-FUS is characterised by FUS positive cytoplasmic inclusions, intra nuclear inclusions, and neuritic threads.

For diagnostic purposes, magnetic resonance imaging (MRI) and ([18F] fluorodeoxyglucose) positron emission tomography (FDG-PET) may be used. They measure either atrophy or reductions in glucose utilization. The three clinical subtypes of frontotemporal lobar degeneration, frontotemporal dementia, semantic dementia and progressive nonfluent aphasia, are characterized by impairments in specific neural networks. The first subtype with behavioral deficits, frontotemporal dementia, mainly affects a frontomedian network discussed in the context of social cognition. Semantic dementia is mainly related to the inferior temporal poles and amygdalae; brain regions that have been discussed in the context of conceptual knowledge, semantic information processing, and social cognition, whereas progressive nonfluent aphasia affects the whole left frontotemporal network for phonological and syntactical processing.

### Autism Spectrum Disorder (ASD)

In some embodiments, a disease or disorder includes autism (also referred to as autism spectrum disorder (ASD)). ASD may be characterized by deficits in social communication and social interaction, and repetitive or restricted patterns of behaviors, interests, or activities, which can include hyper- and hyporeactivity to sensory input. Autism may manifest very differently in each person. For example, some are nonspeaking, while others have proficient spoken language. Because of this, there is wide variation in the support needs of people across the autism spectrum.

### Common Signs of ASD May Include any of the Following:

avoidance of eye-contact
little or no babbling as an infant
not showing interest in indicated objects
delayed language skills (e.g. having a smaller vocabulary than peers or difficulty expressing themselves in words)
reduced interest in other children or caretakers, possibly with more interest in objects
difficulty playing reciprocal games (e.g. peek-a-boo)
hyper- or hypo-sensitivity to or unusual response to the smell, texture, sound, taste, or appearance of things
resistance to changes in routine
repetitive, limited, or otherwise unusual usage of toys (e.g. lining up toys)
repetition of words or phrases (echolalia)
repetitive motions or movements, including stimming
self-harming

Autism Spectrum Disorder (ASD) is associated with widespread recruitment of abnormal cryptic exons. Cryptic exon inclusion often disrupts the normal reading frame. Up to 10% ASD/ID patients may have de novo cryptic splice site mutations (˜100,000 individuals in the USA).

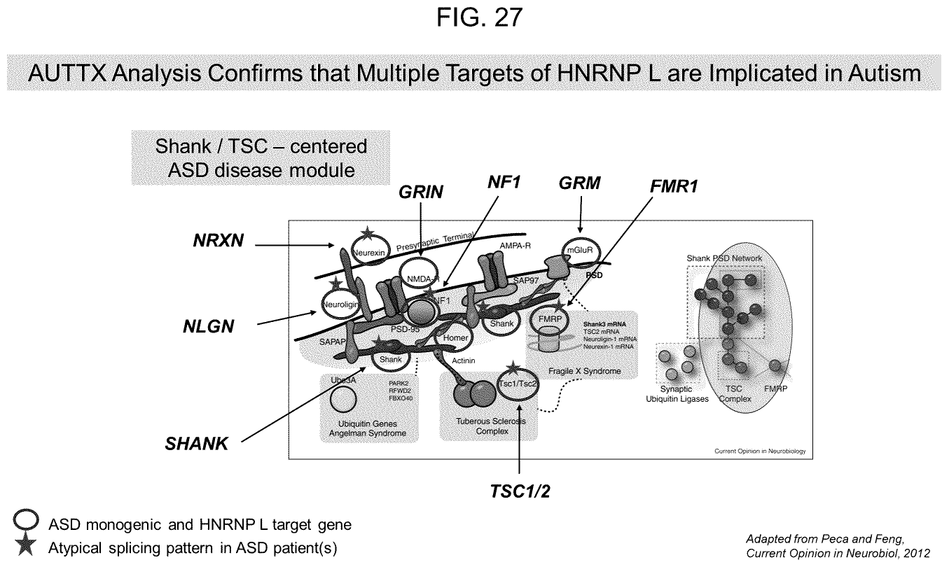

Some aspects that may relate to ASD are included in FIG. 27. In some embodiments, the subject with ASD, or a subject to be treated, has a mutation or defect in a gene in FIG. 27.

Some embodiments include any aspect as described in WO2019236750, as it relates to autism or ASD, which is incorporated herein in its entirety. In some embodiments, the subject with ASD has a mutation in a gene in any of Tables 11-16.

### Alzheimer's Disease

In some embodiments, a disease or disorder includes Alzheimer's disease, a progressive disorder that causes brain cells to waste away (degenerate) and die. Alzheimer's disease is the most common cause of dementia—a continuous decline in thinking, behavioral and social skills that disrupts a person's ability to function independently.

The early signs of the disease may be forgetting recent events or conversations. As the disease progresses, a person with Alzheimer's disease will develop severe memory impairment and lose the ability to carry out everyday tasks.

Current Alzheimer's disease medications may temporarily improve symptoms or slow the rate of decline. These treatments can sometimes help people with Alzheimer's disease maximize function and maintain independence for a time. Different programs and services can help support people with Alzheimer's disease and their caregivers.

There is no treatment that cures Alzheimer's disease or alters the disease process in the brain. In advanced stages of the disease, complications from severe loss of brain function-such as dehydration, malnutrition or infection-result in death.

Memory loss is the key symptom of Alzheimer's disease. An early sign of the disease is usually difficulty remembering recent events or conversations. As the disease progresses, memory impairments worsen and other symptoms develop.

Brain changes associated with Alzheimer's disease lead to growing trouble with: memory, thinking and reasoning, making judgments and decisions, planning and performing familiar tasks, changes in personality and behavior, etc.

The exact causes of Alzheimer's disease aren't fully understood, but at its core are problems with brain proteins that fail to function normally, disrupt the work of brain cells (neurons) and unleash a series of toxic events. Neurons are damaged, lose connections to each other and eventually die.

The damage most often starts in the region of the brain that controls memory, but the process begins years before the first symptoms. The loss of neurons spreads in a somewhat predictable pattern to other regions of the brains. By the late stage of the disease, the brain has shrunk significantly.

Interest may be focused on the role of two proteins: plaques or tangles.

Plaques. Beta-amyloid is a leftover fragment of a larger protein. When these fragments cluster together, they appear to have a toxic effect on neurons and to disrupt cell-to-cell communication. These clusters form larger deposits called amyloid plaques, which also include other cellular debris.

Tangles. Tau proteins play a part in a neuron's internal support and transport system to carry nutrients and other essential materials. In Alzheimer's disease, tau proteins change shape and organize themselves into structures called neurofibrillary tangles. The tangles disrupt the transport system and are toxic to cells.

### Lewy Body Dementia (LBD)

In some embodiments, a disease or disorder includes Lewy body dementia, also known as dementia with Lewy bodies, the second most common type of progressive dementia after Alzheimer's disease dementia. Protein deposits, called Lewy bodies, develop in nerve cells in the brain regions involved in thinking, memory and movement (motor control).

Lewy body dementia causes a progressive decline in mental abilities. People with Lewy body dementia may experience visual hallucinations and changes in alertness and attention. Other effects include Parkinson's disease-like signs and symptoms such as rigid muscles, slow movement and tremors.

Lewy body dementia signs and symptoms may include: visual hallucinations, movement disorders, poor regulation of body functions (autonomic nervous system), cognitive problems, sleep difficulties, fluctuating attention, depression, apathy, etc.

Lewy body dementia is characterized by the abnormal buildup of proteins into masses known as Lewy bodies. This protein is also associated with Parkinson's disease. People who have Lewy bodies in their brains also have the plaques and tangles associated with Alzheimer's disease.

### Hippocampal Sclerosis (HS)

In some embodiments, a disease or disorder includes Hippocampal sclerosis (HS), a neuropathological condition with severe neuronal cell loss and gliosis in the hippocampus, specifically in the CA-1 (Cornu Ammonis area 1) and subiculum of the hippocampus. It was first described in 1880 by Wilhelm Sommer. Hippocampal sclerosis is a frequent pathologic finding in community-based dementia. Hippocampal sclerosis can be detected with autopsy or MRI. Individuals with hippocampal sclerosis have similar initial symptoms and rates of dementia progression to those with Alzheimer's disease (AD) and therefore are frequently misclassified as having Alzheimer's Disease. But clinical and pathologic findings suggest that hippocampal sclerosis has characteristics of a progressive disorder although the underlying cause remains elusive. A diagnosis of hippocampal sclerosis has a significant effect on the life of patients because of the notable mortality, morbidity and social impact related to epilepsy, as well as side effects associated with antiepileptic treatments.

Histopathological hallmarks of hippocampal sclerosis include segmental loss of pyramidal neurons, granule cell dispersion and reactive gliosis. This means that pyramidal neuronal cells are lost, granule cells are spread widely or driven off, and glial cells are changed in response to damage to the central nervous system (CNS). Generally, hippocampal sclerosis may be seen in some cases of epilepsy, particularly temporal lobe epilepsy. It is important to clarify the nature of insults that most likely have caused the hippocampal sclerosis and have initiated the epileptogenic process. Presence of hippocampal sclerosis and duration of epilepsy longer than 10 years were found to cause parasympathetic autonomic dysfunction, whereas seizure refractoriness was found to cause sympathetic autonomic dysfunction. Apart from its association with the chronic nature of epilepsy, hippocampal sclerosis was shown to have an important role in internal cardiac autonomic dysfunction. Patients with left hippocampal sclerosis had more severe parasympathetic dysfunction as compared with those with right hippocampal sclerosis. In young individuals, mesial temporal sclerosis is commonly recognized with temporal lobe epilepsy (TLE). On the other hand, it is an often unrecognized cause of cognitive decline, typically presenting with severe memory loss.

Mesial temporal sclerosis is a specific pattern of hippocampal neuron cell loss. There are 3 specific patterns of cell loss. Cell loss might involve sectors CA1 and CA4, CA4 alone, or CA1 to CA4. Associated hippocampal atrophy and gliosis is common. MRI scan commonly displays increased T2 signal and hippocampal atrophy. Mesial temporal sclerosis might occur with other temporal lobe abnormalities (dual pathology). Mesial temporal sclerosis is the most common pathological abnormality in temporal lobe epilepsy. It has been linked to abnormalities in TDP-43 (Aoki et al. Acta Neuropathol. 2015; 129:53-64).

### Corticobasal Degeneration

In some embodiments, a disease or disorder includes Corticobasal degeneration (CBD), a rare condition that can cause gradually worsening problems with movement, speech, memory and swallowing. It's often also called corticobasal syndrome (CBS). CBD is caused by increasing numbers of brain cells becoming damaged or dying over time. Most cases of CBD develop in adults aged between 50 and 70.

The symptoms of CBD get gradually worse over time. They are very variable and many people only have a few of them. Symptoms can include: difficulty controlling your limb on one side of the body (a “useless” hand), muscle stiffness, shaking (tremors), jerky movements and spasms (dystonia), problems with balance and co-ordination, slow and slurred speech, symptoms of dementia, such as memory and visual problems, slow, effortful speech, and difficulty swallowing. One limb is usually affected at first, before spreading to the rest of the body. The rate at which the symptoms progress varies widely from person to person.

CBD occurs when brain cells in certain parts of the brain are damaged as a result of a build-up of a protein called tau. The surface of the brain (cortex) is affected, as well as a deep part of the brain called the basal ganglia. Tau occurs naturally in the brain and is usually broken down before it reaches high levels. In people with CBD, it is not broken down properly and forms harmful clumps in brain cells. CBD has been linked to changes in certain genes, but these genetic links are weak and the risk to other family members is very low.

There is no single test for CBD. Instead, the diagnosis is based on the pattern of the symptoms. A brain scan may be needed to look for other possible causes of the symptoms, as well as tests of memory, concentration and ability to understand language.

### Huntington Disease

In some embodiments, a disease or disorder includes Huntington's disease, a rare, inherited disease that causes the progressive breakdown (degeneration) of nerve cells in the brain. Huntington's disease has a broad impact on a person's functional abilities and usually results in movement, thinking (cognitive) and psychiatric disorders.

Huntington's disease symptoms can develop at any time, but they often first appear when people are in their 30s or 40s. If the condition develops before age 20, it's called juvenile Huntington's disease. When Huntington's develops early, symptoms are somewhat different and the disease may progress faster.

Medications are available to help manage the symptoms of Huntington's disease. But treatments can't prevent the physical, mental and behavioral decline associated with the condition.

Huntington's disease usually causes movement, cognitive and psychiatric disorders with a wide spectrum of signs and symptoms. Which symptoms appear first varies greatly from person to person. Some symptoms appear more dominant or have a greater effect on functional ability, but that can change throughout the course of the disease.

Huntington's disease is caused by an inherited defect in a single gene. Huntington's disease is an autosomal dominant disorder, which means that a person needs only one copy of the defective gene to develop the disorder.

With the exception of genes on the sex chromosomes, a person inherits two copies of every gene—one copy from each parent. A parent with a defective gene could pass along the defective copy of the gene or the healthy copy. Each child in the family, therefore, has a 50% chance of inheriting the gene that causes the genetic disorder.

### Parkinson's Disease

In some embodiments, a disease or disorder includes Parkinson's disease, a progressive nervous system disorder that affects movement. Symptoms start gradually, sometimes starting with a barely noticeable tremor in just one hand. Tremors are common, but the disorder also commonly causes stiffness or slowing of movement.

In the early stages of Parkinson's disease, the face of the patient may show little or no expression. The arms may not swing during walking. Speech may become soft or slurred. Parkinson's disease symptoms worsen as the condition progresses over time.

Parkinson's disease signs and symptoms can be different for everyone. Early signs may be mild and go unnoticed. Symptoms often begin on one side of your body and usually remain worse on that side, even after symptoms begin to affect both sides.

Parkinson's signs and symptoms may include: tremor, slowed movement (bradykinesia), rigid muscles, impaired posture and balance, loss of automatic movements, speech changes, and writing changes.

In Parkinson's disease, certain nerve cells (neurons) in the brain gradually break down or die. Many of the symptoms are due to a loss of neurons that produce a chemical messenger in your brain called dopamine. When dopamine levels decrease, it causes abnormal brain activity, leading to impaired movement and other symptoms of Parkinson's disease.

The cause of Parkinson's disease is unknown, but several factors appear to play a role, including:

Genes. Researchers have identified specific genetic mutations that can cause Parkinson's disease. But these are uncommon except in rare cases with many family members affected by Parkinson's disease. However, certain gene variations appear to increase the risk of Parkinson's disease but with a relatively small risk of Parkinson's disease for each of these genetic markers.

Environmental triggers. Exposure to certain toxins or environmental factors may increase the risk of later Parkinson's disease, but the risk is relatively small.

Researchers have also noted that many changes occur in the brains of people with Parkinson's disease, although it's not clear why these changes occur. These changes include:

The presence of Lewy bodies. Clumps of specific substances within brain cells are microscopic markers of Parkinson's disease. These are called Lewy bodies, and researchers believe these Lewy bodies hold an important clue to the cause of Parkinson's disease.

Alpha-synuclein found within Lewy bodies. Although many substances are found within Lewy bodies, scientists believe an important one is the natural and widespread protein called alpha-synuclein (a-synuclein). It's found in all Lewy bodies in a clumped form that cells can't break down. This is currently an important focus among Parkinson's disease researchers.

### Argyrophilic Grain Disease (AGD)

In some embodiments, a disease or disorder includes Argyrophilic grain disease (AGD), an under-recognized, distinct, highly frequent sporadic tauopathy, with a prevalence reaching 31.3% in centenarians. The most common AGD manifestation is slowly progressive amnestic mild cognitive impairment, accompanied by a high prevalence of neuropsychiatric symptoms. AGD diagnosis can only be achieved postmortem based on the finding of its three main pathologic features: argyrophilic grains, oligodendrocytic coiled bodies and neuronal pretangles. AGD is frequently seen together with Alzheimer's disease-type pathology or in association with other neurodegenerative diseases. Recent studies suggest that AGD may be a defense mechanism against the spread of other neuropathological entities, particularly Alzheimer's disease. This review aims to provide an in-depth overview of the current understanding on AGD. For a review of AGD, see Rodriguez and Grinberg, Dement Neuropsychol. 2015; 9:2-8.

### Chronic Traumatic Encephalopathy (CTE)

In some embodiments, a disease or disorder includes Chronic traumatic encephalopathy (CTE), a term used to describe brain degeneration likely caused by repeated head traumas. CTE is a diagnosis made only at autopsy by studying sections of the brain. CTE is a very rare disorder that is not yet well understood. CTE is not related to the immediate consequences of a late-life episode of head trauma. CTE has a complex relationship to head traumas such as post-concussion syndrome and second impact syndrome that occur earlier in life.

Experts are still trying to understand how repeated head traumas—including how many head injuries and the severity of those injuries—and other factors might contribute to the changes in the brain that result in CTE.

CTE has been found in the brains of people who played football and other contact sports, including boxing. It may also occur in military personnel who were exposed to explosive blasts. Some signs and symptoms of CTE are thought to include difficulties with thinking (cognition), physical problems, emotions and other behaviors. It's thought that these develop years to decades after head trauma occurs.

CTE cannot be made as a diagnosis during life except in those rare individuals with high-risk exposures. Researchers do not yet know the frequency of CTE in the population and do not understand the causes. There is no cure for CTE.

There are no specific symptoms that have been clearly linked to CTE. Some of the possible signs and symptoms of CTE can occur in many other conditions, but in the few people with proven CTE, symptoms have included: difficulty thinking (cognitive impairment), impulsive behavior, depression or apathy, short-term memory loss, difficulty planning and carrying out tasks (executive function), emotional instability, substance misuse, and suicidal thoughts or behavior.

Repetitive head trauma is likely the cause of CTE. Football and ice hockey players, as well as military personnel serving war zones, have been the focus of most CTE studies, though other sports and factors such as physical abuse also can lead to repetitive head injuries.

However, not all athletes and not everyone who experiences repeated concussions, including military personnel, go on to develop CTE. Some studies have shown no increased incidence of CTE in people exposed to repeated head injuries.

CTE is thought to cause areas of the brain to waste away (atrophy). Injuries to the section of nerve cells that conduct electrical impulses affect communication between cells.

It's possible that people with CTE may show signs of another neurodegenerative disease, including Alzheimer's disease, amyotrophic lateral sclerosis (ALS)—also known as Lou Gehrig's disease—Parkinson's disease or frontotemporal lobar degeneration (frontotemporal dementia).

### Perry Syndrome

In some embodiments, a disease or disorder includes Perry syndrome, a progressive brain disease that is characterized by four major features: a pattern of movement abnormalities known as parkinsonism, psychiatric changes, weight loss, and abnormally slow breathing (hypoventilation). These signs and symptoms typically appear in a person's forties or fifties.

Parkinsonism and psychiatric changes are usually the earliest features of Perry syndrome. Signs of parkinsonism include unusually slow movements (bradykinesia), stiffness, and tremors. These movement abnormalities are often accompanied by changes in personality and behavior. The most frequent psychiatric changes that occur in people with Perry syndrome include depression, a general loss of interest and enthusiasm (apathy), withdrawal from friends and family, and suicidal thoughts. Many affected individuals also experience significant, unexplained weight loss early in the disease.

Hypoventilation is a later feature of Perry syndrome. Abnormally slow breathing most often occurs at night, causing affected individuals to wake up frequently. As the disease worsens, hypoventilation can result in a life-threatening lack of oxygen and respiratory failure.

People with Perry syndrome typically survive for about 5 years after signs and symptoms first appear. Most affected individuals ultimately die of respiratory failure or pneumonia. Suicide is another cause of death in this condition.

Perry syndrome results from mutations in the DCTN1 gene. This gene provides instructions for making a protein called dynactin-1, which is involved in the transport of materials within cells. To move materials, dynactin-1 interacts with other proteins and with a track-like system of small tubes called microtubules. These components work together like a conveyer belt to move materials within cells. This transport system appears to be particularly important for the normal function and survival of nerve cells (neurons) in the brain.

Mutations in the DCTN1 gene alter the structure of dynactin-1, making it less able to attach (bind) to microtubules and transport materials within cells. This abnormality causes neurons to malfunction and ultimately die. A gradual loss of neurons in areas of the brain that regulate movement, emotion, and breathing underlies the signs and symptoms of Perry syndrome.

### Alexander Disease

In some embodiments, a disease or disorder includes Alexander disease, a very rare autosomal dominant leukodystrophy, which are neurological conditions caused by anomalies in the myelin which protects nerve fibers in the brain. The most common type is the infantile form that usually begins during the first 2 years of life. Symptoms include mental and physical developmental delays, followed by the loss of developmental milestones, an abnormal increase in head size and seizures. The juvenile form of Alexander disease has an onset between the ages of 2 and 13 years. These children may have excessive vomiting, difficulty swallowing and speaking, poor coordination, and loss of motor control. Adult-onset forms of Alexander disease are less common. The symptoms sometimes mimic those of Parkinson's disease or multiple sclerosis, or may present primarily as a psychiatric disorder.

Alexander disease is a genetic disorder affecting the midbrain and cerebellum of the central nervous system. It is caused by mutations in the gene for glial fibrillary acidic protein (GFAP) that maps to chromosome 17q21. It is inherited in an autosomal dominant manner, such that the child of a parent with the disease has a 50% chance of inheriting the condition, if the parent is heterozygotic. However, most cases arise de novo as the result of sporadic mutations.

Alexander disease belongs to leukodystrophies, a group of diseases that affect the growth or development of the myelin sheath. The destruction of white matter in the brain is accompanied by the formation of fibrous, eosinophilic deposits known as Rosenthal fibers. Rosenthal fibers appear not to be present in healthy people, but occur in specific diseases, like some forms of cancer, Alzheimer's, Parkinson's, Huntington's, and ALS. The Rosenthal fibers found in Alexander disease do not share the distribution or concentration of other diseases and disorders.

Detecting the signs of Alexander disease is possible with magnetic resonance imaging (MRI), which looks for specific changes in the brain that may be tell-tale signs for the disease. It is even possible to detect adult-onset Alexander disease with MRI. Alexander disease may also be revealed by genetic testing for its known cause. A rough diagnosis may also be made through revealing of clinical symptoms, including enlarged head size, along with radiological studies, and negative tests for other leukodystrophies.

### Multisystem Proteinopathy (MSP)

In some embodiments, a disease or disorder includes Multisystem proteinopathy (MSP), a dominantly inherited, pleiotropic, degenerative disorder of humans that can affect muscle, bone, and/or the central nervous system. MSP can manifest clinically as classical amyotrophic lateral sclerosis (ALS), frontotemporal dementia (FTD), inclusion body myopathy (IBM), Paget's disease of bone (PDB), or as a combination of these disorders. Historically, several different names have been used to describe MSP, most commonly “inclusion body myopathy with early-onset Paget disease and frontotemporal dementia (IBMPFD)” or “inclusion body myopathy with frontotemporal dementia. Paget's disease of bone, and amyotrophic lateral sclerosis (IBMPFD/ALS).” However, IBMPFD and IBMPFD/ALS are now considered outdated classifications and are more properly referred to as MSP, as the disease is clinically heterogeneous and its phenotypic spectrum extends beyond IBM, PDB, FTD, and ALS to include motor neuron disease, Parkinson's disease features, and ataxia features. Although MSP is rare, growing interest in this syndrome derives from the molecular insights the condition provides into the etiological relationship between common age-related degenerative diseases of muscle, bone, and brain.

A useful operational definition of MSP is dominantly inherited degeneration that includes neurological involvement (either motor neuron disease or dementia) in combination with either distal myopathy or Pagetic bone degeneration. Most MSP patients present with weakness, and of these, approximately 65% present with motor neuron involvement. Although rare, MSP can also include involvement of cardiac, hepatic, visual, auditory, sensory, and autonomic systems. The histopathology of tissues affected by MSP includes ubiquitin-positive cytoplasmic inclusions of RNA-binding proteins, such as TDP-43, HNRNPA1, HNRNPA2B1, and other components of RNA granules.

MSP is a dominantly inherited and genetically heterogeneous disease. The most common genetic cause of MSP is missense mutations affecting the valosin-containing protein (VCP) gene, which causes a subtype of MSP known as MSP1. Other pathogenic variants have been identified in HNRNPA2B1 and HNRNPA1, which cause MSP2 and MSP3, respectively. Additional genes linked to MSP include MATR3, OPTN, and p62/SQSTM1.

### Attention-Deficit/Hyperactivity Disorder (ADHD)

In some embodiments, a disease or disorder includes attention-deficit/hyperactivity disorder (ADHD), a disorder marked by an ongoing pattern of inattention and/or hyperactivity-impulsivity that interferes with functioning or development. Inattention means a person wanders off task, lacks persistence, has difficulty sustaining focus, and is disorganized; and these problems are not due to defiance or lack of comprehension. Hyperactivity means a person seems to move about constantly, including in situations in which it is not appropriate; or excessively fidgets, taps, or talks. In adults, it may be extreme restlessness or wearing others out with constant activity. Impulsivity means a person makes hasty actions that occur in the moment without first thinking about them and that may have a high potential for harm, or a desire for immediate rewards or inability to delay gratification. An impulsive person may be socially intrusive and excessively interrupt others or make important decisions without considering the long-term consequences.

Inattention and hyperactivity/impulsivity are the key behaviors of ADHD. Some people with ADHD only have problems with one of the behaviors, while others have both inattention and hyperactivity-impulsivity. Most children have the combined type of ADHD.

In preschool, the most common ADHD symptom is hyperactivity. It is normal to have some inattention, unfocused motor activity, and impulsivity, but for people with ADHD, these behaviors: are more severe, occur more often, and/or interfere with or reduce the quality of how they function socially, at school, or in a job.

Diagnosis of ADHD requires a comprehensive evaluation by a licensed clinician, such as a pediatrician, psychologist, or psychiatrist with expertise in ADHD. For a person to receive a diagnosis of ADHD, the symptoms of inattention and/or hyperactivity-impulsivity must be chronic or long-lasting, impair the person's functioning, and cause the person to fall behind typical development for his or her age. The doctor will also ensure that any ADHD symptoms are not due to another medical or psychiatric condition. Most children with ADHD receive a diagnosis during the elementary school years. For an adolescent or adult to receive a diagnosis of ADHD, the symptoms need to have been present before age 12.

ADHD symptoms can appear as early as between the ages of 3 and 6 and can continue through adolescence and adulthood. Symptoms of ADHD can be mistaken for emotional or disciplinary problems or missed entirely in quiet, well-behaved children, leading to a delay in diagnosis. Adults with undiagnosed ADHD may have a history of poor academic performance, problems at work, or difficult or failed relationships.

ADHD symptoms can change over time as a person ages. In young children with ADHD, hyperactivity-impulsivity is the most predominant symptom. As a child reaches elementary school, the symptom of inattention may become more prominent and cause the child to struggle academically. In adolescence, hyperactivity seems to lessen and may show more often as feelings of restlessness or fidgeting, but inattention and impulsivity may remain. Many adolescents with ADHD also struggle with relationships and antisocial behaviors. Inattention, restlessness, and impulsivity tend to persist into adulthood.

### Dyslexia

In some embodiments, a disease or disorder includes dyslexia, a learning disorder that involves difficulty reading due to problems identifying speech sounds and learning how they relate to letters and words (decoding). Also called reading disability, dyslexia affects areas of the brain that process language.

People with dyslexia have normal intelligence and usually have normal vision. Most children with dyslexia can succeed in school with tutoring or a specialized education program. Emotional support also plays an important role.

Though there's no cure for dyslexia, early assessment and intervention result in the best outcome. Sometimes dyslexia goes undiagnosed for years and isn't recognized until adulthood, but it's never too late to seek help.

Signs of dyslexia can be difficult to recognize before your child enters school, but some early clues may indicate a problem. Once a child reaches school age, the child's teacher may be the first to notice a problem. Severity varies, but the condition often becomes apparent as a child starts learning to read. Some aspects that may present in subjects with dyslexia include the following.

### Before School

Signs that a Young Child May be at Risk of Dyslexia Include:
Late talking;
Learning new words slowly;
Problems forming words correctly, such as reversing sounds in words or confusing words that sound alike;
Problems remembering or naming letters, numbers and colors; and/or
Difficulty learning nursery rhymes or playing rhyming games.

### School Age

### Once a Child is in School, Dyslexia Signs and Symptoms May Become More Apparent, Including:

Reading well below the expected level for age;
Problems processing and understanding what he or she hears;
Difficulty finding the right word or forming answers to questions;
Problems remembering the sequence of things;
Difficulty seeing (and occasionally hearing) similarities and differences in letters and words;
Inability to sound out the pronunciation of an unfamiliar word;
Difficulty spelling;
Spending an unusually long time completing tasks that involve reading or writing; and/or
Avoiding activities that involve reading;

### Teens and Adults

Dyslexia Signs in Teens and Adults are Similar to Those in Children. Some Common Dyslexia Signs and Symptoms in Teens and Adults Include:
Difficulty reading, including reading aloud;
Slow and labor-intensive reading and writing;
Problems spelling;
Avoiding activities that involve reading;
Mispronouncing names or words, or problems retrieving words;
Trouble understanding jokes or expressions that have a meaning not easily understood from the specific words (idioms), such as “piece of cake” meaning “easy”;
Spending an unusually long time completing tasks that involve reading or writing;
Difficulty summarizing a story;
Trouble learning a foreign language;
Difficulty memorizing; and/or
Difficulty doing math problems

### Epilepsy

In some embodiments, a disease or disorder includes epilepsy, a central nervous system (neurological) disorder in which brain activity becomes abnormal, causing seizures or periods of unusual behavior, sensations, and sometimes loss of awareness.

Anyone can develop epilepsy. Epilepsy affects both males and females of all races, ethnic backgrounds and ages.

Seizure symptoms can vary widely. Some people with epilepsy simply stare blankly for a few seconds during a seizure, while others repeatedly twitch their arms or legs. Having a single seizure doesn't mean you have epilepsy. At least two unprovoked seizures are generally required for an epilepsy diagnosis.

Treatment with medications or sometimes surgery can control seizures for the majority of people with epilepsy. Some people require lifelong treatment to control seizures, but for others, the seizures eventually go away. Some children with epilepsy may outgrow the condition with age.

Because epilepsy is caused by abnormal activity in the brain, seizures can affect any process your brain coordinates. Seizure signs and symptoms may include:
Temporary confusion;
A staring spell;
Uncontrollable jerking movements of the arms and legs;
Loss of consciousness or awareness; and/or
Psychic symptoms such as fear, anxiety or deja vu.

Symptoms vary depending on the type of seizure. In most cases, a person with epilepsy will tend to have the same type of seizure each time, so the symptoms will be similar from episode to episode. Doctors generally classify seizures as either focal or generalized, based on how the abnormal brain activity begins.

Epilepsy has no identifiable cause in about half the people with the condition. In the other half, the condition may be traced to various factors, including:

Genetic influence. Some types of epilepsy, which are categorized by the type of seizure you experience or the part of the brain that is affected, run in families. In these cases, it's likely that there's a genetic influence.

Researchers have linked some types of epilepsy to specific genes, but for most people, genes are only part of the cause of epilepsy. Certain genes may make a person more sensitive to environmental conditions that trigger seizures.

Head trauma. Head trauma as a result of a car accident or other traumatic injury can cause epilepsy.

Brain conditions. Brain conditions that cause damage to the brain, such as brain tumors or strokes, can cause epilepsy. Stroke is a leading cause of epilepsy in adults older than age 35.

Infectious diseases. Infectious diseases, such as meningitis, AIDS and viral encephalitis, can cause epilepsy.

Prenatal injury. Before birth, babies are sensitive to brain damage that could be caused by several factors, such as an infection in the mother, poor nutrition or oxygen deficiencies. This brain damage can result in epilepsy or cerebral palsy.

Developmental disorders. Epilepsy can sometimes be associated with developmental disorders, such as autism and neurofibromatosis.

### Bipolar Disorder

In some embodiments, a disease or disorder includes bipolar disorder, formerly called manic depression, a mental health condition that causes extreme mood swings that include emotional highs (mania or hypomania) and lows (depression).

Depression may result in a feeling of sadness or hopelessness and a loss of interest or pleasure in most activities. When the mood shifts to mania or hypomania (less extreme than mania), a patient may feel euphoric, full of energy or unusually irritable. These mood swings can affect sleep, energy, activity, judgment, behavior and the ability to think clearly.

Episodes of mood swings may occur rarely or multiple times a year. While most people will experience some emotional symptoms between episodes, some may not experience any.

Although bipolar disorder is a lifelong condition, you can manage your mood swings and other symptoms by following a treatment plan. In most cases, bipolar disorder is treated with medications and psychological counseling (psychotherapy).

There are several types of bipolar and related disorders. They may include mania or hypomania and depression. Symptoms can cause unpredictable changes in mood and behavior, resulting in significant distress and difficulty in life.

Bipolar I disorder: At least one manic episode that may be preceded or followed by hypomanic or major depressive episodes. In some cases, mania may trigger a break from reality (psychosis).

Bipolar II disorder: At least one major depressive episode and at least one hypomanic episode, without a manic episode.

Cyclothymic disorder: At least two years—or one year in children and teenagers—of many periods of hypomania symptoms and periods of depressive symptoms (though less severe than major depression).

Other types. These include, for example, bipolar and related disorders induced by certain drugs or alcohol or due to a medical condition, such as Cushing's disease, multiple sclerosis or stroke.

Bipolar II disorder is not a milder form of bipolar I disorder, but a separate diagnosis. While the manic episodes of bipolar I disorder can be severe and dangerous, individuals with bipolar II disorder can be depressed for longer periods, which can cause significant impairment.

Although bipolar disorder can occur at any age, typically it's diagnosed in the teenage years or early 20s. Symptoms can vary from person to person, and symptoms may vary over time.

The exact cause of bipolar disorder is unknown, but several factors may be involved, such as:

Biological differences. People with bipolar disorder appear to have physical changes in their brains. The significance of these changes is still uncertain but may eventually help pinpoint causes.

Genetics. Bipolar disorder is more common in people who have a first-degree relative, such as a sibling or parent, with the condition. Researchers are trying to find genes that may be involved in causing bipolar disorder.

### Schizophrenia

In some embodiments, a disease or disorder includes schizophrenia, a serious mental disorder in which people interpret reality abnormally. Schizophrenia may result in some combination of hallucinations, delusions, and extremely disordered thinking and behavior that impairs daily functioning, and can be disabling.

People with schizophrenia require lifelong treatment. Early treatment may help get symptoms under control before serious complications develop and may help improve the long-term outlook.

Schizophrenia involves a range of problems with thinking (cognition), behavior and emotions. Signs and symptoms may vary, but usually involve delusions, hallucinations or disorganized speech, and reflect an impaired ability to function. Symptoms may include: delusions, hallucinations, disorganized thinking (speech), extremely disorganized or abnormal motor behavior, and negative symptoms.

Symptoms can vary in type and severity over time, with periods of worsening and remission of symptoms. Some symptoms may always be present.

In men, schizophrenia symptoms typically start in the early to mid-20s. In women, symptoms typically begin in the late 20s. It's uncommon for children to be diagnosed with schizophrenia and rare for those older than age 45.

Schizophrenia symptoms in teenagers are similar to those in adults, but the condition may be more difficult to recognize. This may be in part because some of the early symptoms of schizophrenia in teenagers are common for typical development during teen years, such as:

Withdrawal from friends and family, a drop in performance at school, trouble sleeping, irritability or depressed mood, and lack of motivation.

Also, recreational substance use, such as marijuana, methamphetamines or LSD, can sometimes cause similar signs and symptoms. Compared with schizophrenia symptoms in adults, teens may be: less likely to have delusions, and/or more likely to have visual hallucinations.

### Parkinsonism-Dementia Complex of Guam

In some embodiments, a disease or disorder includes a Parkinsonism dementia complex. On Guam and in two other Pacific locales, indigenous residents and immigrants are prone to familial neurodegeneration that manifests as atypical Parkinsonism, dementia, motor neuron disease, or a combination of these three phenotypes. This progressive and fatal disease of the Mariana islands, the Kii peninsula of Japan, and the coastal plain of West New Guinea is similar and the pathological features have close affiliation with universal tauopathies, including progressive supranuclear palsy, Alzheimer's disease, and amyotrophic lateral sclerosis. The Chamorros of Guam call the disease lytico-bodig, and neuroscientists refer to it as the amyotrophic lateral sclerosis/Parkinsonism-dementia complex. During recent decades, its prevalence has declined progressively, and the age at onset has steadily increased. In 2004, motor neuron disease, once 100 times more common than elsewhere is rare, atypical Parkinsonism is declining, and only dementia remains unusually common in elderly females. The cause of this obscure malady remains uncertain, despite 60 years of international research, but its ending implicates environmental influences rather than genetic predisposition. For a detailed description of parkinsonism-dementia complex of Guam, see Steele, Mov Disord. 2005 Suppl 12: S99-S107, which is incorporated herein by reference in its entirety.

### Dementia Pugilistica

In some embodiments, a disease or disorder includes dementia pugilistica. Extensive exposure of boxers to neurotrauma in the early 20th century led to the so-called punch drunk syndrome, which was formally recognized in the medical literature in 1928. “Punch drunk” terminology was replaced by the less derisive ‘dementia pugilistica’ in 1937. In the early case material, the diagnosis of dementia pugilistica required neurological deficits, including slurring dysarthria, ataxia, pyramidal signs, extrapyramidal signs, memory impairment, and personality changes, although the specific clinical substrate has assumed lesser importance in recent years with a shift in focus on molecular pathogenesis. The postmortem neuropathology of dementia pugilistica has also evolved substantially over the past 90 years, from suspected concussion-related hemorrhages to diverse structural and neurofibrillary changes to geographic tauopathy. Progressive neurodegenerative tauopathy is among the prevailing theories for disease pathogenesis currently, although this may be overly simplistic. Careful examination of historical cases reveals both misdiagnoses and a likelihood that dementia pugilistica at that time was caused by cumulative structural brain injury. More recent neuropathological studies indicate subclinical and possibly static tauopathy in some athletes and non-athletes. Indeed, it is unclear from the literature whether retired boxers reach the inflection point that tends toward progressive neurodegeneration in the manner of Alzheimer's disease due to boxing. Even among historical cases with extreme levels of exposure, progressive disease was exceptional. For a detailed description of dementia pugilistica, see Castellani and Perry, J Alzheimers Dis. 2017; 60:1209-1221, which is incorporated herein by reference in its entirety.

Diffuse Neurofibrillary Tangles with Calcification

In some embodiments, a disease or disorder includes neurofibrillary tangles, which may be diffused and have calcification. The term “diffuse neurofibrillary tangles with calcification” (DNTC) is proposed for a new form of presenile dementia. It is characterised by slowly progressive cortical dementia in the presenium, localized temporal or temporofrontal lobar atrophy, numerous neurofibrillary tangles widespread in the cerebral cortex, and pronounced calcareous deposits; 16 cases of DNTC, have been reported. For a detailed description of DNTC, see Kosaka, J Neurol Neurosurg Psychiatry. 1994; 57 (5): 594-596, which is incorporated herein by reference in its entirety.

### Down's Syndrome

In some embodiments, a disease or disorder includes Down syndrome, a genetic disorder caused when abnormal cell division results in an extra full or partial copy of chromosome 21. This extra genetic material causes the developmental changes and physical features of Down syndrome.

Down syndrome varies in severity among individuals, causing lifelong intellectual disability and developmental delays. It's the most common genetic chromosomal disorder and cause of learning disabilities in children. It also commonly causes other medical abnormalities, including heart and gastrointestinal disorders.

Better understanding of Down syndrome and early interventions can greatly increase the quality of life for children and adults with this disorder and help them live fulfilling lives.

Each person with Down syndrome is an individual-intellectual and developmental problems may be mild, moderate or severe. Some people are healthy while others have significant health problems such as serious heart defects.

Children and adults with Down syndrome have distinct facial features. Though not all people with Down syndrome have the same features, some of the more common features include: flattened face, small head, short neck, protruding tongue, upward slanting eye lids (palpebral fissures), unusually shaped or small ears, poor muscle tone, broad, short hands with a single crease in the palm, relatively short fingers and small hands and feet, excessive flexibility, tiny white spots on the colored part (iris) of the eye called Brushfield's spots, and short height. Infants with Down syndrome may be average size, but typically they grow slowly and remain shorter than other children the same age.

Human cells normally contain 23 pairs of chromosomes. One chromosome in each pair comes from your father, the other from your mother.

Down syndrome results when abnormal cell division involving chromosome 21 occurs. These cell division abnormalities result in an extra partial or full chromosome 21. This extra genetic material is responsible for the characteristic features and developmental problems of Down syndrome. Any one of three genetic variations can cause Down syndrome:

Trisomy 21. About 95 percent of the time, Down syndrome is caused by trisomy 21—the person has three copies of chromosome 21, instead of the usual two copies, in all cells. This is caused by abnormal cell division during the development of the sperm cell or the egg cell.

Mosaic Down syndrome. In this rare form of Down syndrome, a person has only some cells with an extra copy of chromosome 21. This mosaic of normal and abnormal cells is caused by abnormal cell division after fertilization.

Translocation Down syndrome. Down syndrome can also occur when a portion of chromosome 21 becomes attached (translocated) onto another chromosome, before or at conception. These children have the usual two copies of chromosome 21, but they also have additional genetic material from chromosome 21 attached to another chromosome.

### Familial British Dementia

In some embodiments, a disease or disorder includes a form of dementia such as Familial British, which was first reported by Cecil Charles Worster-Drought in 1933 and is therefore also known as Worster-Drought syndrome. It is caused by a mutation in the ITM2B gene (also known as BRI2); a different mutation of the same gene causes the similar syndrome of familial Danish dementia. The combination of amyloid pathology and neurofibrillary tangles has led to comparison with the pathology of Alzheimer's disease.

### Familial Danish Dementia

In some embodiments, a disease or disorder includes familial Danish dementia (FDD), which may be pathologically characterized by widespread cerebral amyloid angiopathy (CAA), parenchymal protein deposits, and neurofibrillary degeneration. FDD is associated with a mutation of the BRI2 gene located on chromosome 13. In FDD there is a decamer duplication, which abolishes the normal stop codon, resulting in an extended precursor protein and the release of an amyloidogenic fragment, ADan. The aim of this study was to describe the major neuropathological changes in FDD and to assess the distribution of ADan lesions, neurofibrillary pathology, glial, and microglial response using conventional techniques, immunohistochemistry, confocal microscopy, and immunoelectron microscopy. We showed that ADan is widely distributed in the central nervous system (CNS) in the leptomeninges, blood vessels, and parenchyma. A predominance of parenchymal pre-amyloid (non-fibrillary) lesions was found. Abeta was also present in a proportion of both vascular and parenchymal lesions. There was severe neurofibrillary pathology, and tau immunoblotting revealed a triplet electrophoretic migration pattern comparable with PHF-tau. FDD is a novel form of CNS amyloidosis with extensive neurofibrillary degeneration occurring with parenchymal, predominantly pre-amyloid rather than amyloid, deposition. These findings support the notion that parenchymal amyloid fibril formation is not a prerequisite for the development of neurofibrillary tangles. The significance of concurrent ADan and Abeta deposition in FDD is under further investigation.

For a detailed description of FDD, see Holton et al., J Neuropathol Exp Neurol. 2002; 61 (3): 254-267, which is incorporated herein by reference in its entirety.

### Fragile X Syndrome

In some embodiments, a disease or disorder includes fragile X syndrome. Fragile X syndrome (FXS) is a genetic disorder that may be characterized by mild-to-moderate intellectual disability. The average IQ in males with FXS is under 55, while about two thirds of affected females are intellectually disabled. Physical features may include a long and narrow face, large ears, flexible fingers, and large testicles. About a third of those affected have features of autism such as problems with social interactions and delayed speech. Hyperactivity is common, and seizures occur in about 10%. Males are usually more affected than females.

This disorder and finding of fragile X syndrome has an X-linked dominant inheritance. It is typically caused by an expansion of the CGG triplet repeat within the FMR1 (fragile X messenger ribonucleoprotein 1) gene on the X chromosome. This may result in silencing (methylation) of this part of the gene and a deficiency of the resultant protein (FMRP), which is required for the normal development of connections between neurons. Diagnosis may include genetic testing to determine the number of CGG repeats in the FMR1 gene. Normally, there are between 5 and 40 repeats; fragile X syndrome occurs with more than 200. A premutation is said to be present when the gene has between 55 and 200 repeats; females with a premutation have an increased risk of having an affected child. Testing for premutation carriers may allow for genetic counseling.

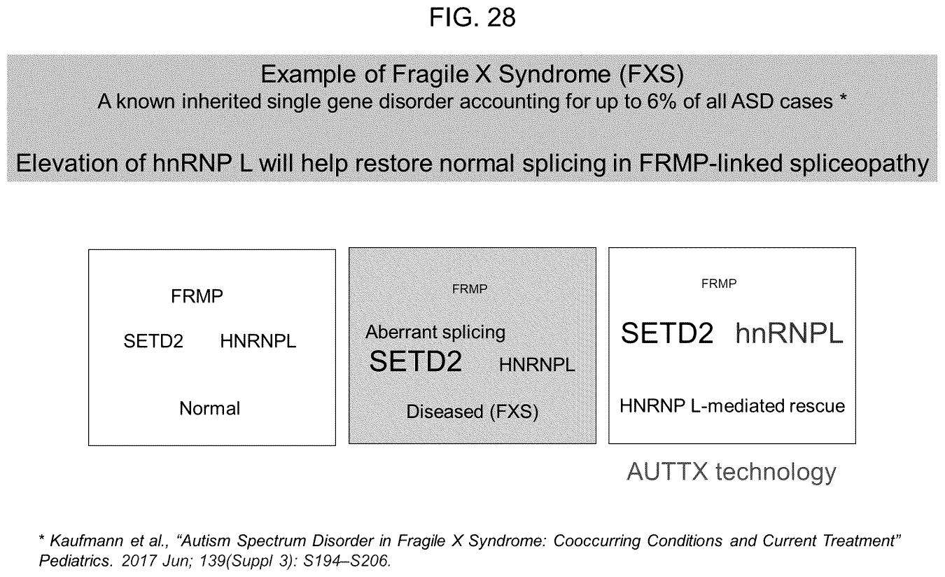

Some aspects that may relate to fragile X syndrome are included in FIG. 28. In some embodiments, the subject with fragile X syndrome, or a subject to be treated, has a mutation or defect in a gene in FIG. 28.

### Gerstmann-Straussler-Scheinker Disease

In some embodiments, a disease or disorder includes Gerstmann-Sträussler-Scheinker syndrome (GSS), an extremely rare, usually familial, fatal neurodegenerative disease that affects patients from 20 to 60 years in age. It is exclusively heritable, and is found in only a few families all over the world (according to NINDS). It is, however, classified with the transmissible spongiform encephalopathies (TSE) due to the causative role played by PRNP, the human prion protein. GSS was first reported by the Austrian physicians Josef Gerstmann, Ernst Sträussler and Ilya Scheinker in 1936. Familial cases are associated with autosomal-dominant inheritance.

Certain symptoms are common to GSS, such as progressive ataxia, pyramidal signs, and even adult-onset dementia; they progress more as the disease progresses.

Symptoms start with slowly developing dysarthria (difficulty speaking) and cerebellar truncal ataxia (unsteadiness) and then the progressive dementia becomes more evident. Loss of memory can be the first symptom of GSS. Extrapyramidal and pyramidal symptoms and signs may occur and the disease may mimic spinocerebellar ataxias in the beginning stages. Myoclonus (spasmodic muscle contraction) is less frequently seen than in Creutzfeldt-Jakob disease. Many patients also exhibit nystagmus (involuntary movement of the eyes), visual disturbances, and even blindness or deafness. The neuropathological findings of GSS include widespread deposition of amyloid plaques composed of abnormally folded prion protein.

Four clinical phenotypes are recognised: typical GSS, GSS with areflexia and paresthesia, pure dementia GSS and Creutzfeldt-Jakob disease-like GSS.

GSS is one of a small number of diseases that are caused by prions, a class of pathogenic proteins highly resistant to proteases. A change in codon 102 from proline to leucine has been found in the prion protein gene (PRNP, on chromosome 20) of most affected individuals. Therefore, it appears this genetic change is usually required for the development of the disease.

GSS can be identified through genetic testing. Testing for GSS involves a blood and DNA examination in order to attempt to detect the mutated gene at certain codons. If the genetic mutation is present, the patient will eventually be afflicted by GSS, and, due to the genetic nature of the disease, the offspring of the patient are predisposed to a higher risk of inheriting the mutation.

### Globular Glial Tauopathies

In some embodiments, a disease or disorder includes a tauopathy such as a globular glial tauopathy. Recent studies have highlighted a group of 4-repeat (4R) tauopathies that are characterised neuropathologically by widespread, globular glial inclusions (GGIs). Tau immunohistochemistry reveals 4R immunoreactive globular oligodendroglial and astrocytic inclusions and the latter are predominantly negative for Gallyas silver staining. These cases are associated with a range of clinical presentations, which correlate with the severity and distribution of underlying tau pathology and neurodegeneration. Their heterogeneous clinicopathological features combined with their rarity and under-recognition have led to cases characterised by GGIs being described in the literature using various and redundant terminologies. In this report, a group of neuropathologists form a consensus on the terminology and classification of cases with GGIs. After studying microscopic images from previously reported cases with suspected GGIs (n=22), this panel of neuropathologists with extensive experience in the diagnosis of neurodegenerative diseases and a documented record of previous experience with at least one case with GGIs, agreed that (1) GGIs were present in all the cases reviewed; (2) the morphology of globular astrocytic inclusions was different to tufted astrocytes and finally that (3) the cases represented a number of different neuropathological subtypes. They also agreed that the different morphological subtypes are likely to be part of a spectrum of a distinct disease entity, for which they recommend that the overarching term globular glial tauopathy (GGT) should be used. Type I cases typically present with frontotemporal dementia, which correlates with the fronto-temporal distribution of pathology. Type II cases are characterised by pyramidal features reflecting motor cortex involvement and corticospinal tract degeneration. Type III cases can present with a combination of frontotemporal dementia and motor neuron disease with fronto-temporal cortex, motor cortex and corticospinal tract being severely affected. Extrapyramidal features can be present in Type II and III cases and significant degeneration of the white matter is a feature of all GGT subtypes. Improved detection and classification will be necessary for the establishment of neuropathological and clinical diagnostic research criteria in the future.

For a detailed description of globular glial tauopathies, see Ahmed et al., Acta Neuropathol. 2013; 126 (4): 537-544, which is incorporated herein by reference in its entirety.

White Matter Tauopathy with Globular Glial Inclusions

In some embodiments, a disease or disorder includes a white matter tauopathy with globular glial inclusions. Frontotemporal lobar degenerations are a group of disorders characterized by circumscribed degeneration of the frontal and temporal lobes and diverse histopathological features. We report clinical, neuropathological, ultrastructural, biochemical and genetic data on seven individuals with a four-repeat (4R) tauopathy characterized by the presence of globular glial inclusions (GGIs) in brain white matter. Clinical manifestations were compatible with the behavioral variant of frontotemporal dementia (FTD) and included motor neuron symptoms; there was prominent neuronal loss in the frontal and temporal cortex, subiculum and amygdala. The surrounding white matter showed abundant GGIs composed of abnormal filaments present mostly in oligodendrocytes. The severity of white matter tau abnormalities correlated with a reduction in myelin and axons and with microglial activation. Western blotting of sarkosyl-insoluble tau demonstrated the presence of two major tau bands of 64 and 68 kDa. No mutations in the microtubule-associated protein tau (MAPT) gene were detected in two affected individuals. We propose that 4R tau-immunoreactive GGIs are the neuropathologic hallmark of a distinct sporadic tauopathy with variable clinical presentations that include FTD and occasionally upper motor neuron disease. This type of tauopathy with GGIs expands the group of neurodegenerative disorders in which oligodendroglial pathology predominates, beyond the synucleinopathy multiple system atrophy disorders.

For a detailed description of white matter tauopathy with globular glial inclusions, see Kovacs et al., J Neuropathol Exp Neurol. 2008; 67(10): 963-975, which is incorporated herein by reference in its entirety.

Guadeloupian Parkinsonism with Dementia

In some embodiments, a disease or disorder includes a Guadeloupian Parkinsonism with dementia. In Guadeloupe, there is an abnormally high frequency of atypical Parkinsonism. Only one-third of the patients that develop parkinsonian symptoms were reported to present the classical features of idiopathic Parkinson disease and one-third a syndrome resembling progressive supranuclear palsy (PSP). The others were unclassifiable, according to established criteria. We carried out a cross-sectional study of 160 parkinsonian patients to: (i) define more precisely the clinical phenotypes of the PSP-like syndrome and the parkinsonism that was considered unclassifiable in comparison with previously known disorders; (ii) define the neuropsychological and brain imaging features of these patients; (iii) evaluate to what extent a candidate aetiological factor, the mitochondrial complex I inhibitor annonacin contained in the fruit and leaves of the tropical plant Annona muricata (soursop) plays a role in the neurological syndrome. Neuropsychological tests and MRI were used to classify the patients into those with Parkinson's disease (31%), Guadeloupian PSP-like syndrome (32%), Guadeloupian parkinsonism-dementia complex (PDC, 31%) and other parkinsonism-related disorders (6%). Patients with a PSP-like syndrome developed levodopa-resistant Parkinsonism, associated with early postural instability and supranuclear oculomotor dysfunction. They differed, however, from classical PSP patients by the frequency of tremor (>50%), dysautonomia (50%) and the occurrence of hallucinations (59%). PDC patients had levodopa-resistant Parkinsonism associated with frontosubcortical dementia, 52% of these patients had hallucinations, but, importantly, none had oculomotor dysfunction. The pattern of neuropsychological deficits was similar in both subgroups. Cerebral atrophy was seen in the majority of the PSP-like and PDC patients, with enlargement of the third ventricle and marked T2-hypointensity in the basal ganglia, particularly the substantia nigra. Consumption of soursop was significantly greater in both PSP-like and PDC patients than in controls and Parkinson's disease patients. In conclusion, atypical Guadeloupian parkinsonism comprises two forms of parkinsonism and dementia that differ clinically by the presence of oculomotor signs, but have similar cognitive profiles and neuroimaging features, suggesting that they may constitute a single disease entity, and both were similarly exposed to annonaceous neurotoxins, notably annonacin.

For a detailed description of Guadeloupian Parkinsonism with dementia, see Lannuzel et al., Brain. 2007; 130(Pt 3):816-27, which is incorporated herein by reference in its entirety.

### Guadeloupian Progressive Supranuclear Palsy

In some embodiments, a disease or disorder includes a Guadeloupian progressive supranuclear palsy. An unusually high frequency of atypical Parkinson syndrome has been delineated over the last 5 years in the French West Indies. Postural instability with early falls, prominent frontal lobe dysfunction and pseudo-bulbar palsy were common and three-quarters of the patients were L-dopa unresponsive. One-third of all patients seen had probable progressive supranuclear palsy (PSP). This new focus of atypical Parkinsonism is reminiscent of the one described in Guam and may be linked to exposure to tropical plants containing mitochondrial complex I inhibitors (quinolines, acetogenins, rotenoids). Two hundred and twenty consecutive patients with Parkinson's syndrome seen by the neurology service at Pointe á Pitre, Guadeloupe University Hospital were studied. Currently accepted operational clinical criteria for Parkinson's syndromes were applied. The pathological findings of three patients who came to autopsy are reported. Fifty-eight patients had probable PSP, 96 had undetermined Parkinsonism and 50 had Parkinson's disease, 15 had amyotrophic lateral sclerosis with Parkinsonism and one had probable multiple system atrophy. All three PSP patients in whom post-mortem study was performed had early postural instability, gaze palsy and parkinsonian symptoms, followed by a frontolimbic dementia and corticobulbar signs. Neuropathological examination showed an accumulation of tau proteins, predominating in the midbrain. There was an exceptionally large accumulation of neuropil threads in Case 1. Biochemical studies detected a major doublet of pathological tau at 64 and 69 kDa in brain tissue homogenates. All cases were homozygous for the H1 tau haplotype, but no mutation of the tau gene was observed. Clinical, neuropathological and biochemical features were compatible with the diagnosis of PSP, although some unusual pathological features were noted in Case 1. A cluster of cases presenting with atypical Parkinsonism is reported. Guadeloupian Parkinsonism may prove to be a tauopathy identical or closely related to PSP.

For a detailed description of Guadeloupian progressive supranuclear palsy, see Caparros-Lefebvre et al., Brain. 2002; 125(Pt 4):801-811, which is incorporated herein by reference in its entirety.

Neurodegeneration with Brain Iron Accumulation

In some embodiments, a disease or disorder includes neurodegeneration. The neurodegeneration may include a neurodegeneration with brain iron accumulation (NBIA), which is a group of inherited neurologic disorders characterized by abnormal accumulation of iron in the basal ganglia (most often in the globus pallidus and/or substantia nigra). Generalized cerebral atrophy and cerebellar atrophy are frequently observed. The hallmark clinical manifestations of NBIA are progressive dystonia and dysarthria, spasticity, Parkinsonism, neuropsychiatric abnormalities, and optic atrophy or retinal degeneration. Although cognitive decline occurs in some genetic types, more often cognition is relatively spared. Onset ranges from infancy to adulthood. Progression can be rapid or slow with long periods of stability.

The quality of the neuroimaging, including magnet strength and spacing of image slices, can limit the ability to accurately identify abnormal brain iron. Iron-sensitive sequences, such as SWI, GRE, and T2*, should be used as a first-line diagnostic investigation to identify the characteristic changes in NBIA. By the time clear neurologic features are present, the brain MRI almost always shows characteristic changes, although iron may be visible only later in the disease course.

Neuropathologic findings include axonal spheroids in the CNS and, in some types, in peripheral nerves.

For a detailed description of NBIA, see Gregory and Hayflick, GeneReviews® [Internet] at the World Wide Web website of ncbi.nlm.nih.gov/books/NBK121988/, which is incorporated herein by reference in its entirety.

### Hallervorden-Spatz Disease

In some embodiments, a disease or disorder includes Hallervorden-Spatz disease, which is now more commonly known as Pantothenate kinase-associated neurodegeneration (PKAN), and is a rare autosomal recessive neurodegenerative disorder associated with iron accumulation in the brain nuclei and characterized by progressive extrapyramidal dysfunction and dementia.

Diagnosis of Hallervorden-Spatz disease may be done by CT imaging, brain MRI, SWI/T2*, SPECT scanning, and/or antenatal diagnosis.

For a detailed description of Hallervorden-Spatz disease, see Bokhari et al., StatPearls [Internet] at the World Wide Web website of ncbi.nlm.nih.gov/books/NBK430689/, which is incorporated herein by reference in its entirety.

### Pantothenate Kinase-Associated Neurodegeneration

In some embodiments, a disease or disorder includes a pantothenate kinase-associated neurodegeneration (PKAN), which is a type of neurodegeneration with brain iron accumulation (NBIA). The phenotypic spectrum of PKAN includes classic PKAN and atypical PKAN. Classic PKAN is characterized by early childhood onset of progressive dystonia, dysarthria, rigidity, and choreoathetosis. Pigmentary retinal degeneration is common. Atypical PKAN is characterized by later onset (age >10 years), prominent speech defects, psychiatric disturbances, and more gradual progression of disease.

The diagnosis of PKAN is established in a proband with the characteristic clinical features and the “eye of the tiger” sign identified on brain MRI (a central region of hyperintensity surrounded by a rim of hypointensity on coronal or transverse T2-weighted images of the globus pallidus). Identification of biallelic PANK2 pathogenic variants on molecular genetic testing confirms the diagnosis.

For a detailed description of PKAN, see Gregory and Hayflick, GeneReviews® [Internet] at the World Wide Web website of ncbi.nlm.nih.gov/books/NBK1490/, which is incorporated herein by reference in its entirety.

### Neurofibrillary Tangle-Predominant Dementia

In some embodiments, a disease or disorder includes neurofibrillary tangle predominant dementia (NFTPD), which is a subset of late onset dementia, clinically different from traditional “plaque and tangle” Alzheimer disease (AD): later onset, shorter duration, less severe cognitive impairment, and almost absence of ApoE epsilon. Neuropathology reveals abundant allocortical neurofibrillary pathology with no or few isocortical tau lesions, absence of neuritic plaques, absence or scarcity of amyloid deposits, but neurofibrillary changes comprising both 3 and 4 repeat (3R and 4R) tau immunohistochemistry are not significantly different from those in classical AD.

For a detailed description of NFTPD, see Jellinger and Attems, Acta Neuropathol. 2007; 113(2):107-117, which is incorporated herein by reference in its entirety.

### Niemann-Pick Disease

In some embodiments, a disease or disorder includes Niemann-Pick disease, a rare, inherited disease that affects the body's ability to metabolize fat (cholesterol and lipids) within cells. These cells malfunction and, over time, die. Niemann-Pick disease can affect the brain, nerves, liver, spleen, bone marrow and, in severe cases, lungs.

People with this condition experience symptoms related to progressive loss of function of nerves, the brain and other organs.

Niemann-Pick can occur at any age but mainly affects children. The disease has no known cure and is sometimes fatal. Treatment is focused on helping people live with their symptoms.

Niemann-Pick signs and symptoms may include: clumsiness and difficulty walking, excessive muscle contractions (dystonia) or eye movements, sleep disturbances, difficulty swallowing and eating, and/or recurrent pneumonia

The three main types of Niemann-Pick are types A, B and C. The signs and symptoms you experience depend on the type and severity of your condition. Some infants with type A will show signs and symptoms within the first few months of life. Those with type B may not show signs for years and have a better chance of surviving to adulthood. People with type C may not experience any symptoms until adulthood.

Niemann-Pick is caused by mutations in specific genes related to how the body metabolizes fat (cholesterol and lipids). The Niemann-Pick gene mutations are passed from parents to children in a pattern called autosomal recessive inheritance. This means that both the mother and the father must pass on the defective form of the gene for the child to be affected.

Niemann-Pick is a progressive disease, and there is no cure. It can occur at any age.

### There are at Least Three Types of Niemann-Pick Disease:

### Types A and B

Types A and B are caused by a missing or malfunctioning enzyme called sphingomyelinase. This affects the body's ability to metabolize fat (cholesterol and lipids), resulting in a buildup of fat in cells. This causes cell dysfunction and, over time, cell death. Type A occurs mainly in infants, who show severe, progressive brain disease. There is no cure, so most children do not live beyond their first few years. Type B usually occurs later in childhood and is not associated with primary brain disease. Most people affected with type B survive into adulthood.

### Type C

Niemann-Pick type C is a rare inherited disease. The genetic mutations of this type cause cholesterol and other fats to accumulate in the liver, spleen or lungs. The brain is eventually affected too.

### Prosencephalitic Parkinsonism

In some embodiments, a disease or disorder includes a post-encephalitic Parkinsonism, a disease believed to be caused by a viral illness that triggers degeneration of the nerve cells in the substantia nigra. Overall, this degeneration leads to clinical Parkinsonism. Historically, starting in 1917 an epidemic of encephalitis lethargica, also called von Economo's encephalitis or “sleepy-disease” occurred, possibly related to the 1918 Spanish flu pandemic; however, even with the use of modern molecular diagnostic tests on appropriate corpses no firm link between encephalitis lethargica with influenza has been made. Although Parkinsonism was occasionally seen during the acute encephalitic phase of encephalitis lethargica, it was often encountered in the post-encephalitic phase. The onset of post encephalitic Parkinsonism can be delayed by several years from the resolution of encephalitis lethargica.

The brain regions affected contain neurofibrillary tangles, similar to those seen in Alzheimer's disease. Nevertheless, the senile plaques common in Alzheimer's disease are not found.

For a detailed description of Prosencephalitic Parkinsonism, see Evidente and Gwinn, J Neurol Neurosurg Psychiatry. 64 (1): 5, which is incorporated herein by reference in its entirety.

### Prion Protein Cerebral Amyloid Angiopathy

In some embodiments, a disease or disorder includes a prion disease such as a prion protein (PrP) cerebral amyloid angiopathy. Deposition of PrP amyloid in cerebral vessels in conjunction with neurofibrillary lesions is the neuropathologic hallmark of the dementia associated with a stop mutation at codon 145 of PRNP, the gene encoding the PrP. In this disorder, the vascular amyloid in tissue sections and the approximately 7.5-kDa fragment extracted from amyloid are labeled by antibodies to epitopes located in the PrP sequence including amino acids 90-147. Amyloid-laden vessels are also labeled by antibodies against the C terminus, suggesting that PrP from the normal allele is involved in the pathologic process. Abundant neurofibrillary lesions are present in the cerebral gray matter. They are composed of paired helical filaments, are labeled with antibodies that recognize multiple phosphorylation sites in tau protein, and are similar to those observed in Alzheimer disease. A PrP cerebral amyloid angiopathy has not been reported in diseases caused by PRNP mutations or in human transmissible spongiform encephalopathies; named PrP cerebral amyloid angiopathy (PrP-CAA).

For a detailed description of Prp-CAA, see Ghetti et al., PNAS 1996 93:744-748, which is incorporated herein by reference in its entirety.

### Subacute Sclerosing Panencephalitis

In some embodiments, a disease or disorder includes a subacute sclerosing panencephalitis, a progressive and usually fatal brain disorder, which is a rare complication of measles that appears months or years later and causes mental deterioration, muscle jerks, and seizures.

Subacute sclerosing panencephalitis results from a long-term brain infection with the measles virus. The virus sometimes enters the brain during a measles infection. It may cause immediate symptoms of brain infection (encephalitis), or it may remain in the brain for a long time without causing problems.

Subacute sclerosing panencephalitis occurs because the measles virus reactivates. In the past in the United States, for reasons that are not known, the disorder occurred in about 7 to 300 people per million people who had measles infection and in about 1 person per million people who received the measles vaccine. However, doctors think the people who developed subacute sclerosing panencephalitis after vaccination likely had a mild, undiagnosed case of measles before they were vaccinated and that the vaccine did not cause the subacute sclerosing panencephalitis.

Subacute sclerosing panencephalitis is rare in the United States and Western Europe because of widespread measles vaccination. However, analyses of more recent measles outbreaks suggest that the incidence of subacute sclerosing panencephalitis may be higher than previously thought.

Males are affected more often than females. The risk of developing subacute sclerosing panencephalitis is highest in people who contract measles before they are 2 years of age. Subacute sclerosing panencephalitis usually begins in children or young adults, usually before age 20.

The first symptoms of subacute sclerosing panencephalitis may be poor performance in schoolwork, forgetfulness, temper outbursts, distractibility, sleeplessness, and hallucinations. Sudden muscular jerks of the arms, head, or body may occur. Eventually, seizures may occur, together with abnormal uncontrollable muscle movements. Intellect and speech continue to deteriorate.

Later, the muscles become increasingly rigid, and swallowing may become difficult. The swallowing difficulty sometimes causes people to choke on their saliva, resulting in pneumonia. People may become blind. In the final phases, the body temperature may rise, and the blood pressure and pulse become abnormal.

Tests on cerebrospinal fluid or blood and/or imaging tests may be used for diagnosis.

A doctor suspects subacute sclerosing panencephalitis in young people who have mental deterioration and muscle jerks and a previous history of measles. The diagnosis may be confirmed by examination of cerebrospinal fluid, a blood test that reveals high levels of antibody to the measles virus, by an abnormal electroencephalogram (EEG), and by magnetic resonance imaging (MRI) or computed tomography (CT) that shows brain abnormalities. A biopsy of the brain may need to be done if the tests cannot reveal a cause.

For a detailed description of subacute sclerosing panencephalitis, see the review from Tesini, published on the World Wide Web website of merckmanuals.com/home/children-s-health-issues/viral-infections-in-infants-and-children/subacute-sclerosing-panencephalitis-sspe, which is incorporated herein by reference in its entirety.

### Isoprenoid Antibiotics

Disclosed herein, in some embodiments, are antibiotics such as isoprenoid antibiotics. In some embodiments, a subject having a disease or disorder is treated with or administered the antibiotic. Isoprenoid antibiotics, including but not limited to the compounds ascochlorin, and its derivatives/analogues (i.e. natural and synthetic related compounds, e.g., ascofuranone (AF) and AF analogs or derivatives described herein, ascofuranol, MAC, AS-6, cylindrol A5, vertihemipterin A, vertihemipterin A aglycone, 8′-hydroxyascochlorin, 8′,9′-dehydroaschchlorin, 8′-acetoxyascochlorin, colletochlorin) can be used directly, and/or as chemical template structures, to treat ALS and FTD, and other diseases described herein, including but not limited to, autism, autism spectrum disorder and related neurological and psychiatric disorders, such as mental retardation, learning disability, attention deficit hyperactivity disorder, dyslexia, epilepsy, bipolar disorder, and schizophrenia.

Proteome analysis has demonstrated that ascochlorin treatment of human osteosarcoma cells (U2OS) results in a ≥10 fold increase in the levels of three proteins, including the splicing factor hnRNP L.

Neurological disorders characterized by an hnRNP L binding site aberration-mediated spliceopathy are treated using isoprenoid (prenyl-phenol) antibiotics, including but not limited to the compounds ascochlorin, its derivatives and analogs (e.g., ascofuranone (AF) and AF analogs or derivatives described herein, ascofuranol, MAC, AS-6, cylindrol A₅, vertihemipterin A, vertihemipterin A aglycone, 8′-hydroxyascochlorin, 8′,9′-dehydroaschchlorin, 8′-acetoxyascochlorin, colletochlorin) which can be used directly, and/or as chemical template structures, to help treat neurological disorders in humans. Relevant neurological and psychiatric disorders include, but are not limited to, Amyotrophic Lateral Sclerosis (ALS), Frontotemporal Dementia (FTD), or other diseases described herein.

### Natural Sources of the Isoprenoid Antibiotic Compounds

Isoprenoid antibiotics were originally isolated from the phytopathogenic fungus Ascochyta viciae. (Sasaki, H. et al. J Antibiot (Tokyo), 1973, 26:676-680). Among them, ascochlorin and ascofuranone have been shown to be non-toxic compounds. Structurally related compounds have been subsequently isolated from other fungi (e.g., Fusarium sp., Cylindrocladium sp., Cylindrocladium ilicicola, Nectria coccinea, Nectria galligena, Colletotrichum nicotianae, Acremonium sp., Ascochyta viciae, Ascochyta viciae, Acremonium luzulae, Acremonium egyptiacum, Cephalosporium diospyri, Verticillium sp., Cylindrocarpon lucidum, Nigrosabulum globosum, and the insect pathogenic fungus Verticillium hemipterigenum). (See Hosono, K. et al. J Antibiot (Tokyo), 2009, 62:571-574; phonkai, P. et al. J Antibiot (Tokyo), 2004, 57:10-16).

### Physiological Properties of the Isoprenoid Antibiotic Compounds

Studies have demonstrated that the methylated derivative of ascochlorin, 4-O-methylascochlorin (MAC), increases the expression of vascular endothelial growth factor (VEGF) and glucose transporter 1 (GLUT-1) (Jeong J. H. et al. Biochem Biophys Res Commun. 2011; 406:353-358). Both VEGF and GLUT-1 RNAs are well-established targets of hnRNP L (Hamilton B. J. et al. Biochem Biophys Res Commun. 1999; 261:646-651; Ray P. S. et al. Nature. 2009; 457:915-919; Shih S. C. et al. J Biol Chem. 1999; 274:1359-1365).

The current data illustrate that Ascochlorin and/or its derivatives promote the maintenance of normal brain physiology by targeting hnRNP L and/or components of the coordinated hnRNP L-regulated pathway(s). The compounds and methods of the disclosure provide pharmacological leads to help treat TDP-proteinopathies (e.g., amyotrophic lateral sclerosis, ALS) and other cryptic/poison exon-induced neurological disorders, CEIND, PEIND). Ascochlorin (ASC) and derivatives (e.g., MAC) as well as analogs (e.g., ascofuranone) display antitumorigenic properties, both in vitro and in vivo (summarized in Table 1 in Min-Wen et al., Adv Protein Chem Struct Biol. 2017; 108:199-225). More recently, the novel ASC derivatives acremochlorin A and 3-bromoascochlorin were shown to display anti-cancer properties (doi.org/10.25135/rnp.329.2204.2437).

In addition to anticancer properties, ascochlorin and its derivatives exhibit additional physiological activities, including antimicrobial/antiviral activity, trypanocidal properties, hypolipidemic activity, suppression of hypertension, improvement of type I and II diabetes, anti-inflammatory, and immunomodulation. (Yabu, Y. et al. Parasitol Int. 2003, 52:155-164; Hosono, K. et al. J Antibiot (Tokyo), 2009, 62:571-574; Lee et al., J Cell Biochem. 2016 April; 117(4):978-87; Shen et al., Eur J Pharmacol. 2016 Nov. 15; 791:205-212).

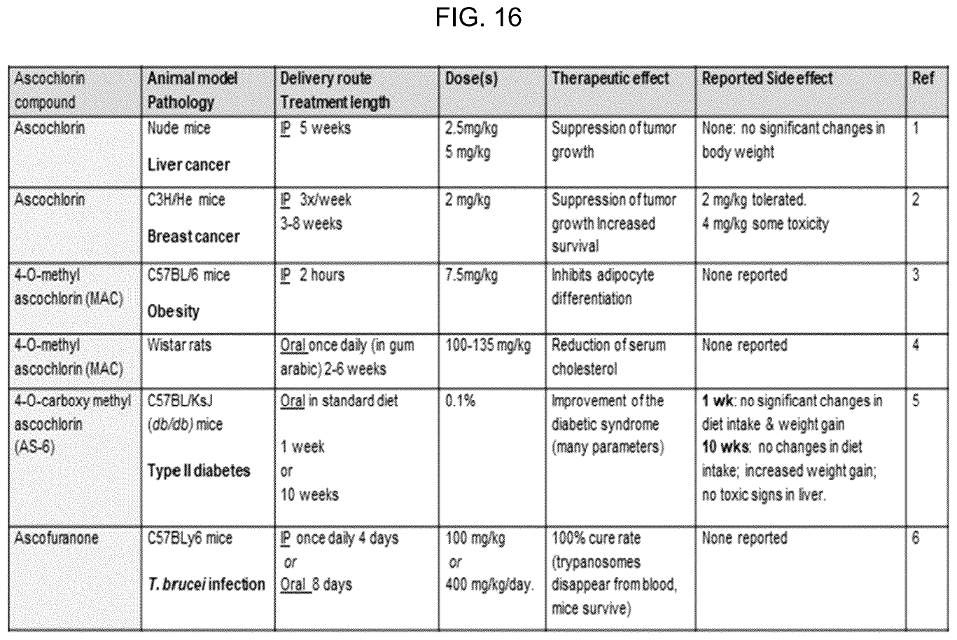

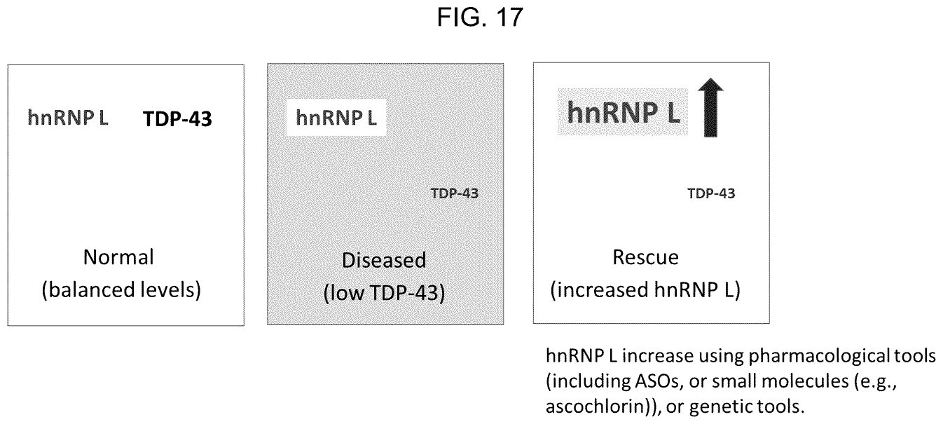

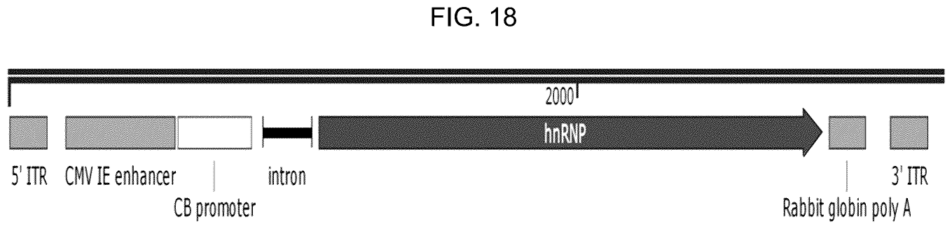

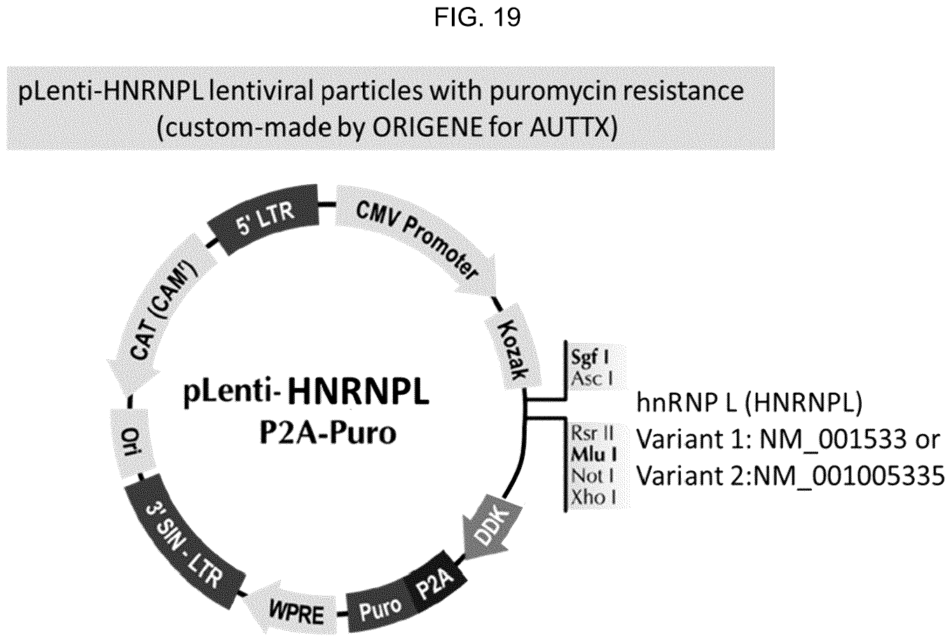

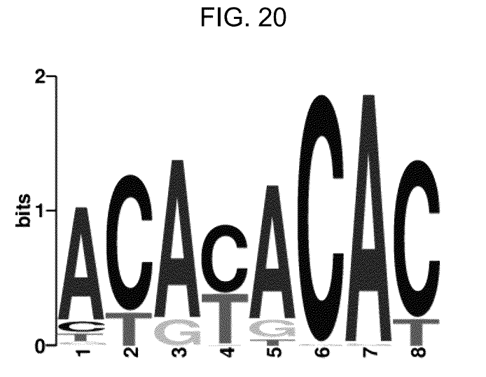

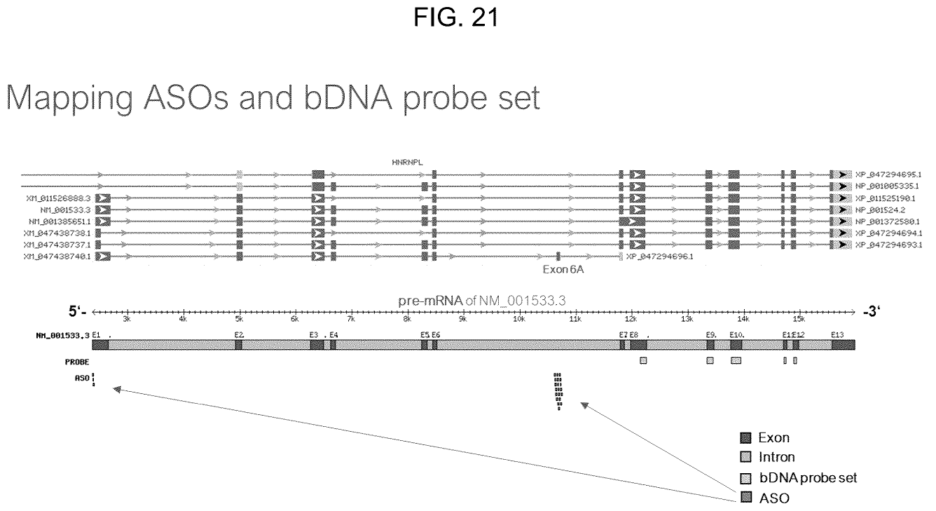

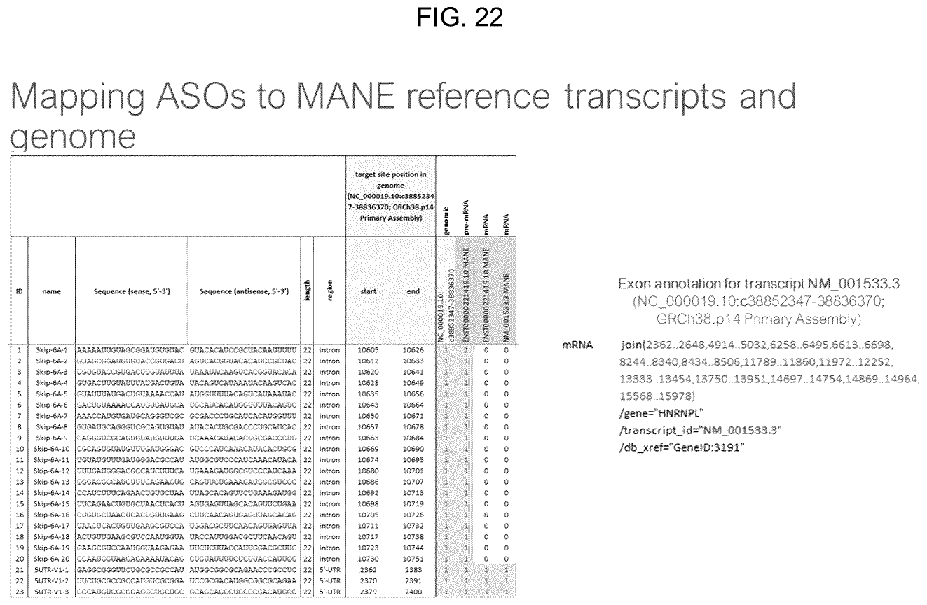

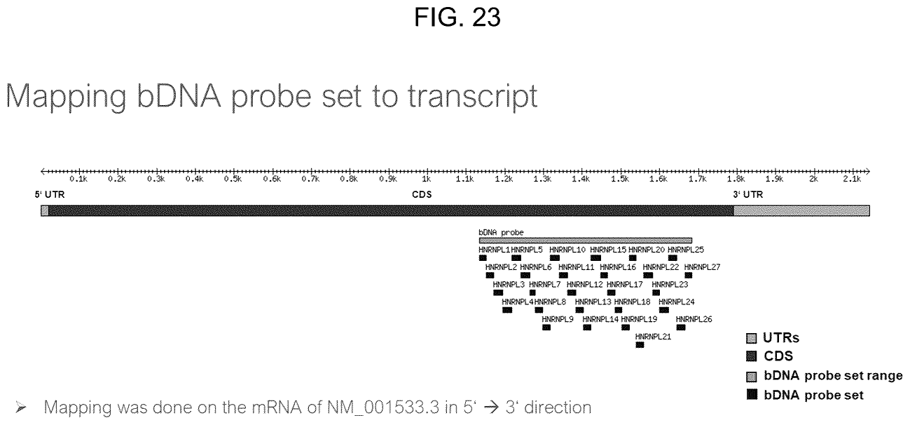

Examples of ascochlorin/derivative treatment effects in various rodent models of disease are also shown in FIG. 16.

### Other Examples of Ascochlorin Derivatives May be Found in:

(i) Pubchem databases: World Wide Web website at ncbi.nlm.nih.gov/pccompound/?term=ascochlorin: by using the “similar compounds” interactive link;
(ii) Pharmaceutical composition and method of using the same. U.S. Pat. No. 3,995,061.
(iii) Ascochlorin derivatives, and pharmaceutical composition containing the same. U.S. Pat. No. 4,500,544;
(iv) Pyridyl carbonyl ascochlorin derivatives and pharmaceutical compositions containing the same. U.S. Pat. No. 4,542,143;
(v) Ligands of nuclear receptor. U.S. Pat. No. 6,605,639.
(vi) Ascochlorin derivative and use thereof as ampk activator. WO 2017/119515.
(vii). R. A. West, et al., African trypanosomiasis: Synthesis & SAR enabling novel drug discovery of ubiquinol mimics for trypanosome alternative oxidase, European Journal of Medicinal Chemistry 141 (2017) 676-689, doi.org/10.1016/j.ejmech.2017.09.067.
(viii). Togashi, M et al., Ascochlorin Derivatives as Ligands for Nuclear Hormone Receptors. J. Med. Chem. 2003; 46:4113-4123.
(ix). Saimoto, H et al., Pharmacophore Identification of Ascofuranone, Potent Inhibitor of Cyanide-Insensitive Alternative Oxidase of Trypanosoma brucei. J. Biochem. 2013; 153:267-273.

Additional examples of ascochlorin derivatives include an ascochlorin derivative from Cylindrocarpon sp. FKI-4602. Kawaguchi et al., J Antibiot (Tokyo). 2013 January; 66 (1): 23-9; ascochlorin derivatives from the leafhopper pathogenic fungus Microcera sp. BCC 17074. Isaka et al., J Antibiot (Tokyo). 2015 January; 68(1):47-5; and competitive Hdhodh inhibitorsm Shen et al., Eur J Pharmacol. 2016 Nov. 15; 791:205-212. The contents of each of the foregoing references is hereby incorporated by reference.

### Compounds for Treatment of Neurologic Disorders

Disclosed herein, in some embodiments, are compounds useful for treatment of a disease or disorder such as a neurologic disorder. The compound may be administered to a subject or used to treat the disease or disorder. The compound may be or include an oligonucleotide (e.g., an antisense oligonucleotide). The compound may be included among the formulas below.

The potential of ascochlorin (which increases hnRNP L levels ˜12× (Kang J. H. et al. J Proteome Res. 2006; 5:2620-2631)) and its derivatives as for the treatment of autism spectrum disorder is underscored by the observation that hnRNP L directly interacts with FOX1. Pharmacological stabilization of the hnRNP L-FOX1 complex may be beneficial in cases where a decrease in the levels of FOX1 (˜5.9×) is known to cause autism (Voineagu I. et al. Nature. 2011; 474:380-384). Further highlighting the potential of ascochlorin and its derivatives for the treatment neurological disorders is evidence that some antibiotics have ancillary neuroprotective effects (Stock M. L. et al. Neuropharmacology. 2013; 73C: 174-182).

Other ascochlorin derivatives or analogs include, for example, vertihemipterin A, 4-O-methyl ascochlorin (MAC), vertuhemipterin A aglycone, AS-6,8′-hydroxyascochlorin, cylindrol A5,8′,9′-dehydroascochlorin, ascofuranol, LL-Z1272ζ (8′-acetoxyascochlorin), ascofuranone (AF) and AF analogs or derivatives described in West et al. European Journal of Medicinal Chemistry 2017; 141:676-689, the content of which is incorporated by reference herein in its entirety, etc. In some embodiments, the ascochlorin derivatives or analogs described herein comprise a small molecule compound comprising at least one of compounds described in WO2019053159 and WO2017119515, both of which incorporated by references herein in their entities. Ascochlorin and derivatives thereof can also be found in or produced by fungal species, for example, Acremonium sp., Acremonium lacunae, Acremonium egyptiacum, Ascochyta viciae, Ascochyta viciae, Cephalosporium diospyri, Colletotrichum nicotianae, Cylindrocladium sp., Cylindrocladium ilicicola, Cylindrocarpon lucidum, Fusarium sp., Nectria galligena, Nectria coccinea, Nigrosabulum globosum, Verticillium hemipterigenum, or Verticillium sp. Other ascochlorin derivatives or analogs include, for example, neostigmine bromide, irinotecan, captopril, proscillaridin, digoxin or 0179445-0000 (DSigDB), vandetanib, amantadine, Phenethyl Isothiocyanate, Astemizole, Lansoprazole, Docetaxel, Paclitaxel, or other FDA- or EMA-approved compound that elevates the levels of hnRNP L. In some embodiments, the agents for treating of neurologic disorders described herein, including ascochlorin derivatives or analogs, comprise any one of the exemplary structures as shown in Formula 1-77 below, or a pharmaceutically acceptable salt thereof.

TABLE 2
Compounds of Formula 1
Formula 1

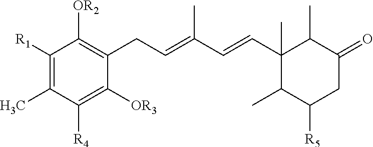

| Compound | | | | | |
|---|---|---|---|---|---|
| # | R₁ | R₂ | R₃ | R₄ | R₅ |
| 1 | CHO | H | H | Cl | H |
| 2 | CHO | H | H | Cl | OAc |
| 3 | CHO | H | H | Br | H |
| 4 | CHO | H | H | H | H |
| 5 | CHO | H | CH₃CO | Cl | H |
| 6 | CHO | H | CH₃ | Cl | H |
| 7 | CHO | CH₃ | CH₃ | Cl | H |
| 8 | CHO | CH₃CO | CH₃ | Cl | H |
| 9 | CHO | CH₃ | CH₃CO | Cl | H |
| 10 | CHO | CH₃ | H | Cl | H |
| 11 | CHO | H | CH₃CH₂ | Cl | H |
| 12 | CHO | H | Allyl | Cl | H |
| 13 | CHO | H | Butyl | Cl | H |
| 14 | CHO | H | CH₂COOH | Cl | H |
| 15 | CHO | H | (CH₂)₂COOH | Cl | H |
| 16 | CHO | H | (CH₂)₃COOH | Cl | H |
| 17 | CHO | H | (CH₂)₄COOH | Cl | H |
| 18 | CHO | H | CH₂COOCH₃ | Cl | H |
| 19 | CHO | H | Nicotinoyl | Cl | H |
| 20 | CHO | H | Benzoyl | Cl | H |
| 21 | CHO | H | Isonicotinoyl | Cl | H |
| 22 | CHO | H | CH₂COOC₂H₅ | Cl | H |
| 23 | CHO | H | CH₂COOCH₃ | Cl | H |
| 24 | CHO | H | CH₂COOH | Cl | H |
| 25 | CHO | H | CHCH₃COOC₂H₅ | Cl | H |
| 26 | CHO | H | CHCH₃COOC₄H₉ | Cl | H |
| 27 | CHO | H | CHCH₂CH₃COOC₂H₅ | Cl | H |
| 28 | CHO | H | (CH₂)₃COOC₂H₅ | Cl | H |
| 29 | CHO | H | CHCH₃COOH | Cl | H |
| 30 | CHO | H | (CH₂)₃COOH | Cl | H |
| 31 | CHO | H | Nicotinoyl | Cl | H |
| 32 | CHO | H | COC₆H₄OCH₃ | Cl | H |
| 33 | CHO | H | COC₆H₄COOCH₃ | Cl | H |
| 34 | CHO | H | CON(C₂H₅) | Cl | H |
| 35 | CHO | H | COCH₂OC₆H₄Cl | Cl | H |
| 36 | CHO | H | Isonicotinoyl | Cl | H |
| 37 | CHO | H | Picolinoyl | Cl | H |
| 38 | CHO | H | CH₃ | Cl | H |
| 39 | C₂H₂COCH₃ | H | H | Cl | O |
| 40 | CHO | H | CH₃CO | H | H |
| 41 | CHO | H | CH₃ | H | H |
| 42 | C₂H₂COCH₃ | H | CH₂COOH | Cl | O |
| 43 | CHO | CH₃CO | CH₃CO | Cl | H |
| 44 | C(OCH₃)₂ | H | CH₃CO | Cl | H |
| 45 | C(OCH₂CH₃)₂ | H | CH₃CO | Cl | H |
| 46 | C(OCH₂CH₃)₂ | H | CH₃ | Cl | H |
| 47 | C(O(CH₂)₃CH₃)₂ | H | CH₃ | Cl | H |
| 48 | 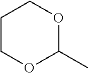 | H | CH₃ | Cl | H |
| 49 | CHO | H | (CH₂)₃CH₃ | Cl | H |
| 50 | CHO | H | CH₂CH₃ | Cl | H |
| 51 | CHO | H | CH₂CHCH₂ | Cl | H |
| 52 | CO₂H | H | H | Cl | H |

TABLE 3
Compounds of Formula 2
Formula 2

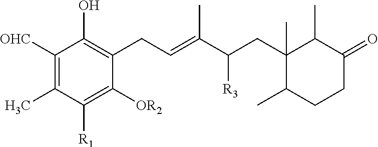

| Compound | | | |
|---|---|---|---|
| # | R₁ | R₂ | R₃ |
| 53 | Cl | H | H |
| 54 | H | H | H |
| 55 | Cl | H | OH |
| 56 | Cl | H | OAc |
| 57 | Cl | CH₃ | H |
| 58 | Cl | CH₃CO | H |
| 59 | H | CH₃ | H |
| 60 | H | CH₃CO | H |
| 61 | Cl | H | OCO(CH₃)₂ |
| 62 | Cl | H | OCOCH₂C(CH₃)₂ |

### Additional Compounds Include, but are not Limited to:

3-chloro-4,6-dihydroxy-2-methyl-5-((2E,4E)-3-methyl-5-((1R,2R,6R)-1,2,6-trimethyl-3-oxocyclohexyl) penta-2,4-dien-1-yl)benzaldehyde;
3-chloro-6-hydroxy-4-methoxy-2-methyl-5-((2E,4E)-3-methyl-5-((1R,2R,6R)-1,2,6-trimethyl-3-oxocyclohexyl) penta-2,4-dien-1-yl)benzaldehyde;
2-(2-chloro-4-formyl-5-hydroxy-3-methyl-6-((2E,4E)-3-methyl-5-((1R,2R,6R)-1,2,6-trimethyl-3-oxocyclohexyl) penta-2,4-dien-1-yl) phenoxy) acetic acid;
3-chloro-5-((2E,6E)-7-((S)-5,5-dimethyl-4-oxotetrahydrofuran-2-yl)-3-methylocta-2,6-dien-1-yl)-4,6-dihydroxy-2-methylbenzaldehyde;
(R,E)-5-(3-chloro-5-formyl-2,6-dihydroxy-4-methylphenyl)-3-methyl-1-((1S,2R,6R)-1,2,6-trimethyl-3-oxocyclohexyl) pent-3-en-2-yl butyrate;
3-chloro-4,6-dihydroxy-5-((2E,6E)-7-((2R,3S)-3-hydroxy-5,5-dimethyl-4-oxotetrahydrofuran-2-yl)-3-methylocta-2,6-dien-1-yl)-2-methylbenzaldehyde;
3-chloro-5-((R,E)-4-(((2R,3R,4R,5S,6R)-3,4-dihydroxy-6-(hydroxymethyl)-5-methoxytetrahydro-2H-pyran-2-yl)oxy)-3-methyl-5-((1S,2R,6R)-1,2,6-trimethyl-3-oxocyclohexyl) pent-2-en-1-yl)-4,6-dihydroxy-2-methylbenzaldehyde;
3-chloro-4,6-dihydroxy-5-((R,E)-4-hydroxy-3-methyl-5-((1S,2R,6R)-1,2,6-trimethyl-3-oxocyclohexyl) pent-2-en-1-yl)-2-methylbenzaldehyde;
3-chloro-4,6-dihydroxy-5-((2E,4E)-5-((1S,2S,3S,6R)-3-hydroxy-1,2,6-trimethyl-5-oxocyclohexyl)-3-methylpenta-2,4-dien-1-yl)-2-methylbenzaldehyde;
3-chloro-4,6-dihydroxy-2-methyl-5-((2E,4E)-3-methyl-5-((1S,2R,6R)-1,2,6-trimethyl-5-oxocyclohex-3-en-1-yl) penta-2,4-dien-1-yl)benzaldehyde;
3-chloro-4,6-dihydroxy-5-((2E,4E)-5-((1S,2S,3S,6R)-3-hydroxy-1,2,6-trimethyl-5-oxocyclohexyl)-3-methylpenta-2,4-dien-1-yl)-2-methylbenzaldehyde;
(E)-3-chloro-5-(3,7-dimethylocta-2,6-dien-1-yl)-4,6-dihydroxy-2-methylbenzaldehyde; cefacetrile; cefotaxime; ciproflaxin; netilimicine; or a quinolone/fluoroquinolone compound.

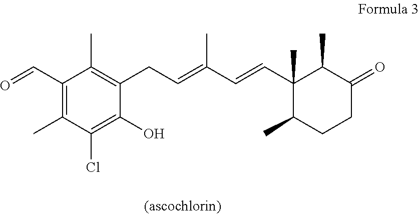 [SMILES: CC(/C=C/[C@@]1(C)[C@H](C)CCC(=O)[C@@H]1C)=C\Cc1c(C)c(C=O)c(C)c(Cl)c1O]

Formula 3 can be also displayed as Formula 3′, both of which refer to ascochlorin.

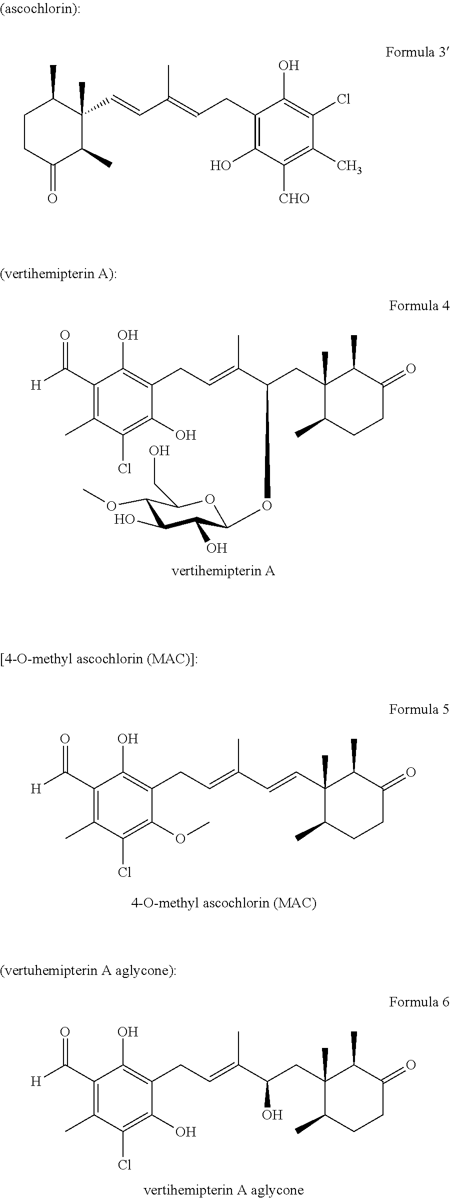

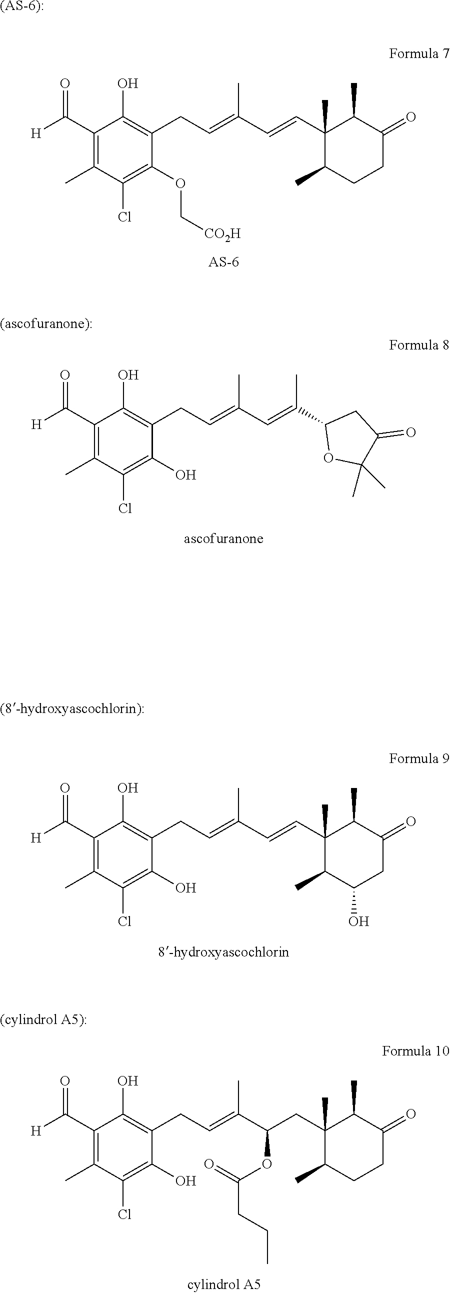

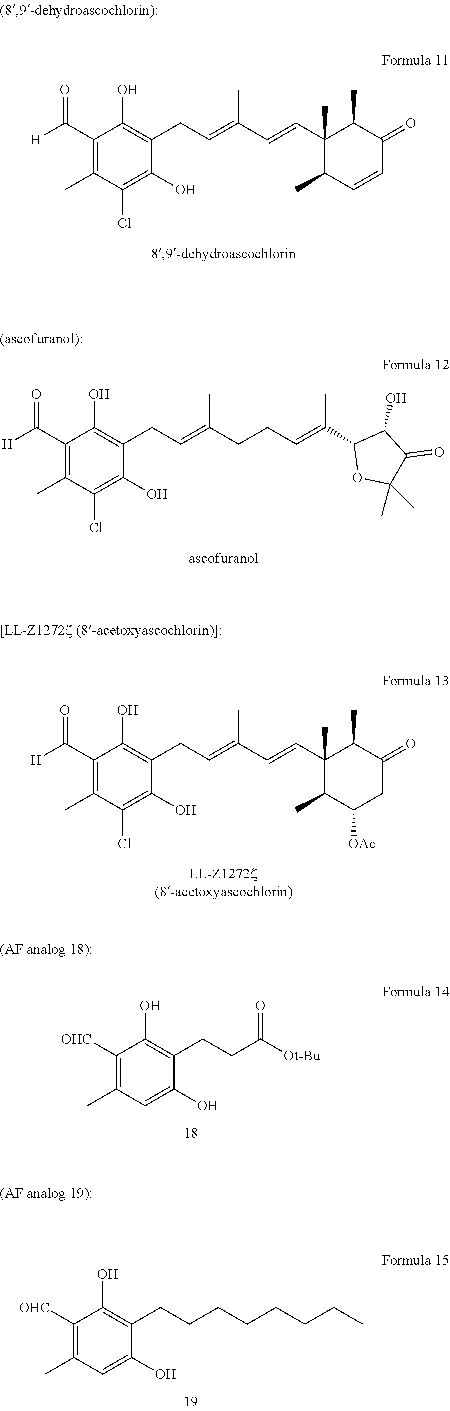 [SMILES: CC(/C=C/[C@@]1(C)[C@H](C)C=CC(=O)[C@@H]1C)=C\Cc1c(O)c(Cl)c(C)c(C=O)c1O ; C/C(=C\Cc1c(O)c(Cl)c(C)c(C=O)c1O)CC/C=C(\C)[C@H]1OC(C)(C)C(=O)[C@H]1O ; CC(=O)O[C@H]1CC(=O)[C@H](C)[C@@](C)(/C=C/C(C)=C/Cc2c(O)c(Cl)c(C)c(C=O)c2O)[C@@H]1C ; Cc1cc(O)c(CCC(=O)OC(C)(C)C)c(O)c1C=O ; CCCCCCCCc1c(O)cc(C)c(C=O)c1O]

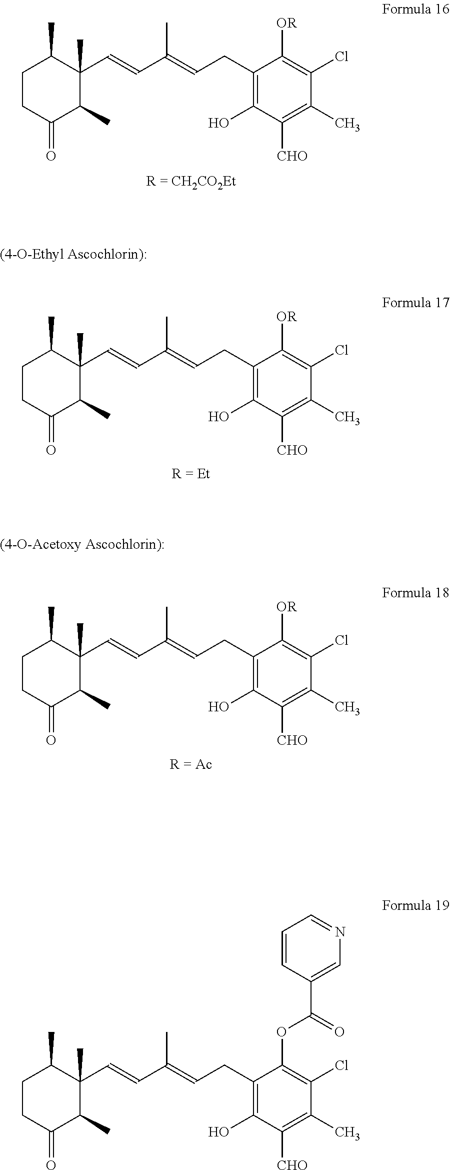

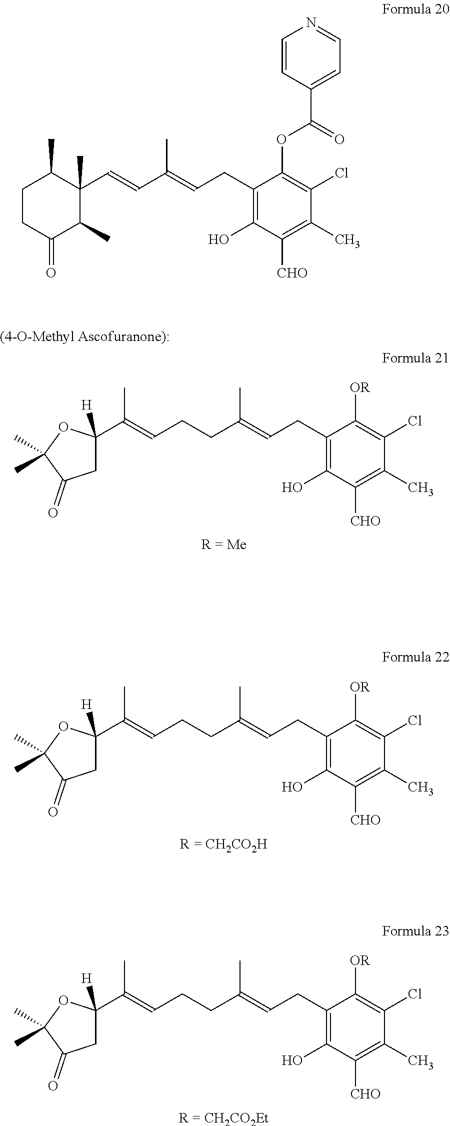

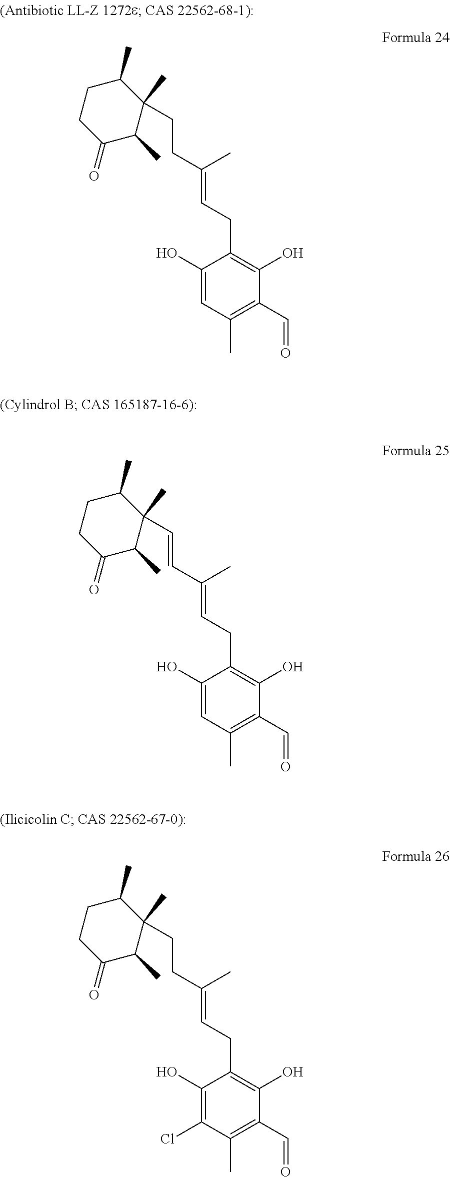 [SMILES: C/C(=C\Cc1c(O)cc(C)c(C=O)c1O)CC[C@@]1(C)[C@H](C)CCC(=O)[C@@H]1C ; CC(/C=C/[C@@]1(C)[C@H](C)CCC(=O)[C@@H]1C)=C\Cc1c(O)cc(C)c(C=O)c1O ; C/C(=C\Cc1c(O)c(Cl)c(C)c(C=O)c1O)CC[C@@]1(C)[C@H](C)CCC(=O)[C@@H]1C]

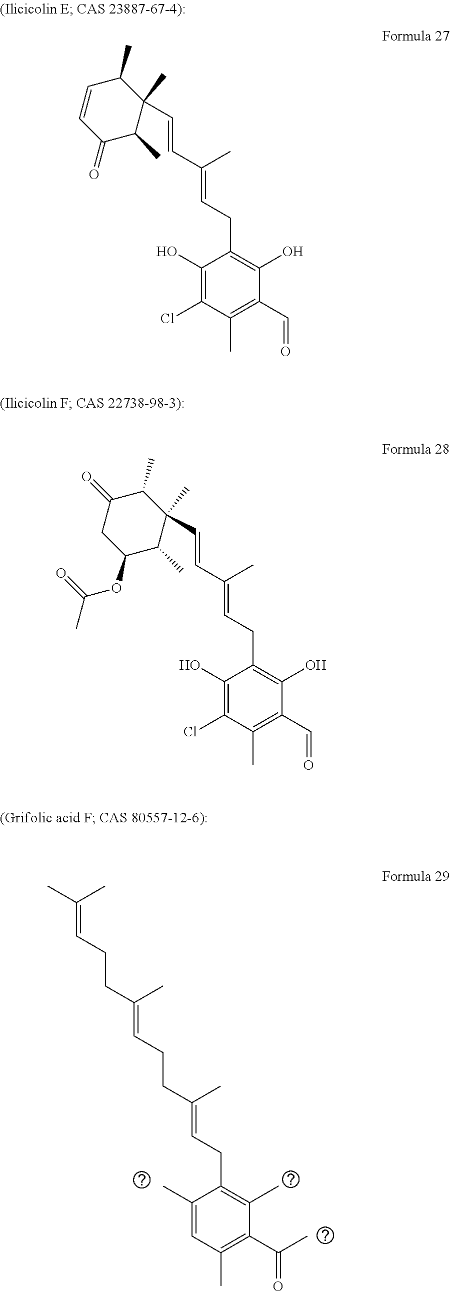 [SMILES: CC(/C=C/[C@@]1(C)[C@H](C)C=CC(=O)[C@@H]1C)=C\Cc1c(O)c(Cl)c(C)c(C=O)c1O ; CC(=O)O[C@H]1CC(=O)[C@H](C)[C@@](C)(/C=C/C(C)=C/Cc2c(O)c(Cl)c(C)c(C=O)c2O)[C@@H]1C ; CC(=O)c1c(C)cc(C)c(C/C=C(\C)CC/C=C(\C)CCC=C(C)C)c1C]

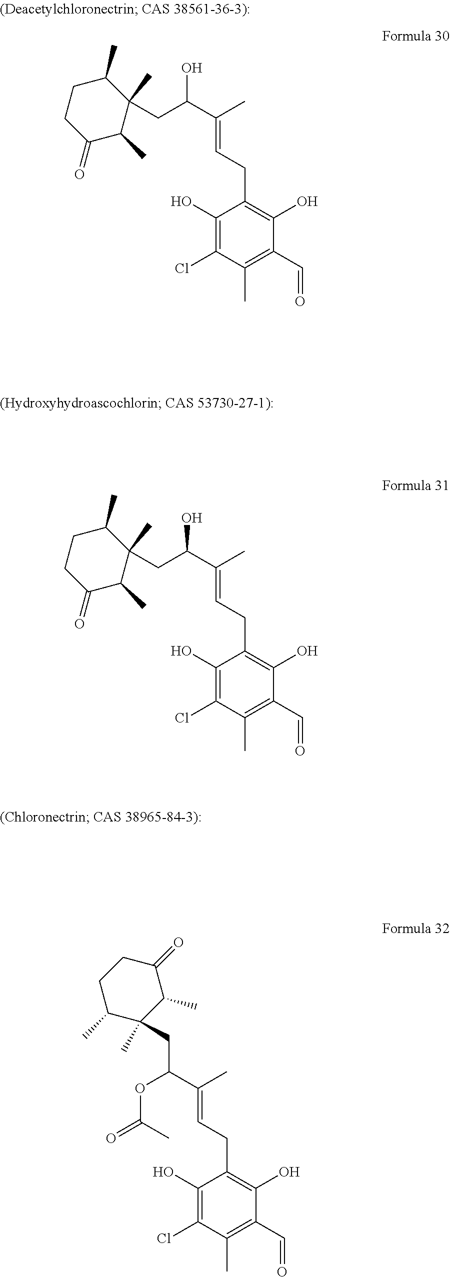 [SMILES: C/C(=C\Cc1c(O)c(Cl)c(C)c(C=O)c1O)C(O)C[C@@]1(C)[C@H](C)CCC(=O)[C@@H]1C ; C/C(=C\Cc1c(O)c(Cl)c(C)c(C=O)c1O)[C@H](O)C[C@@]1(C)[C@H](C)CCC(=O)[C@@H]1C ; CC(=O)OC(C[C@@]1(C)[C@H](C)CCC(=O)[C@@H]1C)/C(C)=C/Cc1c(O)c(Cl)c(C)c(C=O)c1O]

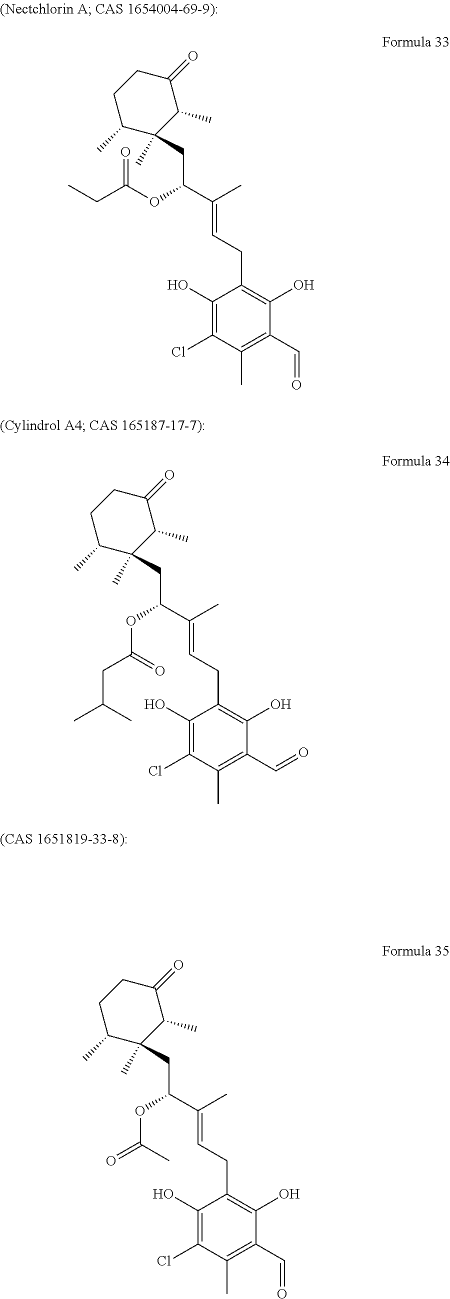 [SMILES: CCC(=O)O[C@H](C[C@@]1(C)[C@H](C)CCC(=O)[C@@H]1C)/C(C)=C/Cc1c(O)c(Cl)c(C)c(C=O)c1O ; C/C(=C\Cc1c(O)c(Cl)c(C)c(C=O)c1O)[C@@H](C[C@@]1(C)[C@H](C)CCC(=O)[C@@H]1C)OC(=O)CC(C)C ; CC(=O)O[C@H](C[C@@]1(C)[C@H](C)CCC(=O)[C@@H]1C)/C(C)=C/Cc1c(O)c(Cl)c(C)c(C=O)c1O]

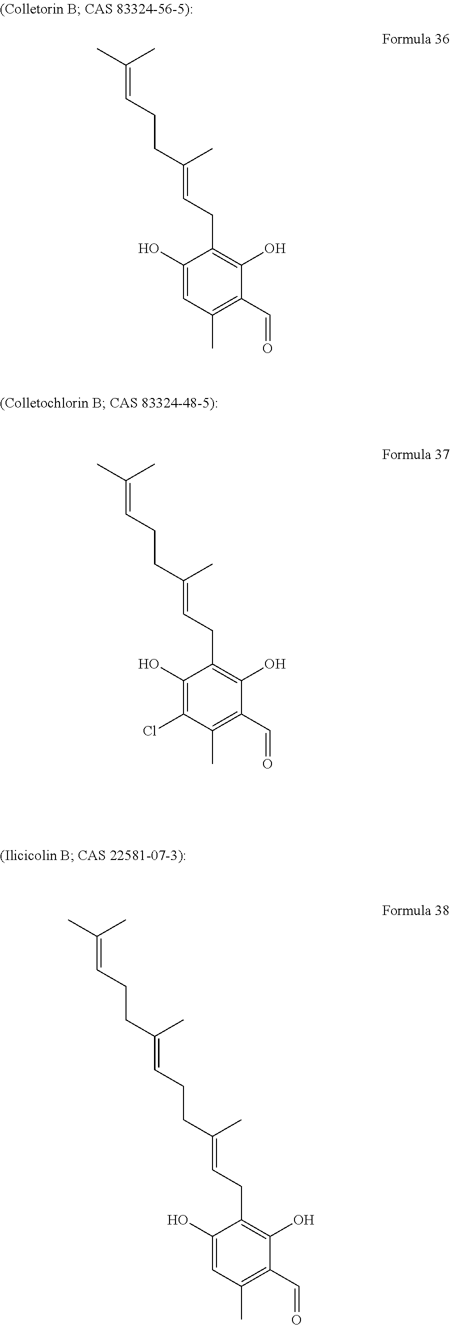 [SMILES: CC(C)=CCC/C(C)=C/Cc1c(O)cc(C)c(C=O)c1O ; CC(C)=CCC/C(C)=C/Cc1c(O)c(Cl)c(C)c(C=O)c1O ; CC(C)=CCC/C(C)=C/CC/C(C)=C/Cc1c(O)cc(C)c(C=O)c1O]

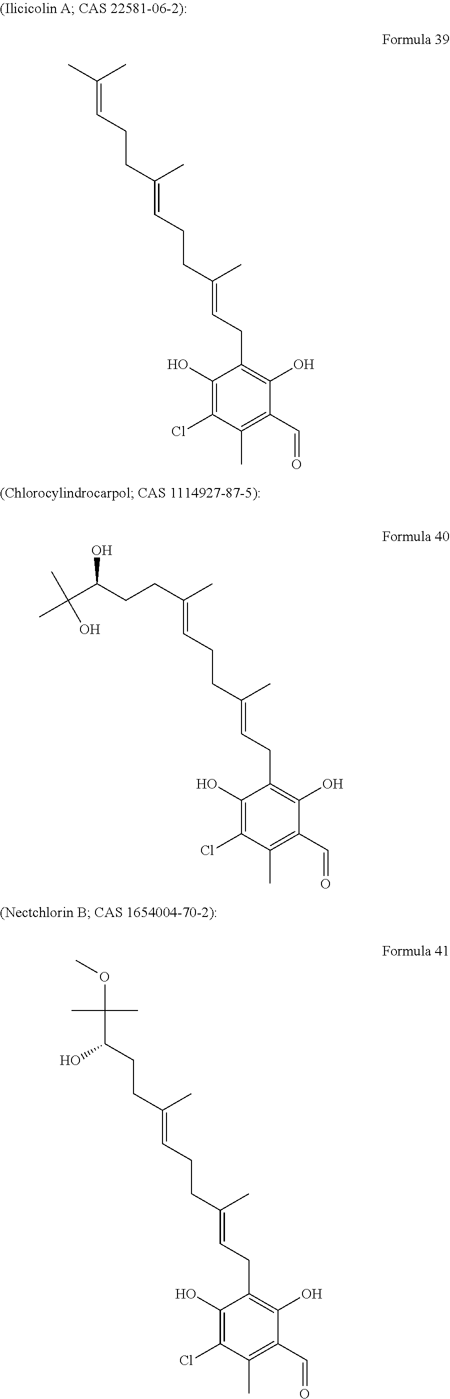 [SMILES: CC(C)=CCC/C(C)=C/CC/C(C)=C/Cc1c(O)c(Cl)c(C)c(C=O)c1O ; C/C(=C\Cc1c(O)c(Cl)c(C)c(C=O)c1O)CC/C=C(\C)CC[C@H](O)C(C)(C)O ; COC(C)(C)[C@@H](O)CC/C(C)=C/CC/C(C)=C/Cc1c(O)c(Cl)c(C)c(C=O)c1O]

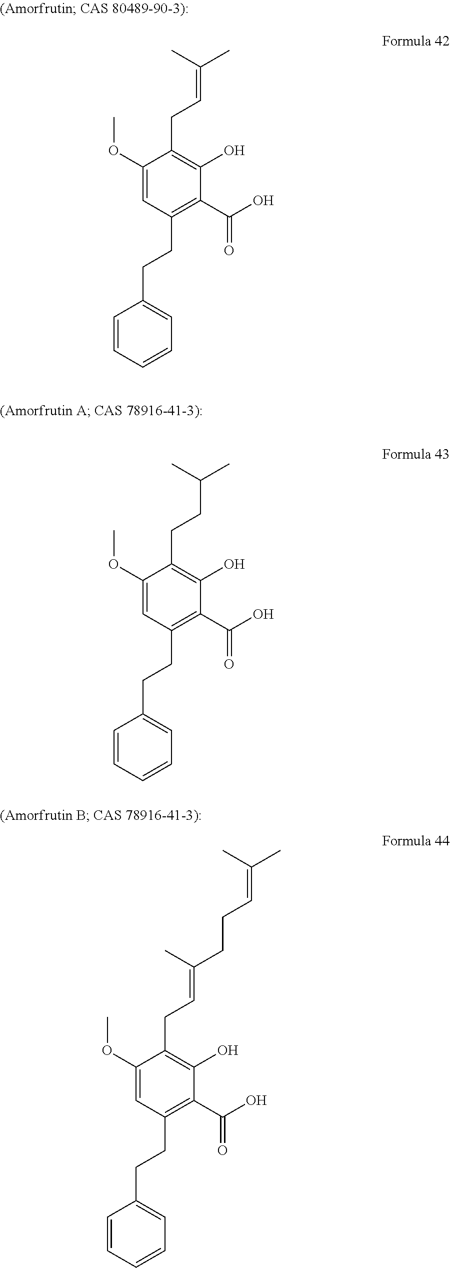 [SMILES: COc1cc(CCc2ccccc2)c(C(=O)O)c(O)c1CC=C(C)C ; COc1cc(CCc2ccccc2)c(C(=O)O)c(O)c1CCC(C)C ; COc1cc(CCc2ccccc2)c(C(=O)O)c(O)c1C/C=C(\C)CCC=C(C)C]

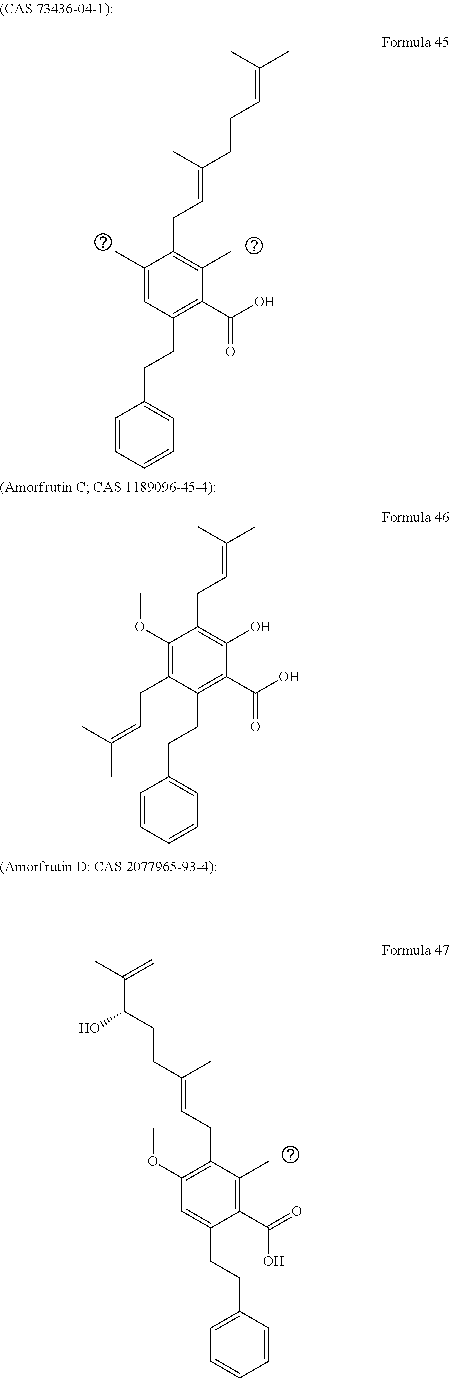

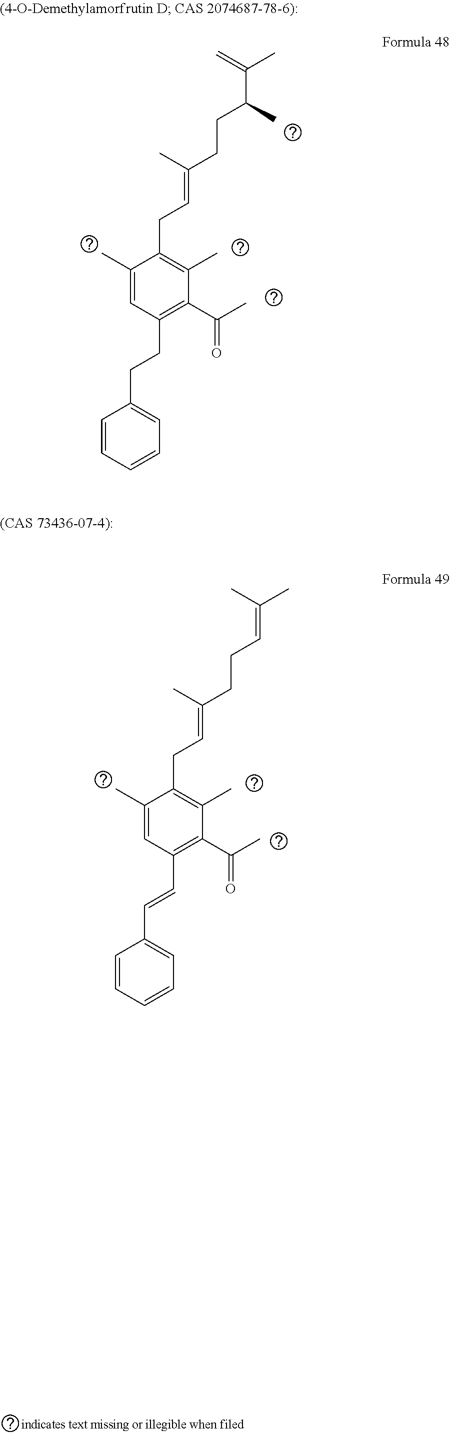 [SMILES: C=C(C)[C@@H](C)CC/C(C)=C/Cc1c(C)cc(CCc2ccccc2)c(C(C)=O)c1C ; CC(=O)c1c(/C=C/c2ccccc2)cc(C)c(C/C=C(\C)CCC=C(C)C)c1C]

Formulas 50-77 are based on the structure in Table 4 below (No. 1 is Table 4=Formula 50; No. 28 in Table 4=Formula 77):

TABLE 4
More ASC derivatives based on the Formula below:

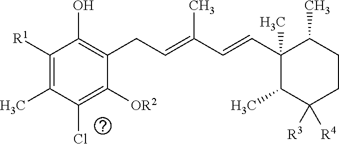

| No. | R¹ | R² | R³ | R⁴ |
|---|---|---|---|---|
| 1 | —CHO | Me | Me | OH |
| 2 | —CHO | Me | ═N—OH (E-form) | |
| 3 | —CH═N—OH | Me | ═N—OH (E-form) | |
| | (E-form) | | | |
| 4 | —CH═N—OH | CH₂F | ═N—OH (E-form) | |
| | (E-form) | | | |
| 5 | —CHO | CH₂F | ═N—OH (E-form) | |
| 6 | —CHO | Me | ═N—OC(═O)CH₂NMe₂ | |
| | | | (E-form) | |
| 7 | —CH═N—OH | Me | Me | OH |
| | (E-form) | | | |
| 8 | —CH═N—OMe | Me | ═N—OMe (E-form) | |
| | (E-form) | | | |
| 9 | —CH═N—OMe | CH₂F | ═N—OMe (B-form) | |
| | (E-form) | | | |
| 10 | —CH═N—OMe | Me | ═N—OH (E-form) | |
| | (E-form) | | | |
| 11 | —CH═N—OMe | CH₂F | ═N—OH (E-form) | |
| | (E-form) | | | |
| 12 | —CHO | CH₂F | 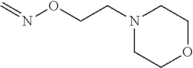 | |
| 13 | —CHO | CH₂F | H | 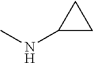 |
| 14 | —CH═N—OMe | Me | Me | OH |
| | (E-form) | | | |
| 15 | —CH═N—OMe | Me | ═N—O(CH₂)₂NMe₂ (E-form) | |
| | (E-form) | | | |
| 16 | —CH═N—OMe (E-form) | Me | 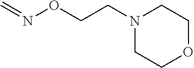 | |
| 17 | —CH═N—OMe (E-form) | Me | 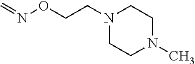 | |
| 18 | —CH═N—OMe | Me | ═N—OCH₂—CO₂Et (E-form) | |
| | (E-form) | | | |
| 19 | —CH═N—OMe | Me | ═N—OCH₂—CO₂H (E-form) | |
| | (E-form) | | | |
| 20 | —CHO | Me | H | NHEt |
| 21 | —CH═N—OMe | Me | H | NHEt |
| | (E-form) | | | |
| 22 | —CHO | Me | H | 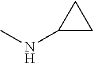 |
| 23 | —CH═N—OMe (E-form) | Me | H | 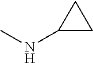 |
| 24 | —CH═N—OMe | Me | H | NH₂ |
| | (E-form) | | | |
| 25 | —CH═N—OMe (E-form) | Me | H | 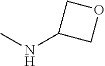 |

| | | | |
|---|---|---|---|
| 26 | —CHO | CH₂F | ═N—OCH₂—CO₂H (E-form) |
| 27 | —CH═N—OH | CH₂F | ═N—OCH₂—CO₂H (E-form) |
| | (E-form) | | |
| 28 | —CH═N—OH | Me | ═N—OCH₂—CO₂H (E-form) |
| | (E-form) | | |

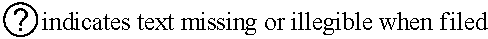

A compound may be or include an agent, and may comprise a pharmaceutically effective amount. A pharmaceutically effective amount of the agent described herein may comprise about 0.1 to about 100 mg/kg, about 0.1 to about 50 mg/kg, about 0.1 to about 20 mg/kg, about 0.1 to about 10 mg/kg, about 0.1 to about 5 mg/kg, about 0.5 to about 100 mg/kg, about 0.5 to about 50 mg/kg, about 0.5 to about 20 mg/kg, about 0.5 to about 10 mg/kg, about 0.5 to about 5 mg/kg, about 1 to about 100 mg/kg, about 1 to about 50 mg/kg, about 1 to about 20 mg/kg, about 1 to about 10 mg/kg, about 1 to about 5 mg/kg, about 5 to about 10 mg/kg, about 5 to about 20 mg/kg, about 5 to about 50 mg/kg, about 5 to about 100 mg/kg, or other amount of the agent.

### Compositions

The present disclosure provides a composition comprising an agent to increase expression levels and/or stability of hnRNP L. Such agent may be a small molecule compound described herein, such as ASC and its derivatives or analogs, as described herein. Such agent may include, for example, vertihemipterin A, 4-O-methyl ascochlorin (MAC), vertuhemipterin A aglycone, AS-6,8′-hydroxyascochlorin, cylindrol A5,8′,9′-dehydroascochlorin, ascofuranol, LL-Z1272ζ (8′-acetoxyascochlorin), ascofuranone (AF) and AF analogs or derivatives described in West et al. European Journal of Medicinal Chemistry 2017; 141:676-689, the content of which is incorporated by reference herein in its entirety, or bioactive ascochlorin analogs described in Subko et al. Marine Drugs 2021; doi.org/10.3390/md19020046, the content of which is incorporated by reference herein, etc. In some embodiments, the agent described herein comprises a small molecule compound comprising at least one of compounds described in WO2019053159 and WO2017119515, both of which incorporated by references herein in their entities.

Such agent may also be found in or produced by fungal species, for example, Acremonium sp., Acremonium lacunae, Acremonium egyptiacum, Ascochyta viciae, Ascochyta viciae, Cephalosporium diospyri, Colletotrichum nicotianae, Cylindrocladium sp., Cylindrocladium ilicicola, Cylindrocarpon lucidum, Fusarium sp., Nectria galligena, Nectria coccinea, Nigrosabulum globosum, Verticillium hemipterigenum, or Verticillium sp. Other agents include, for example, neostigmine bromide, irinotecan, captopril, proscillaridin, digoxin or 0179445-0000 (DSigDB), vandetanib, amantadine, Phenethyl Isothiocyanate, Astemizole, Lansoprazole, Docetaxel, Paclitaxel, or other FDA- or EMA-approved compound that elevates the levels of hnRNP L. In some embodiments, the agent described herein comprises any one of the exemplary structures as shown in Formulas 1-77, or a pharmaceutically acceptable salt thereof. The composition may be used for treating a disease or disorder. The composition may be administered to a subject.

Such agent may also be at least one of a nucleic acid, an enzyme, a protein, a polypeptide, an antibody or a functional fragment thereof, an aptamer, a small interfering RNA, a microRNA, a small hairpin RNA, an antisense nucleic acid, and a PNA, as described herein. Such agent may also be a polypeptide of, or a polynucleotide encoding such polypeptide of, hnRNP L or its biologically active fragments, as described herein. The agent may be or include a small molecule, a nucleic acid, an enzyme, a protein, a polypeptide, an antibody or a functional fragment thereof, an aptamer, a small interfering RNA, a microRNA, a long noncoding RNA (lncRNA), a small hairpin RNA, an antisense nucleic acid (e.g., ASO), a tricyclo-DNA (tcDNA), locked nucleic acid (LNA), a peptide nucleic acid (PNA), or a phosphorodiamidate morpholino oligomer (PMO).

In some embodiments, the composition comprises a recombinant nucleic acid molecule, which encodes a polypeptide of, or a polynucleotide encoding such polypeptide of, hnRNP L or its biologically active fragments. In some embodiments, the recombinant nucleic acid molecule is further defined as an expression cassette or a vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure includes a coding sequence for the hnRNP L polypeptide or its biologically active fragments as disclosed herein, which is operably linked to expression control elements, such as a promoter, and optionally, any other sequences or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the vector is a vector derived from a lentivirus, an adeno virus, an adeno-associated virus, a baculovirus, or a retrovirus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

An example related to viral vector use is provided in Example 5.

### Oligonucleotide Compositions

Disclosed herein, in some embodiments, are compositions comprising an oligonucleotide. In some embodiments, the composition comprises an oligonucleotide that targets hnRNP L or a poison exon of hnRNP L. For example, the oligonucleotide may be or include an antisense oligonucleotide that targets hnRNP L, and increases hnRNP L expression (protein or RNA such as mRNA). The oligonucleotide may target a poison exon of hnRNP L. The oligonucleotide may target a region near a poison exon of hnRNP L, that affects splicing or inclusion of the hnRNP L poison exon in a mature transcript or mRNA. In some embodiments, the composition consists of an oligonucleotide that targets hnRNP L or a poison exon of hnRNP L. In some embodiments, the oligonucleotide reduces hnRNP L mRNA expression in the subject, for example if the mRNA contains the poison exon or part of an intronic sequence. In some embodiments, the oligonucleotide increases hnRNP L mRNA expression in the subject. In some embodiments, the oligonucleotide increases hnRNP L protein expression in the subject. In some embodiments, the oligonucleotide reduces hnRNP L protein expression in the subject, though in most preferred embodiments the oligonucleotide is useful for increasing hnRNP L mRNA and/or protein expression. The oligonucleotide may include a small interfering RNA (siRNA). The oligonucleotide may include an antisense oligonucleotide (ASO) described herein. In some embodiments, a composition described herein is used in a method of treating a disorder in a subject in need thereof. Some embodiments relate to a composition comprising an oligonucleotide for use in a method of treating a disorder as described herein. Some embodiments relate to use of a composition comprising an oligonucleotide, in a method of treating a disorder (e.g., neurological) as described herein.

Some embodiments include a composition comprising an oligonucleotide that targets hnRNP L or a poison exon of hnRNP L and when administered to a subject in an effective amount increases hnRNP L mRNA or protein levels in a cell (e.g., neuron, glia), fluid (e.g., blood, serum, plasma, or cerebrospinal fluid (CSF)), tissue (e.g., brain), or organ (e.g., the brain, the spinal cord).

In some embodiments, the composition comprises an oligonucleotide that targets hnRNP L or a poison exon of hnRNP L and when administered to a subject in an effective amount increases hnRNP L mRNA levels in a cell or tissue. In some embodiments, the cell is a neuron. In some embodiments, the tissue is neural tissue. In some embodiments, the neural tissue is CNS tissue. In some embodiments, the neural tissue is brain tissue (e.g., neuronal, glia, or endothelial tissue). In some embodiments, the fluid is CSF. In some embodiments, the hnRNP L mRNA levels are increased by about 2.5% or more, about 5% or more, or about 7.5% or more, as compared to prior to administration. In some embodiments, the hnRNP L mRNA levels are increased by about 10% or more, as compared to prior to administration. In some embodiments, the hnRNP L mRNA levels are increased by about 20% or more, about 30% or more, about 40% or more, about 50% or more, about 60% or more, about 70% or more, about 80% or more, about 90% or more, or about 100% or more, as compared to prior to administration. In some embodiments, the hnRNP L mRNA levels are increased by no more than about 2.5%, no more than about 5%, or no more than about 7.5%, as compared to prior to administration. In some embodiments, the hnRNP L mRNA levels are increased by no more than about 10%, as compared to prior to administration. In some embodiments, the hnRNP L mRNA levels are increased by no more than about 20%, no more than about 30%, no more than about 40%, no more than about 50%, no more than about 60%, no more than about 70%, no more than about 80%, or no more than about 90%, as compared to prior to administration. In some embodiments, the hnRNP L mRNA levels are increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets hnRNP L or a poison exon of hnRNP L and when administered to a subject in an effective amount increases hnRNP L protein levels in a cell, fluid (e.g., CSF) or tissue. In some embodiments, the cell is a hepatocyte. In some embodiments, the cell is a neural cell (e.g., CNS cell (e.g., brain cell)). In some embodiments, the cell is a neuronal cell. In some embodiments, the cell is a glial cell. In some embodiments, the cell is an endothelial cell. In some embodiments, the tissue is neural (e g. CNS (e g., brain)) tissue. In some embodiments, the fluid is CSF. In some embodiments, the hnRNP L protein levels are increased by about 2.5% or more, about 5% or more, or about 7.5% or more, as compared to prior to administration. In some embodiments, the hnRNP L protein levels are increased by about 10% or more, as compared to prior to administration. In some embodiments, the hnRNP L protein levels are increased by about 20% or more, about 30% or more, about 40% or more, about 50% or more, about 60% or more, about 70% or more, about 80% or more, about 90% or more, or about 100% or more, as compared to prior to administration. In some embodiments, the hnRNP L protein levels are increased by no more than about 2.5%, no more than about 5%, or no more than about 7.5%, as compared to prior to administration. In some embodiments, the hnRNP L protein levels are increased by no more than about 10%, as compared to prior to administration. In some embodiments, the hnRNP L protein levels are increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the hnRNP L protein levels are increased by about 1.5-fold or more, about 2-fold or more, about 2.5-fold or more, about 5-fold or more, about 7.5-fold or more, about 10-fold or more, about 12.5-fold or more, or about 15-fold or more, as compared to prior to administration. In some embodiments, the hnRNP L protein levels are increased by no more than about 1.5-fold, no more than about 2-fold, no more than about 2.5-fold, no more than about 5-fold, no more than about 7.5-fold, no more than about 10-fold, no more than about 12.5-fold, or no more than about 15-fold, as compared to prior to administration. In some embodiments, the ASO increases hnRNP L expression by at least 1.05 fold, at least 1.1 fold, at least 1.15 fold, at least 1.2 fold, at least 1.25 fold, at least 1.3 fold, at least 1.35 fold, at least 1.4 fold, at least 1.45 fold, at least 1.5 fold, at least 1.55 fold, at least 1.6 fold, at least 1.65 fold, at least 1.7 fold, at least 1.75 fold, at least 1.8 fold, at least 1.85 fold, at least 1.9 fold, at least 1.95 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 50 fold, at least 60 fold, at least 70 fold, at least 80 fold, at least 90 fold, or at least 100 fold. In some embodiments, the hnRNP L protein levels are increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 500%, 750%, 1000%, 1250%, or 1500%, or by a range defined by any of the two aforementioned percentages.

In some embodiments, the composition comprises an oligonucleotide that targets hnRNP L or a poison exon of hnRNP L and when administered to a subject in an effective amount diminishes a neurological disorder or disease phenotype. A disorder may include a disease.

### ASOs

Disclosed herein, in some embodiments, are compositions comprising an oligonucleotide such as an antisense oligonucleotide (ASO). In some embodiments, the ASO targets hnRNP L or a poison exon of hnRNP L. Targeting hnRNP L may include binding a hnRNP L RNA. Targeting hnRNP L may include having a sequence reverse complementary to a hnRNP L RNA. In some embodiments, the ASO increases the expression of hnRNP L (e.g., a productive isoform of hnRNP L). In some embodiments, the ASO increases expression of hnRNP L. In some embodiments, the ASO targets a poison exon of hnRNP L, or targets an intron immediately upstream, or immediately downstream of the poison exon. In some embodiments, the ASO targets one or more of the hnRNP L binding sites that flank the poison exon. In some embodiments, the poison exon comprises the following sequence:

GGTCGCAGTGTATGTTTGATGGGACGCCATCTTTCAGAACTGTGCTAACTCACTGTTGAA GCGTCCAATG (SEQ ID NO: 102). Note that where a sequence includes T. U is contemplated for an RNA. In some embodiments, the ASO comprises a DNA oligonucleotide. The ASO may be included in a method or composition here.

In some embodiments, the ASO targets (e.g., binds or is complementary to) a region of an hnRNP L RNA (e.g., mRNA or pre-mRNA). The region may be upstream (e.g., 5′) of a poison exon such as exon 6A. The region may be downstream (e.g., 3′) of a poison exon such as exon 6A. The region may be or include a splice junction. The region may be or include a splice donor region. The region may be or include a splice acceptor region. The region may be or include an exon-intron boundary. The region may be or include a splice site. The region may be or include a 5′ splice site. The region may be or include a 3′ splice site. The region may be or include a binding site for hnRNP L. The region may be or include a promoter region. The region may be or include an hnRNP L binding site (e.g., where an hnRNP L protein binds to the hnRNP L RNA). The hnRNP L binding site may be within an intron upstream of poison exon 6A. Some examples of hnRNP L binding sites may include (from 5′ to 3′): CACA, CACCAACACACA, CACCAC, TACA, ACACCACACC, CACA, CATACA, ACAC, TACACA, ACACA, CACC, CACCAA, CACACA, CCACA, CACAC, CACACC, CACCACC, CCAC, or CATACACC. The region may be or be included among an hnRNP L binding motif (e.g. as shown in FIG. 20) or binding site described herein.

The ASO may be 12-50 nucleosides in length, or longer. In some embodiments, the ASO is 10, 11, 12, 13, 14, 15, 16, 17, 18, 19, 20, 21, 22, 23, 24, 25, 26, 27, 28, 29, 30, 35, 40, 45, 50, 55, 60, 70, 75, or 100 nucleosides in length, or a range defined by any of the two aforementioned numbers. The ASO may be about any of these lengths or ranges.

In some embodiments, the ASO comprises a nucleoside sequence at least 70% identical, at least 75% identical, at least 80% identical, at least 85% identical, at least 90% identical, or at least 95% identical, to a sequence of any one of SEQ ID NOs: 24-101. In some embodiments, the ASO comprises a nucleoside sequence less than 70% identical, less than 75% identical, less than 80% identical, less than 85% identical, less than 90% identical, less than 95% identical, or less than 100% identical, to a sequence of any one of SEQ ID NOs: 24-101. In some embodiments, the ASO comprises a nucleoside sequence comprising or consisting of the sequence of any one of SEQ ID NOs: 24-101, or a nucleic acid sequence thereof having 1 or 2 nucleoside substitutions, additions, or deletions. In some embodiments, the ASO sequence comprises a nucleoside sequence comprising or consisting of the sequence of any one of SEQ ID NOs: 24-101, or a nucleic acid sequence thereof having 3 or 4 nucleoside substitutions, additions, or deletions. In some embodiments, the ASO sequence comprises a nucleoside sequence comprising or consisting of the sequence of any one of SEQ ID NOs: 24-101.

In some embodiments, the ASO comprises a nucleoside sequence at least 70% identical, at least 75% identical, at least 80% identical, at least 85% identical, at least 90% identical, or at least 95% identical, to a sequence of any one of SEQ ID NOs: 47-101. In some embodiments, the ASO comprises a nucleoside sequence less than 70% identical, less than 75% identical, less than 80% identical, less than 85% identical, less than 90% identical, less than 95% identical, or less than 100% identical, to a sequence of any one of SEQ ID NOs: 47-101. In some embodiments, the ASO comprises a nucleoside sequence comprising or consisting of the sequence of any one of SEQ ID NOs: 47-101, or a nucleic acid sequence thereof having 1 or 2 nucleoside substitutions, additions, or deletions. In some embodiments, the ASO sequence comprises a nucleoside sequence comprising or consisting of the sequence of any one of SEQ ID NOs: 47-101, or a nucleic acid sequence thereof having 3 or 4 nucleoside substitutions, additions, or deletions. In some embodiments, the ASO sequence comprises a nucleoside sequence comprising or consisting of the sequence of any one of SEQ ID NOs: 47-101.

In some embodiments, the ASO comprises a nucleoside sequence at least 70% identical, at least 75% identical, at least 80% identical, at least 85% identical, at least 90% identical, or at least 95% identical, to a sequence of any one of SEQ ID NOs: 47-69. In some embodiments, the ASO comprises a nucleoside sequence less than 70% identical, less than 75% identical, less than 80% identical, less than 85% identical, less than 90% identical, less than 95% identical, or less than 100% identical, to a sequence of any one of SEQ ID NOs: 47-69. In some embodiments, the ASO comprises a nucleoside sequence comprising or consisting of the sequence of any one of SEQ ID NOs: 47-69, or a nucleic acid sequence thereof having 1 or 2 nucleoside substitutions, additions, or deletions. In some embodiments, the ASO sequence comprises a nucleoside sequence comprising or consisting of the sequence of any one of SEQ ID NOs: 47-69, or a nucleic acid sequence thereof having 3 or 4 nucleoside substitutions, additions, or deletions. In some embodiments, the ASO sequence comprises a nucleoside sequence comprising or consisting of the sequence of any one of SEQ ID NOs: 47-69.

In some embodiments, the ASO targets (e.g., binds or is complementary to) an untranslated region (UTR) such as a 5′ UTR of hnRNP L. In some embodiments, the ASO comprises a nucleoside sequence at least 70% identical, at least 75% identical, at least 80% identical, at least 85% identical, at least 90% identical, or at least 95% identical, to a sequence of any one of SEQ ID NOs: 24-26. In some embodiments, the ASO comprises a nucleoside sequence less than 70% identical, less than 75% identical, less than 80% identical, less than 85% identical, less than 90% identical, less than 95% identical, or less than 100% identical, to a sequence of any one of SEQ ID NOs: 24-26. In some embodiments, the ASO comprises a nucleoside sequence comprising or consisting of the sequence of any one of SEQ ID NOs: 24-26, or a nucleic acid sequence thereof having 1 or 2 nucleoside substitutions, additions, or deletions. In some embodiments, the ASO sequence comprises a nucleoside sequence comprising or consisting of the sequence of any one of SEQ ID NOs: 24-26, or a nucleic acid sequence thereof having 3 or 4 nucleoside substitutions, additions, or deletions. In some embodiments, the ASO sequence comprises a nucleoside sequence comprising or consisting of the sequence of any one of SEQ ID NOs: 24-26.

In some embodiments, the ASO targets (e.g., binds or is complementary to) a region upstream (e.g., 5′) of a poison exon of hnRNP L. The poison exon may be within a hnRNP L RNA such as a hnRNP L pre-mRNA. The poison exon may be within a hnRNP L mature mRNA. The upstream region may be or include an intron. The intron may be immediately upstream of the poison exon. In some embodiments, the ASO comprises a nucleoside sequence at least 70% identical, at least 75% identical, at least 80% identical, at least 85% identical, at least 90% identical, or at least 95% identical, to a sequence of any one of SEQ ID NOs: 27-35. In some embodiments, the ASO comprises a nucleoside sequence less than 70% identical, less than 75% identical, less than 80% identical, less than 85% identical, less than 90% identical, less than 95% identical, or less than 100% identical, to a sequence of any one of SEQ ID NOs: 27-35. In some embodiments, the ASO comprises a nucleoside sequence comprising or consisting of the sequence of any one of SEQ ID NOs: 27-35, or a nucleic acid sequence thereof having 1 or 2 nucleoside substitutions, additions, or deletions. In some embodiments, the ASO sequence comprises a nucleoside sequence comprising or consisting of the sequence of any one of SEQ ID NOs: 27-35, or a nucleic acid sequence thereof having 3 or 4 nucleoside substitutions, additions, or deletions. In some embodiments, the ASO sequence comprises a nucleoside sequence comprising or consisting of the sequence of any one of SEQ ID NOs: 27-35.

In some embodiments, the ASO targets (e.g., binds or is complementary to) a poison exon of hnRNP L. The poison exon may be within a hnRNP L RNA such as a hnRNP L mRNA. The mRNA may be or include a pre-mRNA. The mRNA may be or include a mature mRNA. In some embodiments, the ASO comprises a nucleoside sequence at least 70% identical, at least 75% identical, at least 80% identical, at least 85% identical, at least 90% identical, or at least 95% identical, to a sequence of any one of SEQ ID NOs: 36-46. In some embodiments, the ASO comprises a nucleoside sequence less than 70% identical, less than 75% identical, less than 80% identical, less than 85% identical, less than 90% identical, less than 95% identical, or less than 100% identical, to a sequence of any one of SEQ ID NOs: 36-46. In some embodiments, the ASO comprises a nucleoside sequence comprising or consisting of the sequence of any one of SEQ ID NOs: 36-46, or a nucleic acid sequence thereof having 1 or 2 nucleoside substitutions, additions, or deletions. In some embodiments, the ASO sequence comprises a nucleoside sequence comprising or consisting of the sequence of any one of SEQ ID NOs: 36-46, or a nucleic acid sequence thereof having 3 or 4 nucleoside substitutions, additions, or deletions. In some embodiments, the ASO sequence comprises a nucleoside sequence comprising or consisting of the sequence of any one of SEQ ID NOs: 36-46.

In some embodiments, the ASO comprises a nucleoside sequence at least 70% identical, at least 75% identical, at least 80% identical, at least 85% identical, at least 90% identical, or at least 95% identical, to a sequence in any of Tables 6-7B. In some embodiments, the ASO comprises a nucleoside sequence less than 70% identical, less than 75% identical, less than 80% identical, less than 85% identical, less than 90% identical, less than 95% identical, or less than 100% identical, to a sequence in any of Tables 6-7B. In some embodiments, the ASO comprises a sequence in any of Tables 6-7B, or a nucleic acid sequence thereof having 3 or 4 nucleoside substitutions, additions, or deletions. In some embodiments, the ASO comprises a sequence in any of Tables 6-7B, or a sequence thereof having 1 or 2 nucleoside substitutions, additions, or deletions. In some embodiments, the ASO comprises a sequence in any of Tables 6-7B. The ASO may include one or more internucleoside linkages, or one or more nucleoside modifications. The ASO or oligonucleotide may avoid a criteria or flag in Table 8.

In some embodiments, the ASO increases hnRNP L expression (e.g., mRNA or protein), as determined by an assay. The expression may include an mRNA level or amount. The expression may include a protein level or amount. In some embodiments, the ASO increases hnRNP L expression by at least 5%, at least 10%, at least 15%, or at least 20%. In some embodiments, the ASO comprises the base sequence of an ASO that increased max hnRNP L expression by at least 5%, at least 10%, at least 15%, or at least 20% in Table 10, or a sequence thereof having 3 or 4 insertions, substitutions, or deletions. In some embodiments, the ASO comprises the base sequence of an ASO that increased max hnRNP L expression by at least 5%, at least 10%, at least 15%, or at least 20% in Table 10, or a sequence thereof having 1 or 2 insertions, substitutions, or deletions. In some embodiments, the ASO comprises the base sequence of an ASO that increased max hnRNP L expression by at least 5%, at least 10%, at least 15%, or at least 20% in Table 10. In some embodiments, the ASO comprises the base sequence of an ASO that increased max hnRNP L expression by at least 5%, at least 10%, at least 15%, or at least 20% in Table 9B, or a sequence thereof having 3 or 4 insertions, substitutions, or deletions. In some embodiments, the ASO comprises the base sequence of an ASO that increased max hnRNP L expression by at least 5%, at least 10%, at least 15%, or at least 20% in Table 9B, or a sequence thereof having 1 or 2 insertions, substitutions, or deletions. In some embodiments, the ASO comprises the base sequence of an ASO that increased max hnRNP L expression by at least 5%, at least 10%, at least 15%, or at least 20% in Table 9B. In some embodiments, the ASO increased hnRNP L expression by at least 25%, at least 30%, at least 35%, at least 40%, at least 45%, at least 50%, at least 55%, at least 60%, at least 65%, or at least 70%. In some embodiments, the ASO comprises the base sequence of an ASO that increased max hnRNP L expression by at least 25%, at least 30%, at least 35%, at least 40%, at least 45%, at least 50%, at least 55%, at least 60%, at least 65%, or at least 70% in Table 9B, or a sequence thereof having 3 or 4 insertions, substitutions, or deletions. In some embodiments, the ASO comprises the base sequence of an ASO that increased max hnRNP L expression by at least 25%, at least 30%, at least 35%, at least 40%, at least 45%, at least 50%, at least 55%, at least 60%, at least 65%, or at least 70% in Table 9B, or a sequence thereof having 1 or 2 insertions, substitutions, or deletions. In some embodiments, the ASO comprises the base sequence of an ASO that increased max hnRNP L expression by at least 25%, at least 30%, at least 35%, at least 40%, at least 45%, at least 50%, at least 55%, at least 60%, at least 65%, or at least 70% in Table 9B. In some embodiments, the ASO comprises the base sequence of an ASO that increased hnRNP L by at least 25%, at least 30%, at least 35%, at least 40%, at least 45%, at least 50%, at least 55%, at least 60%, at least 65%, at least 70%, at least 75%, or at least 80% in Table 10, or a sequence thereof having 3 or 4 insertions, substitutions, or deletions. In some embodiments, the ASO comprises the base sequence of an ASO that increased hnRNP L by at least 25%, at least 30%, at least 35%, at least 40%, at least 45%, at least 50%, at least 55%, at least 60%, at least 65%, at least 70%, at least 75%, or at least 80% in Table 10, or a sequence thereof having 1 or 2 insertions, substitutions, or deletions. In some embodiments, the ASO comprises the base sequence of an ASO that increased hnRNP L by at least 25%, at least 30%, at least 35%, at least 40%, at least 45%, at least 50%, at least 55%, at least 60%, at least 65%, at least 70%, at least 75%, or at least 80% in Table 10.

In some embodiments, the ASO has an EC50 value below 1000 nM, below 750 nM, below 500 nM, below 250 nM, below 200 nM, below 150 nM, below 100 nM, below 75 nM, below 50 nM, below 25 nM, below 20 nM, below 15 nM, below 14 nM, below 13 nM, below 12 nM, below 11 nM, below 10 nM, below 9 nM, below 8 nM, below 7 nM, below 6 nM, below 5 nM, below 4 nM, or below 3 nM, as determined by an assay such as an in vitro assay. In some embodiments, the ASO has an EC50 value of at least 1000 nM, at least 750 nM, at least 500 nM, at least 250 nM, at least 200 nM, at least 150 nM, at least 100 nM, at least 75 nM, at least 50 nM, at least 25 nM, at least 20 nM, at least 15 nM, at least 14 nM, at least 13 nM, at least 12 nM, at least 11 nM, at least 10 nM, at least 9 nM, at least 8 nM, at least 7 nM, at least 6 nM, at least 5 nM, at least 4 nM, or at least 3 nM. In some embodiments, the ASO comprises the base sequence of an ASO that had an EC50 value below 15 nM, below 14 nM, below 13 nM, below 12 nM, below 11 nM, below 10 nM, below 9 nM, below 8 nM, below 7 nM, below 6 nM, below 5 nM, below 4 nM, or below 3 nM in Table 9B, or a sequence thereof having 3 or 4 insertions, substitutions, or deletions. In some embodiments, the ASO comprises the base sequence of an ASO that had an EC50 value below 15 nM, below 14 nM, below 13 nM, below 12 nM, below 11 nM, below 10 nM, below 9 nM, below 8 nM, below 7 nM, below 6 nM, below 5 nM, below 4 nM, or below 3 nM in Table 9B, or a sequence thereof having 1 or 2 insertions, substitutions, or deletions. In some embodiments, the ASO comprises the base sequence of an ASO that had an EC50 value below 15 nM, below 14 nM, below 13 nM, below 12 nM, below 11 nM, below 10 nM, below 9 nM, below 8 nM, below 7 nM, below 6 nM, below 5 nM, below 4 nM, or below 3 nM in Table 9B.

### Oligonucleotide Modifications

Described herein, in some embodiments, are oligonucleotides such as ASOs. The ASO may target hnRNP L or a poison exon of hnRNP L. In some embodiments, the ASO is modified (e.g., includes modified nucleosides or internucleoside linkages).

In some embodiments, the oligonucleotide comprises a modification comprising a modified nucleoside and/or a modified internucleoside linkage. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. A phosphorothioate may include a nonbridging oxygen atom in a phosphate backbone of the oligonucleotide that is replaced by sulfur. Modified internucleoside linkages may be included in siRNAs or ASOs. In some embodiments, the ASO comprises an internucleoside linkage modification. In some embodiments, the internucleoside linkage modification comprises a phosphorothioate linkage. Benefits of the modified internucleoside linkage may include decreased toxicity or improved pharmacokinetics.

In some embodiments, the composition comprises an oligonucleotide that targets hnRNP L or a poison exon of hnRNP L, wherein the oligonucleotide comprises a modified internucleoside linkage, wherein the oligonucleotide comprises 1, 2, 3, 4, 5, 6, 7, 8, 9, 10, 11, 12, 13, 14, 15, 16, 17, 18, 19, or 20 modified internucleoside linkages, or a range of modified internucleoside linkages defined by any two of the aforementioned numbers. In some embodiments, the oligonucleotide comprises no more than 18 modified internucleoside linkages. In some embodiments, the oligonucleotide comprises no more than 20 modified internucleoside linkages. In some embodiments, the oligonucleotide comprises 2 or more modified internucleoside linkages, 3 or more modified internucleoside linkages, 4 or more modified internucleoside linkages, 5 or more modified internucleoside linkages, 6 or more modified internucleoside linkages, 7 or more modified internucleoside linkages, 8 or more modified internucleoside linkages, 9 or more modified internucleoside linkages, 10 or more modified internucleoside linkages, 11 or more modified internucleoside linkages, 12 or more modified internucleoside linkages, 13 or more modified internucleoside linkages, 14 or more modified internucleoside linkages, 15 or more modified internucleoside linkages, 16 or more modified internucleoside linkages, 17 or more modified internucleoside linkages, 18 or more modified internucleoside linkages, 19 or more modified internucleoside linkages, or 20 or more modified internucleoside linkages.

In some embodiments, the composition comprises an oligonucleotide that targets hnRNP L or a poison exon of hnRNP L, wherein the oligonucleotide comprises a modified nucleoside. In some embodiments, the modified nucleoside comprises a sugar modification. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-fluoro, or 2-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises HLA. In some embodiments, the modified nucleoside comprises CeNA. In some embodiments, the modified nucleoside comprises a 2′-methoxyethyl group. In some embodiments, the modified nucleoside comprises a 2′-O-alkyl group. In some embodiments, the modified nucleoside comprises a 2′-O-allyl group. In some embodiments, the modified nucleoside comprises a 2′-fluoro group. In some embodiments, the modified nucleoside comprises a 2′-deoxy group. In some embodiments, the modified nucleoside comprises a 2′-O-methyl nucleoside, 2′-deoxyfluoro nucleoside, 2′-O—N-methylacetamido (2-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises a 2′-O-methyl nucleoside. In some embodiments, the modified nucleoside comprises a 2′-deoxyfluoro nucleoside. In some embodiments, the modified nucleoside comprises a 2′-O-NMA nucleoside. In some embodiments, the modified nucleoside comprises a 2′-O-DMAEOE nucleoside. In some embodiments, the modified nucleoside comprises a 2′-O-aminopropyl (2′-O-AP) nucleoside. In some embodiments, the modified nucleoside comprises 2′-ara-F. In some embodiments, the modified nucleoside comprises one or more 2 fluoro modified nucleosides. In some embodiments, the modified nucleoside comprises a 2′ O-alkyl modified nucleoside. In some embodiments, the modified nucleoside comprises a modified base such as 5′-methyl cytosine (5′-methyl C) in place of one or more (e.g., all) cytosines. In some embodiments, the ASO comprises a nucleoside modification. In some embodiments, the nucleoside modification comprises 2-O-methoxyethyl (MOE). In some embodiments, the nucleoside modification comprises 5′-methyl C. In some embodiments, the oligonucleotide includes a tricyclo-DNA (tcDNA), locked nucleic acid (LNA), peptide nucleic acid (PNA), or phosphorodiamidate morpholino oligomer (PMO) modification. Some embodiments include a modification such as 5-methylcytidine, 5-methyluridine, abasic RNA, 2′-O-methoxy, 2′-O-methoxyethyl, 2′-fluoro, locked nucleic acid, constrained ethyl bridged nucleic acid, ethylene-bridged nucleic acid, phosphorodiamidate morpholino oligonucleotide, peptide nucleic acid, or tricycloDNA, such as is described in Roberts et al., Advances in oligonucleotide drug delivery. 2020 October; 19 (10): 673-694. doi: 10.1038/s41573-020-0075-7. Benefits of the modified nucleoside may include decreased toxicity or improved pharmacokinetics.

In some embodiments, the oligonucleotide comprises 1, 2, 3, 4, 5, 6, 7, 8, 9, 10, 11, 12, 13, 14, 15, 16, 17, 18, 19, 20, or 21 modified nucleosides, or a range of nucleosides defined by any two of the aforementioned numbers. In some embodiments, the oligonucleotide comprises no more than 19 modified nucleosides. In some embodiments, the oligonucleotide comprises no more than 21 modified nucleosides. In some embodiments, the oligonucleotide comprises 2 or more modified nucleosides, 3 or more modified nucleosides, 4 or more modified nucleosides, 5 or more modified nucleosides, 6 or more modified nucleosides, 7 or more modified nucleosides, 8 or more modified nucleosides, 9 or more modified nucleosides, 10 or more modified nucleosides, 11 or more modified nucleosides, 12 or more modified nucleosides, 13 or more modified nucleosides, 14 or more modified nucleosides, 15 or more modified nucleosides, 16 or more modified nucleosides, 17 or more modified nucleosides, 18 or more modified nucleosides, 19 or more modified nucleosides, 20 or more modified nucleosides, or 21 or more modified nucleosides.

### Combinational Therapy

In some embodiments, the agent described herein (e.g., ASC and its derivatives or analogs, or a polypeptide of, or a polynucleotide encoding such polypeptide of, hnRNP L or its biologically active fragments, or an ASO that increases hnRNP L levels) may be combined together or with an additional agent capable of rescuing UNC13A, UNC13B, STMN2, SORT1, GPSM2, ATG4B. TDP-43, and/or hnRNP L defects in a subject with a neurological disease, as described herein. A composition comprising ASC or its derivatives or analogs and an inhibitor of another signaling pathway may be used to treat the neurological diseases described herein (e.g., ALS or FTD).

### Methods of Treatment and Use

The present specification provides a method of delivery of a bioactive composition or formulation (e.g., the agents described herein) by an administration route including, but not limited to, oral, nasal, intravenous, intra-arterial, intramuscular, intraperitoneal, subcutaneous, intramuscular, intrathecal and/or direct injection into the central nervous system, and topical administration, or combinations thereof. Administration may be by nasogastric tube, or via percutaneous endoscopic gastrostomy tube. The disclosure includes, but is not limited to, administering by a medical professional and self-administering. For example, delivery vehicles may include, e.g., liposomes, virus, nanoparticles, or other methods known in the art, such as gene therapies.

Disclosed herein, in some embodiments, are methods of administering a composition described herein to a subject. Some embodiments relate to use a composition described herein, such as administering the composition to a subject.

Some embodiments relate to a method of treating a disease or disorder (e.g., mental disorder (e.g., neurological disorder), or a CEIND or PEIND) in a subject in need thereof. Some embodiments relate to use of a composition described herein in the method of treatment. Some embodiments include administering a composition described herein to a subject with the disorder. In some embodiments, the administration treats the disorder in the subject. In some embodiments, the composition treats the disorder in the subject. A cryptic exon-induced neurological disease (CEIND) or poison exon-induced neurological disease (PEIND) may be treated in a subject by administration of a composition herein. Some examples of CEIND or PEIND may be found in Stephan J Sanders et al., “A framework for the investigation of rare genetic disorders in neuropsychiatry” Review Nat Med. 2019 October; 25(10):1477-1487.

In some embodiments, the treatment comprises prevention, slowing, attenuation, inhibition, or reversion of the disorder (e.g., neurological disorder, or a CEIND or PEIND) in the subject. Some embodiments relate to use of a composition described herein in the method of preventing, inhibiting, or reversing the disorder. Some embodiments relate to a method of preventing, inhibiting, or reversing a disorder in a subject in need thereof. Some embodiments include administering a composition described herein to a subject with the disorder. In some embodiments, the administration prevents, slows, attenuates, inhibits, or reverses the disorder in the subject. In some embodiments, the composition prevents, inhibits, or reverses the disorder in the subject.

Some embodiments relate to a method of preventing a disorder (e.g., neurological disorder, or a CEIND or PEIND) in a subject in need thereof. Some embodiments relate to use of a composition described herein in the method of preventing the disorder. Some embodiments include administering a composition described herein to a subject with the disorder. In some embodiments, the administration prevents the disorder in the subject. In some embodiments, the composition prevents the disorder in the subject.

Some embodiments relate to a method of inhibiting a disorder (e.g., neurological disorder, or a CEIND or PEIND) in a subject in need thereof. Some embodiments relate to use of a composition described herein in the method of inhibiting the disorder. Some embodiments include administering a composition described herein to a subject with the disorder. In some embodiments, the administration inhibits the disorder in the subject. In some embodiments, the composition inhibits the disorder in the subject. In some embodiments, the composition slows the disorder in the subject. In some embodiments, the composition attenuates the disorder in the subject.

Some embodiments relate to a method of reversing a disorder (e.g., neurological disorder, or a CEIND or PEIND) in a subject in need thereof. Some embodiments relate to use of a composition described herein in the method of reversing the disorder. Some embodiments include administering a composition described herein to a subject with the disorder. In some embodiments, the administration reverses the disorder in the subject. In some embodiments, the composition reverses the disorder in the subject.

In some embodiments, the administration is systemic. In some embodiments, the administration is intravenous. In some embodiments, the administration is by injection.

Some embodiments of the methods described herein include treating a disorder in a subject in need thereof. In some embodiments, the disorder is a mental disorder. In some embodiments, the mental disorder is a psychiatric disorder or neurological disorder. The psychiatric disorder or neurological disorder may comprise a disorder, a brain disorder, a CNS disorder, a CSF disorder, or a combination thereof. In some embodiments, the disorder is a CEIND or PEIND. In some embodiments, the subject has fragile X syndrome.

Some embodiments of the methods described herein include treatment of a subject. Non-limiting examples of subjects include vertebrates, animals, mammals, dogs, cats, cattle, horses, pigs, rabbits, rodents, mice, rats, primates, monkeys, and humans. In some embodiments, the subject is a vertebrate. In some embodiments, the subject is an animal. In some embodiments, the subject is a mammal. In some embodiments, the subject is a dog. In some embodiments, the subject is a cat. In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human.

In some embodiments, the subject is male. In some embodiments, the subject is female. In some embodiments, the subject is an adult (e.g., at least 18 years old). In some embodiments, the subject is 45 years old or greater. In some embodiments, the subject is 50 years old or greater. In some embodiments, the subject is 55 years old or greater. In some embodiments, the subject is 60 years old or greater. In some embodiments, the subject is 65 years old or greater. In some embodiments, the subject is 70 years old or greater. In some embodiments, the subject is 75 years old or greater. In some embodiments, the subject is 80 years old or greater. In some embodiments, the subject is 85 years old or greater.

In some embodiments, the subject has ASD. In some embodiments, the subject to be treated has a splicing defect. In some embodiments, the subject to be treated has a splicing defect in an ASD-associated gene. In some embodiments, the subject to be treated has a splicing defect in any ASD-associated genes that is a target of hnRNP L as described herein. For example, the subject may have a splicing defect in the genes in Table 11 that is a target of hnRNP L. Table 11 includes the SFARI (Simons Foundation Autism Research Initiative) list of autism genes (881 genes). SFARI genes may include genes associated with ASD from an evolving database for the autism research community. More particularly, the subject may have a splicing defect in genes listed in Table 12, which lists a subset of SFARI genes that have a high-scoring hnRNP L motif within 500 bp of one of the Castle splice sites (see, for example, Castle, et al., Nature Genetics 40(12):1416-25, 2008) (338 genes). Table 13 includes a subset of SFARI genes that have a very high scoring putative hnRNP L-binding motif within 500 bp of one of the Castle splice sites (152 genes). Genes listed in Table 14 include a subset of SFARI genes with hnRNP L binding sites near splice events specifically observed in autism (78 genes). Genes listed in Table 15 include a subset of genes bearing hnRNP L binding sites within the SHANK-TSC-mTOR-ERK ASD disease module (27 genes). Genes listed in Table 16 include a subset of genes bearing hnRNP L binding sites that also comprise the SHANK-TSC ASD disease module (18 genes). In some embodiments, the subject comprises or has a mutation in an hnRNP L target gene which results in spliceopathy. In some embodiments, the mutation is identified or confirmed in the subject before or after treatment or administration of a compound or composition herein. In some embodiments, the subject has a disease or disorder in Table 15 or Table 16. Any similar concepts in WO2019236750 to those in this paragraph are incorporated herein by reference, and WO2019236750 is incorporated herein by reference in its entirety.

### Effects

In some embodiments, the composition or administration of the composition affects a measurement such as mental disorder (e.g., neurological disorder, or a CEIND or PEIND) measurement. The measurement may be affected in relation to a baseline or control measurement.

In some embodiments, the composition or administration of the composition affects a measurement such as neurological measurement. In some embodiments, the composition or administration of the composition affects a measurement, such as neurological measurement, relative to a baseline measurement. In some embodiments, the neurological measurement includes a cognitive assessment. In some embodiments, the neurological measurement includes a pathology measurement. In some embodiments, the neurological measurement includes a motor neuro-mediated physiology or motor function measurement.

In some embodiments, the measurement indicates that the disorder has been treated. In some embodiments, the measurement indicates that the severity of the disorder has decreased. In some embodiments, the measurement indicates that the severity of a sign or symptom of the disorder has decreased. In some embodiments, the measurement indicates that the frequency of a sign or symptom of the disorder has decreased.

Some embodiments of the methods described herein include obtaining the measurement from a subject. For example, the measurement may be obtained from the subject after treating the subject. In some embodiments, the measurement is obtained in a sample (such as a fluid or tissue sample described herein) obtained from the subject after the composition is administered to the subject. In some embodiments, the measurement is an indication that the disorder has been treated.

In some embodiments, the measurement is obtained directly from the subject. In some embodiments, the measurement is obtained noninvasively using an imaging device. In some embodiments, the measurement is obtained in a sample from the subject. In some embodiments, the measurement is obtained in one or more histological tissue sections. In some embodiments, the measurement is obtained by performing an assay on the sample obtained from the subject. In some embodiments, the measurement is obtained by an assay, such as an assay described herein. In some embodiments, the assay is an immunoassay, a colorimetric assay, a fluorescence assay, a chromatography (e.g., HPLC) assay, a branched DNA assay, or a PCR assay. In some embodiments, the measurement is obtained by an assay such as an immunoassay, a colorimetric assay, a fluorescence assay, or a chromatography (e.g., HPLC) assay. In some embodiments, the measurement is obtained by PCR. In some embodiments, the measurement is obtained by histology. In some embodiments, the measurement is obtained by observation. In some embodiments, additional measurements are made, such as in a 2nd sample, 3rd sample, a 4th sample, or a fifth sample.

In some embodiments, the measurement is obtained within 1 hour, within 2 hours, within 3 hours, within 4 hours, within 5 hours, within 6 hours, within 12 hours, within 18 hours, or within 24 hours after the administration of the composition. In some embodiments, the measurement is obtained within 1 day, within 2 days, within 3 days, within 4 days, within 5 days, within 6 days, or within 7 days after the administration of the composition. In some embodiments, the measurement is obtained within 1 week, within 2 weeks, within 3 weeks, within 1 month, within 2 months, within 3 months, within 6 months, within 1 year, within 2 years, within 3 years, within 4 years, or within 5 years after the administration of the composition. In some embodiments, the measurement is obtained after 1 hour, after 2 hours, after 3 hours, after 4 hours, after 5 hours, after 6 hours, after 12 hours, after 18 hours, or after 24 hours after the administration of the composition. In some embodiments, the measurement is obtained after 1 day, after 2 days, after 3 days, after 4 days, after 5 days, after 6 days, or after 7 days after the administration of the composition. In some embodiments, the measurement is obtained after 1 week, after 2 weeks, after 3 weeks, after 1 month, after 2 months, after 3 months, after 6 months, after 1 year, after 2 years, after 3 years, after 4 years, or after 5 years, following the administration of the composition.

In some embodiments, the composition increases the measurement relative to the baseline measurement. For example, a protective psychiatric or neurological phenotype may be increased upon administration of the composition. In some embodiments, the increase is measured in a sample obtained from the subject after administering the composition to the subject. In some embodiments, the increase is measured directly in the subject after administering the composition to the subject. In some embodiments, the measurement is increased by about 2.5% or more, about 5% or more, or about 7.5% or more, relative to the baseline measurement. In some embodiments, the measurement is increased by about 10% or more, relative to the baseline measurement. In some embodiments, the measurement is increased by about 20% or more, about 30% or more, about 40% or more, about 50% or more, about 60% or more, about 70% or more, about 80% or more, about 90% or more, relative to the baseline measurement. In some embodiments, the measurement is increased by about 100% or more, increased by about 250% or more, increased by about 500% or more, increased by about 750% or more, or increased by about 1000% or more, relative to the baseline measurement. In some embodiments, the measurement is increased by no more than about 2.5%, no more than about 5%, or no more than about 7.5%, relative to the baseline measurement. In some embodiments, the measurement is increased by no more than about 10%, relative to the baseline measurement. In some embodiments, the measurement is increased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is increased by no more than about 100%, increased by no more than about 250%, increased by no more than about 500%, increased by no more than about 750%, or increased by no more than about 1000%, relative to the baseline measurement. In some embodiments, the measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages. In some embodiments, the measurement includes a hnRNP L RNA measurement such as mRNA. In some embodiments, the measurement includes a hnRNP L protein measurement. The measurement may relate to a productive isoform of hnRNP L RNA or protein.

In some embodiments, the composition reduces the measurement relative to the baseline measurement. For example, an adverse phenotype of a neurological disorder may be reduced upon administration of the composition. In some embodiments, the reduction is measured in a sample obtained from the subject after administering the composition to the subject. In some embodiments, the reduction is measured directly in the subject after administering the composition to the subject. In some embodiments, the measurement is decreased by about 2.5% or more, about 5% or more, or about 7.5% or more, relative to the baseline measurement. In some embodiments, the measurement is decreased by about 10% or more, relative to the baseline measurement. In some embodiments, the measurement is decreased by about 20% or more, about 30% or more, about 40% or more, about 50% or more, about 60% or more, about 70% or more, about 80% or more, about 90% or more, relative to the baseline measurement. In some embodiments, the measurement is decreased by no more than about 2.5%, no more than about 5%, or no more than about 7.5%, relative to the baseline measurement. In some embodiments, the measurement is decreased by no more than about 10%, relative to the baseline measurement. In some embodiments, the measurement is decreased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is decreased by 2.5%, 5%, 7.5%, 10%, 20%, 30%, 40%, 50%, 60%, 70%, 80%, 90%, or 100%, or by a range defined by any of the two aforementioned percentages. The measurement may relate to a non-productive isoform of hnRNP L RNA or protein. The measurement may relate to a poison exon of hnRNP L such as exon 6A.

Some embodiments of the methods described herein include obtaining a sample from a subject. In some embodiments, a measurement is obtained in a sample obtained from the subject. In some embodiments, the sample is obtained from the subject prior to administration or treatment of the subject with a composition described herein. In some embodiments, a baseline measurement is obtained in a sample obtained from the subject prior to administering the composition to the subject. In some embodiments, the sample comprises a fluid. In some embodiments, the sample is a fluid sample. In some embodiments, the sample is a blood, plasma, buffy coat, peripheral blood mononuclear cell (PBMC), or serum sample. In some embodiments, the sample comprises blood. In some embodiments, the sample is a blood sample. In some embodiments, the sample is a whole-blood sample. In some embodiments, the blood is fractionated or centrifuged. In some embodiments, the sample comprises plasma. In some embodiments, the sample comprises a buffy coat. In some embodiments, the sample comprises a PBMC sample. In some embodiments, the sample is a plasma sample. A blood sample may be a plasma sample. In some embodiments the sample comprises the buffy coat. In some embodiments the sample is the PBMC fraction. In some embodiments, the sample comprises serum. In some embodiments, the sample is a serum sample. A blood sample may be a serum sample. In some embodiments, the sample is a CSF sample. In some embodiments the sample includes a CSF sample. In some embodiments, the sample is a CNS sample. In some embodiments the sample includes a CNS sample.

In some embodiments, the sample comprises a tissue. In some embodiments, the sample is a tissue sample. In some embodiments, the tissue comprises brain tissue. In some embodiments, the tissue comprises neural tissue. In some embodiments, the tissue comprises neuronal tissue. In some embodiments, the tissue comprises neurons. In some embodiments, the tissue comprises glial cells. In some embodiments, the tissue comprises epithelial cells. In some embodiments, the tissue comprises brain tissue.

In some embodiments, the sample includes cells. In some embodiments, the sample comprises a cell. In some embodiments, the cell is a brain cell. In some embodiments, the cell is a neuron. In some embodiments, the cell is a glial cell. The cell may include a blood cell. The cell may include a PBMC.

### Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B:” “one or more of A and B:” and “A and/or B” are each intended to mean “A alone, B alone, or A and B together.” A similar interpretation is also intended for lists including three or more items. For example, the phrases “at least one of A, B, and C:” “one or more of A, B, and C:” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible

It is understood that where a parameter range is provided, all integers within that range, and tenths thereof, are also provided by the disclosure. For example, “0.2-5 mg” is a disclosure of 0.2 mg. 0.3 mg, 0.4 mg, 0.5 mg, 0.6 mg etc. up to and including 5.0 mg.

A small molecule is a compound that is less than 2000 Daltons in mass. The molecular mass of the small molecule is preferably less than 1000 Daltons, more preferably less than 600 Daltons, e.g., the compound is less than 500 Daltons, 400 Daltons, 300 Daltons, 200 Daltons, or 100 Daltons.

As used herein, “kits” are understood to contain at least one non-standard laboratory reagent, such as the agent described herein, for use in the methods of the disclosure in appropriate packaging, optionally containing instructions for use. The kit can further include any other components required to practice the method of the disclosure, as dry powders, concentrated solutions, or ready to use solutions. In some embodiments, the kit comprises one or more containers that contain reagents for use in the methods of the disclosure; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

The composition and/or the dosage formulation described herein is in the form of a tablet, a capsule, a powder, a beverage, or an infant formula.

By “agent” is meant any small molecule chemical compound, antibody, nucleic acid molecule, or polypeptide, or fragments thereof. In some embodiments, the agent comprises ASC or its derivatives or analogs. In some embodiments, the agent comprises a structure selected from the group consisting of Formula 1, Formula 2, Formula 3, Formula 3′, Formula 4, Formula 5, Formula 6, Formula 7, Formula 8, Formula 9, Formula 10, Formula 11, Formula 12, Formula 13, Formula 14, Formula 15, Formula 16, Formula 17, Formula 18, Formula 19, Formula 20, Formula 21, Formula 22, Formula 23, Formula 24, Formula 25, Formula 26, Formula 27, Formula 28, Formula 29, Formula 30. Formula 31, Formula 32, Formula 33, Formula 34, Formula 35, Formula 36, Formula 37, Formula 38, Formula 39, Formula 40, Formula 41, Formula 42, Formula 43, Formula 44, Formula 45, Formula 46, Formula 47, Formula 48, Formula 49, Formula 50, Formula 51, Formula 52, Formula 53, Formula 54, Formula 55, Formula 56, Formula 57, Formula 58, Formula 59, Formula 60, Formula 61, Formula 62, Formula 63, Formula 64, Formula 65, Formula 66, Formula 67, Formula 68, Formula 69, Formula 70, Formula 71, Formula 72, Formula 73, Formula 74, Formula 75, Formula 76, and Formula 77. Optionally, the agent comprises a pharmaceutically acceptable salt of at least one of Formulas 1 to 77.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated protein or polypeptide is free of the amino acid sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

The transitional term “comprising.” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “subject,” “patient,” “individual,” and the like as used herein are not intended to be limiting and can be generally interchanged. That is, an individual described as a “patient” does not necessarily have a given disease, but may be merely seeking medical advice.

The term “subject” as used herein includes a patient with a neurological disease. More particularly, the “subject” may include a patient with ALS, FTD, or other neurological diseases described herein. In some embodiments, the “patient” may have a loss of function or altered function of a putative gene which includes hnRNP L binding sites (e.g., UNC13A). In some embodiments, the “patient” may have a mutation in the target gene which results in spliceopathy (e.g., UNC13A, UNC13B, STMN2, SORT1, GPSM2, or ATG4B). In some embodiments, the “patient” may include known cohorts with ALS, FTD, or other neurological diseases described herein, who carry a mutation resulting in spliceopathy of the target gene (e.g., UNC13A, UNC13B, STMN2, SORT1, GPSM2, or ATG4B) and carry a clinical diagnosis of ALS, FTD, or other neurological diseases described herein. Based on sequencing of ALS or FTD cohorts, it may be anticipated that there will be an expanding number of ALS or FTD patient subgroups who fulfill the criteria listed above and are thus candidates for a therapeutic response to ascochlorin and derivatives or analogs.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

The terms “treating” and “treatment” as used herein refer to the administration of an agent or formulation to a clinically symptomatic individual afflicted with an adverse condition, disorder, or disease, so as to affect a reduction in severity and/or frequency of symptoms, eliminate the symptoms and/or their underlying cause, and/or facilitate improvement or remediation of damage. The terms “preventing” and “prevention” refer to the administration of an agent or composition to a clinically asymptomatic individual who is susceptible to a particular adverse condition, disorder, or disease, and thus relates to the prevention of the occurrence of symptoms and/or their underlying cause.

The term “reduce,” “attenuate,” “promote,” or “increase” is meant to alter negatively or positively, respectively, by at least 5%. An alteration may be by 5%, 10%, 25%, 30%, 50%, 75%, 90%, 100%, or even more (for positive alternations).

As used herein, a “symptom” associated with a disorder includes any clinical or laboratory manifestation associated with the disorder, and is not limited to what the subject can feel or observe.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the compounds described herein to the subject.

Examples are provided below to facilitate a more complete understanding of the aspects herein. The following examples illustrate the exemplary modes of making and practicing aspects provided herein. However, the scope of the disclosure is not limited to specific embodiments disclosed in these Examples, which are for purposes of illustration only, since alternative methods can be utilized to obtain similar results.

The term, “neurological disorder or disease” as used herein refers to a disorder, disease or condition which directly or indirectly affects the normal functioning or anatomy of a subject's nervous system, including, but not limited to, the brain. In one embodiment, the neurological disorder or disease is a neurodevelopmental disorder.

An example of a neurological disorder or disease is Amyotrophic Lateral Sclerosis (ALS) or Frontotemporal Dementia (FTD). In other examples, the neurological disorder or disease is Alzheimer's disease, Autism Spectrum Disorder, Pick's disease, hippocampal sclerosis, corticobasal degeneration, Argyrophilic grain disease, Huntington disease, or Fragile X syndrome.

The phrase, “treating a neurological disorder or disease” as used herein includes, but is not limited to, reversing, alleviating or inhibiting the progression of a neurological disorder or disease or conditions associated with a neurological disorder or disease. As used herein, and as well understood in the art, “to treat” or “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

Treating a neurological disorder or disease includes preventing the occurrence of a neurological disorder or disease or symptoms or conditions associated with a neurological disorder or disease or preventing worsening of the severity of a neurological disorder or disease or conditions associated with a neurological disorder or disease.

The term, “neurological function” as used herein refers to the functioning and/or activity of a subject's nervous system.

The term, “improving neurological function,” as used herein refers to improving the structure, function and/or activity of a subject's nervous system. In one embodiment, improving neurological function includes improving neurodevelopment and/or improving behavior.

The term “subject” as used herein refers to any member of the animal kingdom, such as a mammal. In one embodiment, the subject is a human. In another embodiment, the subject is a rodent, e.g., mouse or rat, or another animal such as animal model for Amyotrophic Lateral Sclerosis (ALS), Frontotemporal Dementia (FTD), or other diseases described herein.

The term “a cell” includes a single cell as well as a plurality or population of cells. Administering a modulator or an agent to a cell includes both in vitro and in vivo administrations.

The modulators and agents described herein may be formulated into pharmaceutical compositions for administration to subjects and/or use in subjects in a biologically compatible form suitable for administration in vivo. The compositions described herein can be prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, 20th ed., Mack Publishing Company, Easton, Pa., USA, 2000). On this basis, the compositions include, albeit not exclusively, solutions of the substances in association with one or more pharmaceutically acceptable vehicles or diluents, and contained in buffered solutions with a suitable pH and iso-osmotic with the physiological fluids.

Modulators and agents described herein are formulated into pharmaceutical compositions for administration to the brain or central nervous system of a subject. Modulators, agents and pharmaceutical compositions which cannot penetrate the blood-brain barrier can be effectively administered by an intraventricular route or other appropriate delivery system suitable for administration to the brain.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. Proteins may be supplied, for example but not by way of limitation, as a lyophilized powder which is reconstituted with sterile water or saline prior to administration to the patient.

Pharmaceutical compositions may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1 (2,3-dioleyloxy) propyl) N,N,N-trimethylammonium chloride (DOTMA), dioleoylphosphatidyl-ethanolamine (DOPE), and liposomes. Such compositions should contain a therapeutically effective amount of the compound, together with a suitable amount of carrier so as to provide the form for direct administration to the patient.

The compositions may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The modulators, agents and/or pharmaceutical compositions described herein may be administered to, or used in, living organisms including humans, and animals.

Administration of an “effective amount” of the modulators, agents and/or pharmaceutical compositions is defined as an amount effective, at dosages and for periods of time necessary to achieve the desired result. For example, an effective amount of a substance may vary according to factors such as the disease state, age, sex, and weight of the individual, and the ability of the recombinant protein to elicit a desired response in the individual. Dosage regime may be adjusted to provide the optimum therapeutic response. For example, several divided doses may be administered daily or the dose may be proportionally reduced as indicated by the exigencies of the therapeutic situation.

The disclosure includes sequences such as nucleotide sequences. Any inconsistency between the sequence listing and the sequences in the written description should be resolved in favor of the written description.

### NUMBERED EMBODIMENTS

Some aspects include or relate to any of the following embodiments:
1. A method of treating a subject with Cryptic Exon Induced Neurological Disease (CEIND) or Poison Exon Induced Neurological Disease (PEIND), comprising administering to said subject an agent to increase expression levels and/or stability of hnRNP L, thereby attenuating the expression of cryptic or poison exons.
2. The method of embodiment 1, wherein the neurological disease is associated with a splicing defect caused by one or more TDP-43 proteinopathies.
3. The method of embodiment 2, wherein the one or more TDP-43 proteinopathies are caused by a loss of or altered TDP-43 function.
4. The method of embodiment 3, wherein the loss of or altered TDP-43 function is due to
(i) a mutation in the TDP-43 gene or open reading frame; and/or
(ii) an altered TDP-43 function due to one or more of nuclear clearance, cytoplasmic inclusions, nuclear inclusions, aggregation, abnormal modification, and neuronal propagation in a “prion-like” manner.
5. The method of embodiment 4, wherein the mutation comprises at least one selected from the group consisting of: D169G, K263E, N267S, G287S, G290A, S292N, G294A, G294V, G295R, G295S, G298S, M311V, A315T, A321V, A321G, Q331K, S332N, G335D, M337V, Q343R, N345K, G348C, G348V, N352S/T, R361S, P363A, Y374X, N378D, S379P, S379C, A382P, A382T, I383V, G384R, N390D, N390S, and S393L.
6. The method of embodiment 3, wherein said loss of or altered TDP-43 function promotes cryptic exon inclusion.
7. The method of embodiment 3, wherein said loss of or altered TDP-43 function reduces expression levels of the normal transcript(s) of STMN2, SORT1, GPSM2, and/or ATG4B.
8. The method of embodiment 3, wherein said loss of or altered TDP-43 function promotes a splicing defect in a STMN2, SORT1, GPSM2, and/or ATG4B gene.
9, The method of embodiment 3, wherein said loss of or altered TDP-43 function inhibits neurite and/or axon growth.
10. The method of any one of embodiments 1 to 9, wherein the neurological disease comprises at least one of cryptic exon-induced neurological diseases (CEIND).
11. The method of embodiment 10, wherein the cryptic exon-induced neurological diseases (CEIND) comprise Amyotrophic Lateral Sclerosis (ALS), Frontotemporal lobar degeneration (FTLD), Frontotemporal Dementia (FTD), myotonic dystrophy type 1 (DM1), Alzheimer's disease, Lewy body dementia (LBD), Pick's disease, Hippocampal sclerosis, Corticobasal degeneration, Huntington disease, Parkinson's disease, Argyrophilic grain disease, Chronic traumatic encephalopathy (CTE), Perry syndrome, Alexander disease, Multisystem proteinopathy (MSP), intellectual disability. ADHD, dyslexia, epilepsy, bipolar disorder, depression and schizophrenia, parkinsonism-dementia complex of Guam, dementia pugilistica, diffuse neurofibrillary tangles with calcification, Down's syndrome, familial British dementia, familial Danish dementia, frontal lobe dementia, Gerstmann-Straussler-Scheinker disease, globular glial tauopathies, white matter tauopathy with globular glial inclusions, Guadeloupian parkinsonism with dementia, Guadeloupian progressive supranuclear palsy, multiple system atrophy, myotonic dystrophy, neurodegeneration with brain iron accumulation, Hallervorden-Spatz disease, pantothenate kinase-associated neurodegeneration, neurofibrillary tangle-predominant dementia. Niemann-Pick disease, prosencephalitic parkinsonism, prion protein cerebral amyloid angiopathy, progressive supranuclear palsy, SLCI A6-related mental retardation, and subacute sclerosing panencephalitis.
12. The method of any one of embodiments 1 to 11, wherein the increased expression levels and/or stability of hnRNP L reduces the splicing defect caused by TDP-43 proteinopathies.
13. The method of any one of embodiments 1 to 12, wherein the agent comprises at least one of a small molecule, a nucleic acid, an enzyme, a protein, a polypeptide, an antibody or a functional fragment thereof, an aptamer, a small interfering RNA, a microRNA, a small hairpin RNA, an antisense nucleic acid, and a PNA.
14. The method of embodiment 13, wherein the agent comprises an hnRNP L polypeptide or a polynucleotide encoding said polypeptide.
15. The method of embodiment 13, wherein the agent comprises a small molecule compound selected from the group consisting of ascochlorin (ASC), vertihemipterin A, 4-O-methyl ascochlorin (MAC), vertuhemipterin A aglycone, AS-6,8′-hydroxyascochlorin, cylindrol A5,8′,9′-dehydroascochlorin, ascofuranol, LL-Z1272ζ (8′-acetoxyascochlorin), ascofuranone (AF), and AF derivatives.
16. The method of embodiment 13, wherein the agent comprises a small molecule compound comprising at least one chemical structure of Formulas 1 to 77.
17. The method of any one of embodiments 1 to 16, wherein the agent comprises ascochlorin (ASC), an ascochlorin derivative, or an ascochlorin analogue.
18. A composition comprising an agent capable of increasing expression levels and/or stability of hnRNP L, for treating a subject with a neurological disease associated with a splicing defect caused by TDP-43 proteinopathies.
19. The composition of embodiment 18, wherein said subject has a loss of or altered TDP-43 function.
20. The composition of embodiment 19, wherein said loss of or altered TDP-43 function promotes cryptic exon inclusion.
21. The composition of embodiment 19 or 20, wherein said loss of or altered TDP-43 function reduces expression levels of the normal transcript(s) of STMN2, SORT1, GPSM2, and/or ATG4B.
22. The composition of any one of embodiments 19 to 21, wherein said loss of or altered TDP-43 function promotes a splicing defect in a STMN2, SORT1, GPSM2, and/or ATG4B gene.
23. The composition of any one of embodiments 19 to 22, wherein said loss of or altered TDP-43 function inhibits neurite and/or axon growth.
24. The composition of any one of embodiments 18 to 23, wherein the neurological disease comprises at least one of cryptic exon-induced neurological diseases (CEIND) or poison exon-induced neurological disease (PEIND).
25. The method of embodiment 24, wherein the cryptic exon-induced neurological diseases (CEIND) comprise Amyotrophic Lateral Sclerosis (ALS), Frontotemporal lobar degeneration (FTLD), Frontotemporal Dementia (FTD), myotonic dystrophy type 1 (DM1), Alzheimer's disease, Lewy body dementia (LBD), Pick's disease, Hippocampal sclerosis, Corticobasal degeneration, Huntington disease, Parkinson's disease, Argyrophilic grain disease, Chronic traumatic encephalopathy (CTE), Perry syndrome, Alexander disease, Multisystem proteinopathy (MSP), intellectual disability, ADHD, dyslexia, epilepsy, bipolar disorder, depression and schizophrenia, parkinsonism-dementia complex of Guam, dementia pugilistica, diffuse neurofibrillary tangles with calcification, Down's syndrome, familial British dementia, familial Danish dementia, frontal lobe dementia, Gerstmann-Straussler-Scheinker disease, globular glial tauopathies, white matter tauopathy with globular glial inclusions, Guadeloupian parkinsonism with dementia, Guadeloupian progressive supranuclear palsy, multiple system atrophy, myotonic dystrophy, neurodegeneration with brain iron accumulation, Hallervorden-Spatz disease, pantothenate kinase-associated neurodegeneration, neurofibrillary tangle-predominant dementia, Niemann-Pick disease, prosencephalitic parkinsonism, prion protein cerebral amyloid angiopathy, progressive supranuclear palsy, SLCI A6-related mental retardation, and subacute sclerosing panencephalitis
26. The composition of any one of embodiments 18 to 25, wherein the increased expression levels and/or stability of hnRNP L reduces the splicing defect caused by TDP-43 proteinopathies.
27. The composition of any one of embodiments 18 to 26, wherein the agent comprises at least one of a small molecule, a nucleic acid, an enzyme, a protein, a polypeptide, an antibody or a functional fragment thereof, an aptamer, a small interfering RNA, a microRNA, a small hairpin RNA, an antisense nucleic acid, and a PNA.
28. The composition of embodiment 27, wherein the agent comprises an hnRNP L polypeptide or a polynucleotide encoding said polypeptide.
29. The composition of embodiment 27, wherein the agent comprises a small molecule compound selected from the group consisting of ascochlorin (ASC), vertihemipterin A, 4-O-methyl ascochlorin (MAC), vertuhemipterin A aglycone, AS-6,8′-hydroxyascochlorin, cylindrol A5,8′,9′-dehydroascochlorin, ascofuranol, LL-Z1272ζ (8′-acetoxyascochlorin), ascofuranone (AF) and AF derivatives.
30. The composition of embodiment 27, wherein the agent comprises a small molecule compound comprising at least one chemical structure of Formulas 1 to 77.
31. The composition of any one of embodiments 18 to 30, wherein the agent comprises ascochlorin (ASC), an ascochlorin derivative, or an ascochlorin analogue.
32. The composition of any one of embodiments 18 to 31, wherein the agent comprises at least one of neostigmine bromide, irinotecan, captopril, proscillaridin, digoxin, and 0179445-0000 (DSigDB).
33. A pharmaceutical composition for treating a subject with a neurological disease associated with a splicing defect caused by TDP-43 proteinopathies, comprising the composition of any one of embodiments 18-32 and a pharmaceutically acceptable carrier.
34. A kit comprising a composition of any one of embodiments 18-32 or a pharmaceutical composition of embodiment 32.
35. A method of treating a subject with a hnRNP L proteinopathy-dependent neurological disease, comprising administering to said subject an agent to increase expression levels and/or stability of hnRNP L, thereby repairing said splicing defect.
36. A method of treating a subject with a cryptic exon-, poison exon- or intron retention-dependent neurological disease, comprising administering to said subject an agent to increase expression levels and/or stability of hnRNP L, thereby repairing said cryptic exon or intron retention defect.
37. A method of mitigating age-induced neurological disease, or neurologic function decline, comprising administering to said subject an agent to increase expression levels and/or stability of hnRNP L.
38. A method for mitigating neuronal hypoxia-induced neurological disease including amyotrophic lateral sclerosis, comprising administering to said subject an agent to increase expression levels and/or stability of hnRNP L.

### EXAMPLES

### Example 1

The Examples described herein describe exemplary experimentation to illustrate a therapeutic entry for Amyotrophic Lateral Sclerosis (ALS) and other TDP-43 proteinopathies. TDP-43 and hnRNP L are major repressors of toxic cryptic exons. Etiology of these diseases include cryptic exon-induced or poison exon-induced pathology resulting from decreased TDP-43 function. Thus, a therapeutical method is provided herein to enhance hnRNP L function using pharmacological or genetic tools. A methodology is used to define the subset of cryptic/poison exon induced disease where elevation of hnRNP L may provide a therapeutic entry point.

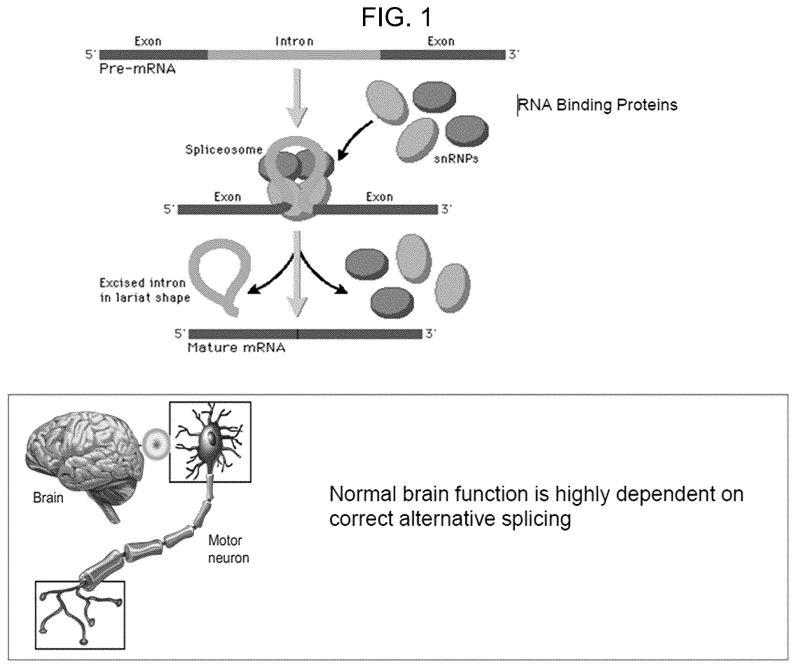

FIG. 1 is a set of graphs illustrating the normal RNA splicing as an essential process. A graph on the upper panel shows the normal process of RNA splicing, involving spliceosome and RNA binding protein snRNPs to cleave off introns from pre-mRNAs to produce mature mRNAs. The bottom panel illustrates that normal brain function is highly dependent on correct alternative splicing. Alternative splicing generates different transcripts/proteins.

Due to the important role of alternative splicing in the function of the nervous system, aberrant recruitment of cryptic or poison exons can result in severe pathologies. The vast majority of cryptic exons (about 97%) are predicted to induce premature termination codons. Aberrant splicing (at canonical sites or cryptic exon sites) can lead to severe pathologies, such as spliceopathy-induced neurological diseases. For example, altered splicing in Friedreich Ataxia (FRDA) affects canonical splice sites (Kumari and Usdin Clin Epigenetics 2012; 4:2).

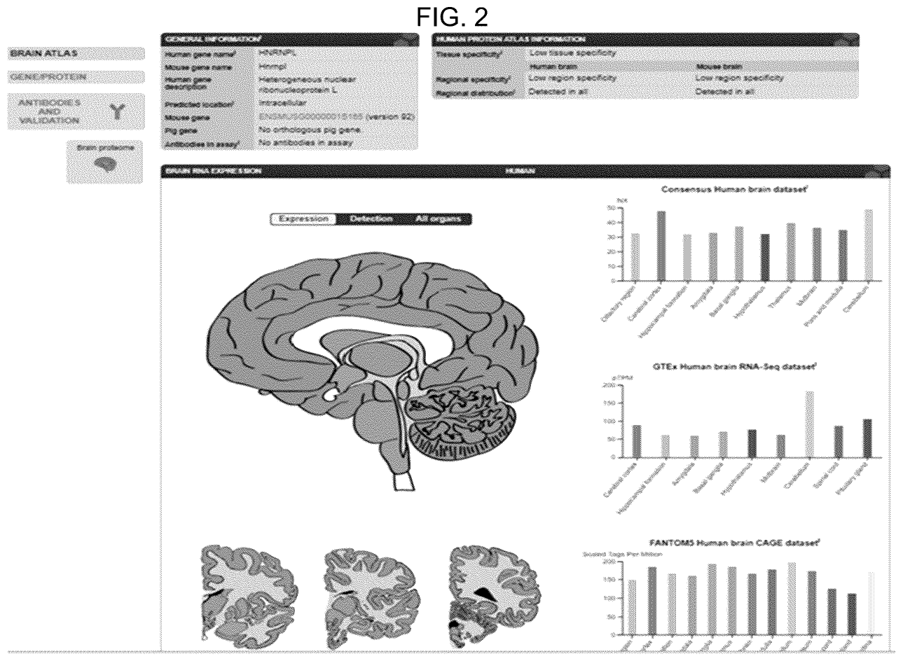

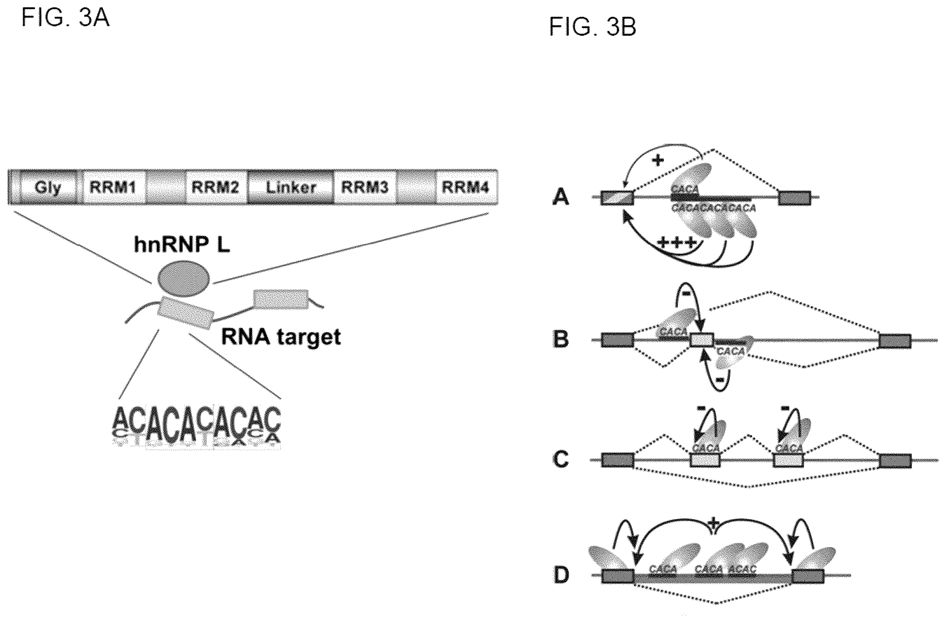

HnRNP L is a cryptic exon repressor. As shown in FIG. 2, hnRNP L, as a splicing factor, is widely expressed and distributed in the human brain. As a highly conserved protein, hnRNP L recognizes CA-repeat sequences and CA-rich motifs on targets (FIGS. 3A and 3B).

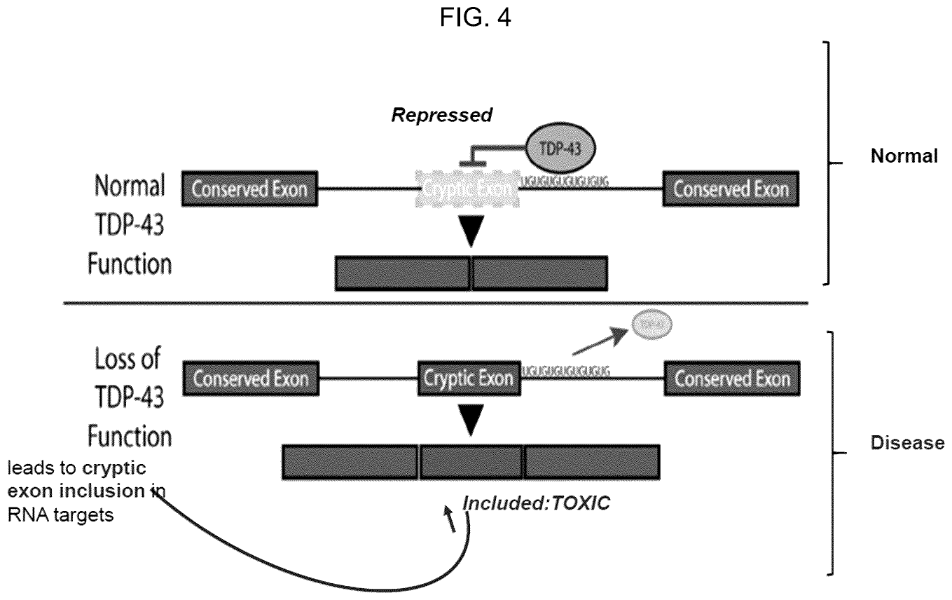

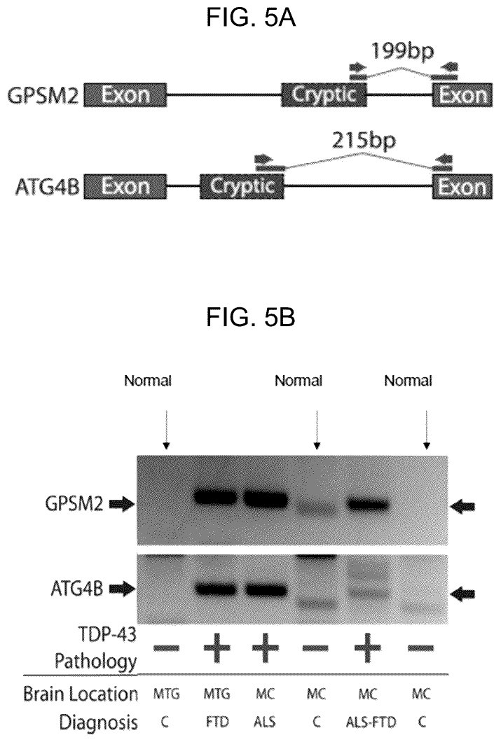

TDP-43 also acts as a repressor of cryptic exons (FIG. 4). TDP-43 aggregation, disruption [e.g., loss of function by cytoplasmic inclusions in sporadic Amyotrophic Lateral Sclerosis (ALS) and Frontotemporal Dementia (FTD)], or mutations (in familial ALS) leads to cryptic exon inclusions in target RNAs (e.g., GPSM2 and ATG4B validated in diseased brains) (FIG. 5A; also see Ling et al. Science 2015; 349:650-655). As shown in FIG. 5B, both FTD and ALS lead to cleavage and truncation of GPSM2 and ATG4B DNA. Cryptic exons are found in postmortem brain tissue RNA isolated from ALS/FTD patients with TDP-43 proteinopathy.

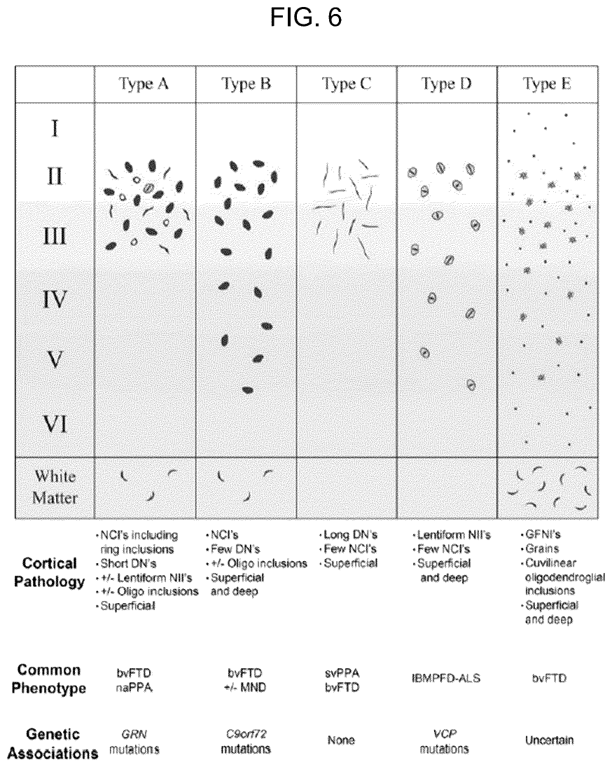

Examples of TDP-43 pathologies associated with Frontotemporal Dementia (sporadic or familial FTD), as well as the common phenotypes and genetic associations are shown in FIG. 6. Specifically, TDP-43 aggregate morphology is defined as FTLD-TDP Types A, B, C, D, and E. Type A is characterized by crescentic to oval/ring-like neuronal cytoplasmic inclusions and many short dystrophic neurites involving superficial neocortical layers. Lentiform neuronal intra-nuclear inclusions and oligodendroglial (oligo) inclusions may also be observed. Type B is characterized by neuronal cytoplasmic inclusions affecting superficial and deep neocortical layers with a paucity of dystrophic neurites. Oligodendroglial inclusions may be observed. Type C is characterized by long dystrophic neurites predominantly in superficial layers with a paucity of neuronal cytoplasmic inclusions. Type D is characterized by frequent lentiform neuronal intranuclear inclusions with short dystrophic neurites. Type E is characterized by granulofilamentous neuronal inclusions and very fine, dot-like neuropil aggregates affecting all neocortical layers in addition to curvilinear oligodendroglial inclusions in the white matter.

### Example 2

This Example illustrates that hnRNP L and TDP-43 play an essential role in preventing cryptic exon inclusion.

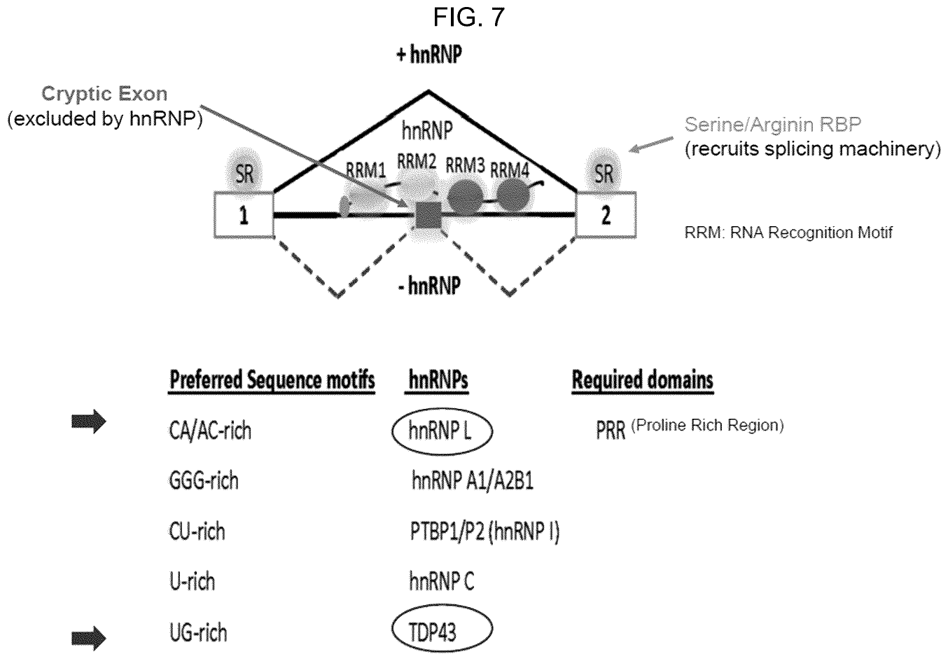

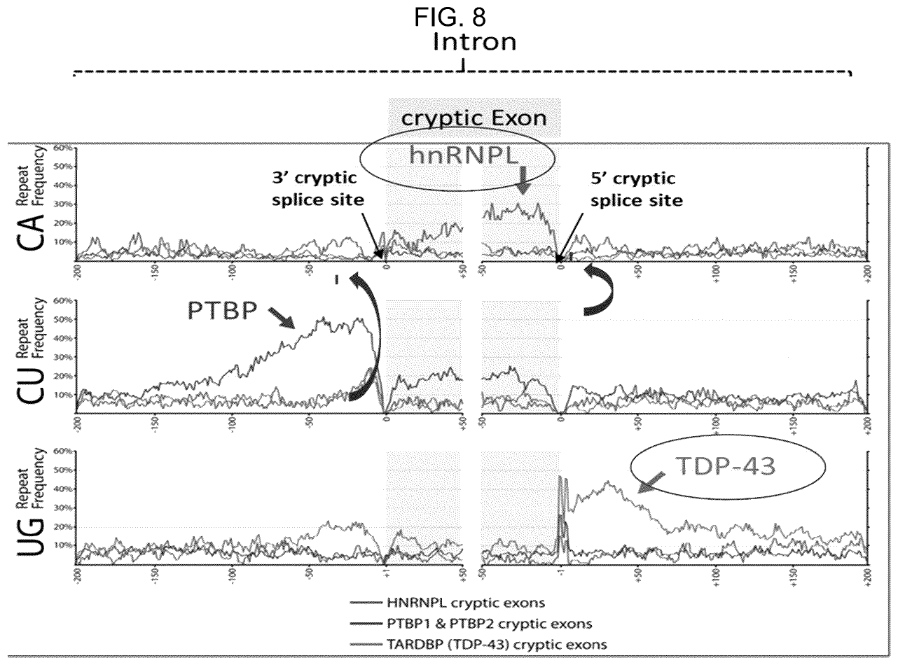

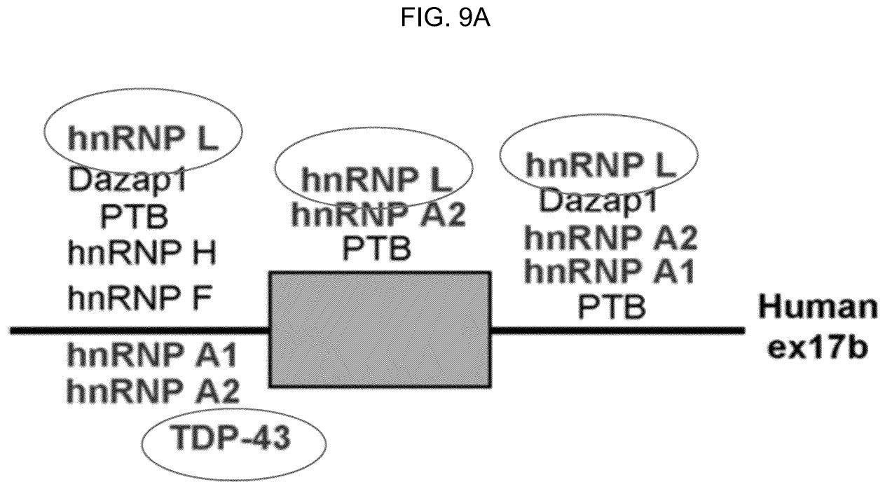

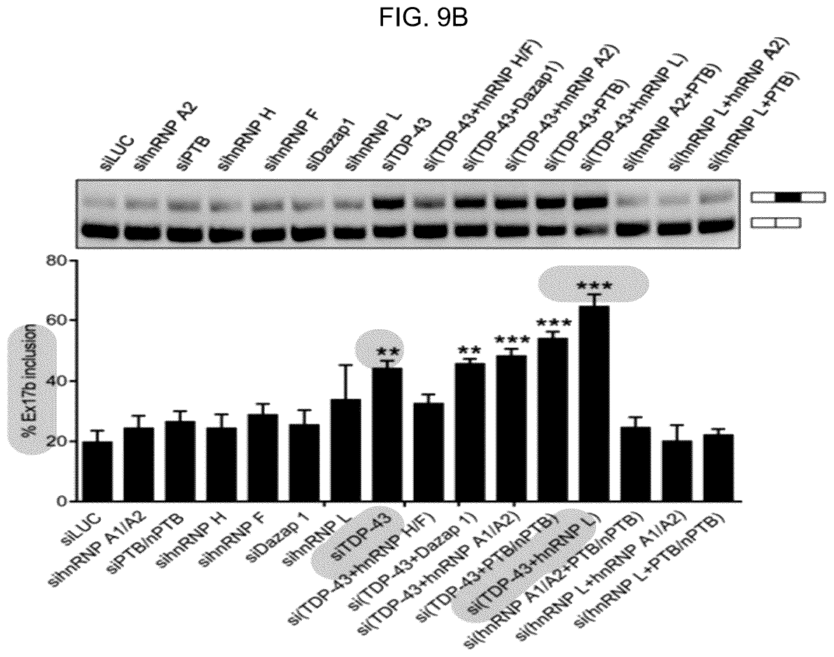

As shown in FIG. 7, transcriptome integrity is protected by different hnRNPs. For example, hnRNP L binds to CA/AC-rich motifs to inhibit cryptic exons (see also Das et al. RNA Biology 2019; 16:155-159). HnRNP L acts in concert with selected splicing factors: TDP-43, PTBP1/P2 (hnRNP I), hnRNP C, and hnRNP A1/A2B1 to repress cryptic exons. Drug treatment elevates hnRNP L expression. Promising cohorts are be found to assess the efficacy of an agent that increases hnRNP L levels. As shown in FIG. 8, HnRNP L, TDP-43 and PTBP are important repressors of cryptic (pathogenic) exons. TDP-43 and hnRNP L repress a cryptic exon associated with Frontotemporal dementia (FTD) (FIGS. 9A and 9B). Specifically, loss of TDP-43 promotes inclusion of toxic exon17b, leading to a toxic SORT1 isoform elevated in FTLD-TDP. Knockdown of hnRNP L and TDP-43 leads to significant inclusion of endogenous Sort1 Ex17b in Neuro 2a cells (FIG. 9B).

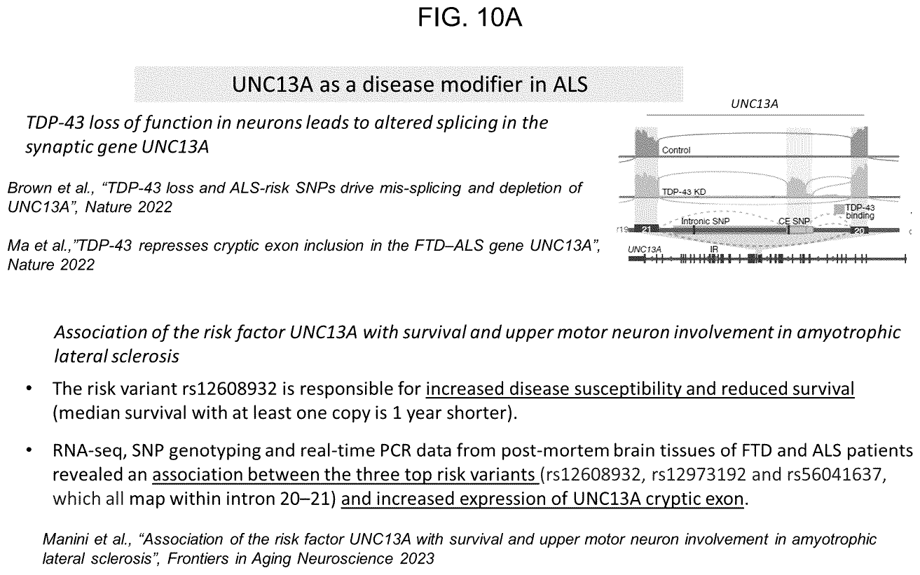

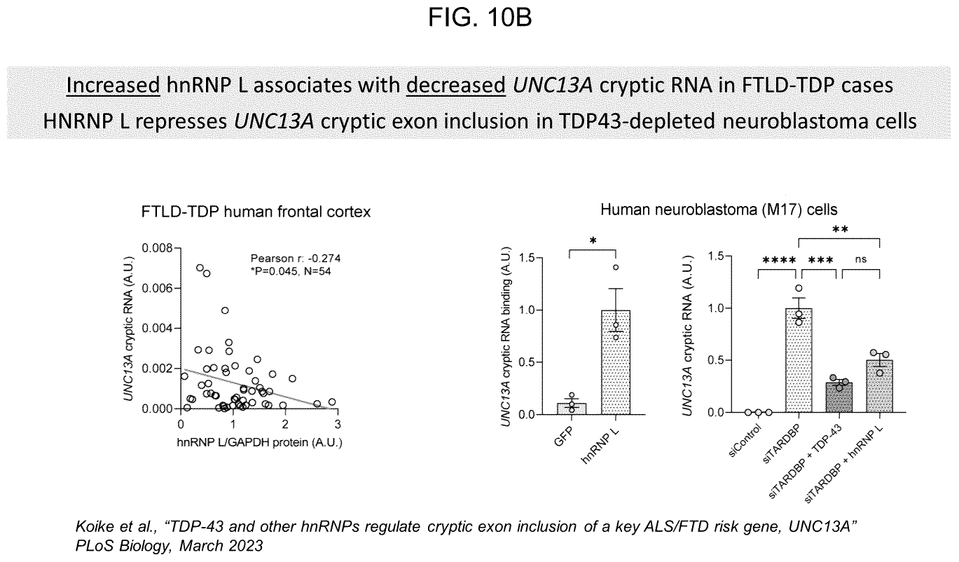

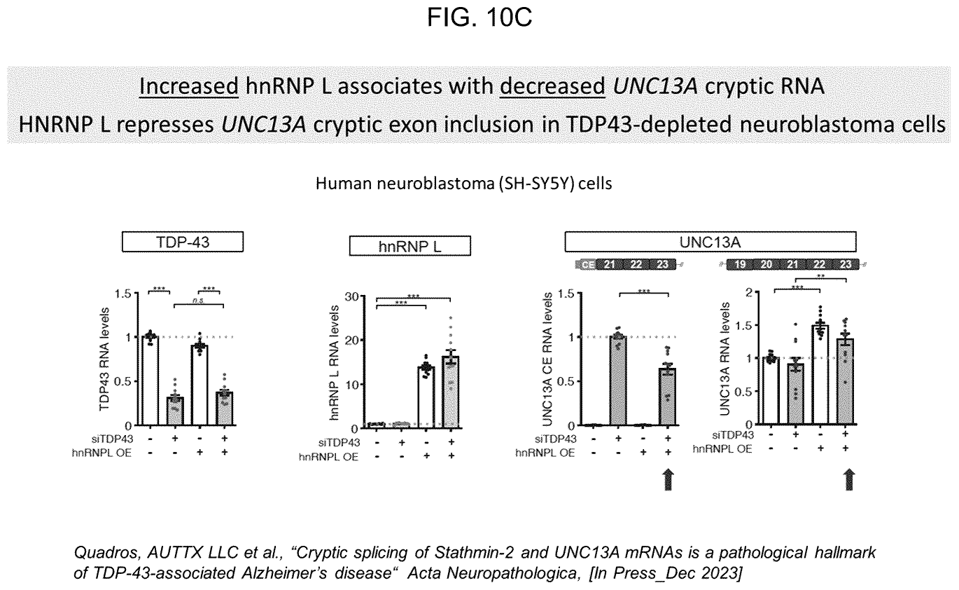

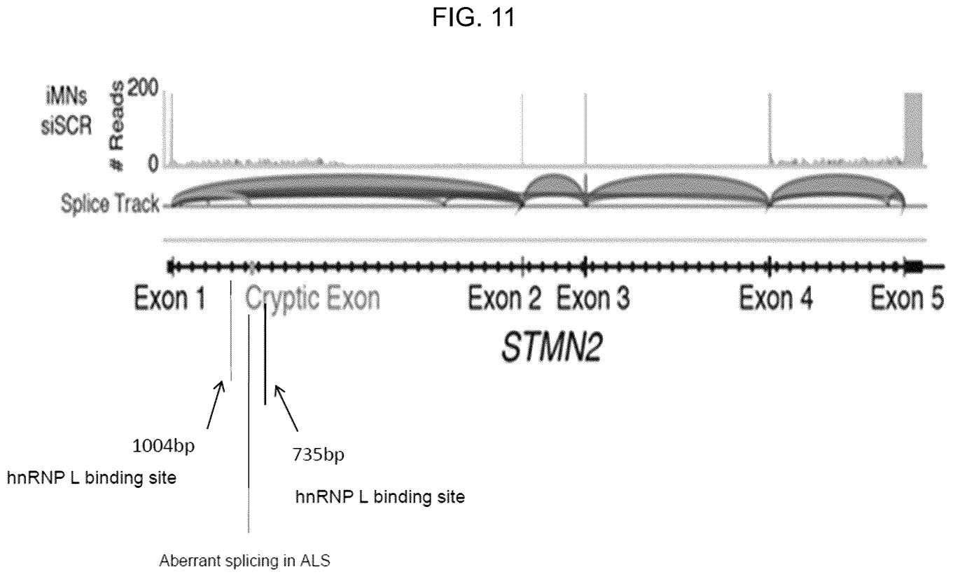

TDP-43 mutations cause Amyotrophic Lateral Sclerosis (ALS) (FIG. 10A, 10B, and FIG. 11). While TDP-43 regulates normal splicing of UNC13A transcript (FIGS. 10A, 10B, and 10C), or of STMN2 (FIG. 11) transcript, loss of TDP-43 leads to inclusion of cryptic exon. Mutations (and/or decreased functional expression or aggregation) of TDP-43 lead to expression of cryptic exons (splice abnormalities) in selected target genes resulting in inclusions leading to several neurological diseases including ALS and Frontotemporal dementia (FTD) (see Ma et al., Nature 2022, volume 603, pages 124-130; Brown et al., Nature 2022, volume 603, pages 131-137; Klim et al., Nature Neuroscience 2019; 22:167-179). Increased hnRNP L levels lead to decreased inclusion of UNC13A cryptic exon (FIG. 10B, 10C). Thus, elevating hnRNP L is an alternative means to repress cryptic exon.

Specifically, the STMN2 gene (FIG. 11) or the UNC13A gene have hnRNP L binding sites in proximity to the cryptic exon. TDP-43 deficiency in cells results in expression of a cryptic exon, which is rescued with hnRNP L elevation (achieved by either pharmacological or genetic means). Such hnRNP L treatment leads to suppression of the pathological cryptic exon.

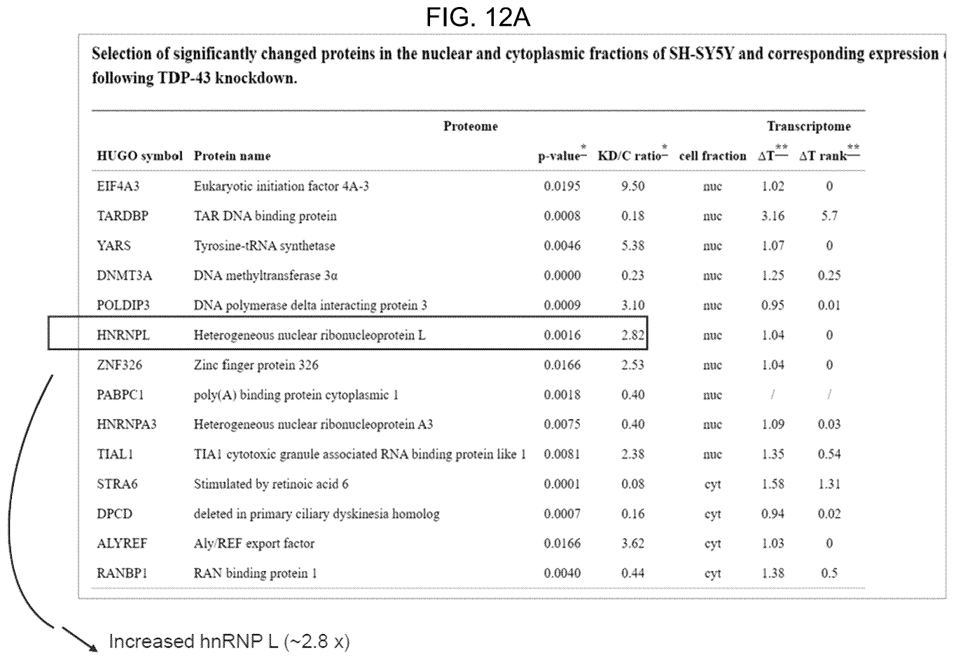

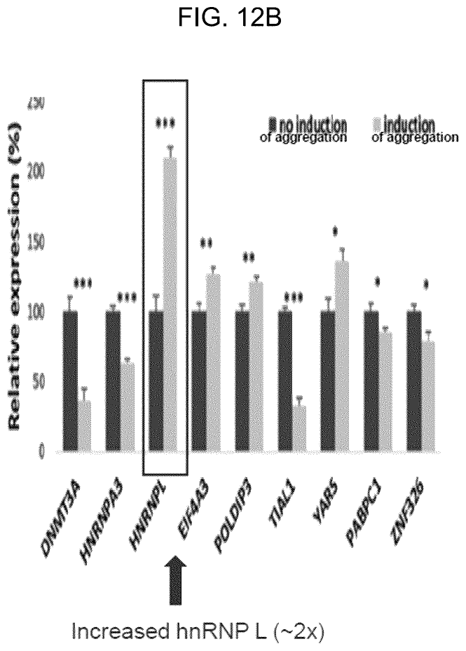

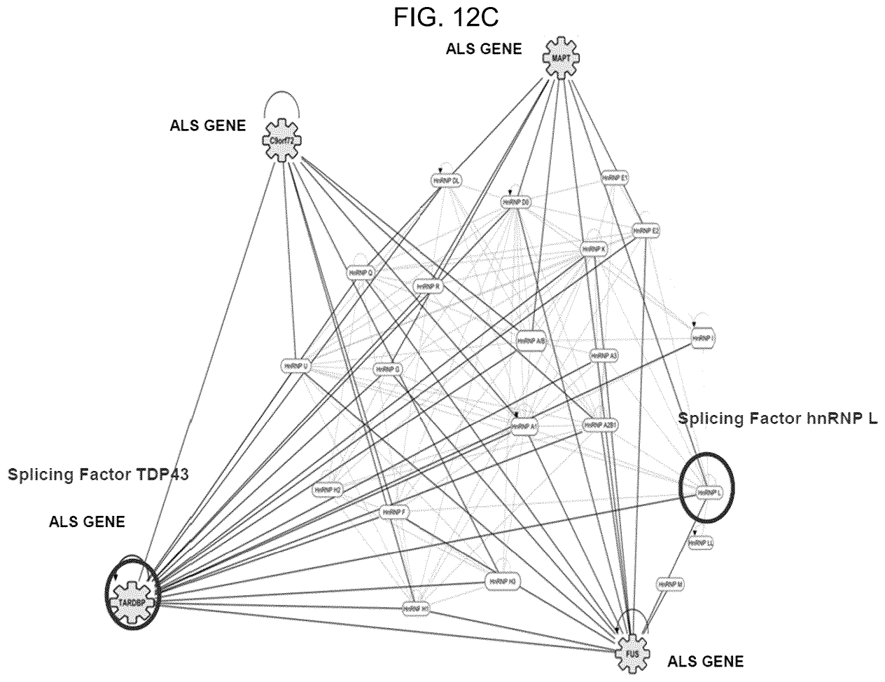

An increase in hnRNP L levels (using either pharmacological or genetic tools) may compensate for TDP-43 deficiency in FTD, ALS and other neurological disorders. TDP-43 knockdown in SH-SY5Y cells results in significant changes in the nuclear and cytoplasmic fractions, including an increased expression of hnRNP L levels to about 2.8 folds (2.8×) (FIG. 12A). Similarly, TDP-43 aggregation in HEK293 cells leads to multiple changes, including an increased expression of hnRNP L levels to about 2 folds (2×) (FIG. 12B). TDP-43 aggregation is seen in 60% of FTLD and 90-95% of ALS cases.

HnRNP L and TDP-43 interact with each other (FIG. 12C).

### Example 3

This Example illustrates exemplary experimentation to test whether normal splicing can be restored, by elevation of hnRNP L, in a spliceopathy-induced neurological disorder background. A new candidate mechanism for ALS therapeutics is illustrated herein.

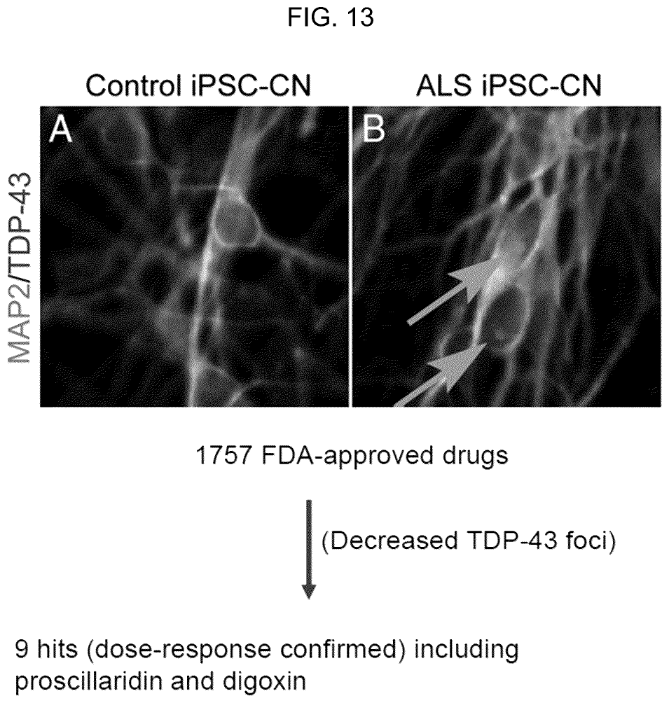

A cellular model for sporadic ALS using patient-derived induced pluripotent stem cells was reported by Burkhardt et al. Mol Cell Neurosci 2013; 56:355-364 (FIG. 13). iPS cell derived-motor neurons from patient with sporadic ALS/TDP-43 aggregation were used for a high throughput screen for potential ALS therapeutics. Nine hits confirmed in dose response assays, including proscillaridin and digoxin. The data indicates that this rescue is attributed to inhibition of Na/K ATPase in cardiac cells.

About ˜60% of frontotemporal lobar degeneration (FTLD) and ˜90% of ALS cases are characterized by the formation of TDP-43 cytoplasmic inclusions. TDP-43 aggregation mirrors TDP-43 knockdown, affecting the expression levels of a common set of proteins, as those listed in Mihevc et al., Scientific Reports 2016; 6:33996 [including increased hnRNP L (˜2×)]. In the Drug Signatures Database (DSigDB) database, six drugs are associated with increased hnRNP L levels, including proscillaridin, digoxin, captopril, 0179445-000, neostigmine bromide, and irinotecan (Table 4; also see Yoo et al. Bioinformatics 2015; 31:3069-3071). Thus, these drugs may be used to increase hnRNP L levels, which in turn rescues the abnormal splicing caused by a functional decrease in TDP-43 (e.g., from mutant TDP-43, decreased TDP-43 expression, TDP-43 aggregation, etc.). This rescue is due to increased hnRNP L in motor neurons.

TABLE 4
Drugs in the DSigDB database associated
with increased hnRNP L levels

| Gene | Source | Chemical Name |
|---|---|---|
| HNRNPL | D3(UP) | captopril |
| HNRNPL | D3(UP) | proscillaridin |
| HNRNPL | D3(UP) | digoxin |
| HNRNPL | D3(UP) | 0179445-000 |
| HNRNPL | D3(UP) | neostigmine bromide |
| HNRNPL | D3(UP) | irinotecan |

Further to the above-mentioned chemicals, a family of small molecules were identified that elevate hnRNP L levels. These compounds offer therapeutics for ALS and other TDP-43 proteinopathies (and/or neurological diseases linked to cryptic/poison exon inclusion).

This family of small molecules include Ascochlorin (ASC) and its analogs and/or variants (e.g., Formulas 1-77). ACS has a structure as shown in Formula 3 or 3′, with a molecule weight of 404.93. As a small molecule, ASC is capable of crossing the blood-brain barrier and entering brain.

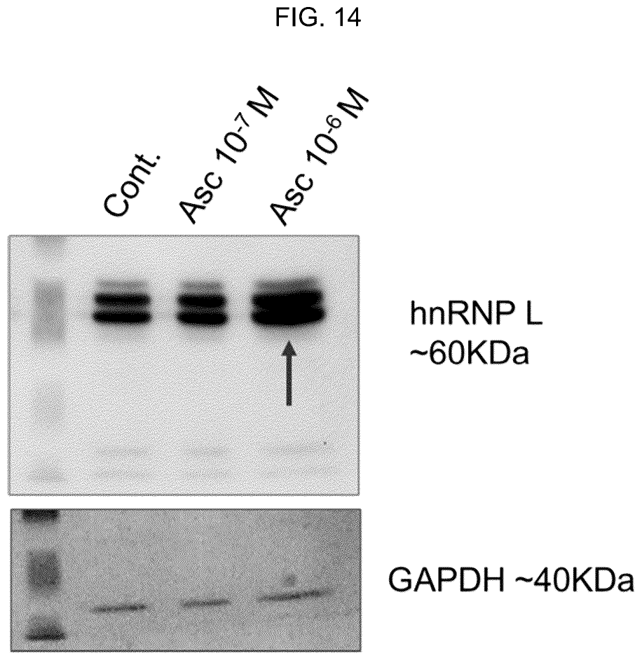

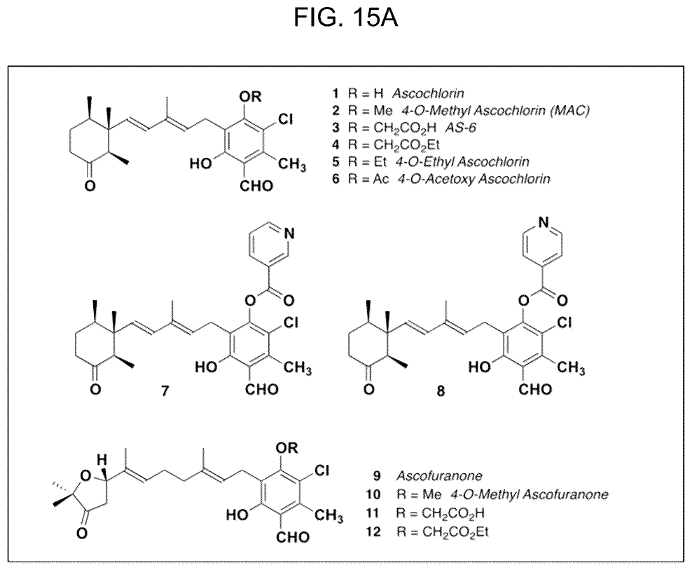

ASC treatment increased hnRNP L expression levels in rat primary cortical neurons (FIG. 14), and human differentiated myoblasts (about 6-fold). Such function of ASC was also found in human osteosarcoma cells (about 10-fold) and multiple disease models, see PCT publication no. WO/2019/236750, which is incorporated by reference herein in its entirety.

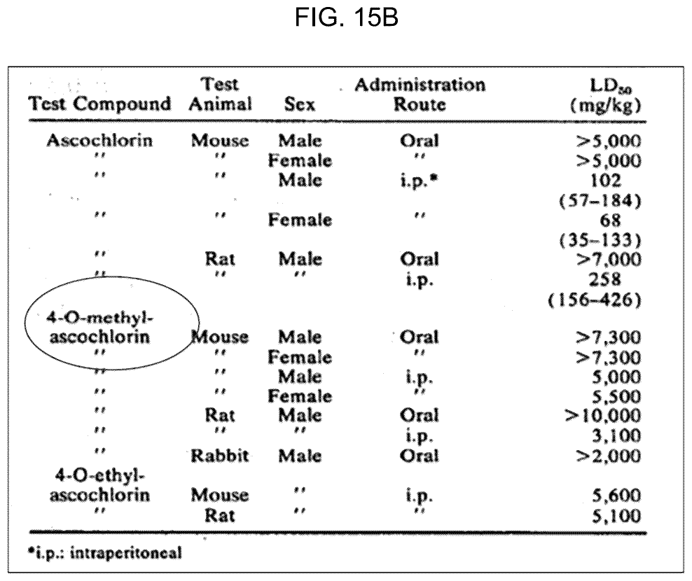

Various ascochlorin derivatives or analogs were tested for toxicity. As shown in FIGS. 15A and 15B, many compounds have low toxicity, such as ascochlorin derivatives 4-O-methyl-ascochlorin (MAC) and 4-O-ethyl-ascochlorin. Oral administration of 4-O-methyl-ascochlorin (MAC) showed low toxicity to mouse and rat. U.S. Pat. No. 3,995,061 describes the ascochlorin derivatives 4-O-methyl-ascochlorin (MAC) and 4-O-ethyl-ascochlorin have low toxicity when administered orally to human. Ascochlorin and ascofuranone can cross the blood brain barrier (thus being promising anti-trypanosomal/sleeping sickness agents). The complete biosynthesis of ascochlorin and ascofuranone was recently achieved (see Araki et al. PNAS 2019; 116:8269-8274 and Yuan et al. Eur J Med Chem 2020; 202:112502). FIG. 16 further shows absence of significant toxicity with administration of ascochlorin/derivatives in rodent disease models (in the reference list: 1. Dai et al. Molecular Oncology. 2015; 9:818-833; 2. Nakajima et al., J. Antibiot. 2007; 60:682-689; 3. Kim et al. Arch Biochem Biophys. 2015; 583:79-86; 4. Hosokawa et al. Lipids. 1981; 16:433-438; 5. Hosokawa et al., Diabetes. 1985; 34:267-274; and 6. Yabu et al., Parasitology International. 2003; 52:155-164).

In summary, the present application covers any agent that increases hnRNP L as a therapeutic for TDP-43 proteinopathies and other neurological diseases (as listed in this application).

### Example 4

TDP-43 proteinopathies can be rescued using pharmacologic or genetic tools that elevate hnRNP L levels.

Wild-type TDP-43 inclusions are found in 90-95% ALS and 60% FTLD cases (termed TDP-43 proteinopathies). Aggregation of TDP-43 is most probably the root cause of ALS/FTLD. TDP-43 positive cytoplasmic inclusions have also been described in, at least, 57% of Alzheimer's disease cases, 20% of Dementia with Lewy Bodies, Pick's disease, hippocampal sclerosis, corticobasal degeneration, Huntington disease, Parkinson's disease, Argyrophilic grain disease, Chronic traumatic encephalopathy (CTE), Perry syndrome, Alexander disease, Multisystem proteinopathy (MSP), as well as other diseases or disorders described in this specification. These diseases have similar histology: TDP-43 aggregates in neurons or glia. Thus, ASC therapy or any agent that elevates hnRNP L may be used to alleviate or treat these diseases by compensating TDP-43 aggregation or mutations (FIG. 17).

### Example 5

As a specific example, a lentiviral vector encoding hnRNP L is discussed below.

Insert RC219240 Human HNRNPL
(NM_001533, transcript variant 1) ORF into
PS100069 pLenti-C-Myc-DDK-IRES-Puro
vector backbone, The stop codon should be
included for untagged hnRNP L protein
Custom lentiviral particles produced from
CW307794 [Human HNRNPL (NM_001533)-
IRES-Puro] lenti plasmid,,
200 ul, >10\hat{ }8 TU/ml
Insert RC209067 Human HNRNPL
(NM_001005335, transcript variant 2) ORF
into PS100069 pLenti-C-Myc-DDK-IRES-Puro
vector backbone, The stop codon should be
included for untagged hnRNP L protein, 10 ug
Custom lentiviral particles produced from
CW307795 [Human HNRNPL
(NM_001005335)-IRES-Puro] lenti plasmid,
200 ul, >10\hat{ }8 TU/ml
hnRNP L variant 1: NM_001533.3
hnRNP L variant 2: NM_001005335.2
TARDBP: NM_007375.4

As a specific example, AAV9 encoding hnRNP-L is discussed below:

### AAV9-hnRNP L Synthesis Steps

1. hnRNP synthesis (ENSEMBLE: hnRNP L-201, 2142 basepairs encoding protein isoform 589 aa, transcript ID ENST00000221419.10)
2. sub-cloning of pAAV.hnRNP
3. Maxi Prep of pAAV.hnRNP
4. Production of AAV9.hnRNP

A chicken beta actin promoter is used to enhance expression. AAV-9 is given by the ICV, IT and intracerebral injection routes to animal models that demonstrate features of Amyotrophic Lateral Sclerosis (ALS), or of other cryptic/poison exon-induced neurological diseases (CEIND, PEIND), and/or display abnormal expression of a target gene due to cryptic exon/poison exon incorporation. Two to -eight weeks post injection, animals and corresponding controls are functionally assessed (motor, processing, lifespan) and then sacrificed. Target gene (expressing cryptic or poison exon) expression levels and corresponding transcript sequence are compared using PCR and RNAseq analysis. Return to normal function and/or expression of a normal target transcript after administration of AAV-hnRNP L to a diseased animal reflects efficacy of the construct in the corresponding disease. The hnRNP L treatment may be efficacious for cryptic/poison exon induced disease, spliceopathy induced diseased and/or age-related decline in neuronal function.

An in vitro strategy to define cryptic/poison exon induced disease sensitive to treatment with hnRNP L relies on the use of neurons or neuronal cell lines (primary, iPS derived WT or with disease mimicking mutations; neuronal cell lines). Corresponding cells are treated with doses of small molecules, AAV-hnRNP L or lentivirus-hnRNP L resulting in an elevation of hnRNP L. This is done in the presence and absence of cycloheximide which prevents nonsense mediated decay. After treatment for 2-10 days, restoration of the normal target transcript sequence i.e., absence of the cryptic or poison exon, as assessed by PCR or RNA seq serves as an index of therapeutic efficacy.

Restoration of the normal basal levels of poison/cryptic expression serves as an index of therapeutic efficacy.

Restoration of the normal levels of expression, or normal isoform, of the protein encoded by the target transcript serves an index of therapeutic efficacy.

A reporter gene construct may also be used to determine transcript expression and integrity (inclusion of a cryptic/poison exon).

Alternatively, cells are treated for 2-10 days, after which RNAseq (>100 million reads) is done comparing the resulting transcripts obtained with the different cellular conditions (control, hnRNP L elevated +/−cycloheximide). Elevation in the basal level of cryptic/poison exon expression defines a subset of candidate disease producing inclusions. Correction of the defective transcript in the presence of elevated hnRNP L suggests treatment with corresponding small molecules, AAV-hnRNP L or lentivirus-hnRNP L as human therapeutics. To enhance the likelihood of response to hnRNP L elevation, a bioinformatic search for hnRNP L binding sites in the vicinity (within 200 nucleotides, within 500 nucleotides, within 1000 nucleotides, etc.) of the cryptic/poison exon is done.

### Example 6. HNRNP L-Mediated Rescue of Aberrant Splicing

As a specific example, the experimental approach assessing hnRNP L-mediated rescue of aberrant splicing linked to a neurologic disorder is outlined below

### Cell Line

TDP-43 siRNA-treated SH-SY5Y. SH-SY5Y neuroblastoma cells (ATCC) where SiRNA treatment reduces TDP-43 mRNA levels by >80% (vs. levels in control siRNA-treated cells). Upon TDP-43 depletion, reduction in STMN2 levels is observed by qPCR and WB (8-fold reduction). Other TDP-43 gene targets such as UNC13A may be similarly affected as STMN2.

### Alternate Cell Line

CRISPR-Cas9 genetically engineered SH-SY5Y cells expressing the familial ALS-causing mutation TDP-43^{N352S}. QPCR and WB analysis of the isogenic wild-type and mutant lines show reduced STMN2 levels (1.7-fold reduction in mRNA, and >2-fold reduction in protein) in TDP-43N352S mutant cells.

Further details of the cell-based models of ALS are provided in doi.org/10.1038/s41593-018-0293-z.

### Compounds and Treatment

Cells are plated cells into 96-well plates. Serial 5-fold dilutions of the compounds are added the following day. The starting concentration is 5 10\hat{ }−6 M. A dose response assessment (5 points) is done. Vehicle alone (DMSO, or water, depending on the compound) is used as a control. The compounds (≥98% pure/HPLC) 10 mM stock solutions are made in DMSO, or water (depending on the compound's solubility).

The cells are incubated with the compound at 37° C. in the dark, for the following period of time:
(i) 72 hours (short-term treatment, the compounds are not refreshed during this period)
(ii) 10 days (long-term treatment, the compounds are refreshed twice, on day 4 and day 7)

### Readout

Compound treatment results in a quantifiable decrease in STMN2 exon 2a inclusion, and/or an increase in either full-length STMN2 mRNA, or in STMN2 protein levels (in TDP-43 depleted or TDP-43 mutated cells).

### Lentivirus Treatment

HnRNP L lentiviral particles (i-iii, below, and describe above).
(i) hnRNP L transcript variant 1 ORF human lentiviral particle (pLenti-hnRNP L 1) 200 μl, 10⁷ TU/ml;
(ii) hnRNP L transcript variant 2 ORF human lentiviral particle (pLenti-hnRNP L 2) 200 μl, 10⁷ TU/ml;
(iii) Empty pLenti-hnRNP L-Myc-DDK vector (control) 200 μl, 10⁷ TU/ml.

siRNA-treated SH-SY5Y cells, or CRISPR-Cas9 modified SH-SY5Y cells (cells lines as described above) are plated cells into 6-well plate and cultured to maturation. Following plating, the cells are infected with the lentiviral particle encoding human hnRNP L at 10⁷ TU/ml, or 106 TU/ml, in the presence of polybrene for 96 hours. The corresponding empty lentiviral particle is used as a control.

Positive control: In parallel, the modified SH-SY5Y are cultured and infected with lentiviral particles encoding stathmin-2 in the presence of polybrene.

### Readouts:

Splicing, and mRNA Levels

The cells are lysed and RT will be added to produce cDNA from each well. PCR and/or qPCR reactions are carried out to assess (i) STMN2 exon 2a inclusion/exclusion, (ii) total STMN2 mRNA levels, (iii) total hnRNP L mRNA levels and (iv) total TPD-43 mRNA levels.

Controls: RT-PCR of the CENP-A transcript are used as loading control for RT-PCR, and TFRC and GAPDH are used as qPCR normalizers.

### Other Readouts

Immunoblot analysis: Total-cells protein extracts are prepared. Equal amounts of proteins are separated in 10% acrylamide gel, transferred to PVDF and incubated with the following primary antibodies: anti-TDP-43 (1:1,000) (ProteinTech, 10782-2-AP), anti-stathmin-2 (1:2,000) (nbp1-49461, NovusBiologicals), anti-tubulin (control, 1:10,000) (DMIA, Abcam) and anti-hnRNP L (clone 4D11 Sigma-Aldrich R 4903).

For the lentiviral constructs studies, an anti-DDK tag antibody (Origene) will be used (to monitor expression of the tagged human hnRNP L).

The secondary horseradish peroxidase-conjugated antibody is used.

Further details of the readouts are provided in doi.org/10.1038/s41593-018-0293-z.

In some embodiments, the agents of the disclosure can be incorporated into compositions, including pharmaceutical compositions. Such compositions generally include the agents as described herein and a pharmaceutically acceptable carrier. Accordingly, in one aspect, some embodiments of the disclosure relate to pharmaceutical compositions for treating, preventing, ameliorating, reducing or delaying the onset of a health condition, for example, a neurological disease associated with a splicing defect. Such splicing defect may be caused by one or more TDP-43 proteinopathies. Such TDP-43 proteinopathies may be caused by a loss of or altered TDP-43 function, as described herein.

Alternatively, independent of abnormal TDP-43, an increase in hnRNP L expression may restore normal expression of genes in which a cryptic/poison exon is expressed resulting in disease. Target genes where this applies are suggested by the experiment outlined above.

### Example 7. Screening of Nucleic Acids Targeting HNRNP L

Antisense oligonucleotides (ASOs) targeting hnRNP L were generated. Some example target sequences are shown in Table 5. Some of the examples are within or near poison exon 6A of hnRNP L. Exon 6A is an example of a poison exon where inclusion of the exon results in truncated hnRNP L, and so it is preferable in some situations to block inclusion of the poison exon, or to degrade or block transcription of mature mRNA molecules that include the poison exon.

TABLE 5
Example target sequences

| hnRNP L Target Sequence (5′ to 3′) | SEQ ID NO: | Corresponding ASO | Region |
|---|---|---|---|
| GAGGCGGGTTCTGCGCCGCCAT | 24 | 5UTR-V1-1 | 5′ UTR |
| TTCTGCGCCGCCATGTCGCGGA | 25 | 5UTR-V1-2 | 5′ UTR |
| GCCATGTCGCGGAGGCTGCTGC | 26 | 5UTR-V1-3 | 5′ UTR |
| AAAAATTGTAGCGGATGTGTAC | 27 | Skip-6A-1 | Upstream Intron |
| GTAGCGGATGTGTACCGTGACT | 28 | Skip-6A-2 | Upstream Intron |
| TGTGTACCGTGACTTGTATTTA | 29 | Skip-6A-3 | Upstream Intron |
| GTGACTTGTATTTATGACTGTA | 30 | Skip-6A-4 | Upstream Intron |
| GTATTTATGACTGTAAAACCAT | 31 | Skip-6A-5 | Upstream Intron |
| GACTGTAAAACCATGTGATGCA | 32 | Skip-6A-6 | Upstream Intron |
| AAACCATGTGATGCAGGGTCGC | 33 | Skip-6A-7 | Upstream Intron/Exon |
| GTGATGCAGGGTCGCAGTGTAT | 34 | Skip-6A-8 | Upstream Intron/Exon |
| CAGGGTCGCAGTGTATGTTTGA | 35 | Skip-6A-9 | Upstream Intron/Exon |
| CGCAGTGTATGTTTGATGGGAC | 36 | Skip-6A-10 | Exon |
| TGTATGTTTGATGGGACGCCAT | 37 | Skip-6A-11 | Exon |
| TTTGATGGGACGCCATCTTTCA | 38 | Skip-6A-12 | Exon |
| GGGACGCCATCTTTCAGAACTG | 39 | Skip-6A-13 | Exon |
| CCATCTTTCAGAACTGTGCTAA | 40 | Skip-6A-14 | Exon |
| TTCAGAACTGTGCTAACTCACT | 41 | Skip-6A-15 | Exon |
| CTGTGCTAACTCACTGTTGAAG | 42 | Skip-6A-16 | Exon |
| TAACTCACTGTTGAAGCGTCCA | 43 | Skip-6A-17 | Exon |
| ACTGTTGAAGCGTCCAATGGTA | 44 | Skip-6A-18 | Exon |
| GAAGCGTCCAATGGTAAGAGAA | 45 | Skip-6A-19 | Exon |
| CCAATGGTAAGAGAAAATACAG | 46 | Skip-6A-20 | Exon |

Some example ASO sequences targeting hnRNP L are shown in Table 6 to Table 8. The sequences in Table 6 were used in experiments herein. In the experiments that were conducted with the base sequences in Table 6, the sequences were fully modified DNA where every nucleoside included 2′-O-methoxyethyl (MOE), every C base was modified as 5′-methyl C, and phosphorothioate linkages were included between every nucleoside. Some additional examples of ASO base sequences that may be used to target hnRNP L are shown in Table 7A and Table 7B. ASOs were designed to avoid flags outlined in Table 8.

TABLE 6
ASO base sequences

| ASO Name | SEQ ID NO: | Base Sequence (5′ to 3′) |
|---|---|---|
| 5UTR-V1-1 | 47 | ATGGCGGCGCAGAACCCGCCTC |
| 5UTR-V1-2 | 48 | TCCGCGACATGGCGGCGCAGAA |
| 5UTR-V1-3 | 49 | GCAGCAGCCTCCGCGACATGGC |
| Skip-6A-1 | 50 | GTACACATCCGCTACAATTTTT |
| Skip-6A-2 | 51 | AGTCACGGTACACATCCGCTAC |
| Skip-6A-3 | 52 | TAAATACAAGTCACGGTACACA |
| Skip-6A-4 | 53 | TACAGTCATAAATACAAGTCAC |
| Skip-6A-5 | 54 | ATGGTTTTACAGTCATAAATAC |
| Skip-6A-6 | 55 | TGCATCACATGGTTTTACAGTC |
| Skip-6A-7 | 56 | GCGACCCTGCATCACATGGTTT |
| Skip-6A-8 | 57 | ATACACTGCGACCCTGCATCAC |
| Skip-6A-9 | 58 | TCAAACATACACTGCGACCCTG |
| Skip-6A-10 | 59 | GTCCCATCAAACATACACTGCG |
| Skip-6A-11 | 60 | ATGGCGTCCCATCAAACATACA |
| Skip-6A-12 | 61 | TGAAAGATGGCGTCCCATCAAA |
| Skip-6A-13 | 62 | CAGTTCTGAAAGATGGCGTCCC |
| Skip-6A-14 | 63 | TTAGCACAGTTCTGAAAGATGG |
| Skip-6A-15 | 64 | AGTGAGTTAGCACAGTTCTGAA |
| Skip-6A-16 | 65 | CTTCAACAGTGAGTTAGCACAG |
| Skip-6A-17 | 66 | TGGACGCTTCAACAGTGAGTTA |
| Skip-6A-18 | 67 | TACCATTGGACGCTTCAACAGT |
| Skip-6A-19 | 68 | TTCTCTTACCATTGGACGCTTC |
| Skip-6A-20 | 69 | CTGTATTTTCTCTTACCATTGG |

TABLE 7A
Additional ASO base sequences

| ASO Name | SEQ ID NO: | Base Sequence (5′ to 3′) |
|---|---|---|
| 5UTR-V2-1 | 70 | CGGATCGAAAAGAGCAGACACT |
| 5UTR-V2-2 | 71 | CGGCCGTCCCGGATCGAAAAGA |
| 5UTR-V2-3 | 72 | CGAGCCTGACCGGCCGTCCCGG |
| 5UTR-V2-4 | 73 | CTCGGCGGCGAGCCTGACCGGC |
| 5UTR-V2-5 | 74 | TAGTTCTCCAGCTCGGCGGCGA |
| 5UTR-V2-6 | 75 | CGGGTCATCGTAGTTCTCCAGC |
| 5UTR-V2-7 | 76 | GGGTTTTGTGCGGGTCATCGTA |
| 5UTR-V2-8 | 77 | CTGGGGAGGCAGGGGTTTTGTG |
| 5UTR-V2-9 | 78 | TGTGGACAACTGGGGAGGCAGG |
| 5UTR-V2-10 | 79 | GCCCCTGATGTGGACAACTGGG |
| 5UTR-V2-11 | 80 | CGTCAATCAGGCCCCTGATGTG |
| 5UTR-V2-12 | 81 | TCCACCACACCGTCAATCAGGC |
| 5UTR-V2-13 | 82 | GGTCTGCTTCCACCACACCGTC |
| 5UTR-V2-14 | 83 | AAGGCCTCCACAAGGTCTGCTT |
| 5UTR-V2-15 | 84 | AAACTCCTGCAAGGCCTCCACA |
| 5UTR-V2-16 | 85 | TGATGGGTCCAAACTCCTGCAA |
| 5UTR-V2-17 | 86 | ACCACATAGCTGATGGGTCCAA |
| 5UTR-V2-18 | 87 | AGGCATTACCACCACATAGCTG |

TABLE 7B
Additional ASO base sequences

| ASO Name | SEQ ID NO: | Base Sequence (5′ to 3′) |
|---|---|---|
| Gap-6A-1 | 88 | CCCTTGTCCACTGAGATTGG |
| Gap-6A-2 | 89 | CGACCCTTGTCCACTGAGAT |
| Gap-6A-3 | 90 | TGCGACCCTTGTCCACTGAG |
| Gap-6A-4 | 91 | CACTGCGACCCTTGTCCACT |
| Gap-6A-5 | 92 | ATACACTGCGACCCTTGTCC |
| Gap-6A-6 | 93 | AACATACACTGCGACCCTTG |
| Gap-6A-7 | 94 | TCAAACATACACTGCGACCC |
| Gap-6A-8 | 95 | ACCATTGGACGCTTCAACAG |
| Gap-6A-9 | 96 | GTCACCATTGGACGCTTCAA |
| Gap-6A-10 | 97 | GGGTCACCATTGGACGCTTC |
| Gap-6A-11 | 98 | CCAGGGTCACCATTGGACGC |
| Gap-6A-12 | 99 | CTGCCAGGGTCACCATTGGA |
| Gap-6A-13 | 100 | TGCTGCCAGGGTCACCATTG |
| Gap-6A-14 | 101 | GTTGCTGCCAGGGTCACCAT |

TABLE 8

| Flag | Criteria | Meaning |
|---|---|---|
| FailCCCC | CCCC present in guide | Guide sequence |
| | | contains CCCC, |
| | | meaning passenger |
| | | will contain GGGG - |
| | | see below |
| FailCpG | >2 CG present in | CpG are considered |
| | sequence | immunogenic and have |
| | | been shown to be toxic |
| | | in ASO and, to a lesser |
| | | extent, in siRNA. This |
| | | effect can potentially |
| | | be mitigated by |
| | | chemical |
| | | modifications, so I |
| | | allow up to 2 to be |
| | | present in the |
| | | sequence. |
| FailGGGG/ | GGGG present in guide | GGGG quadruplex can |
| FailCCCC | or passenger | be challenging to |
| | | synthesize & purify, |
| | | and has been reported |
| | | to be toxic in some |
| | | cases. |
| FailHumanRefseqXX | presence of an mRNA | Off-target |
| | OT hit to 19mer of | |
| | guide, with 0 or 1 | |
| | mm, or 2 mm with | |
| | contiguous 17mer | |
| | match | |
| FailmiRNA1 | As for | Off-target |
| | FailHumanRefseq3MM, | |
| | specfically searching | |
| | miRNA | |
| FailLncRNA1 | As for | Off-target |
| | FailHumanRefseq3MM, | |
| | specfically searching | |
| | lncRNA | |
| FailPalindrome | Presence of a | Palindromic sequences |
| | palindromic sequence | increase self- |
| | of 8 bases or more. | dimerization potential |
| | | and have been |
| | | observed to be |
| | | immunogenic |
| FailPolyX | Presence of a | Low complexity & |
| | homopolymer run of 6 | increased OT potential |
| | bases or more | |
| FailRepeat | Guide overlaps a | Increases specificity. |
| | repetitive element | Filter can be removed |
| | | in cases where limited |
| | | candidates exist. |
| FailSNPOverlap | Overlap of SNP of 1% | Avoid targeting known |
| | or higher MAF | SNPs |
| FailSplice | Sequence is not | Target all known |
| | present in all | (high-quality) splice |
| | required splice | forms |
| | variants. | |

ASOs from Table 6 with the modifications as described were tested in vitro. BE(2)-M17 cells were seeded at a density of 15.000 cells/well in regular 96-well tissue culture plates, followed by transfection of cells with in total 23 ASOs of interest in a dose response setup (highest final test concentration was 120 nM, going down in four 4-fold serial dilution steps) using Lipofectamine3000 (LF3000) as a transfection reagent. Cells were incubated post-treatment for 24 h at 37° C./5% CO2 in a humidified incubator, followed by cell lysis and branched DNA (bDNA) analysis to quantify mRNA levels of interest, namely human hnRNP L (hsHNRNP L), hsGAPDH and hsAHSA-1. Negative control treatment of cells included mock treatment (transfection reagent only w/o any oligo) as well as transfection of cells with two ASOs targeting hsAHSA-1 (X35906 and X15080) and R-Luc (X22986) at the highest test concentration on each plate.

Data were generated in biological quadruplicates. A mean ratio of hsHNRNP L/hsGAPDH mRNA in negative control treated cells was artificially set to 100% and used for normalization of all other sample treatments and data. An Excel add-in XLfit software tool was used to calculate EC50 values in nM. No significant decrease in hsGAPDH mRNA levels was observed. Exceptions included slight reductions in GAPDH values for 3 ASOs (X171924, X171927 and X171928), though these slight reductions did not significantly affect the results.

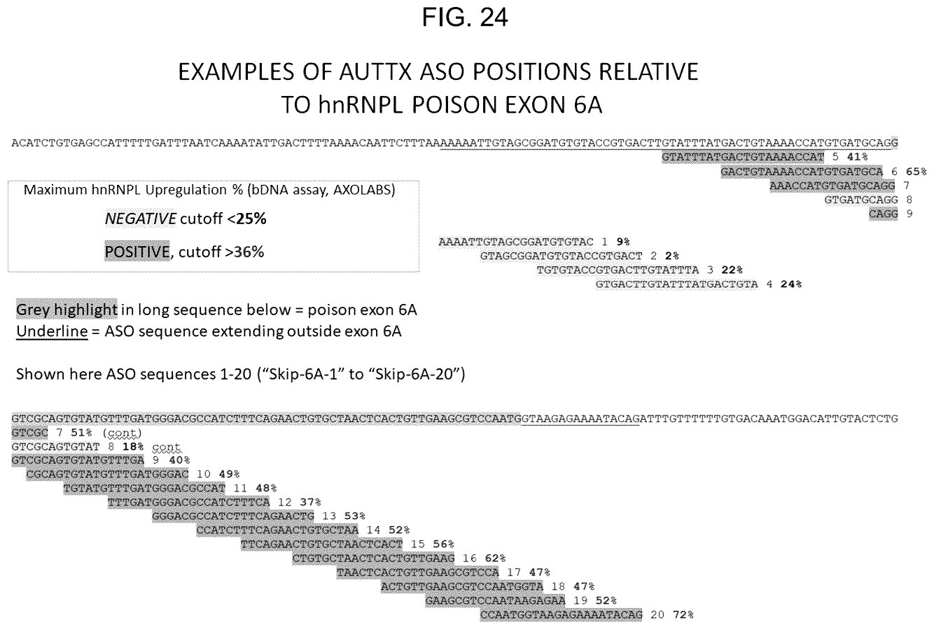

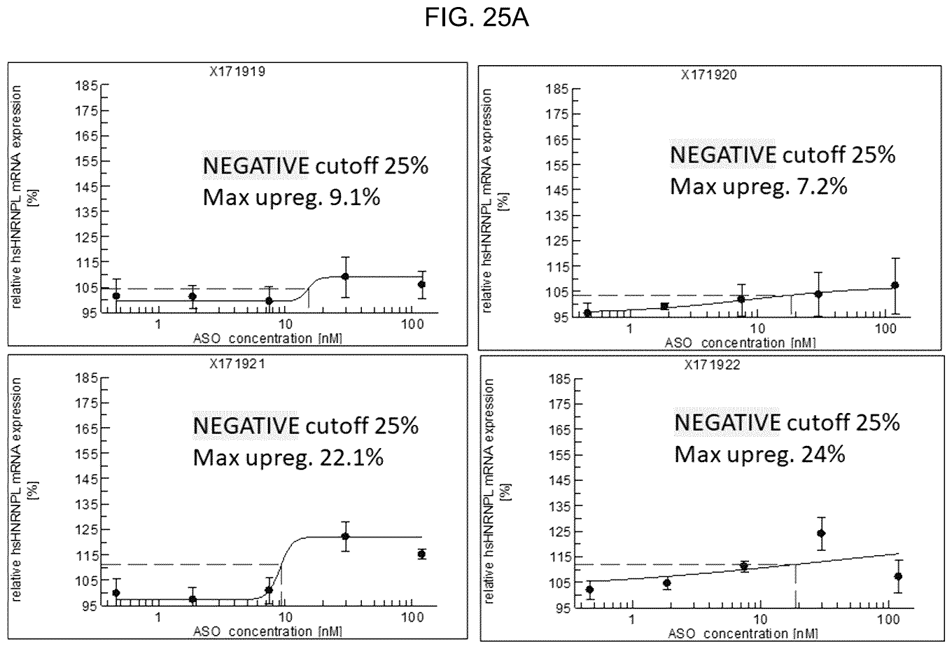

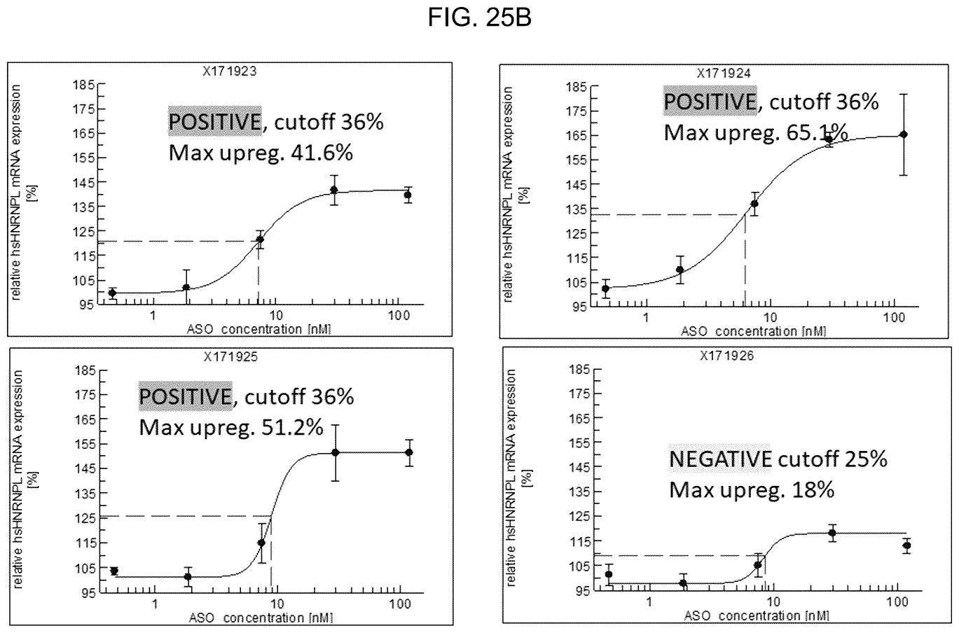

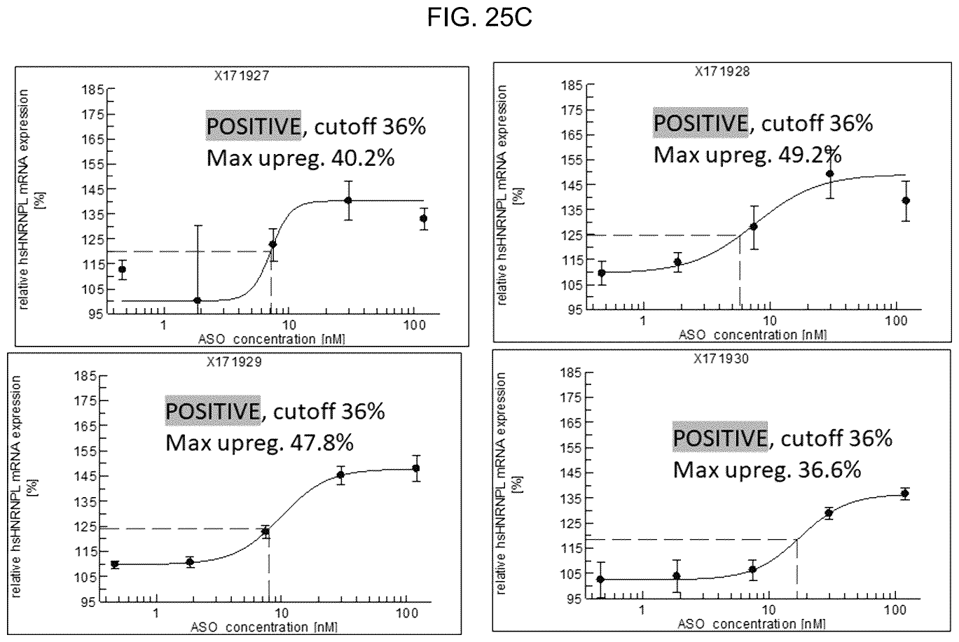

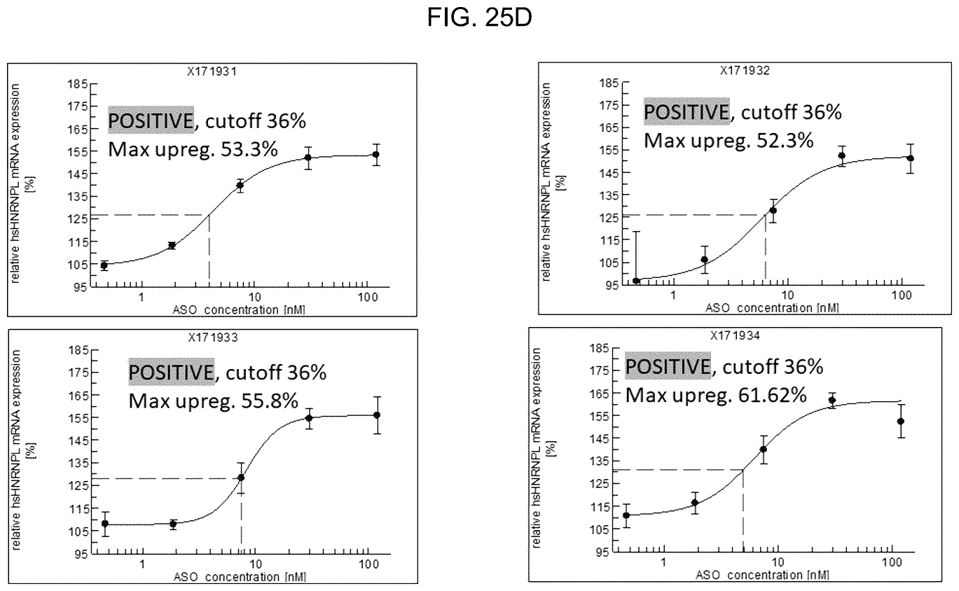

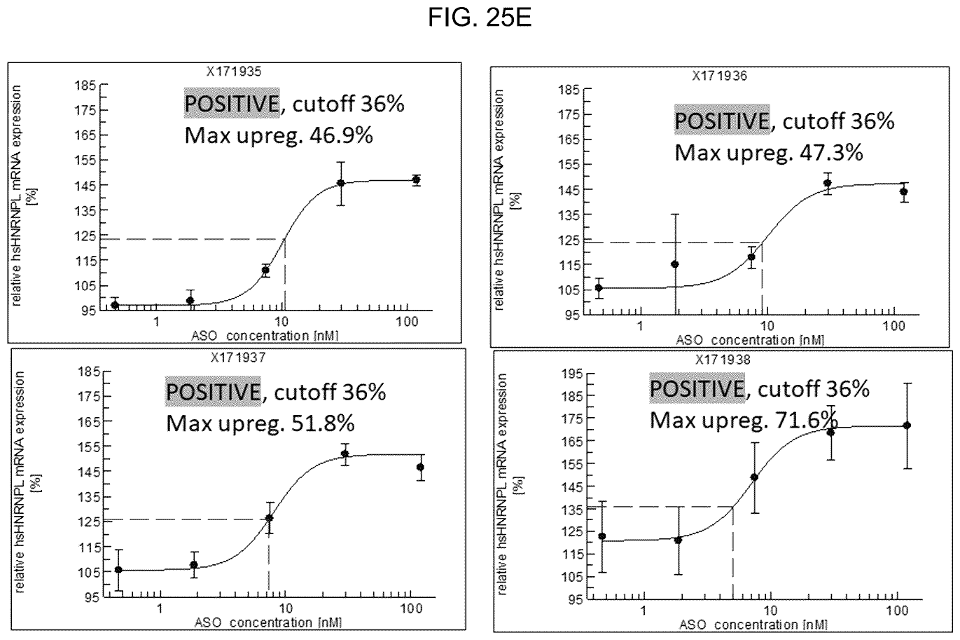

Details and data are shown in Table 9A-9B, FIG. 24, and FIG. 25A-25E. In some cases, there was dose dependent hsHNRNP L on-target mRNA upregulation to varying degrees when cells were transfected with ASO compounds of interest.

As a positive control, cells were transfected with an active ASO targeting hsAHSA-1. This analysis served as a control for transfection efficiency on each plate. Transfection of cells with the positive control ASO followed by quantification of remaining hsAHSA-1 mRNA levels resulted in robust and homogenous on-target mRNA silencing. This demonstrates high, robust and reproducible transfection efficacy on each plate.

TABLE 9A
5-point DRC ASOs

| ASO Name | Alternative ASO Name | Type | target |
|---|---|---|---|
| — | X35906 | positive control A | AHSA1 |
| — | X15080 | control B | AHSA1 |
| — | X22986 | control C | R-Luc |
| Skip-6A-1 | X171919 | sample | hsHNRNP L |
| Skip-6A-2 | X171920 | sample | hsHNRNP L |
| Skip-6A-3 | X171921 | sample | hsHNRNP L |
| Skip-6A-4 | X171922 | sample | hsHNRNP L |
| Skip-6A-5 | X171923 | sample | hsHNRNP L |
| Skip-6A-6 | X171924 | sample | hsHNRNP L |
| Skip-6A-7 | X171925 | sample | hsHNRNP L |
| Skip-6A-8 | X171926 | sample | hsHNRNP L |
| Skip-6A-9 | X171927 | sample | hsHNRNP L |
| Skip-6A-10 | X171928 | sample | hsHNRNP L |
| Skip-6A-11 | X171929 | sample | hsHNRNP L |
| Skip-6A-12 | X171930 | sample | hsHNRNP L |
| Skip-6A-13 | X171931 | sample | hsHNRNP L |
| Skip-6A-14 | X171932 | sample | hsHNRNP L |
| Skip-6A-15 | X171933 | sample | hsHNRNP L |
| Skip-6A-16 | X171934 | sample | hsHNRNP L |
| Skip-6A-17 | X171935 | sample | hsHNRNP L |
| Skip-6A-18 | X171936 | sample | hsHNRNP L |
| Skip-6A-19 | X171937 | sample | hsHNRNP L |
| Skip-6A-20 | X171938 | sample | hsHNRNP L |
| 5UTR-V1-1 | X171939 | sample | hsHNRNP L |
| 5UTR-V1-2 | X171940 | sample | hsHNRNP L |
| 5UTR-V1-3 | X171941 | sample | hsHNRNP L |

TABLE 9B
5-point DRC data

| | max. upregulation | Standard | |
|---|---|---|---|
| | [%] (MV normalized | Deviation | EC50 |
| ASO Name | hsHNRNP L/hsGAPDH) | (SD) | [nM] |
| Skip-6A-1 | 9.1 | 8.1 | 15.1978 |
| Skip-6A-2 | 7.2 | 8.6 | 18.45822 |
| Skip-6A-3 | 22.1 | 5.7 | 9.292248 |
| Skip-6A-4 | 24.0 | 6.6 | 18.94446 |
| Skip-6A-5 | 41.6 | 5.9 | 7.252912 |
| Skip-6A-6 | 65.1 | 3.0 | 6.229787 |
| Skip-6A-7 | 51.2 | 11.3 | 8.914073 |
| Skip-6A-8 | 18.0 | 3.4 | 8.543073 |
| Skip-6A-9 | 40.2 | 7.8 | 7.154743 |
| Skip-6A-10 | 49.2 | 9.8 | 5.792782 |
| Skip-6A-11 | 47.8 | 3.7 | 7.933137 |
| Skip-6A-12 | 36.6 | 2.2 | 16.74958 |
| Skip-6A-13 | 53.3 | 5.0 | 3.969427 |
| Skip-6A-14 | 52.3 | 4.5 | 6.408461 |
| Skip-6A-15 | 55.8 | 4.6 | 7.447406 |
| Skip-6A-16 | 61.6 | 3.4 | 4.928646 |
| Skip-6A-17 | 46.9 | 8.7 | 10.5632 |
| Skip-6A-18 | 47.3 | 4.3 | 9.096056 |
| Skip-6A-19 | 51.8 | 4.3 | 7.355806 |
| Skip-6A-20 | 71.6 | 12.0 | 5.037865 |
| 5UTR-V1-1 | 11.4 | 3.1 | — |
| 5UTR-V1-2 | 16.2 | 3.7 | — |
| 5UTR-V1-3 | 22.4 | 5.7 | — |

### Example 8. Follow-Up Screening of Nucleic Acids Targeting HNRNP L

An additional time-course experiment was performed as follows, using the same ASOs as in Example 7. BE(2)-M17 cells were seeded at a cell density of 15.000 cells/well. Subsequently, the cells were transfected with a total of 20 selected ASOs at a concentration of 30 nM. Lipofectamine3000 (LF3000) was again used as the transfection agent. The transfection mixture was prepared identically on three plates. Cells were incubated post treatment for 24 h, 48 h and 72 h at 37° C./5% CO2 in a humidified incubator, followed by cell lysis and branched DNA (bDNA) analysis to quantify mRNA levels of interest, namely hsHNRNP L, hsGAPDH and hsAHSA-1. Negative control treatment of cells included mock treatment (transfection reagent only w/o any oligo) as well as transfection of cells with two ASOs targeting hsAHSA-1 (X35906 and X15080) and R-Luc (X22986) at the test concentration on each plate. Raw values for hsHNRNP L and hsGAPDH were obtained and processed. In this case, no outliers were removed. Data were generated in biological quadruplicates.

Transfection of the cells with an active ASO targeting hsAHSA1 (=X35906) and subsequent quantification of an hsAHSA1 mRNA level served again as a control of the transfection efficiency. A high, robust and homogeneous on-target mRNA silencing demonstrated transfection efficiency on each plate.

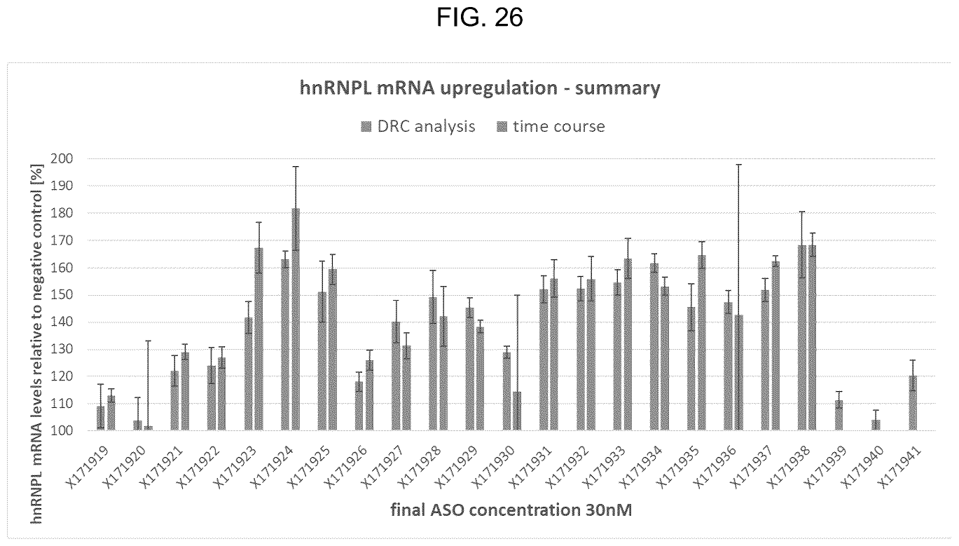

Details and data are shown in Table 10 and FIG. 26. FIG. 26 includes a comparison of results from this study next to results from Example 7. Observing the results after 24 h incubation, similar data from the analysis in Example 7 were reproduced here.

In this experiment, the mean ratio of hsHNRNP L/hsGAPDH mRNA level in mock controls only was artificially set to 100% and used for normalization of all other sample treatments and data. Note that “hs” here stands for “Homo sapiens.” The reason for this was a steady decrease in the hsGAPDH values of cells treated with ASOs in the plates with longer incubation times, which may be due to some toxicity, though overall the experiments showed successful effects of some ASOs on affecting productive hnRNP L RNA levels.

TABLE 10
Time course data

| ASO Name | MV normalized hsHNRNP L/hsGAPDH | SD |
|---|---|---|
| Skip-6A-1 | 13.0 | 2.4 |
| Skip-6A-2 | 1.8 | 31.2 |
| Skip-6A-3 | 29.0 | 2.8 |
| Skip-6A-4 | 26.9 | 4.0 |
| Skip-6A-5 | 67.3 | 9.2 |
| Skip-6A-6 | 81.7 | 15.3 |
| Skip-6A-7 | 59.3 | 5.6 |
| Skip-6A-8 | 26.0 | 3.6 |
| Skip-6A-9 | 31.3 | 4.8 |
| Skip-6A-10 | 42.1 | 10.9 |
| Skip-6A-11 | 38.3 | 2.3 |
| Skip-6A-12 | 14.5 | 35.4 |
| Skip-6A-13 | 56.0 | 6.8 |
| Skip-6A-14 | 55.8 | 8.2 |
| Skip-6A-15 | 63.4 | 7.4 |
| Skip-6A-16 | 53.2 | 3.3 |
| Skip-6A-17 | 64.5 | 4.9 |
| Skip-6A-18 | 42.7 | 55.1 |
| Skip-6A-19 | 62.4 | 2.0 |
| Skip-6A-20 | 68.3 | 4.2 |

Overall, the results in this and the previous example show that targeting hnRNP L (e.g., targeting exon 6A, the upstream intron of exon 6A in hnRNP L), or an overlapping region of both, may be effective for increasing expression of hnRNP L mRNA. The data here show that targeting a poison exon of hnRNP L may increase hnRNP L mRNA levels, and show surprising effects of some ASO sequences.

### Example 9. Example Genes Associated with Autism or HNRNP L

Some embodiments involve treating a subject having a mutation in gene in any of Tables 11-16 (taken from WO2019236750, which is incorporated by reference herein in its entirety). The mutation may result in a splicing defect. The subject may have a disease in Table 15 or 16.

TABLE 11
(881 Genes) - SFARI list of autism genes

| Human Gene | Ensembl Identifier | Accession Number* |
|---|---|---|
| ABAT | ENSG00000183044 | NP_001120920.1 |
| ABCA10 | ENSG00000154263 | NP_525021.3 |
| ABCA7 | ENSG00000064687 | NP_061985.2 |
| ACE | ENSG00000159640 | NP_000780.1 |
| ACHE | ENSG00000087085 | NP_000656.1 |
| ACTN4 | ENSG00000130402, | NP_004915.2 |
| | ENSG00000282844 | |
| ACY1 | ENSG00000243989 | NP_001185824.1 |
| ADA | ENSG00000196839 | NP_000013.2 |
| ADAMTS18 | ENSG00000140873 | NP_955387.1 |
| ADARB1 | ENSG00000197381 | NP_056648.1 |
| ADCY5 | ENSG00000173175 | NP_899200.1 |
| ADK | ENSG00000156110 | NP_001114.2 |
| ADNP | ENSG00000101126 | NP_056154.1 |
| ADORA2A | ENSG00000128271 | NP_000666.2 |
| ADORA3 | ENSG00000282608 | NP_065734.5 |
| ADRB2 | ENSG00000169252 | NP_000015.1 |
| ADSL | ENSG00000239900 | NP_000017.1 |
| AFF2 | ENSG00000155966 | NP_002016.2 |
| AFF4 | ENSG00000072364 | NP_055238.1 |
| AGAP1 | ENSG00000157985 | NP_001032208.1 |
| AGBL4 | ENSG00000186094 | NP_116174.3 |
| AGMO | ENSG00000187546 | NP_001004320.1 |
| AGTR2 | ENSG00000180772 | NP_000677.2 |
| AHDC1 | ENSG00000126705 | NP_001025053.1 |
| AHI1 | ENSG00000135541 | NP_001128302.1 |
| AKAP9 | ENSG00000127914 | NP_005742.4 |
| ALDH1A3 | ENSG00000184254 | NP_000684.2 |
| ALDH5A1 | ENSG00000112294 | NP_733936.1 |
| ALG6 | ENSG00000088035 | NP_037471.2 |
| ALOX5AP | ENSG00000132965 | NP_001191335.1 |
| AMPD1 | ENSG00000116748 | NP_000027.2 |
| AMT | ENSG00000145020 | NP_000472.2 |
| ANK2 | ENSG00000145362 | NP_001139.3 |
| ANK3 | ENSG00000151150 | NP_066267.2 |
| ANKRD11 | ENSG00000167522 | NP_001243111.1 |
| ANKS1B | ENSG00000185046 | NP_690001.3 |
| ANXA1 | ENSG00000135046 | NP_000691.1 |
| AP1S2 | ENSG00000182287 | NP_003907.3 |
| APBA2 | ENSG00000034053, | NP_005494.2 |
| | ENSG00000276495 | |
| APC | ENSG00000134982 | NP_001120982.1 |
| APH1A | ENSG00000117362 | NP_001071096.1 |
| APP | ENSG00000142192 | NP_000475.1 |
| AR | ENSG00000169083 | NP_000035.2 |
| ARHGAP11B | ENSG00000187951 | NP_001034930.1 |
| ARHGAP15 | ENSG00000075884 | NP_060930.3 |
| ARHGAP24 | ENSG00000138639 | NP_001020787.2 |
| ARHGAP32 | ENSG00000134909 | NP_001136157.1 |
| ARHGAP33 | ENSG00000004777 | NP_443180.2 |
| ARHGEF9 | ENSG00000131089 | NP_056000.1 |
| ARID1B | ENSG00000049618 | NP_065783.3 |
| ARNT2 | ENSG00000172379 | NP_055677.3 |
| ARX | ENSG00000004848 | NP_620689.1 |
| ASH1L | ENSG00000116539 | NP_060959.2 |
| ASMT | ENSG00000196433 | NP_004034.2 |
| ASS1 | ENSG00000130707 | NP_446464.1 |
| ASTN2 | ENSG00000148219 | NP_054729.3 |
| ASXL3 | ENSG00000141431 | NP_085135.1 |
| ATG7 | ENSG00000197548 | NP_006386.1 |
| ATP10A | ENSG00000206190 | NP_077816.1 |
| ATP1A3 | ENSG00000105409 | NP_001243143.1 |
| ATP2B2 | ENSG00000157087 | NP_001001331.1 |
| ATP8A1 | ENSG00000124406 | NP_006086.1 |
| ATRNL1 | ENSG00000107518 | NP_997186.1 |
| ATRX | ENSG00000085224 | NP_000480.3 |
| ATXN7 | ENSG00000163635 | NP_001170858.1 |
| AUTS2 | ENSG00000158321 | NP_056385.1 |
| AVP | ENSG00000101200 | NP_000481.2 |
| AVPR1A | ENSG00000166148 | NP_000697.1 |
| AVPR1B | ENSG00000198049 | NP_000698.1 |
| BAIAP2 | ENSG00000175866 | NP_059345.1 |
| BBS4 | ENSG00000140463 | NP_149017.2 |
| BCAS1 | ENSG00000064787 | NP_003648.2 |
| BCKDK | ENSG00000103507 | NP_005872.2 |
| BCL11A | ENSG00000119866 | NP_075044.2 |
| BCL2 | ENSG00000171791 | NP_000624.2 |
| BDNF | ENSG00000176697 | NP_001137282.1 |
| BIN1 | ENSG00000136717 | NP_647593.1 |
| BIRC6 | ENSG00000115760 | NP_057336.3 |
| BRAF | ENSG00000157764 | NP_004324.2 |
| BRCA2 | ENSG00000139618 | NP_000050.2 |
| BRINP1 | ENSG00000078725 | NP_055433.2 |
| BTAF1 | ENSG00000095564 | NP_003963.1 |
| BZRAP1 | ENSG00000005379 | NP_004749.2 |
| C11orf30 | ENSG00000158636 | NP_064578.2 |
| C12orf57 | ENSG00000111678 | NP_612434.1 |
| C15orf43 | ENSG00000167014 | NP_689661.1 |
| C3orf58 | ENSG00000181744 | NP_775823.1 |
| C4B | ENSG00000236625, | NP_001229752.1 |
| | ENSG00000224639, | |
| | ENSG00000228267, | |
| | ENSG00000228454, | |
| | ENSG00000224389 | |
| CA6 | ENSG00000131686 | NP_001257429.1 |
| CACNA1A | ENSG00000141837 | NP_075461.2 |
| CACNA1B | ENSG00000148408 | NP_000709.1 |
| CACNA1C | ENSG00000151067 | NP_955630.3 |
| CACNA1D | ENSG00000157388 | NP_000711.1 |
| CACNA1E | ENSG00000198216 | NP_001192222.1 |
| CACNA1F | ENSG00000102001 | NP_005174.2 |
| CACNA1G | ENSG00000006283 | NP_061496.2 |
| CACNA1H | ENSG00000196557 | NP_066921.2 |
| CACNA1I | ENSG00000100346 | NP_066919.2 |
| CACNA2D3 | ENSG00000157445 | NP_060868.2 |
| CACNB2 | ENSG00000165995 | NP_963890.2 |
| CADM1 | ENSG00000182985 | NP_055148.3 |
| CADM2 | ENSG00000175161 | NP_694854.2 |
| CADPS2 | ENSG00000081803 | NP_001161412.1 |
| CAMK2A | ENSG00000070808 | NP_057065.2 |
| CAMK4 | ENSG00000152495 | NP_001735.1 |
| CAMSAP2 | ENSG00000118200 | NP_982284.1 |
| CAMTA1 | ENSG00000171735 | NP_056030.1 |
| CAPN12 | ENSG00000182472 | NP_653292.2 |
| CAPRIN1 | ENSG00000135387 | NP_005889.3 |
| CASC4 | ENSG00000166734 | NP_612432.2 |
| CASK | ENSG00000147044 | NP_003679.2 |
| CBS | ENSG00000160200 | NP_001171479.1 |
| CC2D1A | ENSG00000132024 | NP_060191.3 |
| CCDC64 | ENSG00000135127 | NP_997194.2 |
| CCDC88C | ENSG00000015133 | NP_001073883.2 |
| CCDC91 | ENSG00000123106 | NP_060788.3 |
| CCT4 | ENSG00000115484 | NP_006421.2 |
| CD38 | ENSG00000004468 | NP_001766.2 |
| CD44 | ENSG00000026508 | NP_000601.3 |
| CD99L2 | ENSG00000102181 | NP_001229543.1 |
| CDC42BPB | ENSG00000198752 | NP_006026.3 |
| CDH10 | ENSG00000040731 | NP_006718.2 |
| CDH11 | ENSG00000140937 | NP_001788.2 |
| CDH22 | ENSG00000149654 | NP_067071.1 |
| CDH8 | ENSG00000150394 | NP_001787.2 |
| CDH9 | ENSG00000113100 | NP_057363.3 |
| CDKL5 | ENSG00000008086 | NP_003150.1 |
| CDKN1B | ENSG00000111276 | NP_004055.1 |
| CECR2 | ENSG00000099954 | NP_113601.2 |
| CELF4 | ENSG00000101489 | NP_064565.1 |
| CELF6 | ENSG00000140488 | NP_443072.3 |
| CEP290 | ENSG00000198707 | NP_079390.3 |
| CEP41 | ENSG00000106477 | NP_061188.1 |
| CGNL1 | ENSG00000128849 | NP_116255.2 |
| CHD2 | ENSG00000173575 | NP_001262.3 |
| CHD7 | ENSG00000171316 | NP_060250.2 |
| CHD8 | ENSG00000100888 | NP_001164100.1 |
| CHKB | ENSG00000100288 | NP_005189.2 |
| CHRM3 | ENSG00000133019 | NP_000731.1 |
| CHRNA7 | ENSG00000175344 | NP_001177384.1 |
| CHRNB3 | ENSG00000147432 | NP_000740.1 |
| CHST5 | ENSG00000135702 | NP_078809.2 |
| CIB2 | ENSG00000136425 | NP_006374.1 |
| CLN8 | ENSG00000182372, | NP_061764.2 |
| | ENSG00000278220 | |
| CLSTN3 | ENSG00000139182 | NP_055533.2 |
| CLTCL1 | ENSG00000070371 | NP_009029.3 |
| CMIP | ENSG00000153815 | NP_938204.2 |
| CNKSR2 | ENSG00000149970 | NP_055742.2 |
| CNOT3 | ENSG00000277600, | NP_055331.1 |
| | ENSG00000274616, | |
| | ENSG00000277114, | |
| | ENSG00000088038, | |
| | ENSG00000273943, | |
| | ENSG00000274941, | |
| | ENSG00000274176, | |
| | ENSG00000277615, | |
| | ENSG00000276082, | |
| | ENSG00000275979 | |
| CNR1 | ENSG00000118432 | NP_001153698.1 |
| CNR2 | ENSG00000188822 | NP_001832.1 |
| CNTN3 | ENSG00000113805 | NP_065923.1 |
| CNTN4 | ENSG00000144619 | NP_783200.1 |
| CNTN5 | ENSG00000149972 | NP_001230199.1 |
| CNTN6 | ENSG00000134115 | NP_055276.1 |
| CNTNAP2 | ENSG00000278728, | NP_054860.1 |
| | ENSG00000174469 | |
| CNTNAP3 | ENSG00000106714 | NP_387504.2 |
| CNTNAP4 | ENSG00000152910 | NP_207837.2 |
| CNTNAP5 | ENSG00000155052 | NP_570129.1 |
| CREBBP | ENSG00000005339 | NP_004371.2 |
| CRHR2 | ENSG00000106113 | NP_001189404.1 |
| CSMD1 | ENSG00000183117 | NP_150094.5 |
| CSNK1D | ENSG00000141551 | NP_001884.2 |
| CTCF | ENSG00000102974 | NP_006556.1 |
| CTNNA3 | ENSG00000183230 | NP_037398.2 |
| CTNNB1 | ENSG00000168036 | NP_001091680.1 |
| CTNND2 | ENSG00000169862 | NP_001323.1 |
| CTTNBP2 | ENSG00000077063 | NP_219499.1 |
| CUL3 | ENSG00000036257 | NP_001244127.1 |
| CUL7 | ENSG00000044090 | NP_001161842.1 |
| CUX1 | ENSG00000257923 | NP_001189472.1 |
| CX3CR1 | ENSG00000168329 | NP_001164645.1 |
| CXCR3 | ENSG00000186810 | NP_001136269.1 |
| CYFIP1 | ENSG00000273749, | NP_055423.1 |
| | ENSG00000280618 | |
| CYLC2 | ENSG00000155833 | NP_001331.1 |
| CYP11B1 | ENSG00000160882 | NP_000488.3 |
| DAB1 | ENSG00000173406 | NP_066566.3 |
| DAGLA | ENSG00000134780 | NP_006124.1 |
| DAPK1 | ENSG00000196730 | NP_004929.2 |
| DAPP1 | ENSG00000070190 | NP_055210.2 |
| DCTN5 | ENSG00000166847 | NP_115875.1 |
| DCUN1D1 | ENSG00000043093 | NP_065691.2 |
| DCX | ENSG00000077279 | NP_000546.2 |
| DDC | ENSG00000132437 | NP_001076440.1 |
| DDX11 | ENSG00000013573 | NP_689651.1 |
| DDX3X | ENSG00000215301 | NP_001347.3 |
| DDX53 | ENSG00000184735 | NP_874358.2 |
| DEAF1 | ENSG00000177030, | NP_066288.2 |
| | ENSG00000282712 | |
| DENR | ENSG00000139726 | NP_003668.2 |
| DEPDC5 | ENSG00000100150 | NP_001229825.1 |
| DGKK | ENSG00000274588 | NP_001013764.1 |
| DGKZ | ENSG00000149091 | NP_001099010.1 |
| DHCR7 | ENSG00000172893 | NP_001351.2 |
| DIAPH3 | ENSG00000139734 | NP_001035982.1 |
| DIP2A | ENSG00000160305 | NP_055966.2 |
| DISC1 | ENSG00000162946 | NP_001158009.1 |
| DIXDC1 | ENSG00000150764 | NP_001033043.1 |
| DLG1 | ENSG00000075711 | NP_004078.2 |
| DLG4 | ENSG00000132535 | NP_001356.1 |
| DLGAP1 | ENSG00000170579 | NP_001229690.1 |
| DLGAP2 | ENSG00000198010, | NP_004736.2 |
| | ENSG00000282152, | |
| | ENSG00000274161, | |
| | ENSG00000282318 | |
| DLGAP3 | ENSG00000116544 | NP_001073887.1 |
| DLX1 | ENSG00000144355 | NP_835221.2 |
| DLX2 | ENSG00000115844 | NP_004396.1 |
| DLX6 | ENSG00000006377 | NP_005213.3 |
| DMD | ENSG00000198947 | NP_003997.1 |
| DMPK | ENSG00000104936 | NP_001075032.1 |
| DMXL2 | ENSG00000104093 | NP_001167587.1 |
| DNAH10 | ENSG00000197653, | NP_997320.2 |
| | ENSG00000281935 | |
| DNAJC19 | ENSG00000205981 | NP_660304.1 |
| DNER | ENSG00000187957 | NP_620711.3 |
| DNM1L | ENSG00000087470 | NP_036192.2 |
| DNMT3A | ENSG00000119772 | NP_072046.2 |
| DOCK1 | ENSG00000150760 | NP_001371.1 |
| DOCK10 | ENSG00000135905 | NP_055504.2 |
| DOCK4 | ENSG00000128512 | NP_055520.3 |
| DOCK8 | ENSG00000107099 | NP_982272.2 |
| DOLK | ENSG00000175283 | NP_055723.1 |
| DPP10 | ENSG00000175497 | NP_001171505.1 |
| DPP4 | ENSG00000197635 | NP_001926.2 |
| DPP6 | ENSG00000130226, | NP_570629.2 |
| | ENSG00000282974 | |
| DPYD | ENSG00000188641 | NP_000101.2 |
| DRD1 | ENSG00000184845 | NP_000785.1 |
| DRD2 | ENSG00000149295 | NP_000786.1 |
| DRD3 | ENSG00000151577 | NP_000787.2 |
| DSCAM | ENSG00000171587 | NP_001380.2 |
| DST | ENSG00000151914 | NP_056363.2 |
| DUSP15 | ENSG00000149599 | NP_542178.2 |
| DUSP22 | ENSG00000112679 | NP_064570.1 |
| DVL1 | ENSG00000107404 | NP_004412.2 |
| DVL3 | ENSG00000161202 | NP_004414.3 |
| DYDC1 | ENSG00000170788 | NP_620167.1 |
| DYDC2 | ENSG00000133665 | NP_001256971.1 |
| DYRK1A | ENSG00000157540 | NP_001387.2 |
| EEF1A2 | ENSG00000101210 | NP_001949.1 |
| EFR3A | ENSG00000132294 | NP_055952.2 |
| EGR2 | ENSG00000122877 | NP_001129649.1 |
| EHMT1 | ENSG00000181090 | NP_079033.4 |
| EIF3G | ENSG00000130811 | NP_003746.2 |
| EIF4E | ENSG00000151247 | NP_001124151.1 |
| EIF4EBP2 | ENSG00000148730 | NP_004087.1 |
| ELAVL2 | ENSG00000107105 | NP_004423.2 |
| ELAVL3 | ENSG00000196361 | NP_001411.2 |
| ELP4 | ENSG00000109911 | NP_061913.3 |
| EML1 | ENSG00000066629 | NP_001008707.1 |
| EN2 | ENSG00000164778 | NP_001418.2 |
| EP300 | ENSG00000100393 | NP_001420.2 |
| EP400 | ENSG00000183495 | NP_056224.3 |
| EPC2 | ENSG00000135999 | NP_056445.3 |
| EPHA6 | ENSG00000080224 | NP_001073917.2 |
| EPHB2 | ENSG00000133216 | NP_004433.2 |
| EPHB6 | ENSG00000275482, | NP_004436.2 |
| | ENSG00000106123 | |
| EPS8 | ENSG00000151491 | NP_004438.3 |
| ERBB4 | ENSG00000178568 | NP_005226.1 |
| ERBIN | ENSG00000112851 | NP_001240628.1 |
| ERG | ENSG00000157554 | NP_001129626.1 |
| ERMN | ENSG00000136541 | NP_001009959.1 |
| ESR1 | ENSG00000091831 | NP_001116212.1 |
| ESR2 | ENSG00000140009 | NP_001428.1 |
| ESRRB | ENSG00000119715 | NP_004443.3 |
| ETFB | ENSG00000105379 | NP_001014763.1 |
| EXOC3 | ENSG00000180104 | NP_009208.2 |
| EXOC5 | ENSG00000070367 | NP_006535.1 |
| EXOC6 | ENSG00000138190 | NP_061926.3 |
| EXOC6B | ENSG00000144036 | NP_056004.1 |
| EXT1 | ENSG00000182197 | NP_000118.2 |
| F13A1 | ENSG00000124491 | NP_000120.2 |
| FABP3 | ENSG00000121769 | NP_004093.1 |
| FABP5 | ENSG00000164687 | NP_001435.1 |
| FABP7 | ENSG00000164434 | NP_001437.1 |
| FAM135B | ENSG00000147724 | NP_056996.2 |
| FAM92B | ENSG00000153789 | NP_940893.1 |
| FAN1 | ENSG00000198690, | NP_055782.3 |
| | ENSG00000276787 | |
| FAT1 | ENSG00000083857 | NP_005236.2 |
| FBN1 | ENSG00000166147 | NP_000129.3 |
| FBXO15 | ENSG00000141665 | NP_001136430.1 |
| FBXO33 | ENSG00000165355 | NP_976046.1 |
| FBXO40 | ENSG00000163833 | NP_057382.2 |
| FER | ENSG00000151422 | NP_005237.2 |
| FEZF2 | ENSG00000153266 | NP_060478.3 |
| FGA | ENSG00000171560 | NP_000499.1 |
| FGD1 | ENSG00000102302 | NP_004454.2 |
| FGFBP3 | ENSG00000174721 | NP_689642.3 |
| FHIT | ENSG00000189283 | NP_001159715.1 |
| FLT1 | ENSG00000102755 | NP_002010.2 |
| FMR1 | ENSG00000102081 | NP_002015.1 |
| FOLH1 | ENSG00000086205 | NP_004467.1 |
| FOXG1 | ENSG00000176165 | NP_005240.3 |
| FOXP1 | ENSG00000114861 | NP_001231739.1 |
| FOXP2 | ENSG00000128573 | NP_683696.2 |
| FRK | ENSG00000111816 | NP_002022.1 |
| FRMPD4 | ENSG00000169933 | NP_055543.2 |
| GABRA1 | ENSG00000022355 | NP_001121115.1 |
| GABRA3 | ENSG00000011677 | NP_000799.1 |
| GABRA4 | ENSG00000109158 | NP_000800.2 |
| GABRA5 | ENSG00000186297 | NP_001158509.1 |
| GABRB1 | ENSG00000163288 | NP_000803.2 |
| GABRB3 | ENSG00000166206 | NP_068712.1 |
| GABRQ | ENSG00000268089 | NP_061028.2 |
| GAD1 | ENSG00000128683 | NP_000808.2 |
| GADD45B | ENSG00000099860 | NP_056490.2 |
| GALNT13 | ENSG00000144278 | NP_443149.2 |
| GALNT14 | ENSG00000158089 | NP_001240755.1 |
| GAN | ENSG00000261609 | NP_071324.1 |
| GAP43 | ENSG00000172020 | NP_001123536.1 |
| GAS2 | ENSG00000148935 | NP_001137302.1 |
| GATM | ENSG00000171766 | NP_001473.1 |
| GDA | ENSG00000119125 | NP_001229434.1 |
| GIGYF1 | ENSG00000146830 | NP_072096.2 |
| GIGYF2 | ENSG00000204120 | NP_001096617.1 |
| GLIS1 | ENSG00000174332 | NP_671726.2 |
| GLO1 | ENSG00000124767 | NP_006699.2 |
| GLRA2 | ENSG00000101958 | NP_001112357.1 |
| GNA14 | ENSG00000156049 | NP_004288.1 |
| GNAS | ENSG00000087460 | NP_536350.2 |
| GNB1L | ENSG00000185838 | NP_443730.1 |
| GPC4 | ENSG00000076716 | NP_001439.2 |
| GPC6 | ENSG00000183098 | NP_005699.1 |
| GPD2 | ENSG00000115159 | NP_001076581.2 |
| GPHN | ENSG00000171723 | NP_065857.1 |
| GPR139 | ENSG00000180269 | NP_001002911.1 |
| GPR37 | ENSG00000170775 | NP_005293.1 |
| GPR85 | ENSG00000164604 | NP_061843.3 |
| GPX1 | ENSG00000233276 | NP_000572.2 |
| GRID1 | ENSG00000182771 | NP_060021.1 |
| GRID2 | ENSG00000152208 | NP_001501.2 |
| GRID2IP | ENSG00000215045 | NP_001138590.1 |
| GRIK2 | ENSG00000164418 | NP_068775.1 |
| GRIK3 | ENSG00000163873 | NP_000822.2 |
| GRIK4 | ENSG00000149403 | NP_055434.2 |
| GRIK5 | ENSG00000105737 | NP_002079.3 |
| GRIN1 | ENSG00000176884 | NP_001172019.1 |
| GRIN2A | ENSG00000183454 | NP_001127879.1 |
| GRIN2B | ENSG00000273079 | NP_000825.2 |
| GRIP1 | ENSG00000155974 | NP_066973.2 |
| GRM1 | ENSG00000152822 | NP_000829.2 |
| GRM4 | ENSG00000124493 | NP_000832.1 |
| GRM5 | ENSG00000168959 | NP_001137303.1 |
| GRM7 | ENSG00000196277 | NP_870989.1 |
| GRM8 | ENSG00000179603 | NP_000836.2 |
| GRPR | ENSG00000126010 | NP_005305.1 |
| GSK3B | ENSG00000082701 | NP_002084.2 |
| GSN | ENSG00000148180 | NP_000168.1 |
| GSTM1 | ENSG00000134184 | NP_000552.2 |
| GTF2I | ENSG00000263001 | NP_127492.1 |
| GUCY1A2 | ENSG00000152402 | NP_001243353.1 |
| HCFC1 | ENSG00000172534 | NP_005325.2 |
| HCN1 | ENSG00000164588 | NP_066550.2 |
| HDAC3 | ENSG00000171720 | NP_003874.2 |
| HDAC4 | ENSG00000068024 | NP_006028.2 |
| HDAC6 | ENSG00000094631 | NP_006035.2 |
| HDC | ENSG00000140287 | NP_002103.2 |
| HECW2 | ENSG00000138411 | NP_065811.1 |
| HEPACAM | ENSG00000165478 | NP_689935.2 |
| HERC2 | ENSG00000276802, | NP_004658.3 |
| | ENSG00000128731, | |
| | ENSG00000277278 | |
| HIVEP3 | ENSG00000127124 | NP_078779.2 |
| HLA-A | ENSG00000235657, | NP_001229687.1 |
| | ENSG00000223980, | |
| | ENSG00000206503, | |
| | ENSG00000206505, | |
| | ENSG00000227715, | |
| | ENSG00000231834, | |
| | ENSG00000224320, | |
| | ENSG00000229215 | |
| HLA-B | ENSG00000223532, | NP_005505.2 |
| | ENSG00000206450, | |
| | ENSG00000234745, | |
| | ENSG00000224608, | |
| | ENSG00000228964, | |
| | ENSG00000232126 | |
| HLA-DRB1 | ENSG00000236884, | NP_001230894.1 |
| | ENSG00000228080, | |
| | ENSG00000206240, | |
| | ENSG00000229074, | |
| | ENSG00000206306, | |
| | ENSG00000196126 | |
| HMGN1 | ENSG00000205581 | NP_004956.5 |
| HNRNPH2 | ENSG00000126945 | NP_001027565.1 |
| HOMER1 | ENSG00000152413 | NP_004263.1 |
| HOXA1 | ENSG00000105991 | NP_005513.1 |
| HOXB1 | ENSG00000120094 | NP_002135.2 |
| HRAS | ENSG00000174775, | NP_001123914.1 |
| | ENSG00000276536 | |
| HS3ST5 | ENSG00000249853 | NP_705840.2 |
| HSD11B1 | ENSG00000117594 | NP_861420.1 |
| HTR1B | ENSG00000135312 | NP_000854.1 |
| HTR2A | ENSG00000102468 | NP_000612.1 |
| HTR3A | ENSG00000166736 | NP_998786.2 |
| HTR3C | ENSG00000178084 | NP_570126.2 |
| HTR7 | ENSG00000148680 | NP_062873.1 |
| HUWE1 | ENSG00000086758 | NP_113584.3 |
| HYDIN | ENSG00000157423, | NP_001257903.1 |
| | ENSG00000283022 | |
| ICA1 | ENSG00000003147 | NP_071682.1 |
| IFNG | ENSG00000111537 | NP_000610.2 |
| IFNGR1 | ENSG00000027697 | NP_000407.1 |
| IL16 | ENSG00000172349 | NP_757366.2 |
| IL17A | ENSG00000112115 | NP_002181.1 |
| IL1R2 | ENSG00000115590 | NP_004624.1 |
| IL1RAPL1 | ENSG00000169306 | NP_055086.1 |
| IL1RAPL2 | ENSG00000189108 | NP_059112.1 |
| IL6 | ENSG00000136244 | NP_000591.1 |
| ILF2 | ENSG00000143621 | NP_004506.2 |
| IMMP2L | ENSG00000184903 | NP_115938.1 |
| INADL | ENSG00000132849 | NP_795352.2 |
| INPP1 | ENSG00000151689 | NP_001122400.1 |
| INTS6 | ENSG00000102786 | NP_036273.1 |
| IQGAP3 | ENSG00000183856 | NP_839943.2 |
| IQSEC2 | ENSG00000124313 | NP_001104595.1 |
| IRF2BPL | ENSG00000119669 | NP_078772.1 |
| ITGA4 | ENSG00000115232 | NP_000876.3 |
| ITGB3 | ENSG00000259207 | NP_000203.2 |
| ITGB7 | ENSG00000139626 | NP_000880.1 |
| ITPR1 | ENSG00000150995 | NP_001161744.1 |
| JAKMIP1 | ENSG00000152969 | NP_001092903.1 |
| JARID2 | ENSG00000008083 | NP_004964.2 |
| JMJD1C | ENSG00000171988 | NP_116165.1 |
| KANK1 | ENSG00000107104 | NP_001243805.1 |
| KAT2B | ENSG00000114166 | NP_003875.3 |
| KAT6A | ENSG00000083168 | NP_001092882.1 |
| KATNAL2 | ENSG00000167216 | NP_112593.2 |
| KCND2 | ENSG00000184408 | NP_036413.1 |
| KCNJ10 | ENSG00000177807 | NP_002232.2 |
| KCNJ12 | ENSG00000184185 | NP_066292.2 |
| KCNJ15 | ENSG00000157551 | NP_002234.2 |
| KCNJ2 | ENSG00000123700 | NP_000882.1 |
| KCNMA1 | ENSG00000156113 | NP_001154824.1 |
| KCNQ2 | ENSG00000281151, | NP_742105.1 |
| | ENSG00000075043 | |
| KCNQ3 | ENSG00000184156 | NP_004510.1 |
| KCNT1 | ENSG00000107147 | NP_065873.2 |
| KCTD13 | ENSG00000174943 | NP_849194.1 |
| KDM4B | ENSG00000127663 | NP_055830.1 |
| KDM5B | ENSG00000117139 | NP_006609.3 |
| KDM5C | ENSG00000126012 | NP_004178.2 |
| KDM6B | ENSG00000132510 | NP_001073893.1 |
| KHDRBS2 | ENSG00000112232 | NP_689901.2 |
| KHDRBS3 | ENSG00000131773 | NP_006549.1 |
| KIAA1586 | ENSG00000168116 | NP_065982.1 |
| KIAA2022 | ENSG00000050030 | NP_001008537.1 |
| KIF13B | ENSG00000197892 | NP_056069.2 |
| KIF5C | ENSG00000276734, | NP_004513.1 |
| | ENSG00000168280 | |
| KIRREL3 | ENSG00000149571 | NP_115920.1 |
| KIT | ENSG00000157404 | NP_000213.1 |
| KLC2 | ENSG00000174996 | NP_073733.1 |
| KMO | ENSG00000117009 | NP_003670.2 |
| KMT2A | ENSG00000118058 | NP_005924.2 |
| KMT2C | ENSG00000055609 | NP_733751.2 |
| KMT2E | ENSG00000005483 | NP_891847.1 |
| KMT5B | ENSG00000110066 | NP_060105.3 |
| KPTN | ENSG00000118162 | NP_008990.2 |
| KRR1 | ENSG00000111615 | NP_008974.5 |
| LAMA1 | ENSG00000101680 | NP_005550.2 |
| LAMB1 | ENSG00000091136 | NP_002282.2 |
| LAMC3 | ENSG00000050555 | NP_006050.3 |
| LEP | ENSG00000174697 | NP_000221.1 |
| LIN7B | ENSG00000104863 | NP_071448.1 |
| LMX1B | ENSG00000136944 | NP_001167617.1 |
| LPL | ENSG00000175445 | NP_000228.1 |
| LRBA | ENSG00000198589 | NP_006717.2 |
| LRFN5 | ENSG00000165379 | NP_689660.2 |
| LRP2 | ENSG00000081479 | NP_004516.2 |
| LRP2BP | ENSG00000109771 | NP_060879.2 |
| LRPPRC | ENSG00000138095 | NP_573566.2 |
| LRRC1 | ENSG00000137269 | NP_060684.4 |
| LRRC7 | ENSG00000033122 | NP_065845.1 |
| LZTR1 | ENSG00000099949 | NP_006758.2 |
| LZTS2 | ENSG00000107816 | NP_115805.1 |
| MACROD2 | ENSG00000172264 | NP_542407.2 |
| MAGED1 | ENSG00000179222 | NP_001005333.1 |
| MAGEL2 | ENSG00000254585 | NP_061939.3 |
| MAL | ENSG00000172005 | NP_002362.1 |
| MAOA | ENSG00000189221 | NP_000231.1 |
| MAOB | ENSG00000069535 | NP_000889.3 |
| MAP2 | ENSG00000078018 | NP_002365.3 |
| MAPK1 | ENSG00000100030 | NP_620407.1 |
| MAPK12 | ENSG00000188130 | NP_002960.2 |
| MAPK3 | ENSG00000102882 | NP_002737.2 |
| MAPK8IP2 | ENSG00000008735 | NP_036456.1 |
| MARK1 | ENSG00000116141 | NP_061120.3 |
| MBD1 | ENSG00000141644 | NP_001191065.1 |
| MBD3 | ENSG00000071655 | NP_003917.1 |
| MBD4 | ENSG00000129071 | NP_003916.1 |
| MBD5 | ENSG00000204406 | NP_060798.2 |
| MBD6 | ENSG00000166987 | NP_443129.3 |
| MBOAT7 | ENSG00000275118, | NP_077274.3 |
| | ENSG00000273592, | |
| | ENSG00000125505, | |
| | ENSG00000276935, | |
| | ENSG00000278519, | |
| | ENSG00000277733, | |
| | ENSG00000278322, | |
| | ENSG00000274194, | |
| | ENSG00000277025, | |
| | ENSG00000277923 | |
| MC4R | ENSG00000166603 | NP_005903.2 |
| MCC | ENSG00000171444 | NP_001078846.1 |
| MCM4 | ENSG00000104738 | NP_877423.1 |
| MCPH1 | ENSG00000147316 | NP_078872.2 |
| MDGA2 | ENSG00000139915 | NP_001106970.2 |
| MECP2 | ENSG00000169057 | NP_001104262.1 |
| MED12 | ENSG00000184634 | NP_005111.2 |
| MED13L | ENSG00000123066 | NP_056150.1 |
| MEF2C | ENSG00000081189 | NP_001180276.1 |
| MET | ENSG00000105976 | NP_001120972.1 |
| MFRP | ENSG00000235718 | NP_113621.1 |
| MIB1 | ENSG00000101752 | NP_065825.1 |
| MKL2 | ENSG00000186260 | NP_054767.3 |
| MNT | ENSG00000070444 | NP_064706.1 |
| MOCOS | ENSG00000075643 | NP_060417.2 |
| MPP6 | ENSG00000105926 | NP_057531.2 |
| MSN | ENSG00000147065 | NP_002435.1 |
| MSNP1AS | ENSG00000251593 | |
| MSR1 | ENSG00000038945 | NP_619729.1 |
| MTF1 | ENSG00000188786 | NP_005946.2 |
| MTHFR | ENSG00000177000 | NP_005948.3 |
| MTOR | ENSG00000198793 | NP_004949.1 |
| MTR | ENSG00000116984 | NP_000245.2 |
| MTX2 | ENSG00000128654 | NP_006545.1 |
| MYH4 | ENSG00000264424 | NP_060003.2 |
| MYO16 | ENSG00000041515, | NP_001185879.1 |
| | ENSG00000282848 | |
| MYO1A | ENSG00000166866 | NP_001242970.1 |
| MYO9B | ENSG00000099331 | NP_004136.2 |
| MYOZ1 | ENSG00000177791 | NP_067068.1 |
| MYT1L | ENSG00000186487 | NP_055840.2 |
| NAA15 | ENSG00000164134 | NP_476516.1 |
| NAALADL2 | ENSG00000177694 | NP_996898.2 |
| NACC1 | ENSG00000160877 | NP_443108.1 |
| NAV2 | ENSG00000166833 | NP_001231892.1 |
| NBEA | ENSG00000172915 | NP_056493.3 |
| NCKAP1 | ENSG00000061676 | NP_995314.1 |
| NCKAP5 | ENSG00000176771 | NP_997246.2 |
| NCKAP5L | ENSG00000167566 | NP_001032895.2 |
| NCOR1 | ENSG00000141027 | NP_006302.2 |
| NDNL2 | ENSG00000185115, | NP_619649.1 |
| | ENSG00000282130 | |
| NDUFA5 | ENSG00000128609 | NP_004991.1 |
| NEFL | ENSG00000277586 | NP_006149.2 |
| NELL1 | ENSG00000165973 | NP_006148.2 |
| NEO1 | ENSG00000067141 | NP_002490.2 |
| NF1 | ENSG00000196712 | NP_001035957.1 |
| NFIA | ENSG00000162599 | NP_001138984.1 |
| NFIX | ENSG00000008441 | NP_001257972.1 |
| NINL | ENSG00000101004 | NP_079452.3 |
| NIPA1 | ENSG00000170113 | NP_653200.2 |
| NIPA2 | ENSG00000140157 | NP_001171818.1 |
| NIPBL | ENSG00000164190 | NP_597677.2 |
| NLGN1 | ENSG00000169760 | NP_055747.1 |
| NLGN2 | ENSG00000169992, | NP_065846.1 |
| | ENSG00000283859 | |
| NLGN3 | ENSG00000196338 | NP_851820.1 |
| NLGN4X | ENSG00000146938 | NP_065793.1 |
| NLGN4Y | ENSG00000165246 | NP_055708.3 |
| NOS1 | ENSG00000089250 | NP_001191147.1 |
| NOS1AP | ENSG00000198929 | NP_055512.1 |
| NOS2A | ENSG00000007171 | NP_000616.3 |
| NPAS2 | ENSG00000170485 | NP_002509.2 |
| NR2F1 | ENSG00000175745 | NP_005645.1 |
| NR3C2 | ENSG00000151623 | NP_000892.2 |
| NRCAM | ENSG00000091129 | NP_001032209.1 |
| NRG1 | ENSG00000157168 | NP_039250.2 |
| NRP2 | ENSG00000118257 | NP_957718.1 |
| NRXN1 | ENSG00000179915 | NP_001129131.1 |
| NRXN2 | ENSG00000110076 | NP_055895.1 |
| NRXN3 | ENSG00000021645 | NP_004787.2 |
| NSD1 | ENSG00000165671 | NP_071900.2 |
| NTNG1 | ENSG00000162631 | NP_001106697.1 |
| NTRK1 | ENSG00000198400 | NP_002520.2 |
| NTRK3 | ENSG00000140538 | NP_001012338.1 |
| NUAK1 | ENSG00000074590 | NP_055655.1 |
| NUP133 | ENSG00000069248 | NP_060700.2 |
| NXF5 | ENSG00000126952 | NP_116564.2 |
| NXPH1 | ENSG00000122584 | NP_689958.1 |
| OCRL | ENSG00000122126 | NP_000267.2 |
| ODF3L2 | ENSG00000181781 | NP_872383.1 |
| OGT | ENSG00000147162 | NP_858058.1 |
| OPHN1 | ENSG00000079482 | NP_002538.1 |
| OPRM1 | ENSG00000112038 | NP_001138751.1 |
| OR1C1 | ENSG00000221888 | NP_036485.2 |
| OR2M4 | ENSG00000171180 | NP_059974.1 |
| OR2T10 | ENSG00000184022 | NP_001004693.1 |
| OR52M1 | ENSG00000197790 | NP_001004137.1 |
| OTX1 | ENSG00000115507 | NP_055377.1 |
| OXT | ENSG00000101405 | NP_000906.1 |
| OXTR | ENSG00000180914 | NP_000907.2 |
| P2RX4 | ENSG00000135124 | NP_001243725.1 |
| P2RX5 | ENSG00000083454 | NP_002552.2 |
| P4HA2 | ENSG00000072682 | NP_001136071.1 |
| PACS1 | ENSG00000175115 | NP_060496.2 |
| PAFAH1B1 | ENSG00000007168 | NP_000421.1 |
| PAH | ENSG00000171759 | NP_000268.1 |
| PARD3B | ENSG00000116117 | NP_689739.4 |
| PARK2 | ENSG00000185345 | NP_004553.2 |
| PAX5 | ENSG00000196092 | NP_057953.1 |
| PAX6 | ENSG00000007372 | NP_001245391.1 |
| PCCA | ENSG00000175198 | NP_000273.2 |
| PCCB | ENSG00000114054 | NP_001171485.1 |
| PCDH10 | ENSG00000138650 | NP_116586.1 |
| PCDH15 | ENSG00000150275 | NP_001136235.1 |
| PCDH19 | ENSG00000165194 | NP_001171809.1 |
| PCDH8 | ENSG00000136099 | NP_002581.2 |
| PCDH9 | ENSG00000184226 | NP_982354.1 |
| PCDHA1 | ENSG00000204970 | NP_061723.1 |
| PCDHA10 | ENSG00000250120 | NP_061724.1 |
| PCDHA11 | ENSG00000249158 | NP_061725.1 |
| PCDHA12 | ENSG00000251664 | NP_061726.1 |
| PCDHA13 | ENSG00000239389 | NP_061727.1 |
| PCDHA2 | ENSG00000204969 | NP_061728.1 |
| PCDHA3 | ENSG00000255408 | NP_061729.1 |
| PCDHA4 | ENSG00000204967 | NP_061730.1 |
| PCDHA5 | ENSG00000204965 | NP_061731.1 |
| PCDHA6 | ENSG00000081842 | NP_061732.1 |
| PCDHA7 | ENSG00000204963 | NP_061733.1 |
| PCDHA8 | ENSG00000204962 | NP_061734.1 |
| PCDHA9 | ENSG00000204961 | NP_114063.1 |
| PCDHAC1 | ENSG00000248383 | NP_061721.2 |
| PCDHAC2 | ENSG00000243232 | NP_061722.1 |
| PCDHGA11 | ENSG00000253873 | NP_061737.1 |
| PDE1C | ENSG00000154678 | NP_001177987.1 |
| PDE4A | ENSG00000065989 | NP_001104777.1 |
| PDE4B | ENSG00000184588 | NP_002591.2 |
| PDZD4 | ENSG00000067840 | NP_115901.2 |
| PECR | ENSG00000115425 | NP_060911.2 |
| PER1 | ENSG00000179094 | NP_002607.2 |
| PEX7 | ENSG00000112357 | NP_000279.1 |
| PGLYRP2 | ENSG00000161031 | NP_443122.3 |
| PHF2 | ENSG00000197724 | NP_005383.3 |
| PHF8 | ENSG00000172943 | NP_001171825.1 |
| PHIP | ENSG00000146247 | NP_060404.4 |
| PHRF1 | ENSG00000070047, | NP_065952.2 |
| | ENSG00000274780 | |
| PIK3CG | ENSG00000105851 | NP_002640.2 |
| PIK3R2 | ENSG00000105647 | NP_005018.1 |
| PINX1 | ENSG00000254093 | NP_060354.4 |
| PITX1 | ENSG00000069011 | NP_002644.4 |
| PLAUR | ENSG00000011422 | NP_002650.1 |
| PLCB1 | ENSG00000182621 | NP_056007.1 |
| PLCD1 | ENSG00000187091 | NP_001124436.1 |
| PLN | ENSG00000198523 | NP_002658.1 |
| PLXNA4 | ENSG00000221866 | NP_065962.1 |
| PLXNB1 | ENSG00000164050 | NP_001123554.1 |
| POGZ | ENSG00000143442 | NP_055915.2 |
| POMGNT1 | ENSG00000085998 | NP_001230695.1 |
| PON1 | ENSG00000005421 | NP_000437.3 |
| POTI | ENSG00000128513 | NP_056265.2 |
| POU3F2 | ENSG00000184486 | NP_005595.2 |
| PPFIA1 | ENSG00000131626 | NP_003617.1 |
| PPP1R1B | ENSG00000131771 | NP_115568.2 |
| PPP1R3F | ENSG00000049769 | NP_149992.3 |
| PPP2R1B | ENSG00000137713 | NP_859050.1 |
| PPP2R5D | ENSG00000112640 | NP_006236.1 |
| PREX1 | ENSG00000124126 | NP_065871.2 |
| PRICKLE1 | ENSG00000139174 | NP_694571.2 |
| PRICKLE2 | ENSG00000163637 | NP_942559.1 |
| PRKCB | ENSG00000166501 | NP_002729.2 |
| PRKD1 | ENSG00000184304 | NP_002733.2 |
| PRKDC | ENSG00000253729 | NP_008835.5 |
| PRODH | ENSG00000100033 | NP_057419.4 |
| PRSS38 | ENSG00000185888 | NP_898885.1 |
| PRUNE2 | ENSG00000106772 | NP_056040.2 |
| PSD3 | ENSG00000156011 | NP_056125.3 |
| PSMD10 | ENSG00000101843 | NP_002805.1 |
| PSMD12 | ENSG00000197170 | NP_002807.1 |
| PTBP2 | ENSG00000117569 | NP_067013.1 |
| PTCHD1 | ENSG00000165186 | NP_775766.2 |
| PTEN | ENSG00000171862 | NP_000305.3 |
| PTGER3 | ENSG00000050628 | NP_942011.1 |
| PTGS2 | ENSG00000073756 | NP_000954.1 |
| PTK7 | ENSG00000112655 | NP_001257327.1 |
| PTPN11 | ENSG00000179295 | NP_002825.3 |
| PTPRB | ENSG00000127329 | NP_001103224.1 |
| PTPRC | ENSG00000262418, | NP_002829.3 |
| | ENSG00000081237 | |
| PTPRT | ENSG00000196090 | NP_573400.3 |
| PVALB | ENSG00000100362, | NP_002845.1 |
| | ENSG00000274665 | |
| PXDN | ENSG00000130508 | NP_036425.1 |
| PYHIN1 | ENSG00000163564 | NP_689714.2 |
| RAB11FIP5 | ENSG00000135631 | NP_056285.1 |
| RAB19 | ENSG00000146955 | NP_001008749.2 |
| RAB2A | ENSG00000104388 | NP_002856.1 |
| RAB39B | ENSG00000155961 | NP_741995.1 |
| RAB43 | ENSG00000172780 | NP_001191812.1 |
| RAI1 | ENSG00000108557 | NP_109590.3 |
| RANBP17 | ENSG00000204764 | NP_075048.1 |
| RAPGEF4 | ENSG00000091428 | NP_008954.2 |
| RASD1 | ENSG00000108551 | NP_057168.1 |
| RASSF5 | ENSG00000266094 | NP_872604.1 |
| RB1CC1 | ENSG00000023287 | NP_055596.3 |
| RBFOX1/ | ENSG00000078328 | NP_665898.1 |
| A2BP1 | | |
| RBM27 | ENSG00000091009 | NP_061862.1 |
| RBM8A | ENSG00000265241 | NP_005096.1 |
| RBMS3 | ENSG00000144642 | NP_001003793.1 |
| REEP3 | ENSG00000165476 | NP_001001330.1 |
| RELN | ENSG00000189056 | NP_005036.2 |
| RERE | ENSG00000142599 | NP_036234.3 |
| RFWD2 | ENSG00000143207 | NP_071902.2 |
| RGS7 | ENSG00000182901 | NP_002915.3 |
| RHOXF1 | ENSG00000101883 | NP_644811.1 |
| RIMS1 | ENSG00000079841 | NP_055804.2 |
| RIMS3 | ENSG00000117016 | NP_055562.2 |
| RIT2 | ENSG00000152214 | NP_002921.1 |
| RLIM | ENSG00000131263 | NP_057204.2 |
| RNF135 | ENSG00000181481 | NP_115698.3 |
| RNPS1 | ENSG00000205937 | NP_542161.1 |
| ROBO1 | ENSG00000169855 | NP_002932.1 |
| ROBO2 | ENSG00000185008 | NP_001122401.1 |
| RORA | ENSG00000069667 | NP_599022.1 |
| RPL10 | ENSG00000147403 | NP_001243506.2 |
| RPP25 | ENSG00000178718 | NP_060263.2 |
| RPS6KA2 | ENSG00000071242 | NP_001006933.1 |
| RPS6KA3 | ENSG00000177189 | NP_004577.1 |
| SAE1 | ENSG00000142230 | NP_005491.1 |
| SATB2 | ENSG00000119042 | NP_001165980.1 |
| SBF1 | ENSG00000100241 | NP_002963.2 |
| SCFD2 | ENSG00000184178 | NP_689753.2 |
| SCN1A | ENSG00000144285 | NP_001159435.1 |
| SCN2A | ENSG00000136531 | NP_066287.2 |
| SCN4A | ENSG00000007314 | NP_000325.4 |
| SCN5A | ENSG00000183873 | NP_001092874.1 |
| SCN7A | ENSG00000136546 | NP_002967.2 |
| SCN8A | ENSG00000196876 | NP_055006.1 |
| SCN9A | ENSG00000169432 | NP_002968.1 |
| SDC2 | ENSG00000169439 | NP_002989.2 |
| SDK1 | ENSG00000146555 | NP_689957.3 |
| SEMA5A | ENSG00000112902 | NP_003957.2 |
| SERPINE1 | ENSG00000106366 | NP_000593.1 |
| SETBP1 | ENSG00000152217 | NP_056374.2 |
| SETD2 | ENSG00000181555 | NP_054878.5 |
| SETD5 | ENSG00000168137 | NP_001073986.1 |
| SETDB1 | ENSG00000143379 | NP_001138887.1 |
| SETDB2 | ENSG00000136169 | NP_114121.2 |
| SEZ6L2 | ENSG00000174938 | NP_001230261.1 |
| SGSH | ENSG00000181523 | NP_000190.1 |
| SGSM3 | ENSG00000100359 | NP_056520.2 |
| SH3KBP1 | ENSG00000147010 | NP_114098.1 |
| SHANK1 | ENSG00000161681 | NP_057232.2 |
| SHANK2 | ENSG00000162105 | NP_036441.2 |
| SHANK3 | ENSG00000251322, | NP_277052.1 |
| | ENSG00000283243 | |
| SHOX | ENSG00000185960 | NP_000442.1 |
| SIK1 | ENSG00000142178 | NP_775490.2 |
| SIN3A | ENSG00000169375 | NP_001138829.1 |
| SLC12A5 | ENSG00000124140 | NP_001128243.1 |
| SLC16A3 | ENSG00000141526 | NP_001035888.1 |
| SLC16A7 | ENSG00000118596 | NP_004722.2 |
| SLC1A1 | ENSG00000106688 | NP_004161.4 |
| SLC1A2 | ENSG00000110436 | NP_004162.2 |
| SLC22A15 | ENSG00000163393 | NP_060890.2 |
| SLC22A9 | ENSG00000149742 | NP_543142.2 |
| SLC24A2 | ENSG00000155886 | NP_065077.1 |
| SLC25A12 | ENSG00000115840 | NP_003696.2 |
| SLC25A14 | ENSG00000102078 | NP_003942.1 |
| SLC25A24 | ENSG00000085491, | NP_037518.3 |
| | ENSG00000284468 | |
| SLC25A27 | ENSG00000153291 | NP_004268.3 |
| SLC27A4 | ENSG00000167114 | NP_005085.2 |
| SLC29A4 | ENSG00000164638 | NP_694979.2 |
| SLC30A3 | ENSG00000115194 | NP_003450.2 |
| SLC30A5 | ENSG00000145740 | NP_075053.2 |
| SLC33A1 | ENSG00000169359 | NP_001177921.1 |
| SLC35A3 | ENSG00000117620 | NP_001258614.1 |
| SLC38A10 | ENSG00000157637 | NP_001033073.1 |
| SLC39A11 | ENSG00000282291, | NP_001153242.1 |
| | ENSG00000133195 | |
| SLC4A10 | ENSG00000144290 | NP_001171486.1 |
| SLC6A1 | ENSG00000157103 | NP_003033.3 |
| SLC6A3 | ENSG00000276996, | NP_001035.1 |
| | ENSG00000142319 | |
| SLC6A4 | ENSG00000108576 | NP_001036.1 |
| SLC6A8 | ENSG00000130821 | NP_005620.1 |
| SLC7A3 | ENSG00000165349 | NP_116192.4 |
| SLC7A5 | ENSG00000103257 | NP_003477.4 |
| SLC9A6 | ENSG00000198689 | NP_001036002.1 |
| SLC9A9 | ENSG00000181804 | NP_775924.1 |
| SLCO1B3 | ENSG00000111700 | NP_062818.1 |
| SLIT3 | ENSG00000184347 | NP_003053.1 |
| SLITRK5 | ENSG00000165300 | NP_056382.1 |
| SMARCA2 | ENSG00000080503 | NP_003061.3 |
| SMARCC2 | ENSG00000139613 | NP_003066.2 |
| SMC3 | ENSG00000108055 | NP_005436.1 |
| SMG6 | ENSG00000070366 | NP_060045.4 |
| SNAP25 | ENSG00000132639 | NP_003072.2 |
| SND1 | ENSG00000197157 | NP_055205.2 |
| SNRPN | ENSG00000128739 | NP_073716.1 |
| SNTG2 | ENSG00000172554, | NP_061841.2 |
| | ENSG00000281486, | |
| | ENSG00000281020 | |
| SNX14 | ENSG00000135317 | NP_722523.1 |
| SNX19 | ENSG00000120451 | NP_055573.2 |
| SOD1 | ENSG00000142168 | NP_000445.1 |
| SOX5 | ENSG00000134532 | NP_008871.3 |
| SPARCL1 | ENSG00000152583 | NP_001121782.1 |
| SPAST | ENSG00000021574 | NP_055761.2 |
| SRCAP | ENSG00000080603 | NP_006653.2 |
| SRD5A2 | ENSG00000277893 | NP_000339.2 |
| SRGAP3 | ENSG00000196220 | NP_055665.1 |
| SRRM4 | ENSG00000139767 | NP_919262.2 |
| SSPO | ENSG00000197558 | NP_940857.2 |
| ST7 | ENSG00000004866 | NP_068708.1 |
| ST8SIA2 | ENSG00000140557 | NP_006002.1 |
| STAG1 | ENSG00000118007 | NP_005853.2 |
| STAT1 | ENSG00000115415 | NP_009330.1 |
| STK39 | ENSG00000198648 | NP_037365.2 |
| STX1A | ENSG00000106089 | NP_004594.1 |
| STXBP1 | ENSG00000136854 | NP_003156.1 |
| STXBP5 | ENSG00000164506 | NP_001121187.1 |
| STYK1 | ENSG00000060140 | NP_060893.2 |
| SUCLG2 | ENSG00000172340 | NP_001171070.1 |
| SYAP1 | ENSG00000169895 | NP_116185.2 |
| SYN1 | ENSG00000008056 | NP_008881.2 |
| SYN2 | ENSG00000157152 | NP_598328.1 |
| SYN3 | ENSG00000185666 | NP_003481.3 |
| SYNE1 | ENSG00000131018 | NP_892006.3 |
| SYNGAP1 | ENSG00000197283, | NP_006763.2 |
| | ENSG00000227460 | |
| SYT17 | ENSG00000103528 | NP_057608.2 |
| SYT3 | ENSG00000213023 | NP_115674.1 |
| TAF1 | ENSG00000147133 | NP_004597.2 |
| TAF1C | ENSG00000103168 | NP_005670.3 |
| TAF1L | ENSG00000122728 | NP_722516.1 |
| TANC2 | ENSG00000170921 | NP_079461.2 |
| TBC1D31 | ENSG00000156787 | NP_663622.2 |
| TBC1D5 | ENSG00000131374 | NP_001127853.1 |
| TBC1D7 | ENSG00000145979 | NP_001137436.1 |
| TBL1X | ENSG00000101849 | NP_001132938.1 |
| TBL1XR1 | ENSG00000177565 | NP_078941.2 |
| TBR1 | ENSG00000136535 | NP_006584.1 |
| TBX1 | ENSG00000184058 | NP_542378.1 |
| TCF20 | ENSG00000276461, | NP_005641.1 |
| | ENSG00000262024, | |
| | ENSG00000100207, | |
| | ENSG00000280467, | |
| | ENSG00000281897, | |
| | ENSG00000283026, | |
| | ENSG00000282892, | |
| | ENSG00000283681 | |
| TCF4 | ENSG00000196628 | NP_001230155.2 |
| TCF7L2 | ENSG00000148737 | NP_001139746.1 |
| TDO2 | ENSG00000151790, | NP_005642.1 |
| | ENSG00000262635 | |
| TERF2 | ENSG00000132604 | NP_005643.2 |
| TERT | ENSG00000164362 | NP_937983.2 |
| TET2 | ENSG00000168769 | NP_001120680.1 |
| TGM3 | ENSG00000125780 | NP_003236.3 |
| TH | ENSG00000180176 | NP_954986.2 |
| THAP8 | ENSG00000161277 | NP_689871.1 |
| THBS1 | ENSG00000137801 | NP_003237.2 |
| THRA | ENSG00000126351 | NP_003241.2 |
| TLK2 | ENSG00000146872 | NP_006843.2 |
| TM4SF20 | ENSG00000168955 | NP_079071.2 |
| TMEM231 | ENSG00000205084 | NP_001070884.1 |
| TMLHE | ENSG00000185973 | NP_060666.1 |
| TNIP2 | ENSG00000168884 | NP_077285.3 |
| TNN | ENSG00000120332 | NP_071376.1 |
| TNRC6B | ENSG00000100354 | NP_001155973.1 |
| TOMM20 | ENSG00000173726 | NP_055580.1 |
| TOP1 | ENSG00000198900 | NP_003277.1 |
| TOP3B | ENSG00000100038 | NP_003926.1 |
| TPH2 | ENSG00000139287 | NP_775489.2 |
| TPO | ENSG00000115705, | NP_000538.3 |
| | ENSG00000277603 | |
| TRIM33 | ENSG00000197323 | NP_056990.3 |
| TRIO | ENSG00000038382 | NP_009049.2 |
| TRIP12 | ENSG00000153827 | NP_004229.1 |
| TRPC6 | ENSG00000137672 | NP_004612.2 |
| TRPM1 | ENSG00000134160, | NP_001238949.1 |
| | ENSG00000274965 | |
| TSC1 | ENSG00000165699 | NP_000359.1 |
| TSC2 | ENSG00000103197 | NP_000539.2 |
| TSHZ3 | ENSG00000121297 | NP_065907.2 |
| TSN | ENSG00000211460 | NP_004613.1 |
| TSPAN7 | ENSG00000156298 | NP_004606.2 |
| TTI2 | ENSG00000129696 | NP_079391.2 |
| TTN | ENSG00000155657 | NP_001254479.1 |
| TUBGCP5 | ENSG00000280807, | NP_001096080.1 |
| | ENSG00000275835, | |
| | ENSG00000276856 | |
| TYR | ENSG00000077498 | NP_000363.1 |
| UBA6 | ENSG00000033178 | NP_060697.4 |
| UBE2H | ENSG00000186591 | NP_003335.1 |
| UBE3A | ENSG00000114062 | NP_000453.2 |
| UBE3B | ENSG00000151148 | NP_904324.1 |
| UBE3C | ENSG00000009335 | NP_055486.2 |
| UBL7 | ENSG00000138629 | NP_116296.1 |
| UBR5 | ENSG00000104517 | NP_056986.2 |
| UBR7 | ENSG00000278787, | NP_786924.2 |
| | ENSG00000012963 | |
| UCN3 | ENSG00000178473 | NP_444277.2 |
| UNC13A | ENSG00000130477 | NP_001073890.2 |
| UNC80 | ENSG00000144406 | NP_115893.1 |
| UPB1 | ENSG00000100024 | NP_057411.1 |
| UPF2 | ENSG00000151461 | NP_056357.1 |
| UPF3B | ENSG00000125351 | NP_542199.1 |
| USP45 | ENSG00000123552 | NP_001073950.1 |
| USP7 | ENSG00000187555 | NP_003461.2 |
| USP9Y | ENSG00000114374 | NP_004645.2 |
| UTRN | ENSG00000152818 | NP_009055.2 |
| VASH1 | ENSG00000071246 | NP_055724.1 |
| VIL1 | ENSG00000127831 | NP_009058.2 |
| VIP | ENSG00000146469 | NP_003372.1 |
| VLDLR | ENSG00000147852 | NP_003374.3 |
| VPS13B | ENSG00000132549 | NP_060360.3 |
| VSIG4 | ENSG00000155659 | NP_009199.1 |
| WAC | ENSG00000095787 | NP_057712.2 |
| WDFY3 | ENSG00000163625 | NP_055806.2 |
| WDR93 | ENSG00000140527 | NP_064597.1 |
| WNK3 | ENSG00000196632 | NP_065973.2 |
| WNT1 | ENSG00000125084 | NP_005421.1 |
| WNT2 | ENSG00000105989 | NP_003382.1 |
| WWOX | ENSG00000186153 | NP_057457.1 |
| XIRP1 | ENSG00000168334 | NP_919269.2 |
| XPC | ENSG00000154767 | NP_004619.3 |
| XPO1 | ENSG00000082898 | NP_003391.1 |
| YEATS2 | ENSG00000163872 | NP_060493.3 |
| YTHDC2 | ENSG00000047188 | NP_073739.3 |
| YWHAE | ENSG00000108953, | NP_006752.1 |
| | ENSG00000274474 | |
| ZBTB16 | ENSG00000109906 | NP_005997.2 |
| ZBTB20 | ENSG00000181722 | NP_001157814.1 |
| ZC3H4 | ENSG00000130749 | NP_055983.1 |
| ZMYND11 | ENSG00000015171 | NP_006615.2 |
| ZNF18 | ENSG00000154957 | NP_653281.2 |
| ZNF292 | ENSG00000188994 | NP_055836.1 |
| ZNF385B | ENSG00000144331 | NP_689733.3 |
| ZNF407 | ENSG00000215421 | NP_060227.2 |
| ZNF462 | ENSG00000148143 | NP_067047.4 |
| ZNF517 | ENSG00000197363 | NP_998770.2 |
| ZNF559 | ENSG00000188321 | NP_001189335.1 |
| ZNF713 | ENSG00000178665 | NP_872439.1 |
| ZNF774 | ENSG00000196391 | NP_001004309.2 |
| ZNF8 | ENSG00000278129 | NP_066575.2 |
| ZNF804A | ENSG00000170396 | NP_919226.1 |
| ZNF827 | ENSG00000151612 | NP_849157.2 |
| ZSWIM5 | ENSG00000162415 | NP_065934.1 |
| ZWILCH | ENSG00000174442 | NP_060445.3 |

*Representative protein in the family encoded by the gene.

TABLE 12
(338 Genes) - SFARI genes that have a high-scoring hnRNP
L motif within 500 bp of one of the Castle splice sites

| | | | Genes with |
|---|---|---|---|
| | | | hnRNP binding |
| | | | sites within |
| | | | 200 base |
| | | | pairs of an |
| | Ensembl | Accession | RBFox1/A2BP1 |
| Human Gene | Identifier | Number* | binding site |
| A2BP1 | ENSG00000078328 | NP_665898.1 | X |
| ABAT | ENSG00000183044 | NP_001120920.1 | |
| ABCA10 | ENSG00000154263 | NP_525021.3 | |
| ABCA7 | ENSG00000064687 | NP_061985.2 | X |
| ACHE | ENSG00000087085 | NP_000656.1 | |
| ACTN4 | ENSG00000130402, | NP_004915.2 | |
| | ENSG00000282844 | | |
| ACY1 | ENSG00000243989 | NP_001185824.1 | |
| ADAMTS18 | ENSG00000140873 | NP_955387.1 | X |
| ADARB1 | ENSG00000197381 | NP_056648.1 | |
| ADK | ENSG00000156110 | NP_001114.2 | |
| ADNP | ENSG00000101126 | NP_056154.1 | X |
| ADSL | ENSG00000239900 | NP_000017.1 | X |
| AHI1 | ENSG00000135541 | NP_001128302.1 | X |
| AKAP9 | ENSG00000127914 | NP_005742.4 | |
| ALG6 | ENSG00000088035 | NP_037471.2 | |
| AMT | ENSG00000145020 | NP_000472.2 | |
| ANK2 | ENSG00000145362 | NP_001139.3 | |
| ANK3 | ENSG00000151150 | NP_066267.2 | X |
| ANKRD11 | ENSG00000167522 | NP_001243111.1 | |
| ANXA1 | ENSG00000135046 | NP_000691.1 | |
| AP1S2 | ENSG00000182287 | NP_003907.3 | |
| APC | ENSG00000134982 | NP_001120982.1 | |
| APP | ENSG00000142192 | NP_000475.1 | X |
| ARHGAP15 | ENSG00000075884 | NP_060930.3 | |
| ARHGEF9 | ENSG00000131089 | NP_056000.1 | |
| ARID1B | ENSG00000049618 | NP_065783.3 | |
| ARNT2 | ENSG00000172379 | NP_055677.3 | |
| ASMT | ENSG00000196433 | NP_004034.2 | X |
| ASTN2 | ENSG00000148219 | NP_054729.3 | |
| ATP10A | ENSG00000206190 | NP_077816.1 | |
| ATP2B2 | ENSG00000157087 | NP_001001331.1 | X |
| ATP8A1 | ENSG00000124406 | NP_006086.1 | |
| ATRNL1 | ENSG00000107518 | NP_997186.1 | X |
| ATRX | ENSG00000085224 | NP_000480.3 | X |
| AUTS2 | ENSG00000158321 | NP_056385.1 | |
| BAIAP2 | ENSG00000175866 | NP_059345.1 | X |
| BCAS1 | ENSG00000064787 | NP_003648.2 | X |
| BCKDK | ENSG00000103507 | NP_005872.2 | |
| BCL11A | ENSG00000119866 | NP_075044.2 | X |
| BIN1 | ENSG00000136717 | NP_647593.1 | X |
| BRCA2 | ENSG00000139618 | NP_000050.2 | |
| BTAF1 | ENSG00000095564 | NP_003963.1 | |
| CACNA1B | ENSG00000148408 | NP_000709.1 | |
| CACNA1C | ENSG00000151067 | NP_955630.3 | X |
| CACNA1D | ENSG00000157388 | NP_000711.1 | X |
| CACNA1E | ENSG00000198216 | NP_001192222.1 | |
| CACNA1F | ENSG00000102001 | NP_005174.2 | |
| CACNA1G | ENSG00000006283 | NP_061496.2 | |
| CACNA1I | ENSG00000100346 | NP_066919.2 | |
| CACNA2D3 | ENSG00000157445 | NP_060868.2 | |
| CACNB2 | ENSG00000165995 | NP_963890.2 | X |
| CADPS2 | ENSG00000081803 | NP_001161412.1 | X |
| CAMTA1 | ENSG00000171735 | NP_056030.1 | X |
| CAPN12 | ENSG00000182472 | NP_653292.2 | |
| CASK | ENSG00000147044 | NP_003679.2 | X |
| CBS | ENSG00000160200 | NP_001171479.1 | |
| CCT4 | ENSG00000115484 | NP_006421.2 | |
| CD38 | ENSG00000004468 | NP_001766.2 | |
| CD44 | ENSG00000026508 | NP_000601.3 | X |
| CD99L2 | ENSG00000102181 | NP_001229543.1 | X |
| CDH11 | ENSG00000140937 | NP_001788.2 | |
| CEP290 | ENSG00000198707 | NP_079390.3 | |
| CHD2 | ENSG00000173575 | NP_001262.3 | X |
| CHD7 | ENSG00000171316 | NP_060250.2 | |
| CHKB | ENSG00000100288 | NP_001164100.1 | |
| CHRNA7 | ENSG00000175344 | NP_001177384.1 | |
| CIB2 | ENSG00000136425 | NP_006374.1 | |
| CLSTN3 | ENSG00000139182 | NP_055533.2 | |
| CLTCL1 | ENSG00000070371 | NP_009029.3 | |
| CMIP | ENSG00000153815 | NP_938204.2 | |
| CNKSR2 | ENSG00000149970 | NP_055742.2 | X |
| CNOT3 | ENSG00000277600, | NP_055331.1 | |
| | ENSG00000274616, | | |
| | ENSG00000277114, | | |
| | ENSG00000088038, | | |
| | ENSG00000273943, | | |
| | ENSG00000274941, | | |
| | ENSG00000274176, | | |
| | ENSG00000277615, | | |
| | ENSG00000276082, | | |
| | ENSG00000275979 | | |
| CNR1 | ENSG00000118432 | NP_001153698.1 | |
| CNTN5 | ENSG00000149972 | NP_001230199.1 | |
| CNTNAP3 | ENSG00000106714 | NP_387504.2 | X |
| CNTNAP5 | ENSG00000155052 | NP_570129.1 | |
| CRHR2 | ENSG00000106113 | NP_001189404.1 | |
| CSNK1D | ENSG00000141551 | NP_001884.2 | X |
| CYLC2 | ENSG00000155833 | NP_001331.1 | |
| DAB1 | ENSG00000173406 | NP_066566.3 | |
| DAPK1 | ENSG00000196730 | NP_004929.2 | X |
| DAPP1 | ENSG00000070190 | NP_055210.2 | X |
| DDC | ENSG00000132437 | NP_001076440.1 | X |
| DDX11 | ENSG00000013573 | NP_689651.1 | |
| DDX3X | ENSG00000215301 | NP_001347.3 | |
| DEAF1 | ENSG00000177030, | NP_066288.2 | |
| | ENSG00000282712 | | |
| DENR | ENSG00000139726 | NP_003668.2 | X |
| DGKZ | ENSG00000149091 | NP_001099010.1 | |
| DHCR7 | ENSG00000172893 | NP_001351.2 | |
| DLG1 | ENSG00000075711 | NP_004078.2 | |
| DLGAP2 | ENSG00000198010, | NP_004736.2 | |
| | ENSG00000282152, | | |
| | ENSG00000274161, | | |
| | ENSG00000282318 | | |
| DMD | ENSG00000198947 | NP_003997.1 | X |
| DMXL2 | ENSG00000104093 | NP_001167587.1 | X |
| DNM1L | ENSG00000087470 | NP_036192.2 | X |
| DNMT3A | ENSG00000119772 | NP_072046.2 | |
| DOCK4 | ENSG00000128512 | NP_055520.3 | |
| DOCK8 | ENSG00000107099 | NP_982272.2 | |
| DPP4 | ENSG00000197635 | NP_001926.2 | X |
| DRD2 | ENSG00000149295 | NP_000786.1 | |
| DSCAM | ENSG00000171587 | NP_001380.2 | |
| DUSP15 | ENSG00000149599 | NP_542178.2 | X |
| DUSP22 | ENSG00000112679 | NP_064570.1 | |
| DYRK1A | ENSG00000157540 | NP_001387.2 | X |
| EGR2 | ENSG00000122877 | NP_001129649.1 | X |
| EIF4E | ENSG00000151247 | NP_001124151.1 | |
| ELAVL2 | ENSG00000107105 | NP_004423.2 | |
| ELP4 | ENSG00000109911 | NP_061913.3 | X |
| EPHB2 | ENSG00000133216 | NP_004433.2 | X |
| ERG | ENSG00000157554 | NP_001129626.1 | |
| ESR1 | ENSG00000091831 | NP_001116212.1 | |
| ESR2 | ENSG00000140009 | NP_001428.1 | X |
| FBN1 | ENSG00000166147 | NP_000129.3 | X |
| FLT1 | ENSG00000102755 | NP_002010.2 | |
| FMR1 | ENSG00000102081 | NP_002015.1 | |
| FOLH1 | ENSG00000086205 | NP_004467.1 | |
| FOXP1 | ENSG00000114861 | NP_001231739.1 | X |
| FOXP2 | ENSG00000128573 | NP_683696.2 | |
| FRK | ENSG00000111816 | NP_002022.1 | |
| GABRB3 | ENSG00000166206 | NP_068712.1 | |
| GALNT14 | ENSG00000158089 | NP_001240755.1 | |
| GAP43 | ENSG00000172020 | NP_001123536.1 | |
| GAS2 | ENSG00000148935 | NP_001137302.1 | |
| GDA | ENSG00000119125 | NP_001229434.1 | X |
| GNAS | ENSG00000087460 | NP_536350.2 | X |
| GNB1L | ENSG00000185838 | NP_443730.1 | X |
| GPD2 | ENSG00000115159 | NP_001076581.2 | X |
| GRID1 | ENSG00000182771 | NP_060021.1 | |
| GRIK2 | ENSG00000164418 | NP_068775.1 | |
| GRIN1 | ENSG00000176884 | NP_001172019.1 | X |
| GRM1 | ENSG00000152822 | NP_000829.2 | X |
| GRM4 | ENSG00000124493 | NP_000832.1 | |
| GRM5 | ENSG00000168959 | NP_001137303.1 | X |
| GRM7 | ENSG00000196277 | NP_870989.1 | X |
| GRM8 | ENSG00000179603 | NP_000836.2 | X |
| GSK3B | ENSG00000082701 | NP_002084.2 | |
| GSN | ENSG00000148180 | NP_000168.1 | |
| GSTM1 | ENSG00000134184 | NP_000552.2 | |
| GTF2I | ENSG00000263001 | NP_127492.1 | |
| GUCY1A2 | ENSG00000152402 | NP_001243353.1 | |
| HDAC3 | ENSG00000171720 | NP_003874.2 | X |
| HDAC4 | ENSG00000068024 | NP_006028.2 | |
| HDAC6 | ENSG00000094631 | NP_006035.2 | |
| HLA-DRB1 | ENSG00000236884, | NP_001230894.1 | X |
| | ENSG00000228080, | | |
| | ENSG00000206240, | | |
| | ENSG00000229074, | | |
| | ENSG00000206306, | | |
| | ENSG00000196126 | | |
| HMGN1 | ENSG00000205581 | NP_004956.5 | |
| HOXA1 | ENSG00000105991 | NP_005513.1 | |
| HRAS | ENSG00000174775, | NP_001123914.1 | |
| | ENSG00000276536 | | |
| HTR3A | ENSG00000166736 | NP_998786.2 | X |
| HTR7 | ENSG00000148680 | NP_062873.1 | |
| ICA1 | ENSG00000003147 | NP_071682.1 | |
| IL16 | ENSG00000172349 | NP_757366.2 | |
| IQSEC2 | ENSG00000124313 | NP_001104595.1 | X |
| ITGA4 | ENSG00000115232 | NP_000876.3 | |
| ITPR1 | ENSG00000150995 | NP_001161744.1 | X |
| JMJD1C | ENSG00000171988 | NP_116165.1 | X |
| KCNMA1 | ENSG00000156113 | NP_001154824.1 | |
| KCNQ2 | ENSG00000281151, | NP_742105.1 | X |
| | ENSG00000075043 | | |
| KCNT1 | ENSG00000107147 | NP_065873.2 | |
| KHDRBS3 | ENSG00000131773 | NP_006549.1 | X |
| KIF13B | ENSG00000197892 | NP_056069.2 | X |
| KIF5C | ENSG00000276734, | NP_004513.1 | |
| | ENSG00000168280 | | |
| KLC2 | ENSG00000174996 | NP_073733.1 | X |
| KMO | ENSG00000117009 | NP_003670.2 | X |
| LAMC3 | ENSG00000050555 | NP_006050.3 | |
| LEP | ENSG00000174697 | NP_000221.1 | |
| LIN7B | ENSG00000104863 | NP_071448.1 | |
| LPL | ENSG00000175445 | NP_000228.1 | X |
| LRBA | ENSG00000198589 | NP_006717.2 | X |
| LRFN5 | ENSG00000165379 | NP_689660.2 | |
| LRRC7 | ENSG00000033122 | NP_065845.1 | X |
| LZTR1 | ENSG00000099949 | NP_006758.2 | |
| MAGED1 | ENSG00000179222 | NP_001005333.1 | |
| MAL | ENSG00000172005 | NP_002362.1 | |
| MAP2 | ENSG00000078018 | NP_002365.3 | X |
| MAPK3 | ENSG00000102882 | NP_002737.2 | |
| MARK1 | ENSG00000116141 | NP_061120.3 | |
| MBD1 | ENSG00000141644 | NP_001191065.1 | X |
| MBD4 | ENSG00000129071 | NP_003916.1 | |
| MBD5 | ENSG00000204406 | NP_060798.2 | |
| MBD6 | ENSG00000166987 | NP_443129.3 | |
| MCC | ENSG00000171444 | NP_001078846.1 | X |
| MED12 | ENSG00000184634 | NP_005111.2 | |
| MEF2C | ENSG00000081189 | NP_001180276.1 | X |
| MET | ENSG00000105976 | NP_001120972.1 | |
| MNT | ENSG00000070444 | NP_064706.1 | |
| MPP6 | ENSG00000105926 | NP_057531.2 | |
| MSR1 | ENSG00000038945 | NP_619729.1 | |
| MTX2 | ENSG00000128654 | NP_006545.1 | |
| MYT1L | ENSG00000186487 | NP_055840.2 | X |
| NAV2 | ENSG00000166833 | NP_001231892.1 | X |
| NBEA | ENSG00000172915 | NP_056493.3 | X |
| NCOR1 | ENSG00000141027 | NP_006302.2 | X |
| NDUFA5 | ENSG00000128609 | NP_004991.1 | |
| NF1 | ENSG00000196712 | NP_001035957.1 | |
| NFIA | ENSG00000162599 | NP_001138984.1 | X |
| NFIX | ENSG00000008441 | NP_001257972.1 | X |
| NIPA2 | ENSG00000140157 | NP_001171818.1 | |
| NIPBL | ENSG00000164190 | NP_597677.2 | |
| NLGN3 | ENSG00000196338 | NP_851820.1 | |
| NLGN4X | ENSG00000146938 | NP_065793.1 | X |
| NLGN4Y | ENSG00000165246 | NP_055708.3 | X |
| NPAS2 | ENSG00000170485 | NP_002509.2 | |
| NRCAM | ENSG00000091129 | NP_001032209.1 | X |
| NRG1 | ENSG00000157168 | NP_039250.2 | X |
| NRXN1 | ENSG00000179915 | NP_001129131.1 | X |
| NRXN2 | ENSG00000110076 | NP_055895.1 | X |
| NRXN3 | ENSG00000021645 | NP_004787.2 | X |
| NSD1 | ENSG00000165671 | NP_071900.2 | |
| NTRK1 | ENSG00000198400 | NP_002520.2 | |
| NTRK3 | ENSG00000140538 | NP_001012338.1 | X |
| NXF5 | ENSG00000126952 | NP_116564.2 | X |
| OCRL | ENSG00000122126 | NP_000267.2 | X |
| OGT | ENSG00000147162 | NP_858058.1 | |
| P4HA2 | ENSG00000072682 | NP_001136071.1 | |
| PACS1 | ENSG00000175115 | NP_060496.2 | |
| PAH | ENSG00000171759 | NP_000268.1 | X |
| PAX5 | ENSG00000196092 | NP_057953.1 | |
| PAX6 | ENSG00000007372 | NP_001245391.1 | X |
| PCCA | ENSG00000175198 | NP_000273.2 | X |
| PCCB | ENSG00000114054 | NP_001171485.1 | |
| PCDH19 | ENSG00000165194 | NP_001171809.1 | |
| PCDH8 | ENSG00000136099 | NP_002581.2 | |
| PECR | ENSG00000115425 | NP_060911.2 | |
| PER1 | ENSG00000179094 | NP_002607.2 | X |
| PHF2 | ENSG00000197724 | NP_005383.3 | |
| PHF8 | ENSG00000172943 | NP_001171825.1 | |
| PHIP | ENSG00000146247 | NP_060404.4 | X |
| PIK3R2 | ENSG00000105647 | NP_005018.1 | |
| PINX1 | ENSG00000254093 | NP_060354.4 | |
| PLAUR | ENSG00000011422 | NP_002650.1 | X |
| PLCB1 | ENSG00000182621 | NP_056007.1 | |
| PLXNB1 | ENSG00000164050 | NP_001123554.1 | |
| POGZ | ENSG00000143442 | NP_055915.2 | |
| PPFIA1 | ENSG00000131626 | NP_003617.1 | X |
| PPP1R3F | ENSG00000049769 | NP_149992.3 | |
| PREX1 | ENSG00000124126 | NP_065871.2 | X |
| PRKDC | ENSG00000253729 | NP_008835.5 | X |
| PRODH | ENSG00000100033 | NP_057419.4 | X |
| PSD3 | ENSG00000156011 | NP_056125.3 | |
| PSMD10 | ENSG00000101843 | NP_002805.1 | |
| PSMD12 | ENSG00000197170 | NP_002807.1 | |
| PTBP2 | ENSG00000117569 | NP_067013.1 | X |
| PTGER3 | ENSG00000050628 | NP_942011.1 | X |
| PTK7 | ENSG00000112655 | NP_001257327.1 | |
| PTPRC | ENSG00000262418, | NP_002829.3 | X |
| | ENSG00000081237 | | |
| PVALB | ENSG00000100362, | NP_002845.1 | |
| | ENSG00000274665 | | |
| RANBP17 | ENSG00000204764 | NP_075048.1 | X |
| RBM27 | ENSG00000091009 | NP_061862.1 | |
| RBM8A | ENSG00000265241 | NP_005096.1 | |
| RERE | ENSG00000142599 | NP_036234.3 | |
| RFWD2 | ENSG00000143207 | NP_071902.2 | X |
| RGS7 | ENSG00000182901 | NP_002915.3 | X |
| RIMS1 | ENSG00000079841 | NP_055804.2 | X |
| RIMS3 | ENSG00000117016 | NP_055562.2 | |
| RIT2 | ENSG00000152214 | NP_002921.1 | |
| RNF135 | ENSG00000181481 | NP_115698.3 | |
| RNPS1 | ENSG00000205937 | NP_542161.1 | |
| RORA | ENSG00000069667 | NP_599022.1 | X |
| RPL10 | ENSG00000147403 | NP_001243506.2 | |
| SAE1 | ENSG00000142230 | NP_005491.1 | |
| SBF1 | ENSG00000100241 | NP_002963.2 | |
| SCFD2 | ENSG00000184178 | NP_689753.2 | X |
| SCN8A | ENSG00000196876 | NP_055006.1 | |
| SCN9A | ENSG00000169432 | NP_002968.1 | X |
| SDK1 | ENSG00000146555 | NP_689957.3 | |
| SETDB1 | ENSG00000143379 | NP_001138887.1 | |
| SEZ6L2 | ENSG00000174938 | NP_001230261.1 | |
| SGSH | ENSG00000181523 | NP_000190.1 | |
| SHANK2 | ENSG00000162105 | NP_036441.2 | X |
| SHANK3 | ENSG00000251322, | NP_277052.1 | X |
| | ENSG00000283243 | | |
| SIN3A | ENSG00000169375 | NP_001138829.1 | X |
| SLC16A7 | ENSG00000118596 | NP_004722.2 | |
| SLC22A9 | ENSG00000149742 | NP_543142.2 | |
| SLC24A2 | ENSG00000155886 | NP_065077.1 | X |
| SLC25A12 | ENSG00000115840 | NP_003696.2 | |
| SLC25A14 | ENSG00000102078 | NP_003942.1 | |
| SLC25A27 | ENSG00000153291 | NP_004268.3 | X |
| SLC30A5 | ENSG00000145740 | NP_075053.2 | |
| SLC33A1 | ENSG00000169359 | NP_001177921.1 | X |
| SLC7A3 | ENSG00000165349 | NP_116192.4 | |
| SMARCA2 | ENSG00000080503 | NP_003061.3 | |
| SMARCC2 | ENSG00000139613 | NP_003066.2 | |
| SNAP25 | ENSG00000132639 | NP_003072.2 | |
| SNRPN | ENSG00000128739 | NP_073716.1 | X |
| SNX14 | ENSG00000135317 | NP_722523.1 | X |
| SOD1 | ENSG00000142168 | NP_000445.1 | |
| SOX5 | ENSG00000134532 | NP_008871.3 | |
| SPAST | ENSG00000021574 | NP_055761.2 | |
| SRCAP | ENSG00000080603 | NP_006653.2 | |
| SRGAP3 | ENSG00000196220 | NP_055665.1 | |
| ST7 | ENSG00000004866 | NP_068708.1 | X |
| ST8SIA2 | ENSG00000140557 | NP_006002.1 | |
| STXBP1 | ENSG00000136854 | NP_003156.1 | X |
| STXBP5 | ENSG00000164506 | NP_001121187.1 | |
| SUCLG2 | ENSG00000172340 | NP_001171070.1 | |
| SYN2 | ENSG00000157152 | NP_598328.1 | |
| SYN3 | ENSG00000185666 | NP_003481.3 | |
| SYNE1 | ENSG00000131018 | NP_892006.3 | X |
| SYT3 | ENSG00000213023 | NP_115674.1 | |
| TAF1 | ENSG00000147133 | NP_004597.2 | |
| TAF1C | ENSG00000103168 | NP_005670.3 | |
| TBC1D5 | ENSG00000131374 | NP_001127853.1 | |
| TBCID7 | ENSG00000145979 | NP_001137436.1 | |
| TBL1X | ENSG00000101849 | NP_001132938.1 | |
| TBR1 | ENSG00000136535 | NP_006584.1 | |
| TCF20 | ENSG00000276461, | NP_005641.1 | |
| | ENSG00000262024, | | |
| | ENSG00000100207, | | |
| | ENSG00000280467, | | |
| | ENSG00000281897, | | |
| | ENSG00000283026, | | |
| | ENSG00000282892, | | |
| | ENSG00000283681 | | |
| TCF4 | ENSG00000196628 | NP_001230155.2 | |
| TCF7L2 | ENSG00000148737 | NP_001139746.1 | |
| TERT | ENSG00000164362 | NP_937983.2 | |
| TH | ENSG00000180176 | NP_954986.2 | |
| THRA | ENSG00000126351 | NP_003241.2 | |
| TOP3B | ENSG00000100038 | NP_003926.1 | X |
| TPO | ENSG00000115705, | NP_000538.3 | X |
| | ENSG00000277603 | | |
| TRIM33 | ENSG00000197323 | NP_056990.3 | X |
| TRIO | ENSG00000038382 | NP_009049.2 | |
| TRIP12 | ENSG00000153827 | NP_004229.1 | |
| TRPC6 | ENSG00000137672 | NP_004612.2 | X |
| TRPM1 | ENSG00000134160, | NP_001238949.1 | |
| | ENSG00000274965 | | |
| TSC1 | ENSG00000165699 | NP_000359.1 | |
| TSC2 | ENSG00000103197 | NP_000539.2 | X |
| TSN | ENSG00000211460 | NP_004613.1 | |
| TTN | ENSG00000155657 | NP_001254479.1 | X |
| TUBGCP5 | ENSG00000280807, | NP_001096080.1 | |
| | ENSG00000275835, | | |
| | ENSG00000276856 | | |
| UBE3A | ENSG00000114062 | NP_000453.2 | |
| UBE3B | ENSG00000151148 | NP_904324.1 | X |
| UBE3C | ENSG00000009335 | NP_055486.2 | |
| UPB1 | ENSG00000100024 | NP_057411.1 | |
| UPF3B | ENSG00000125351 | NP_542199.1 | |
| USP45 | ENSG00000123552 | NP_001073950.1 | |
| USP9Y | ENSG00000114374 | NP_004645.2 | |
| VIP | ENSG00000146469 | NP_003372.1 | |
| VLDLR | ENSG00000147852 | NP_003374.3 | X |
| WAC | ENSG00000095787 | NP_057712.2 | X |
| WDFY3 | ENSG00000163625 | NP_055806.2 | X |
| WNK3 | ENSG00000196632 | NP_065973.2 | X |
| WWOX | ENSG00000186153 | NP_057457.1 | X |
| ZBTB20 | ENSG00000181722 | NP_001157814.1 | X |
| ZMYND11 | ENSG00000015171 | NP_006615.2 | |
| ZNF18 | ENSG00000154957 | NP_653281.2 | |

*Representative protein in the family encoded by the gene.

TABLE 13
(152 Genes) - SFARI genes that have a very high
scoring putative hnRNP L-binding motif within
500bp of one of the Castle splice sites.

| | | | Genes with |
|---|---|---|---|
| | | | hnRNP binding |
| | | | sites within |
| | | | 200 base |
| | | | pairs of an |
| | Ensembl | Accession | RBFox1/A2BP1 |
| Human Gene | Identifier | Number* | binding site |
| A2BP1 | ENSG00000078328 | NP_665898.1 | X |
| ABCA10 | ENSG00000154263 | NP_525021.3 | |
| ACHE | ENSG00000087085 | NP_000656.1 | |
| ADAMTS18 | ENSG00000140873 | NP_955387.1 | |
| ADARB1 | ENSG00000197381 | NP_056648.1 | |
| ADNP | ENSG00000101126 | NP_056154.1 | X |
| AHI1 | ENSG00000135541 | NP_001128302.1 | |
| ANK3 | ENSG00000151150 | NP_066267.2 | |
| ANKRD11 | ENSG00000167522 | NP_001243111.1 | |
| ANXA1 | ENSG00000135046 | NP_000691.1 | |
| ARHGEF9 | ENSG00000131089 | NP_056000.1 | |
| ARID1B | ENSG00000049618 | NP_065783.3 | |
| ARNT2 | ENSG00000172379 | NP_055677.3 | |
| ASMT | ENSG00000196433 | NP_004034.2 | |
| ATP2B2 | ENSG00000157087 | NP_001001331.1 | |
| ATRNL1 | ENSG00000107518 | NP_997186.1 | X |
| ATRX | ENSG00000085224 | NP_000480.3 | |
| AUTS2 | ENSG00000158321 | NP_056385.1 | |
| BCAS1 | ENSG00000064787 | NP_003648.2 | X |
| BCKDK | ENSG00000103507 | NP_005872.2 | |
| BIN1 | ENSG00000136717 | NP_647593.1 | X |
| CACNA1B | ENSG00000148408 | NP_000709.1 | |
| CACNA1C | ENSG00000151067 | NP_955630.3 | |
| CACNA1E | ENSG00000198216 | NP_001192222.1 | |
| CACNA1F | ENSG00000102001 | NP_005174.2 | |
| CACNA1G | ENSG00000006283 | NP_061496.2 | |
| CAMTA1 | ENSG00000171735 | NP_056030.1 | X |
| CD38 | ENSG00000004468 | NP_001766.2 | |
| CD99L2 | ENSG00000102181 | NP_001229543.1 | |
| CHRNA7 | ENSG00000175344 | NP_001177384.1 | |
| CNKSR2 | ENSG00000149970 | NP_055742.2 | X |
| CNTN5 | ENSG00000149972 | NP_001230199.1 | |
| CNTNAP3 | ENSG00000106714 | NP_387504.2 | |
| CRHR2 | ENSG00000106113 | NP_001189404.1 | |
| DAPP1 | ENSG00000070190 | NP_055210.2 | |
| DDC | ENSG00000132437 | NP_001076440.1 | |
| DEAF1 | ENSG00000177030, | NP_066288.2 | |
| | ENSG00000282712 | | |
| DLG1 | ENSG00000075711 | NP_004078.2 | |
| DMD | ENSG00000198947 | NP_003997.1 | |
| DMXL2 | ENSG00000104093 | NP_001167587.1 | |
| DOCK4 | ENSG00000128512 | NP_055520.3 | |
| DOCK8 | ENSG00000107099 | NP_982272.2 | |
| DPP4 | ENSG00000197635 | NP_001926.2 | X |
| DRD2 | ENSG00000149295 | NP_000786.1 | |
| DUSP15 | ENSG00000149599 | NP_542178.2 | X |
| ELAVL2 | ENSG00000107105 | NP_004423.2 | |
| ELP4 | ENSG00000109911 | NP_061913.3 | |
| EPHB2 | ENSG00000133216 | NP_004433.2 | |
| ESR1 | ENSG00000091831 | NP_001116212.1 | |
| ESR2 | ENSG00000140009 | NP_001428.1 | |
| FMR1 | ENSG00000102081 | NP_002015.1 | |
| FOLH1 | ENSG00000086205 | NP_004467.1 | |
| FOXP1 | ENSG00000114861 | NP_001231739.1 | |
| FOXP2 | ENSG00000128573 | NP_683696.2 | |
| FRK | ENSG00000111816 | NP_002022.1 | |
| GABRB3 | ENSG00000166206 | NP_068712.1 | |
| GDA | ENSG00000119125 | NP_001229434.1 | X |
| GNAS | ENSG00000087460 | NP_536350.2 | |
| GTF2I | ENSG00000263001 | NP_127492.1 | |
| HDAC6 | ENSG00000094631 | NP_006035.2 | |
| HLA-DRB1 | ENSG00000236884, | NP_001230894.1 | |
| | ENSG00000228080, | | |
| | ENSG00000206240, | | |
| | ENSG00000229074, | | |
| | ENSG00000206306, | | |
| | ENSG00000196126 | | |
| HTR3A | ENSG00000166736 | NP_998786.2 | X |
| HTR7 | ENSG00000148680 | NP_062873.1 | |
| ICA1 | ENSG00000003147 | NP_071682.1 | |
| IQSEC2 | ENSG00000124313 | NP_001104595.1 | |
| ITPR1 | ENSG00000150995 | NP_001161744.1 | X |
| JMJD1C | ENSG00000171988 | NP_116165.1 | |
| KCNT1 | ENSG00000107147 | NP_065873.2 | |
| KHDRBS3 | ENSG00000131773 | NP_006549.1 | |
| KIF13B | ENSG00000197892 | NP_056069.2 | X |
| KIF5C | ENSG00000276734, | NP_004513.1 | |
| | ENSG00000168280 | | |
| KLC2 | ENSG00000174996 | NP_073733.1 | X |
| KMO | ENSG00000117009 | NP_003670.2 | X |
| LIN7B | ENSG00000104863 | NP_071448.1 | |
| LPL | ENSG00000175445 | NP_000228.1 | X |
| LRFN5 | ENSG00000165379 | NP_689660.2 | |
| LRRC7 | ENSG00000033122 | NP_065845.1 | X |
| MAP2 | ENSG00000078018 | NP_002365.3 | X |
| MARK1 | ENSG00000116141 | NP_061120.3 | |
| MBD1 | ENSG00000141644 | NP_001191065.1 | |
| MBD5 | ENSG00000204406 | NP_060798.2 | |
| MBD6 | ENSG00000166987 | NP_443129.3 | |
| MCC | ENSG00000171444 | NP_001078846.1 | X |
| MED12 | ENSG00000184634 | NP_005111.2 | |
| MEF2C | ENSG00000081189 | NP_001180276.1 | X |
| MET | ENSG00000105976 | NP_001120972.1 | |
| MNT | ENSG00000070444 | NP_064706.1 | |
| MSR1 | ENSG00000038945 | NP_619729.1 | |
| NBEA | ENSG00000172915 | NP_056493.3 | |
| NFIA | ENSG00000162599 | NP_001138984.1 | X |
| NIPA2 | ENSG00000140157 | NP_001171818.1 | |
| NLGN3 | ENSG00000196338 | NP_851820.1 | |
| NLGN4Y | ENSG00000165246 | NP_055708.3 | |
| NPAS2 | ENSG00000170485 | NP_002509.2 | |
| NRCAM | ENSG00000091129 | NP_001032209.1 | |
| NRG1 | ENSG00000157168 | NP_039250.2 | X |
| NRXN1 | ENSG00000179915 | NP_001129131.1 | X |
| NRXN3 | ENSG00000021645 | NP_004787.2 | |
| NTRK3 | ENSG00000140538 | NP_001012338.1 | X |
| OGT | ENSG00000147162 | NP_858058.1 | |
| P4HA2 | ENSG00000072682 | NP_001136071.1 | |
| PAH | ENSG00000171759 | NP_000268.1 | |
| PAX6 | ENSG00000007372 | NP_001245391.1 | X |
| PLXNB1 | ENSG00000164050 | NP_001123554.1 | |
| PSD3 | ENSG00000156011 | NP_056125.3 | |
| PTBP2 | ENSG00000117569 | NP_067013.1 | X |
| PTGER3 | ENSG00000050628 | NP_942011.1 | X |
| PTK7 | ENSG00000112655 | NP_001257327.1 | |
| PTPRC | ENSG00000262418, | NP_002829.3 | X |
| | ENSG00000081237 | | |
| RBM8A | ENSG00000265241 | NP_005096.1 | |
| RERE | ENSG00000142599 | NP_036234.3 | |
| RGS7 | ENSG00000182901 | NP_002915.3 | |
| RIMS1 | ENSG00000079841 | NP_055804.2 | |
| RNPS1 | ENSG00000205937 | NP_542161.1 | |
| RPL10 | ENSG00000147403 | NP_001243506.2 | |
| SAE1 | ENSG00000142230 | NP_005491.1 | |
| SCFD2 | ENSG00000184178 | NP_689753.2 | |
| SCN9A | ENSG00000169432 | NP_002968.1 | |
| SDK1 | ENSG00000146555 | NP_689957.3 | |
| SHANK2 | ENSG00000162105 | NP_036441.2 | |
| SIN3A | ENSG00000169375 | NP_001138829.1 | X |
| SLC24A2 | ENSG00000155886 | NP_065077.1 | X |
| SLC25A14 | ENSG00000102078 | NP_003942.1 | |
| SLC33A1 | ENSG00000169359 | NP_001177921.1 | X |
| SLC7A3 | ENSG00000165349 | NP_116192.4 | |
| SMARCA2 | ENSG00000080503 | NP_003061.3 | |
| SNRPN | ENSG00000128739 | NP_073716.1 | X |
| SPAST | ENSG00000021574 | NP_055761.2 | |
| SRGAP3 | ENSG00000196220 | NP_055665.1 | |
| ST7 | ENSG00000004866 | NP_068708.1 | |
| STXBP5 | ENSG00000164506 | NP_001121187.1 | |
| SYN3 | ENSG00000185666 | NP_003481.3 | |
| SYNE1 | ENSG00000131018 | NP_892006.3 | |
| TAF1 | ENSG00000147133 | NP_004597.2 | |
| TAF1C | ENSG00000103168 | NP_005670.3 | |
| TBL1X | ENSG00000101849 | NP_001132938.1 | |
| TCF20 | ENSG00000276461, | NP_005641.1 | |
| | ENSG00000262024, | | |
| | ENSG00000100207, | | |
| | ENSG00000280467, | | |
| | ENSG00000281897, | | |
| | ENSG00000283026, | | |
| | ENSG00000282892, | | |
| | ENSG00000283681 | | |
| TERT | ENSG00000164362 | NP_937983.2 | |
| THRA | ENSG00000126351 | NP_003241.2 | |
| TOP3B | ENSG00000100038 | NP_003926.1 | X |
| TPO | ENSG00000115705, | NP_000538.3 | |
| | ENSG00000277603 | | |
| TSC2 | ENSG00000103197 | NP_000539.2 | |
| TTN | ENSG00000155657 | NP_001254479.1 | |
| UBE3B | ENSG00000151148 | NP_904324.1 | |
| UPF3B | ENSG00000125351 | NP_542199.1 | |
| USP45 | ENSG00000123552 | NP_001073950.1 | |
| VIP | ENSG00000146469 | NP_003372.1 | |
| WAC | ENSG00000095787 | NP_057712.2 | |
| WWOX | ENSG00000186153 | NP_057457.1 | X |
| ZBTB20 | ENSG00000181722 | NP_001157814.1 | |
| ZMYND11 | ENSG00000015171 | NP_006615.2 | |
| ZNF18 | ENSG00000154957 | NP_653281.2 | |

*Representative protein in the family encoded by the gene.

TABLE 14
(78 Genes) - SFARI gene list with hnRNP L binding sites
near splice events specifically observed in autism.

| | | | Genes with |
|---|---|---|---|
| | | | hnRNP binding |
| | | | sites within |
| | | | 200 base |
| | | | pairs of an |
| | Ensembl | Accession | RBFox1/A2BP1 |
| Human Gene | Identifier | Number* | binding site |
| ADCY5 | ENSG00000173175 | NP_899200.1 | |
| ANK2 | ENSG00000145362 | NP_001139.3 | |
| ANKRD11 | ENSG00000167522 | NP_001243111.1 | |
| ANKS1B | ENSG00000185046 | NP_690001.3 | X |
| APC | ENSG00000134982 | NP_001120982.1 | |
| ASTN2 | ENSG00000148219 | NP_054729.3 | |
| ATG7 | ENSG00000197548 | NP_006386.1 | |
| ATXN7 | ENSG00000163635 | NP_001170858.1 | X |
| BAIAP2 | ENSG00000175866 | NP_059345.1 | |
| BCAS1 | ENSG00000064787 | NP_003648.2 | X |
| CACNA1C | ENSG00000151067 | NP_955630.2 | |
| CACNA1E | ENSG00000198216 | NP_001192222.1 | |
| CACNA1G | ENSG00000006283 | NP_061496.2 | |
| CADM1 | ENSG00000182985 | NP_055148.3 | |
| CADPS2 | ENSG00000081803 | NP_001161412.1 | |
| CAMSAP2 | ENSG00000118200 | NP_982284.1 | |
| CAMTA1 | ENSG00000171735 | NP_056030.1 | X |
| CASC4 | ENSG00000166734 | NP_612432.2 | |
| CSMD1 | ENSG00000183117 | NP_150094.5 | X |
| CSNK1D | ENSG00000141551 | NP_001884.2 | |
| CUL7 | ENSG00000044090 | NP_001161842.1 | |
| DAPK1 | ENSG00000196730 | NP_004929.2 | |
| DST | ENSG00000151914 | NP_056363.2 | |
| ERBB4 | ENSG00000178568 | NP_005226.1 | X |
| GNAS | ENSG00000087460 | NP_536350.2 | X |
| GRIN1 | ENSG00000176884 | NP_001172019.1 | X |
| GSN | ENSG00000148180 | NP_000168.1 | |
| GTF2I | ENSG00000263001 | NP_127492.1 | |
| HDAC3 | ENSG00000171720 | NP_003874.2 | |
| HMGN1 | ENSG00000205581 | NP_004956.5 | |
| HUWE1 | ENSG00000086758 | NP_113584.3 | |
| KCNMA1 | ENSG00000156113 | NP_001154824.1 | |
| KCNQ2 | ENSG00000281151, | NP_742105.1 | |
| | ENSG00000075043 | | |
| KMT2A | ENSG00000118058 | NP_005924.2 | X |
| LAMB1 | ENSG00000091136 | NP_002282.2 | X |
| MAOB | ENSG00000069535 | NP_000889.3 | X |
| MAP2 | ENSG00000078018 | NP_002365.3 | X |
| MARK1 | ENSG00000116141 | NP_061120.3 | |
| MBD1 | ENSG00000141644 | NP_001191065.1 | X |
| MBD6 | ENSG00000166987 | NP_443129.3 | |
| MEF2C | ENSG00000081189 | NP_001180276.1 | X |
| MPP6 | ENSG00000105926 | NP_057531.2 | |
| MTOR | ENSG00000198793 | NP_004949.1 | |
| MYO9B | ENSG00000099331 | NP_004136.2 | X |
| NELL1 | ENSG00000165973 | NP_006148.2 | |
| NEO1 | ENSG00000067141 | NP_002490.2 | X |
| NFIX | ENSG00000008441 | NP_001257972.1 | X |
| NPAS2 | ENSG00000170485 | NP_002509.2 | |
| NRCAM | ENSG00000091129 | NP_001032209.1 | X |
| NRXN1 | ENSG00000179915 | NP_001129131.1 | X |
| NRXN3 | ENSG00000021645 | NP_004787.2 | X |
| OGT | ENSG00000147162 | NP_858058.1 | |
| P4HA2 | ENSG00000072682 | NP_001136071.1 | |
| PAFAH1B1 | ENSG00000007168 | NP_000421.1 | |
| PER1 | ENSG00000179094 | NP_002607.2 | X |
| PRKD1 | ENSG00000184304 | NP_002733.2 | |
| PRUNE2 | ENSG00000106772 | NP_056040.2 | |
| RAPGEF4 | ENSG00000091428 | NP_008954.2 | |
| RBFOX1/ | ENSG00000078328 | NP_665898.1 | |
| A2BP1 | | | |
| RFWD2 | ENSG00000143207 | NP_071902.2 | X |
| RGS7 | ENSG00000182901 | NP_002915.3 | X |
| RIMS1 | ENSG00000079841 | NP_055804.2 | X |
| SCN1A | ENSG00000144285 | NP_001159435.1 | |
| SCN8A | ENSG00000196876 | NP_055006.1 | |
| SETD5 | ENSG00000168137 | NP_001073986.1 | |
| SGSH | ENSG00000181523 | NP_000190.1 | X |
| SMARCC2 | ENSG00000139613 | NP_003066.2 | |
| SNAP25 | ENSG00000132639 | NP_003072.2 | |
| ST7 | ENSG00000004866 | NP_068708.1 | X |
| SYNE1 | ENSG00000131018 | NP_892006.3 | |
| TBC1D5 | ENSG00000131374 | NP_001127853.1 | |
| TBC1D7 | ENSG00000145979 | NP_001137436.1 | |
| TCF4 | ENSG00000196628 | NP_001230155.1 | |
| TLK2 | ENSG00000146872 | NP_006843.2 | |
| TRIM33 | ENSG00000197323 | NP_056990.3 | |
| TSC2 | ENSG00000103197 | NP_000539.2 | |
| WDFY3 | ENSG00000163625 | NP_055806.2 | |
| ZBTB16 | ENSG00000109906 | NP_005997.2 | |

*Representative protein in the family encoded by the gene.

TABLE 15
(27 Genes) - Genes bearing the hnRNP L binding site, that comprise the
SHANK-TSC-mTOR-ERK ASD-associated pathways (pathways described in Goldani
et al., Biomarkers in autism. Front Psychiatry. 2014 Aug. 12;5:100).

| Gene symbol | Table # | Name in Ref Figures | Patient cohort | Comment |
|---|---|---|---|---|
| NRXN1 | 2, 3, 4 | Neurexin, NRX | | |
| NRXN2 | 2 | Neurexin, NRX | | Established target |
| | | | | of hnRNP L (ii) |
| NRXN3 | 2, 3, 4 | Neurexin, NRX | | |
| NLGN3 | 2, 3 | Neuroligin, NLGN | | |
| NLGN4X | 2 | Neuroligin, NLGN | | |
| NLGN4Y | 2, 3 | Neuroligin, NLGN | | |
| SHANK2 | 2, 3 | SHANK | | |
| SHANK3 | 2 | SHANK | | |
| NF1 | 2 | NF1 | Neurofibromatosis | Multiple splice |
| | | | | site mutations |
| TSC1 | 2 | TSC1 | Tuberous sclerosis | Multiple splice |
| | | | | site mutations |
| TSC2 | 2, 3, 4 | TSC2 | Tuberous sclerosis | Multiple splice |
| | | | | site mutations |
| MTOR | 4 | mTOR | | |
| FMR1 | 2, 3 | FMRP, FMR1 | (Fragile X syndrome) | |
| EIF4E | 2 | eiF4E | | |
| CACNA1C | 2, 3, 4 | CACNA1C | (Timothy syndrome) | |
| GRIN1 | 2, 4 | NMDAR | | |
| GRM1 | 2 | mGluR | | |
| DRD2 | 2, 3 | DRD2 | | |
| MAPK3 | 2 | ERK | | |
| GSK3B | 2 | GSK-3β | | |
| GABRB3 | 2, 3 | GABAR | | |
| SCN1A | 4 | Scn1A | | |
| MET | 2, 3 | MET | | |
| HRAS | 2 | Ras | | |
| VLDLR | 2 | VLDLR | | |
| AKAP9 | 2 | PKA | | |
| CADPS2 | 2, 4 | CADPS2 | | presynaptic |

TABLE 16
(18 Genes) - Genes bearing the hnRNP L binding site, that
comprise the SHANK-TSC- ASD disease module (i, ii)

| Gene symbol | Table # | Name in Ref Figures | Patient cohort | Comment |
|---|---|---|---|---|
| NRXN1 | 2, 3, 4 | Neurexin, NRX | | |
| NRXN2 | 2 | Neurexin, NRX | | Established target of |
| | | | | hnRNP L (ii) |
| NRXN3 | 2, 3, 4 | Neurexin, NRX | | |
| NLGN3 | 2, 3 | Neuroligin, NLGN | | |
| NLGN4X | 2 | Neuroligin, NLGN | | |
| NLGN4Y | 2, 3 | Neuroligin, NLGN | | |
| SHANK2 | 2, 3 | SHANK | | |
| SHANK3 | 2 | SHANK | | |
| NF1 | 2 | NF1 | Neurofibromatosis | Multiple splice site |
| | | | | mutations |
| TSC1 | 2 | TSC1 | Tuberous sclerosis | Multiple splice site |
| | | | | mutations |
| TSC2 | 2, 3, 4 | TSC2 | Tuberous sclerosis | Multiple splice site |
| | | | | mutations |
| MTOR | 4 | mTOR | | |
| FMR1 | 2, 3 | FMRP, FMR1 | (Fragile X syndrome) | |
| EIF4E | 2 | eiF4E | | |
| CACNA1C | 2, 3, 4 | CACNA1C | (Timothy syndrome) | |
| GRIN1 | 2, 4 | NMDAR | | |
| GRM1 | 2 | mGluR | | |
| CADPS2 | 2, 4 | CADPS2, CAPS2 | | presynaptic |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

### INCORPORATION BY REFERENCE

All of the cited U.S. patents, U.S. patent application publications and PCT patent applications designating the U.S., are hereby incorporated by reference in their entirety.

### EQUIVALENTS

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto; aspects provided herein may be practiced otherwise than as specifically described and claimed.

What is claimed is:

1. A method of treating a subject with a Cryptic Exon Induced Neurological Disease (CEIND) or Poison Exon Induced Neurological Disease (PEIND), comprising administering to said subject an antisense oligonucleotide (ASO) that binds a region of an hnRNP L mRNA and increases an amount of hnRNP L protein or RNA in the subject relative to a baseline amount, wherein the region comprises poison exon 6A or a surrounding intron region of poison exon 6A.

2. The method of claim 1, wherein the region the ASO binds comprises poison exon 6A.

3. The method of claim 1, wherein the region the ASO binds comprises a sequence of the intron immediately upstream (5′) of poison exon 6A.

4. The method of claim 1, wherein the region the ASO binds comprises a sequence of the intron immediately downstream (3′) of poison exon 6A.

5. The method of claim 1, wherein poison exon 6A comprises or is encoded by the following sequence:
(SEQ ID NO: 102)
GGTCGCAGTGTATGTTTGATGGGACGCCATCTTTCAGAACTGTGCTAAC
TCACTGTTGAAGCGTCCAATG.

6. The method of claim 1, wherein the ASO comprises the base sequence of any one of SEQ ID NOs: 47-69, or a sequence having 1 or 2 insertions, substitutions, or deletions relative to any one of SEQ ID NOs: 47-69.

7. The method of claim 1, wherein the ASO comprises the base sequence of any one of SEQ ID NOs: 47-69.

8. A method of treating a subject with a Cryptic Exon Induced Neurological Disease (CEIND) or Poison Exon Induced Neurological Disease (PEIND), comprising administering to said subject an agent to increase expression levels and/or stability of hnRNP L, thereby attenuating the expression of cryptic or poison exons.

9. The method of claim 8, wherein the neurological disease is associated with a splicing defect caused by one or more TDP-43 proteinopathies.

10. The method of claim 9, wherein the one or more TDP-43 proteinopathies are caused by a loss of or altered TDP-43 function.

11. The method of claim 10, wherein the loss of or altered TDP-43 function is due to
a mutation in the TDP-43 gene or open reading frame; and/or
an altered TDP-43 function due to one or more of nuclear clearance, cytoplasmic inclusions, nuclear inclusions, aggregation, abnormal modification, and neuronal propagation in a “prion-like” manner.

12. The method of claim 8, wherein the mutation comprises at least one selected from the group consisting of: D169G, K263E, N267S, G287S, G290A, S292N, G294A, G294V, G295R, G295S, G298S, M311V, A31ST, A321V, A321G, Q331K, S332N, G335D, M337V, Q343R, N345K, G348C, G348V, N352S/T, R361S, P363A, Y374X, N378D, S379P, S379C, A382P, A382T, I383V, G384R, N390D, N390S, and S393L.

13. The method of claim 10, wherein said loss of or altered TDP-43 function promotes cryptic exon or poison exon inclusion.

14. The method of claim 10, wherein said loss of or altered TDP-43 function reduces expression levels of the normal transcript(s) of UNC13A, UNC13B, STMN2, SORT1, GPSM2, and/or ATG4B.

15. The method of claim 10, wherein said loss of or altered TDP-43 function promotes a splicing defect in a UNC13A, UNC13B, STMN2, SORT1, GPSM2, and/or ATG4B gene.

16. The method of claim 10, wherein said loss of or altered TDP-43 function inhibits neurite and/or axon growth.

17. The method of claim 8, wherein the neurological disease comprises a cryptic exon-induced neurological disease (CEIND) or poison exon-induced neurological disease (PEIND).

18. The method of claim 17, wherein the CEIND or PEIND comprises Autism Spectrum Disorder, Intellectual Disability, Amyotrophic Lateral Sclerosis (ALS), Frontotemporal lobar degeneration (FTLD), Frontotemporal Dementia (FTD), myotonic dystrophy type 1 (DM1), Alzheimer's disease, Lewy body dementia (LBD), Pick's disease, Hippocampal sclerosis, Corticobasal degeneration, Huntington disease, Parkinson's disease, Argyrophilic grain disease, Chronic traumatic encephalopathy (CTE), Perry syndrome, Alexander disease, Multisystem proteinopathy (MSP), intellectual disability, ADHD, dyslexia, epilepsy, bipolar disorder, depression and schizophrenia, parkinsonism-dementia complex of Guam, dementia pugilistica, diffuse neurofibrillary tangles with calcification, Down's syndrome, familial British dementia, familial Danish dementia, frontal lobe dementia, Gerstmann-Straussler-Scheinker disease, globular glial tauopathies, white matter tauopathy with globular glial inclusions, Guadeloupian parkinsonism with dementia, Guadeloupian progressive supranuclear palsy, multiple system atrophy, myotonic dystrophy, neurodegeneration with brain iron accumulation, Hallervorden-Spatz disease, pantothenate kinase-associated neurodegeneration, neurofibrillary tangle-predominant dementia, Niemann-Pick disease, prosencephalitic parkinsonism, prion protein cerebral amyloid angiopathy, progressive supranuclear palsy, SLCI A6-related mental retardation, subacute sclerosing panencephalitis, Alzheimer's disease, chronic traumaticencephalopathy (CTE), limbic predominant, age-related TDP-43 encephalopathy (LATE), and inclusion body myopathy.

19. The method of claim 7, wherein the increased expression levels and/or stability of hnRNP L reduces the splicing defect caused by a TDP-43 proteinopathy.

20. The method of claim 7, wherein the increased expression levels and/or stability of hnRNP L reduces a splicing defect independent of TDP-43.

21. The method of claim 8, wherein the agent comprises at least one of a nucleic acid, an antisense oligonucleotide (ASO), a small interfering RNA, a microRNA, a lncRNA, a small hairpin RNA, an aptamer, a PNA, a small molecule, an enzyme, a protein, a polypeptide, an antibody or a functional fragment thereof.

22. The method of claim 21, wherein the agent comprises an ASO.

23. The method of claim 22, wherein the ASO increases expression of hnRNP L.

24. The method of claim 22, wherein the ASO targets a poison exon of hnRNP L, or targets an intron immediately upstream or immediately downstream of the poison exon.

25. The method of claim 22, wherein the ASO targets an upstream intronic sequence between exon 6 and poison exon 6A of hnRNP L.

26. The method of claim 24, wherein the poison exon comprises or is encoded by the following sequence:
(SEQ ID NO: 102)
GGTCGCAGTGTATGTTTGATGGGACGCCATCTTTCAGAACTGTGCTAAC
TCACTGTTGAAGCGTCCAATG.

27. The method of claim 22, wherein the ASO comprises the base sequence of any one of SEQ ID NOs: 47-101, or a sequence having 1 or 2 insertions, substitutions, or deletions relative to any one of SEQ ID NOs: 47-101.

28. The method of claim 22, wherein the ASO comprises the base sequence of any one of SEQ ID NOs: 47-69, or a sequence having 1 or 2 insertions, substitutions, or deletions relative to any one of SEQ ID NOs: 47-69.

29. The method of claim 22, wherein the ASO increases hnRNP L expression by at least 1.05 fold, at least 1.1 fold, at least 1.15 fold, at least 1.2 fold, at least 1.25 fold, at least 1.3 fold, at least 1.35 fold, at least 1.4 fold, at least 1.45 fold, at least 1.5 fold, at least 1.55 fold, at least 1.6 fold, at least 1.65 fold, at least 1.7 fold, at least 1.75 fold, at least 1.8 fold, at least 1.85 fold, at least 1.9 fold, at least 1.95 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 50 fold, at least 60 fold, at least 70 fold, at least 80 fold, at least 90 fold, or at least 100 fold.

30. The method of claim 22, wherein the ASO comprises the base sequence of an ASO that increased hnRNP L by at least 25%, at least 30%, at least 35%, at least 40%, at least 45%, at least 50%, at least 55%, at least 60%, at least 65%, at least 70%, at least 75%, or at least 80% in Table 9B or 10, or a sequence thereof having 1 or 2 insertions, substitutions, or deletions.

31. The method of claim 22, wherein the ASO has a measured EC50 value below 1000 nM, 750 nM, 500 nM, 250 nM, 100 nM, 75 nM, 50 nM, 25 nM, 20 nM, below 15 nM, below 14 nM, below 13 nM, below 12 nM, below 11 nM, below 10 nM, below 9 nM, below 8 nM, below 7 nM, below 6 nM, below 5 nM, below 4 nM, or below 3 nM (e.g. in Table 9B).

32. The method of claim 22, wherein the ASO comprises a DNA oligonucleotide.

33. The method of claim 22, wherein the ASO comprises a nucleoside modification.

34. The method of claim 33, wherein the nucleoside modification comprises 2′-O-methoxyethyl (MOE).

35. The method of claim 33, wherein the nucleoside modification comprises 5′-methyl C.

36. The method of claim 22, wherein the ASO comprises an internucleoside linkage modification.

37. The method of claim 36, wherein the internucleoside linkage modification comprises a phosphorothioate linkage.

38. A composition comprising an agent capable of increasing expression levels and/or stability of hnRNP L, for treating a subject with a neurological disease associated with a splicing defect.

39. The composition of claim 38, wherein said subject has a loss of or altered TDP-43 function.

40. The composition of claim 39, wherein said loss of or altered TDP-43 function promotes cryptic exon or poison exon inclusion.

41. The composition of claim 39, wherein said loss of or altered TDP-43 function reduces expression levels of the normal transcript(s) of UNC13A, UNC13B, STMN2, SORT1, GPSM2, and/or ATG4B.

42. The composition of claim 39, wherein said loss of or altered TDP-43 function promotes a splicing defect in a UNC13A, UNC13B, STMN2, SORT1, GPSM2, and/or ATG4B gene.

43. The composition of claim 39, wherein said loss of or altered TDP-43 function inhibits neurite and/or axon growth.

44. The composition of claim 38, wherein the neurological disease comprises a CEIND or PEIND.

45. The composition of claim 44, wherein the CEIND or PEIND comprises Autism Spectrum Disorder, Intellectual Disability, Amyotrophic Lateral Sclerosis (ALS), Frontotemporal lobar degeneration (FTLD), Frontotemporal Dementia (FTD), myotonic dystrophy type 1 (DM1), Alzheimer's disease, Lewy body dementia (LBD), Pick's disease, Hippocampal sclerosis, Corticobasal degeneration, Huntington disease, Parkinson's disease, Argyrophilic grain disease, Chronic traumatic encephalopathy (CTE), Perry syndrome, Alexander disease, Multisystem proteinopathy (MSP), intellectual disability, ADHD, dyslexia, epilepsy, bipolar disorder, depression and schizophrenia, parkinsonism-dementia complex of Guam, dementia pugilistica, diffuse neurofibrillary tangles with calcification, Down's syndrome, familial British dementia, familial Danish dementia, frontal lobe dementia, Gerstmann-Straussler-Scheinker disease, globular glial tauopathies, white matter tauopathy with globular glial inclusions, Guadeloupian parkinsonism with dementia, Guadeloupian progressive supranuclear palsy, multiple system atrophy, myotonic dystrophy, neurodegeneration with brain iron accumulation, Hallervorden-Spatz disease, pantothenate kinase-associated neurodegeneration, neurofibrillary tangle-predominant dementia, Niemann-Pick disease, prosencephalitic parkinsonism, prion protein cerebral amyloid angiopathy, progressive supranuclear palsy, SLCI A6-related mental retardation, subacute sclerosing panencephalitis, Alzheimer's disease, chronic traumaticencephalopathy (CTE), limbic predominant, age-related TDP-43 encephalopathy (LATE), and inclusion body myopathy.

46. The composition of claim 38, wherein the increased expression levels and/or stability of hnRNP L reduces the splicing defect caused by a TDP-43 proteinopathy.

47. The composition of claim 38, wherein the increased expression levels and/or stability of hnRNP L reduces a splicing defect independent of TDP-43.

48. The composition of claim 38, wherein the agent comprises at least one of a nucleic acid, an antisense oligonucleotide (ASO), a small interfering RNA, a microRNA, a small hairpin RNA, an aptamer, a PNA, a small molecule, an enzyme, a protein, a polypeptide, or an antibody or a functional fragment thereof.

49. The composition of claim 48, wherein the agent comprises an ASO.

50. The composition of claim 49, wherein the ASO increases expression of hnRNP L.

51. The composition of claim 49, wherein the ASO targets a poison exon of hnRNP L, or targets an intron immediately upstream or downstream of the poison exon.

52. The composition of claim 51, wherein the poison exon comprises the following sequence:
(SEQ ID NO: 102)
GGTCGCAGTGTATGTTTGATGGGACGCCATCTTTCAGAACTGTGCTAAC
TCACTGTTGAAGCGTCCAATG.

53. The composition of claim 49, wherein the ASO comprises the base sequence of any one of SEQ ID NOs: 47-101, or a sequence having 1 or 2 insertions, substitutions, or deletions relative to any one of SEQ ID NOs: 47-101.

54. The composition of claim 49, wherein the ASO comprises the base sequence of any one of SEQ ID NOs: 47-69, or a sequence having 1 or 2 insertions, substitutions, or deletions relative to any one of SEQ ID NOs: 47-69.

55. The composition of claim 49, wherein the ASO increases hnRNP L expression by at least 1.05 fold, at least 1.1 fold, at least 1.15 fold, at least 1.2 fold, at least 1.25 fold, at least 1.3 fold, at least 1.35 fold, at least 1.4 fold, at least 1.45 fold, at least 1.5 fold, at least 1.55 fold, at least 1.6 fold, at least 1.65 fold, at least 1.7 fold, at least 1.75 fold, at least 1.8 fold, at least 1.85 fold, at least 1.9 fold, at least 1.95 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 50 fold, at least 60 fold, at least 70 fold, at least 80 fold, at least 90 fold, or at least 100 fold.

56. The composition of claim 49, wherein the ASO comprises the base sequence of an ASO that increased hnRNP L by at least 25%, at least 30%, at least 35%, at least 40%, at least 45%, at least 50%, at least 55%, at least 60%, at least 65%, at least 70%, at least 75%, or at least 80% in Table 9B or 10, or a sequence thereof having 1 or 2 insertions, substitutions, or deletions.

57. The composition of claim 49, wherein the ASO has a measured EC50 value below 1000 nM, 750 nM, 500 nM, 250 nM, 100 nM, 75 nM, 50 nM, 25 nM, 20 nM, below 15 nM, below 14 nM, below 13 nM, below 12 nM, below 11 nM, below 10 nM, below 9 nM, below 8 nM, below 7 nM, below 6 nM, below 5 nM, below 4 nM, or below 3 nM (e.g. in Table 9B).

58. The composition of claim 49, wherein the ASO comprises a DNA oligonucleotide.

59. The composition of claim 49, wherein the ASO comprises a nucleoside modification.

60. The composition of claim 59, wherein the nucleoside modification comprises 2′-O-methoxyethyl (MOE).

61. The composition of claim 59, wherein the nucleoside modification comprises 5′-methyl C.

62. The composition of claim 49, wherein the ASO comprises an internucleoside linkage modification.

63. The composition of claim 62, wherein the internucleoside linkage modification comprises a phosphorothioate linkage.

64. A pharmaceutical composition for treating a subject with a neurological disease associated with a splicing defect caused by TDP-43 proteinopathies, comprising the composition of claim 38 and a pharmaceutically acceptable carrier.

65. A kit comprising a composition of claim 38 or a pharmaceutical composition of claim 64.

66. A method of treating a subject with a hnRNP L proteinopathy-dependent neurological disease, comprising administering to said subject an agent to increase expression levels and/or stability of hnRNP L, thereby repairing said splicing defect.

67. A method of treating a subject with a cryptic exon-, poison exon- or intron retention-dependent neurological disease, comprising administering to said subject an agent to increase expression levels and/or stability of hnRNP L, thereby repairing said cryptic exon, poison exon, or intron retention defect.

68. A method of mitigating age-induced neurological disease, or neurologic function decline, comprising administering to said subject an agent to increase expression levels and/or stability of hnRNP L.

69. A method for mitigating neuronal hypoxia-induced neurological disease including amyotrophic lateral sclerosis, comprising administering to said subject an agent to increase expression levels and/or stability of hnRNP L.


SEQUENCE LISTING (104 sequences)

SEQ ID NO: 1 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 2 - protein, 298 aa, Homo sapiens
MPQMLAGEIWCMLSTIQKVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 3 - protein, 589 aa, Homo sapiens
MSRRLLPRAEKRRRRLEQRQQPDEQRRRSGAMVKMAAAGGGGGGGRYYGGGSEGGRAPKRLKTDNAGDQHGGGGGGGGGAGAAGGGGGGENYDDPHKTPASPVVHIRGLIDGVVEADLVEALQEFGPISYVVVMPKKRQALVEFEDVLGACNAVNYAADNQIYIAGHPAFVNYSTSQKISRPGDSDDSRSVNSVLLFTILNPIYSITTDVLYTICNPCGPVQRIVIFRKNGVQAMVEFDSVQSAQRAKASLNGADIYSGCCTLKIEYAKPTRLNVFKNDQDTWDYTNPNLSGQGDPGSNPNKRQRQPPLLGDHPAEYGGPHGGYHSHYHDEGYGPPPPHYEGRRMGPPVGGHRRGPSRYGPQYGHPPPPPPPPEYGPHADSPVLMVYGLDQSKMNCDRVFNVFCLYGNVEKVKFMKSKPGAAMVEMADGYAVDRAITHLNNNFMFGQKLNVCVSKQPAIMPGQSYGLEDGSCSYKDFSESRNNRFSTPEQAAKNRIQHPSNVLHFFNAPLEVTEENFFEICDELGVKRPSSVKVFSGKSERSSSGLLEWESKSDALETLGFLNHYQMKNPNGPYPYTLKLCFSTAQHAS

SEQ ID NO: 4 - protein, 456 aa, Homo sapiens
MPKKRQALVEFEDVLGACNAVNYAADNQIYIAGHPAFVNYSTSQKISRPGDSDDSRSVNSVLLFTILNPIYSITTDVLYTICNPCGPVQRIVIFRKNGVQAMVEFDSVQSAQRAKASLNGADIYSGCCTLKIEYAKPTRLNVFKNDQDTWDYTNPNLSGQGDPGSNPNKRQRQPPLLGDHPAEYGGPHGGYHSHYHDEGYGPPPPHYEGRRMGPPVGGHRRGPSRYGPQYGHPPPPPPPPEYGPHADSPVLMVYGLDQSKMNCDRVFNVFCLYGNVEKVKFMKSKPGAAMVEMADGYAVDRAITHLNNNFMFGQKLNVCVSKQPAIMPGQSYGLEDGSCSYKDFSESRNNRFSTPEQAAKNRIQHPSNVLHFFNAPLEVTEENFFEICDELGVKRPSSVKVFSGKSERSSSGLLEWESKSDALETLGFLNHYQMKNPNGPYPYTLKLCFSTAQHAS

SEQ ID NO: 5-13 - skipped (9 entries)

SEQ ID NO: 14 - protein, 831 aa, Homo sapiens
MERPWGAADGLSRWPHGLGLLLLLQLLPPSTLSQDRLDAPPPPAAPLPRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE

SEQ ID NO: 15 - protein, 694 aa, Homo sapiens
MTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCTDLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE

SEQ ID NO: 16 - protein, 684 aa, Homo sapiens
MEENLISMREDHSFHVRYRMEASCLELALEGERLCKSGDCRAGVSFFEAAVQVGTEDLKTLSAIYSQLGNAYFYLHDYAKALEYHHHDLTLARTIGDQLGEAKASGNLGNTLKVLGNFDEAIVCCQRHLDISRELNDKVGEARALYNLGNVYHAKGKSFGCPGPQDVGEFPEEVRDALQAAVDFYEENLSLVTALGDRAAQGRAFGNLGNTHYLLGNFRDAVIAHEQRLLIAKEFGDKAAERRAYSNLGNAYIFLGEFETASEYYKKTLLLARQLKDRAVEAQSCYSLGNTYTLLQDYEKAIDYHLKHLAIAQELNDRIGEGRACWSLGNAYTALGNHDQAMHFAEKHLEISREVGDKSGELTARLNLSDLQMVLGLSYSTNNSIMSENTEIDSSLNGVRPKLGRRHSMENMELMKLTPEKVQNWNSEILAKQKPLIAKPSAKLLFVNRLKGKKYKTNSSTKVLQDASNSIDHRIPNSQRKISADTIGDEGFFDLLSRFQSNRMDDQRCCLQEKNCHTASTTTSSTPPKMMLKTSSVPVVSPNTDEFLDLLASSQSRRLDDQRASFSNLPGLRLTQNSQSVLSHLMTNDNKEADEDFFDILVKCQGSRLDDQRCAPPPATTKGPTVPDEDFFSLILRSQGKRMDEQRVLLQRDQNRDTDFGLKDFLQNNALLEFKNSGKKSADH

SEQ ID NO: 17 - protein, 393 aa, Homo sapiens
MDAATLTYDTLRFAEFEDFPETSEPVWILGRKYSIFTEKDEILSDVASRLWFTYRKNFPAIGGTGPTSDTGWGCMLRCGQMIFAQALVCRHLGRDWRWTQRKRQPDSYFSVLNAFIDRKDSYYSIHQIAQMGVGEGKSIGQWYGPNTVAQVLKKLAVFDTWSSLAVHIAMDNTVVMEEIRRLCRTSVPCAGATAFPADSDRHCNGFPAGAEVTNRPSPWRPLVLLIPLRLGLTDINEAYVETLKHCFMMPQSLGVIGGKPNSAHYFIGYVGEELIYLDPHTTQPAVEPTDGCFIPDESFHCQHPPCRMSIAELDPSIAVGFFCKTEDDFNDWCQQVKKLSLLGGALPMFELVELQPSHLACPDVLNLSLDSSDVERLERFFDSEDEDFEILSL

SEQ ID NO: 18 - protein, 468 aa, Homo sapiens
MAHSVPSDSRTSRRPTTRPHAARGAPRGSRRPGRTPKWRLPRISARAPYRLRRLRRHTYWPPRRPVAASRCWPVGATPLGSVGGRTGKMDAATLTYDTLRFAEFEDFPETSEPVWILGRKYSIFTEKDEILSDVASRLWFTYRKNFPAIGGTGPTSDTGWGCMLRCGQMIFAQALVCRHLGRDWRWTQRKRQPDSYFSVLNAFIDRKDSYYSIHQIAQMGVGEGKSIGQWYGPNTVAQVLKKLAVFDTWSSLAVHIAMDNTVVMEEIRRLCRTSVPCAGATAFPADSDRHCNGFPAGAEVTNRPSPWRPLVLLIPLRLGLTDINEAYVETLKHCFMMPQSLGVIGGKPNSAHYFIGYVGEELIYLDPHTTQPAVEPTDGCFIPDESFHCQHPPCRMSIAELDPSIAVGFFCKTEDDFNDWCQQVKKLSLLGGALPMFELVELQPSHLACPDVLNLSLGESCQVQILLM

SEQ ID NO: 19 - protein, 280 aa, Homo sapiens
MLRCGQMIFAQALVCRHLGRDWRWTQRKRQPDSYFSVLNAFIDRKDSYYSIHQIAQMGVGEGKSIGQWYGPNTVAQVLKKLAVFDTWSSLAVHIAMDNTVVMEEIRRLCRTSVPCAGATAFPADSDRHCNGFPAGAEVTNRPSPWRPLVLLIPLRLGLTDINEAYVETLKHCFMMPQSLGVIGGKPNSAHYFIGYVGEELIYLDPHTTQPAVEPTDGCFIPDESFHCQHPPCRMSIAELDPSIAVGKQGRLVRSLIPWAPRPSSWCAAVLGAAVVMCGTP

SEQ ID NO: 20 - protein, 280 aa, Homo sapiens; identical to SEQ ID NO: 19

SEQ ID NO: 21 - protein, 380 aa, Homo sapiens
MDAATLTYDTLRFAEFEDFPETSEPVWILGRKYSIFTEKDEILSDVASRLWFTYRKNFPAIGGTGPTSDTGWGCMLRCGQMIFAQALVCRHLGRDWRWTQRKRQPDSYFSVLNAFIDRKDSYYSIHQIAQMGVGEGKSIGQWYGPNTVAQVLKKLAVFDTWSSLAVHIAMDNTVVMEEIRRLCRTSVPCAGATAFPADSDRHCNGFPAGAEVTNRPSPWRPLVLLIPLRLGLTDINEAYVETLKHCFMMPQSLGVIGGKPNSAHYFIGYVGEELIYLDPHTTQPAVEPTDGCFIPDESFHCQHPPCRMSIAELDPSIAVGFFCKTEDDFNDWCQQVKKLSLLGGALPMFELVELQPSHLACPDVLNLSLGESCQVQILLM

SEQ ID NO: 22 - protein, 187 aa, Homo sapiens
MAKTAMAYKEKMKELSMLSLICSCFYPEPRNINIYTYDDMEVKQINKRASGQAFELILKPPSPISEAPRTLASPKKKDLSLEEIQKKLEAAEERRKSQEAQVLKQLAEKREHEREVLQKALEENNNFSKMAEEKLILKMEQIKENREANLAAIIERLQEKLVKFISSELKESIESQFLELQREGEKQ

SEQ ID NO: 23 - protein, 179 aa, Homo sapiens
MAKTAMAYKEKMKELSMLSLICSCFYPEPRNINIYTYDDMEVKQINKRASGQAFELILKPPSPISEAPRTLASPKKKDLSLEEIQKKLEAAEERRKSQEAQVLKQLAEKREHEREVLQKALEENNNFSKMAEEKLILKMEQIKENREANLAAIIERLQEKERHAAEVRRNKELQVELSG

SEQ ID NO: 24 - DNA, 22 bp, Homo sapiens
  source 1..22: mol_type = genomic DNA
gaggcgggttctgcgccgccat

SEQ ID NO: 25 - DNA, 22 bp, Homo sapiens
  source 1..22: mol_type = genomic DNA
ttctgcgccgccatgtcgcgga

SEQ ID NO: 26 - DNA, 22 bp, Homo sapiens
  source 1..22: mol_type = genomic DNA
gccatgtcgcggaggctgctgc

SEQ ID NO: 27 - DNA, 22 bp, Homo sapiens
  source 1..22: mol_type = genomic DNA
aaaaattgtagcggatgtgtac

SEQ ID NO: 28 - DNA, 22 bp, Homo sapiens
  source 1..22: mol_type = genomic DNA
gtagcggatgtgtaccgtgact

SEQ ID NO: 29 - DNA, 22 bp, Homo sapiens
  source 1..22: mol_type = genomic DNA
tgtgtaccgtgacttgtattta

SEQ ID NO: 30 - DNA, 22 bp, Homo sapiens
  source 1..22: mol_type = genomic DNA
gtgacttgtatttatgactgta

SEQ ID NO: 31 - DNA, 22 bp, Homo sapiens
  source 1..22: mol_type = genomic DNA
gtatttatgactgtaaaaccat

SEQ ID NO: 32 - DNA, 22 bp, Homo sapiens
  source 1..22: mol_type = genomic DNA
gactgtaaaaccatgtgatgca

SEQ ID NO: 33 - DNA, 22 bp, Homo sapiens
  source 1..22: mol_type = genomic DNA
aaaccatgtgatgcagggtcgc

SEQ ID NO: 34 - DNA, 22 bp, Homo sapiens
  source 1..22: mol_type = genomic DNA
gtgatgcagggtcgcagtgtat

SEQ ID NO: 35 - DNA, 22 bp, Homo sapiens
  source 1..22: mol_type = genomic DNA
cagggtcgcagtgtatgtttga

SEQ ID NO: 36 - DNA, 22 bp, Homo sapiens
  source 1..22: mol_type = genomic DNA
cgcagtgtatgtttgatgggac

SEQ ID NO: 37 - DNA, 22 bp, Homo sapiens
  source 1..22: mol_type = genomic DNA
tgtatgtttgatgggacgccat

SEQ ID NO: 38 - DNA, 22 bp, Homo sapiens
  source 1..22: mol_type = genomic DNA
tttgatgggacgccatctttca

SEQ ID NO: 39 - DNA, 22 bp, Homo sapiens
  source 1..22: mol_type = genomic DNA
gggacgccatctttcagaactg

SEQ ID NO: 40 - DNA, 22 bp, Homo sapiens
  source 1..22: mol_type = genomic DNA
ccatctttcagaactgtgctaa

SEQ ID NO: 41 - DNA, 22 bp, Homo sapiens
  source 1..22: mol_type = genomic DNA
ttcagaactgtgctaactcact

SEQ ID NO: 42 - DNA, 22 bp, Homo sapiens
  source 1..22: mol_type = genomic DNA
ctgtgctaactcactgttgaag

SEQ ID NO: 43 - DNA, 22 bp, Homo sapiens
  source 1..22: mol_type = genomic DNA
taactcactgttgaagcgtcca

SEQ ID NO: 44 - DNA, 22 bp, Homo sapiens
  source 1..22: mol_type = genomic DNA
actgttgaagcgtccaatggta

SEQ ID NO: 45 - DNA, 22 bp, Homo sapiens
  source 1..22: mol_type = genomic DNA
gaagcgtccaatggtaagagaa

SEQ ID NO: 46 - DNA, 22 bp, Homo sapiens
  source 1..22: mol_type = genomic DNA
ccaatggtaagagaaaatacag

SEQ ID NO: 47 - DNA, 22 bp, Homo sapiens
  source 1..22: mol_type = other DNA
atggcggcgcagaacccgcctc

SEQ ID NO: 48 - DNA, 22 bp, Homo sapiens
  source 1..22: mol_type = other DNA
tccgcgacatggcggcgcagaa

SEQ ID NO: 49 - DNA, 22 bp, Homo sapiens
  source 1..22: mol_type = other DNA
gcagcagcctccgcgacatggc

SEQ ID NO: 50 - DNA, 22 bp, Homo sapiens
  source 1..22: mol_type = other DNA
gtacacatccgctacaattttt

SEQ ID NO: 51 - DNA, 22 bp, Homo sapiens
  source 1..22: mol_type = other DNA
agtcacggtacacatccgctac

SEQ ID NO: 52 - DNA, 22 bp, Homo sapiens
  source 1..22: mol_type = other DNA
taaatacaagtcacggtacaca

SEQ ID NO: 53 - DNA, 22 bp, Homo sapiens
  source 1..22: mol_type = other DNA
tacagtcataaatacaagtcac

SEQ ID NO: 54 - DNA, 22 bp, Homo sapiens
  source 1..22: mol_type = other DNA
atggttttacagtcataaatac

SEQ ID NO: 55 - DNA, 22 bp, Homo sapiens
  source 1..22: mol_type = other DNA
tgcatcacatggttttacagtc

SEQ ID NO: 56 - DNA, 22 bp, Homo sapiens
  source 1..22: mol_type = other DNA
gcgaccctgcatcacatggttt

SEQ ID NO: 57 - DNA, 22 bp, Homo sapiens
  source 1..22: mol_type = other DNA
atacactgcgaccctgcatcac

SEQ ID NO: 58 - DNA, 22 bp, Homo sapiens
  source 1..22: mol_type = other DNA
tcaaacatacactgcgaccctg

SEQ ID NO: 59 - DNA, 22 bp, Homo sapiens
  source 1..22: mol_type = other DNA
gtcccatcaaacatacactgcg

SEQ ID NO: 60 - DNA, 22 bp, Homo sapiens
  source 1..22: mol_type = other DNA
atggcgtcccatcaaacataca

SEQ ID NO: 61 - DNA, 22 bp, Homo sapiens
  source 1..22: mol_type = other DNA
tgaaagatggcgtcccatcaaa

SEQ ID NO: 62 - DNA, 22 bp, Homo sapiens
  source 1..22: mol_type = other DNA
cagttctgaaagatggcgtccc

SEQ ID NO: 63 - DNA, 22 bp, Homo sapiens
  source 1..22: mol_type = other DNA
ttagcacagttctgaaagatgg

SEQ ID NO: 64 - DNA, 22 bp, Homo sapiens
  source 1..22: mol_type = other DNA
agtgagttagcacagttctgaa

SEQ ID NO: 65 - DNA, 22 bp, Homo sapiens
  source 1..22: mol_type = other DNA
cttcaacagtgagttagcacag

SEQ ID NO: 66 - DNA, 22 bp, Homo sapiens
  source 1..22: mol_type = other DNA
tggacgcttcaacagtgagtta

SEQ ID NO: 67 - DNA, 22 bp, Homo sapiens
  source 1..22: mol_type = other DNA
taccattggacgcttcaacagt

SEQ ID NO: 68 - DNA, 22 bp, Homo sapiens
  source 1..22: mol_type = other DNA
ttctcttaccattggacgcttc

SEQ ID NO: 69 - DNA, 22 bp, Homo sapiens
  source 1..22: mol_type = other DNA
ctgtattttctcttaccattgg

SEQ ID NO: 70 - DNA, 22 bp, Homo sapiens
  source 1..22: mol_type = other DNA
cggatcgaaaagagcagacact

SEQ ID NO: 71 - DNA, 22 bp, Homo sapiens
  source 1..22: mol_type = other DNA
cggccgtcccggatcgaaaaga

SEQ ID NO: 72 - DNA, 22 bp, Homo sapiens
  source 1..22: mol_type = other DNA
cgagcctgaccggccgtcccgg

SEQ ID NO: 73 - DNA, 22 bp, Homo sapiens
  source 1..22: mol_type = other DNA
ctcggcggcgagcctgaccggc

SEQ ID NO: 74 - DNA, 22 bp, Homo sapiens
  source 1..22: mol_type = other DNA
tagttctccagctcggcggcga

SEQ ID NO: 75 - DNA, 22 bp, Homo sapiens
  source 1..22: mol_type = other DNA
cgggtcatcgtagttctccagc

SEQ ID NO: 76 - DNA, 22 bp, Homo sapiens
  source 1..22: mol_type = other DNA
gggttttgtgcgggtcatcgta

SEQ ID NO: 77 - DNA, 22 bp, Homo sapiens
  source 1..22: mol_type = other DNA
ctggggaggcaggggttttgtg

SEQ ID NO: 78 - DNA, 22 bp, Homo sapiens
  source 1..22: mol_type = other DNA
tgtggacaactggggaggcagg

SEQ ID NO: 79 - DNA, 22 bp, Homo sapiens
  source 1..22: mol_type = other DNA
gcccctgatgtggacaactggg

SEQ ID NO: 80 - DNA, 22 bp, Homo sapiens
  source 1..22: mol_type = other DNA
cgtcaatcaggcccctgatgtg

SEQ ID NO: 81 - DNA, 22 bp, Homo sapiens
  source 1..22: mol_type = other DNA
tccaccacaccgtcaatcaggc

SEQ ID NO: 82 - DNA, 22 bp, Homo sapiens
  source 1..22: mol_type = other DNA
ggtctgcttccaccacaccgtc

SEQ ID NO: 83 - DNA, 22 bp, Homo sapiens
  source 1..22: mol_type = other DNA
aaggcctccacaaggtctgctt

SEQ ID NO: 84 - DNA, 22 bp, Homo sapiens
  source 1..22: mol_type = other DNA
aaactcctgcaaggcctccaca

SEQ ID NO: 85 - DNA, 22 bp, Homo sapiens
  source 1..22: mol_type = other DNA
tgatgggtccaaactcctgcaa

SEQ ID NO: 86 - DNA, 22 bp, Homo sapiens
  source 1..22: mol_type = other DNA
accacatagctgatgggtccaa

SEQ ID NO: 87 - DNA, 22 bp, Homo sapiens
  source 1..22: mol_type = other DNA
aggcattaccaccacatagctg

SEQ ID NO: 88 - DNA, 20 bp, Homo sapiens
  source 1..20: mol_type = other DNA
cccttgtccactgagattgg

SEQ ID NO: 89 - DNA, 20 bp, Homo sapiens
  source 1..20: mol_type = other DNA
cgacccttgtccactgagat

SEQ ID NO: 90 - DNA, 20 bp, Homo sapiens
  source 1..20: mol_type = other DNA
tgcgacccttgtccactgag

SEQ ID NO: 91 - DNA, 20 bp, Homo sapiens
  source 1..20: mol_type = other DNA
cactgcgacccttgtccact

SEQ ID NO: 92 - DNA, 20 bp, Homo sapiens
  source 1..20: mol_type = other DNA
atacactgcgacccttgtcc

SEQ ID NO: 93 - DNA, 20 bp, Homo sapiens
  source 1..20: mol_type = other DNA
aacatacactgcgacccttg

SEQ ID NO: 94 - DNA, 20 bp, Homo sapiens
  source 1..20: mol_type = other DNA
tcaaacatacactgcgaccc

SEQ ID NO: 95 - DNA, 20 bp, Homo sapiens
  source 1..20: mol_type = other DNA
accattggacgcttcaacag

SEQ ID NO: 96 - DNA, 20 bp, Homo sapiens
  source 1..20: mol_type = other DNA
gtcaccattggacgcttcaa

SEQ ID NO: 97 - DNA, 20 bp, Homo sapiens
  source 1..20: mol_type = other DNA
gggtcaccattggacgcttc

SEQ ID NO: 98 - DNA, 20 bp, Homo sapiens
  source 1..20: mol_type = other DNA
ccagggtcaccattggacgc

SEQ ID NO: 99 - DNA, 20 bp, Homo sapiens
  source 1..20: mol_type = other DNA
ctgccagggtcaccattgga

SEQ ID NO: 100 - DNA, 20 bp, Homo sapiens
  source 1..20: mol_type = other DNA
tgctgccagggtcaccattg

SEQ ID NO: 101 - DNA, 20 bp, Homo sapiens
  source 1..20: mol_type = other DNA
gttgctgccagggtcaccat

SEQ ID NO: 102 - RNA, 70 nt, Homo sapiens
  source 1..70: mol_type = mRNA
ggtcgcagtgtatgtttgatgggacgccatctttcagaactgtgctaactcactgttgaagcgtccaatg

SEQ ID NO: 103 - protein, 1703 aa, Homo sapiens
MSLLCVGVKKAKFDGAQEKFNTYVTLKVQNVKSTTIAVRGSQPSWEQDFMFEINRLDLGLTVEVWNKGLIWDTMVGTVWIPLRTIRQSNEEGPGEWLTLDSQVIMADSEICGTKDPTFHRILLDTRFELPLDIPEEEARYWAKKLEQLNAMRDQDEYSFQDEQDKPLPVPSNQCCNWNYFGWGEQHNDDPDSAVDDRDSDYRSETSNSIPPPYYTTSQPNASVHQYSVRPPPLGSRESYSDSMHSYEEFSEPQALSPTGSSRYASSGELSQGSSQLSEDFDPDEHSLQGSDMEDERDRDSYHSCHSSVSYHKDSPRWDQDEEELEEDLEDFLEEEELPEDEEELEEEEEEVPDDLGSYAQREDVAVAEPKDFKRISLPPAAPGKEDKAPVAPTEAPDMAKVAPKPATPDKVPAAEQIPEAEPPKDEESFRPREDEEGQEGQDSMSRAKANWLRAFNKVRMQLQEARGEGEMSKSLWFKGGPGGGLIIIDSMPDIRKRKPIPLVSDLAMSLVQSRKAGITSALASSTLNNEELKNHVYKKTLQALIYPISCTTPHNFEVWTATTPTYCYECEGLLWGIARQGMRCTECGVKCHEKCQDLLNADCLQRAAEKSSKHGAEDRTQNIIMVLKDRMKIRERNKPEIFELIQEIFAVTKTAHTQQMKAVKQSVLDGTSKWSAKISITVVCAQGLQAKDKTGSSDPYVTVQVGKTKKRTKTIYGNLNPVWEENFHFECHNSSDRIKVRVWDEDDDIKSRVKQRFKRESDDFLGQTIIEVRTLSGEMDVWYNLDKRTDKSAVSGAIRLHISVEIKGEEKVAPYHVQYTCLHENLFHFVTDVQNNGVVKIPDAKGDDAWKVYYDETAQEIVDEFAMRYGVESIYQAMTHFACLSSKYMCPGVPAVMSTLLANINAYYAHTTASTNVSASDRFAASNFGKERFVKLLDQLHNSLRIDLSMYRNNFPASSPERLQDLKSTVDLLTSITFFRMKVQELQSPPRASQVVKDCVKACLNSTYEYIFNNCHELYSREYQTDPAKKGEVLPEEQGPSIKNLDFWSKLITLIVSIIEEDKNSYTPCLNQFPQELNVGKISAEVMWNLFAQDMKYAMEEHDKHRLCKSADYMNLHFKVKWLYNEYVTELPAFKDRVPEYPAWFEPFVIQWLDENEEVSRDFLHGALERDKKDGFQQTSEHALFSCSVVDVFSQLNQSFEIIKKLECPDPQIVGHYMRRFAKTISNVLLQYADIISKDFASYCSKEKEKVPCILMNNTQQLRVQLEKMFEAMGGKELDAEASDILKELQVKLNNVLDELSRVFATSFQPHIEECVKQMGDILSQVKGTGNVPASACSSVAQDADNVLQPIMDLLDSNLTLFAKICEKTVLKRVLKELWKLVMNTMEKTIVLPPLTDQTMIGNLLRKHGKGLEKGRVKLPSHSDGTQMIFNAAKELGQLSKLKDHMVREEAKSLTPKQCAVVELALDTIKQYFHAGGVGLKKTFLEKSPDLQSLRYALSLYTQATDLLIKTFVQTQSAQGLGVEDPVGEVSVHVELFTHPGTGEHKVTVKVVAANDLKWQTSGIFRPFIEVNIIGPQLSDKKRKFATKSKNNSWAPKYNESFQFTLSADAGPECYELQVCVKDYCFAREDRTVGLAVLQLRELAQRGSAACWLPLGRRIHMDDTGLTVLRILSQRSNDEVAKEFVKLKSDTRSAEEGGAAPAP

SEQ ID NO: 104 - protein, 1591 aa, Homo sapiens
MSLLCVRVKRAKFQGSPDKFNTYVTLKVQNVKSTTVAVRGDQPSWEQDFMFEISRLDLGLSVEVWNKGLIWDTMVGTVWIALKTIRQSDEEGPGEWSTLEAETLMKDDEICGTRNPTPHKILLDTRFELPFDIPEEEARYWTYKWEQINALGADNEYSSQEESQRKPLPTAAAQCSFEDPDSAVDDRDSDYRSETSNSFPPPYHTASQPNASVHQFPVPVRSPQQLLLQGSSRDSCNDSMQSYDLDYPERRAISPTSSSRYGSSCNVSQGSSQLSELDQYHEQDDDHRETDSIHSCHSSHSLSRDGQAGFGEQEKPLEVTGQAEKEAACEPKEMKEDATTHPPPDLVLQKDHFLGPQESFPEENASSPFTQARAHWIRAVTKVRLQLQEIPDDGDPSLPQWLPEGPAGGLYGIDSMPDLRRKKPLPLVSDLSLVQSRKAGITSAMATRTSLKDEELKSHVYKKTLQALIYPISCTTPHNFEVWTATTPTYCYECEGLLWGIARQGMRCSECGVKCHEKCQDLLNADCLQRAAEKSCKHGAEDRTQNIIMAMKDRMKIRERNKPEIFEVIRDVFTVNKAAHVQQMKTVKQSVLDGTSKWSAKITITVVCAQGLQAKDKTGSSDPYVTVQVSKTKKRTKTIFGNLNPVWEEKFHFECHNSSDRIKVRVWDEDDDIKSRVKQRLKRESDDFLGQTIIEVRTLSGEMDVWYNLEKRTDKSAVSGAIRLQISVEIKGEEKVAPYHVQYTCLHENLFHYLTDIQGSGGVRIPEARGDDAWKVYFDETAQEIVDEFAMRYGIESIYQAMTHFACLSSKYMCPGVPAVMSTLLANINAYYAHTTASTNVSASDRFAASNFGKERFVKLLDQLHNSLRIDLSTYRNNFPAGSPERLQDLKSTVDLLTSITFFRMKVQELQSPPRASQVVKDCVKACLNSTYEYIFNNCHDLYSRQYQLKQELPPEEQGPSIRNLDFWPKLITLIVSIIEEDKNSYTPVLNQFPQELNVGKVSAEVMWHLFAQDMKYALEEHEKDHLCKSADYMNLHFKVKWLHNEYVRDLPVLQGQVPEYPAWFEQFVLQWLDENEDVSLEFLRGALERDKKDGFQQTSEHALFSCSVVDVFTQLNQSFEIIRKLECPDPSILAHYMRRFAKTIGKVLMQYADILSKDFPAYCTKEKLPCILMNNVQQLRVQLEKMFEAMGGKELDLEAADSLKELQVKLNTVLDELSMVFGNSFQVRIDECVRQMADILGQVRGTGNASPDARASAAQDADSVLRPLMDFLDGNLTLFATVCEKTVLKRVLKELWRVVMNTMERMIVLPPLTDQTGTQLIFTAAKELSHLSKLKDHMVREETRNLTPKQCAVLDLALDTIKQYFHAGGNGLKKTFLEKSPDLQSLRYALSLYTQTTDTLIKTFVRSQTTQGSGVDDPVGEVSIQVDLFTHPGTGEHKVTVKVVAANDLKWQTAGMFRPFVEVTMVGPHQSDKKRKFTTKSKSNNWAPKYNETFHFLLGNEEGPESYELQICVKDYCFAREDRVLGLAVMPLRDVTAKGSCACWCPLGRKIHMDETGLTILRILSQRSNDEVAREFVKLKSESRSTEEGS